(19) United States
(12) Patent Application Publication
(10) Pub. No.: US 2012/0009575 A1
(43) Pub. Date: Jan. 12, 2012

(54) INDUCIBLE NUCLEIC ACID TARGETS FOR DETECTION OF PATHOGENS, METHODS AND COMPOSITIONS THEREOF

(75) Inventors: Priya Balachandran, Foster City, CA (US); Craig Cummings, Pacifica, CA (US); Olga Petrauskene, San Carlos, CA (US); Hemant Pawar, Ann Arbor, MI (US); Robert Tebbs, Livermore, CA (US); Manohar Furtado, San Ramon, CA (US)

(73) Assignee: LIFE TECHNOLOGIES CORPORATION, Carlsbad, CA (US)

(21) Appl. No.: 13/174,645

(22) Filed: Jun. 30, 2011

Related U.S. Application Data
(60) Provisional application No. 61/360,337, filed on Jun. 30, 2010.

(51) Int. Cl. C12Q 1/68

(52) U.S. Cl. 435/6.11; 435/6.15; 435/6.12

(57) ABSTRACT

The present application describes compositions, methods and kits for rapid detection and identification of various microorganisms using inducible RNA. Methods for rapidly detecting microorganisms by detecting expression of inducible RNA of target genes following induction of a target gene using an inducer are described. Some embodiments describe methods and workflows for rapidly detecting microbes such as, but not limited to, Salmonella spp, Listeria spp. and Vibrio spp. Compositions and kits comprise primer nucleic acid sequences having hybridization specificity for priming amplification of genes of microorganisms (or gene fragments) that are responsive to one or more RNA-inducing agents. In some embodiments, kits and compositions further comprise probe nucleic acid sequences having hybridization specificity for genes responsive to RNA-inducing agents or fragments thereof.

### CROSS REFERENCE RELATED APPLICATIONS

This patent application claims priority under 35 U.S.C. §119(e) to United States Provisional Patent Application, U.S. Ser. No. 61/360,337, filed Jun. 30, 2010, the entire contents of which are incorporated herein by reference.

### EFS INCORPORATION PARAGRAPH RELATING TO SEQUENCE LISTING

The instant application contains a Sequence Listing which has been submitted in ASCII format via EFS-Web and is hereby incorporated by reference in its entirety. Said ASCII copy, created on Jun. 22, 2011, is named LT0267US.txt and is 404,503 bytes in size.

### FIELD OF DISCLOSURE

The present teachings relate to compositions, methods and kits for rapid detection and identification of various microorganisms using inducible RNA. More particularly, the specification describes compositions and kits comprising primer nucleic acid sequences having hybridization specificity for priming amplification of genes of microorganisms that are responsive to one or more RNA-inducing agents and in some embodiments further comprising probe nucleic acid sequences having hybridization specificity for genes responsive to RNA-inducing agents. Methods for rapidly detecting microorganisms (such as, but not limited to, Salmonella spp, Listeria spp. and Vibrio spp) are also described.

### BACKGROUND

Detection of bacteria, particularly pathogenic bacteria, is an important parameter used to monitor for quality control and consumer safety in the food and pharmaceutical industries as well as in environmental monitoring. Earlier detection is of great benefit for quality and safety testing in industries such as the food industry where a faster time-to-result can greatly reduce total testing time as well as storage time for food prior to release of safe product to the grocery stores.

Advances in technology make detection and identification faster, more convenient, more sensitive, and more specific than traditional culture assays, at least in theory. Such assays include biochemical kits, and antibody-based and DNA-based tests. Use of probes, PCR and bacteriophage has been developed commercially for detecting pathogens. Probe assays generally target ribosomal RNA (rRNA), since rRNA provides a naturally amplified target and greater assay sensitivity. With few exceptions, almost all assays used to detect specific pathogens require some growth of the test sample in an enrichment medium before analysis. Early detection of the presence of pathogens is extremely important both from a public health perspective and from an economic perspective. Therefore, improved methods that result in saving any of time-to-result, labor, and materials are desired.

### SUMMARY OF DISCLOSURE

Embodiments herein demonstrate that detection of inducible RNA targets in addition to or in lieu of detection of corresponding DNA targets can shorten time-to-result when testing samples for presence of microorganisms such as bacteria and fungi.

Some embodiments herein identify inducible RNA target genes and induction conditions for detection of pathogenic microbes such as Listeria, a gram positive bacteria, and Salmonella and Vibrio, both gram negative bacteria.

Some embodiments provide compositions comprising one or more primer pairs operable to amplify one or more inducible RNA target genes. Some embodiments further provide compositions comprising one or more probes that are operable to hybridize to and identify an inducible RNA target gene.

Workflow methods using inducible RNA targets in assays for various bacteria are also described. Fast workflow methods are provided based on inducible transcription response in bacteria. An example workflow of the disclosure includes a shortened enrichment step, a rapid induction step, an automated sample preparation step, and specific detection of induced target genes using a reverse transcriptase-polymerase chain reaction (RT-PCR). Sample prep and RT-PCR in some embodiments are completed in less than 2 hours. Some embodiments comprise using real-time PCR and/or real-time RT-PCR.

A method of detecting presence of a microbe in a sample comprises exposing the sample to an RNA-inducing agent for a time to regulate a gene responsive thereto, detecting presence of RNA corresponding to the gene, and determining presence of the microbe as compared to a control sample based on the detection of RNA presence. In a further embodiment, the sample is cultured in a microbe enrichment medium to form enriched sample, and the enriched sample is exposed to the RNA-inducing agent.

Further embodiments herein include a method of detecting presence of a microbe in a sample, the method comprising culturing the sample in a microbe enrichment medium to form enriched sample, exposing the enriched sample to an inducing agent for a time to regulate a gene responsive thereto, measuring RNA expression levels of the responsive gene, and analyzing said expression levels of RNA to indicate the presence of the microbe as compared to at least one control sample, wherein a time for detecting presence of the microbe is shorter than a control method in which enriched samples are not exposed to the inducing agent.

Such methods of detecting presence of a pathogen are useful for food samples, for pharmaceutical quality control, for environmental samples, as well as in clinical samples and specimens.

In some embodiments, a microbe that is detected may be a gram positive or gram negative bacteria. In some embodiments, a microbe may be a pathogen.

In some embodiments, induced genes are stress responsive genes. In some embodiments, the pathogen is an organism of Salmonella spp. and the RNA-inducing agent-responsive gene is cspH, hilA, hsp60, dnaK, ibpAB, uspA, or agsA. In other embodiments, the pathogen is a Listeria spp. and the RNA-inducing agent-responsive gene is a inlA, a inlB, a inlC, a inlG, a inlJ, a lmo0539, a lmo2158, a lmo0596, a lmo0189, a lmo0880, a lmo1290, a lmo0514, a lmo0670, a bsh, a plcA, a clpE, a cspL, a lmo0699, a lmo0782, a lmo2230, a lmo2522, a opuCA, a cpn60, or a hlyA gene. In further embodiments, the pathogen is a Vibrio spp. and the RNA-inducing agent-responsive gene is a hsp60 gene. An RNA-inducing agent-responsive gene may be either up-regulated or down-regulated in response to exposure to the agent.

Measuring transcription may comprise measuring RNA expression levels using reverse transcriptase RT-PCR. In some embodiments, both RNA and DNA levels are measured using reverse transcriptase RT-PCR. In some embodiments, DNA expression levels are measured by RT-PCR. In further embodiments, the contribution of RNA to a signal can be determined by subtracting a CT value obtained from detecting both RNA and DNA from a CT value obtained from detecting just the DNA for a particular target. In some embodiments of the present methods, inclusion of the reverse transcriptase step improved the real time PCR signal by 4-7Ct's, enabling a shorter time-to-result in a workflow for detecting a microbe in a sample.

Several kits embodiments are also described. An example kit includes at least one primer pair having hybridization specificity for priming amplification of a RNA-inducing agent-responsive gene (or a fragment thereof) of a microbe. A kit may further comprise at least one probe specific to hybridize to and detect a RNA-inducing agent responsive gene or a fragment thereof. A kit of the disclosure may further comprise reagents for PCR, such as a PCR master mix which may include: any one of a reverse transcriptase, a DNA polymerase, dNTP's; ingredients for sample preparation including one or more of: a filtration medium, a surfactant, magnetic beads, spin columns; and various buffers.

One example kit may comprise at least one primer pair having hybridization specificity for priming amplification of a RNA-inducing agent-responsive gene (or a fragment thereof) of a Salmonella where the RNA-inducing agent-responsive gene is a cspH, a hilA, a hsp60, a dnaK, a ibpAB, a uspA, and/or a agsA gene.

Another example kit may comprise at least one primer pair having hybridization specificity for priming amplification of a RNA-inducing agent-responsive gene (or a fragment thereof) of a Listeria wherein the RNA-inducing agent-responsive gene is a inlA, a inlB, a inlC, a inlG, a inlJ, a lmo0539, a lmo2158, a lmo0596, a lmo0189, a lmo0880, a lmo1290, a lmo0514, a lmo0670, a bsh, a plcA, a clpE, a cspL, a lmo0699, a lmo0782, a lmo2230, a lmo2522, a opuCA, a cpn60, or a hlyA gene.

Yet another example kit includes at least one primer pair having hybridization specificity for priming amplification of a RNA-inducing agent-responsive gene (or a fragment thereof) of a Vibrio wherein the RNA-inducing agent-responsive gene is a hsp60 gene.

Exemplary kits described above may further comprise a probe (e.g., a TAQMAN® probe) having hybridization specificity for said RNA-inducing agent-responsive gene.

Use of inducible RNA targets provided by embodiments herein for detection of pathogens has advantages over traditional DNA targets typically used in real-time PCR based detection. For example, for RNA inducible targets, an increased copy number of the target is present upon induction. When DNA is transcribed into RNA, many copies of the target gene are generated and a greater copy number translates into more robust and potentially earlier detection of a pathogen.

Traditional culture methods as well as rapid PCR-based methods rely on an initial pre-enrichment in suitable media to revive stressed organisms in various matrices. The length of the pre-enrichment time depends on the sensitivity and limit of detection of the end assay method. Signal amplification using RNA targets, such as inducible RNA targets, allows for earlier detection of target. Workflow methods provided herein allowed for detection of 1-5 cfu of Salmonella in less then 8 hr and Listeria in less than 12 hr in food samples.

A further advantage of using inducible RNA targets for detection of pathogens is in an assessment of viability. Dead bacteria will not respond to induction. Inducible RNA targets are synthesized by living cells upon sensing a particular stimulus, such as heat, cold, acidic pH or chemical reagents. Use of inducible RNA targets therefore overcomes one disadvantage of using DNA targets, i.e., detection of dead bacteria that are incapable of causing disease. An additional advantage over traditional culture confirmation method is the ability to detect viable but non-culturable bacteria (VBNC) organisms that respond to induction.

These and other features of the present teachings will become more apparent from the description herein.

### BRIEF OF DESCRIPTION OF DRAWINGS

The skilled artisan will understand that the drawings, described below, are for illustration purposes only. The drawings are not intended to limit the scope of the present teachings in any way.

FIG. 1A-FIG. 1B shows data of studies evaluating heat-inducible RNA target genes for detecting Salmonella enterica. FIG. 1A depicts Ct data vs various primer/probe sets specific to some heat-inducible RNA genes for both uninduced (negative control) and heat-induced cultures. Nucleic acids were extracted and assayed for induction by real-time reverse-transcriptase-PCR (hatched bars). To measure contribution due to DNA, control reactions were run without addition of the reverse transcriptase (solid bars). FIG. 1B depicts Delta Ct data vs. various primer/probe sets specific to some heat-inducible RNA genes for both uninduced (negative control) and induced cultures. Transcriptional activity was estimated by subtracting the CT value obtained for DNA and RNA (with reverse transcriptase reactions) from the CT value obtained for DNA alone (without reverse transcriptase reactions) for uninduced (solid bars) and induced (striped bars) samples.

FIG. 2A-FIG. 2B show data on heat-induction of Salmonella response genes in the presence of a food matrix (whole milk). FIG. 2A shows the Ct data vs various primer/probe sets specific to some heat-inducible RNA genes (see X-axis) for both uninduced (negative control) and heat induced cultures. The extracted nucleic acids were assayed for induction by real-time reverse-transcriptase-PCR (hatched bars). To measure contribution due to DNA, control reactions were run without addition of the reverse transcriptase (solid bars). FIG. 2B shows Delta Ct data vs. various primer/probe sets for specific to some heat-inducible RNA genes for both uninduced (negative control) and induced cultures. Transcriptional activity was estimated by subtracting the CT value obtained for DNA and RNA (with reverse transciptase reactions) from the CT value obtained for DNA alone (without reverse transcriptase reactions) for uninduced (solid bars) and induced (striped bars) samples.

FIG. 3A-FIG. 3J provide data for evaluating heat-induction of response genes in Salmonella during growth in a food matrix (whole milk) with respect to enrichment time. Duplicate aliquots of enriched samples were withdrawn after 4 hours, 6 hours, 8 hours and 24 hours of growth. One set was maintained at 37° C. (uninduced) and one set was incubated at 45° C. (induced) for 15 minutes. FIGS. 3A, 3C, 3E, 3G, and 3I show Ct data vs time for uninduced and induced cultures. The extracted nucleic acids were assayed for induction by real-time reverse-transcriptase-PCR (hatched bars). To measure contribution due to DNA, control reactions were run without addition of the reverse transcriptase (solid bars). FIGS. 3B, 3D, 3F, 3H, 3J show Delta Ct data vs. time for uninduced and induced cultures. Transcriptional activity was estimated by subtracting the CT value obtained for DNA and RNA (with reverse transciptase reactions) from the CT value obtained for DNA alone (without reverse transcriptase reactions) for uninduced (solid bars) and induced samples (striped bars).

FIG. 4A and FIG. 4B provide data for evaluating induction of response genes during growth of Salmonella in an additional food matrix (spinach) with respect to enrichment time. Duplicate enriched sample aliquots were withdrawn after 4 hours, 6 hours, 8 hours and 24 hours of growth. One set was maintained at 37° C. (uninduced) and one set was incubated at 45° C. (induced) for 15 minutes. FIG. 4A shows Ct data vs time for uninduced and induced cultures (probe/primer set agsA.6). The extracted nucleic acids were assayed for induction by real-time reverse-transcriptase-PCR (hatched bars). To measure contribution due to DNA, control reactions were run without addition of the reverse transcriptase (solid bars). FIG. 4B shows Delta Ct data vs. time for uninduced and induced cultures. Transcriptional activity was estimated by subtracting the CT value obtained for DNA and RNA (with reverse transciptase reactions) from the CT value obtained for DNA alone (without reverse transcriptase reactions) for uninduced (solid bars) and induced samples (striped and dotted bars).

FIG. 5A-FIG. 5F show data for evaluation of targets under various inducible conditions for various target genes in Listeria. FIG. 5A and FIG. 5B show expression of various inducible target genes (see y-axis for example genes tested) by heat-induction. Ct (FIG. 5A) or Delta Ct (FIG. 5B) values are provided for various target genes for uninduced (37° C.) and induced (48° C.) samples before processing for nucleic acids. FIG. 5C and FIG. 5D show expression of various inducible target genes (see y-axis for example genes tested) in response to activated charcoal induction. Samples were grown in enrichment media treated with 0.2% activated charcoal for 5 hours, along with parallel uninduced samples. FIG. 5E and FIG. 5F show expression of various inducible target genes (see y-axis for example genes tested) in response to high-salt induction.

FIG. 6A-FIG. 6B show data obtained by evaluation of several target inducible genes at different times using activated charcoal as an inducer for Listeria in a food matrix (raw milk). Enriched samples were incubated at 37° C. and withdrawn at 16 and 24 hours post-enrichment. FIG. 6A shows that extracted nucleic acids were assayed for induction by real-time reverse-transcriptase-PCR (hatched bars). To measure contribution due to DNA, control reactions were run without addition of the reverse transcriptase (solid bars). FIG. 6B. shows transcriptional activity was estimated by subtracting the Ct value obtained for DNA and RNA (with reverse transciptase reactions) from the Ct value obtained for DNA alone (without reverse transcriptase reactions) for uninduced (solid bars) and induced (striped bars) samples.

FIG. 7 provides data on use of heat-induction for detection of Vibrio cholerae by inducing a hsp60 target inducible gene. The x-axis numbers 1, 2, and 3 represent replicate samples.

FIG. 8A and FIG. 8B provide data on the use of heat-induction to detect Salmonella by measuring the target hilA.

FIG. 9A and FIG. 9B provide data on the use of heat-induction to detect Listeria monocytogenes by measuring the target hlyA.

FIG. 10A depicts an example workflow according to one embodiment of the disclosure.

FIGS. 10B and 10C demonstrate two example workflows for Salmonella that took less than 8 hours and where detection was 100% and enrichment time was 6 hours.

FIG. 11A and FIG. 11B demonstrate that methods of the disclosure comprising detection of inducible RNA targets detect only live cells and not dead cells. FIG. 11A shows live and viable Salmonella cells responding to the heat-induction by increase in target mRNA production, while in FIG. 11B heat-killed Salmonella cells do not increase target mRNA production.

### DETAILED DESCRIPTION OF EMBODIMENTS

It is to be understood that both the foregoing general description and the following detailed description are exemplary and explanatory only and are not intended to limit the scope of the current teachings. In this application, the use of the singular includes the plural unless specifically stated otherwise. Also, the use of “comprise”, “contain”, and “include”, or modifications of those root words, for example but not limited to, “comprises”, “contained”, and “including”, are not intended to be limiting. Use of “or” means “and/or” unless stated otherwise. The term “and/or” means that the terms before and after can be taken together or separately. For illustration purposes, but not as a limitation, “X and/or Y” can mean “X” or “Y” or “X and Y”.

Whenever a range of values is provided herein, the range is meant to include the starting value and the ending value and any value or value range there between unless otherwise specifically stated. For example, “from 0.2 to 0.5” means 0.2, 0.3, 0.4, 0.5; ranges there between such as 0.2-0.3, 0.3-0.4, 0.2-0.4; increments there between such as 0.25, 0.35, 0.225, 0.335, 0.49; increment ranges there between such as 0.26-0.39; and the like.

The section headings used herein are for organizational purposes only and are not to be construed as limiting the subject matter described in any way. All literature and similar materials cited in this application including, but not limited to, patents, patent applications, articles, books, treatises, and internet web pages, regardless of the format of such literature and similar materials, are expressly incorporated by reference in their entirety for any purpose. In the event that one or more of the incorporated literature and similar materials defines or uses a term in such a way that it contradicts that term's definition in this application, this application controls. While the present teachings are described in conjunction with various embodiments, it is not intended that the present teachings be limited to such embodiments. On the contrary, the present teachings encompass various alternatives, modifications, and equivalents, as will be appreciated by those of skill in the art.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, ACB, CBA, BCA, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context. The term “surrogate” as used herein means a product that is indicative of presence of another product. For example, an amplification product is a surrogate for a nucleic acid that has been amplified.

Various embodiments herein provide compositions, methods and kits for detection of microorganisms comprising detecting inducible RNA target genes in microbes that may be induced (transcribed) in response to an inducer (e.g., heat, pH and/or a chemical or biological agent) and thereby be detectable much faster than detecting traditional DNA targets comprised of unique signature sequences. Some embodiments relate to detection of pathogenic microbes. Some exemplary candidate bacterial pathogens that may be detected by the methods and compositions of this disclosure include gram positive and gram negative bacteria including, but not limited to, species of E. coli, Salmonella, Shigella, Campylobacter, Yersinia, Vibrio, Listeria, Staphylococcus, Bacillus, Clostridium, Pseudomonas or Cronobacter.

The term “target gene” or “inducible target gene” or “inducible RNA target gene” refers to a gene of a microorganism that has been identified as a gene that can be induced (e.g., gene expressed, RNA expressed, transcription is induced) in the microorganism in response to an inducer (an inducing agent or inducing condition). Some exemplary inducible target genes identified in the present specification include: Salmonella target genes that are responsive to one or more RNA-inducing agents such as a cspH, a hilA, a hsp60, a dnaK, a ibpAB, a uspA, and/or a agsA gene; Listeria target genes that are responsive to one or more RNA-inducing agents such as a inlA, a inlB, a inlC, a inlG, a inlJ, a lmo0539, a lmo2158, a lmo0596, a lmo0189, a lmo0880, a lmo1290, a lmo0514, a lmo0670, a bsh, a plcA, a clpE, a cspL, a lmo0699, a lmo0782, a lmo2230, a lmo2522, a opuCA, a cpn60, or a hlyA gene; and Vibrio target genes that are responsive to one or more RNA-inducing agents such as hsp60. Accordingly, one or more compositions, methods and kits of the disclosure are described in relation to these inducible genes and to the detection of these organisms. However, as will be recognized by one of skill in the art, the teachings of specification are not limited to these exemplary embodiments, and any microorganism that has an inducible gene that is inducible by an inducer may be detected by the methods described in this application. In some embodiments, induced genes are stress responsive genes of a microbe, that are induced quickly to allow the microbe to adapt and/or survive the stress (extreme heat/cold; lack of nutrients etc.).

“Induction,” or “induce a gene responsive to an RNA-inducing agent” as used herein refers to exposing and/or contacting and/or adding and/or “subjecting to” an inducing agent and/or condition (collectively referred to as an “inducer”) to a sample, containing or suspected of containing a microbe, and/or to an enriched culture, containing or suspected of containing the microbe, in response to which an inducible target gene is stimulated and/or induced to express RNA (e.g. transcription is induced) corresponding to that target gene. Inducible target genes are normally not induced without presence of the inducing agent or condition. Exemplary “inducers” may include, but are not limited to, thermal conditions including heat or cold (i.e., temperatures that an microorganism normally is not exposed to during its normal growth), activated charcoal, protein or peptide inducers, inducing chemicals (such as but not limited to homoserine lactones), metal chelators, high salt concentrations, different salts, pH values (acidic or basic conditions), nutrient concentrations, or to a physical effect such as pressure, presence of chemotactants, or the presence of a suboptimal environment, or a stress agent.

When temperature is used as an inducer, induction by heat depends on the organism whose presence is being tested for; for example, Vibrio cholerae grows normally at 30° C., therefore, heat-induction may occur at any temperature greater than 30° C., such as 37° C., or 42° C. In another example. Salmonella normally grows at 37° C., therefore, heat induction may occur at any temperature greater than 37° C., such as 42° C., 45° C., 48° C., 50° C., or 55° C. To expose a microbe to a heat inducer cultures can be transferred to incubators at higher temperatures, or cultures can be grown at the higher temperatures when the induction is desired. In some embodiments, a culture may be exposed to a cold temperature to induce genes that may be induced by colder temperatures than an organism usually grow in.

Some embodiments describe designing probes and primers of the disclosure following the identification of target inducible genes. Primer and probe sequences of the disclosure were designed using a rigorous bioinformatics assay design pipeline and are described in the nucleic acid comprised in SEQ ID NO: 1-SEQ ID NO: 1769.

Some embodiments of the present disclosure describes various nucleic acid compositions such as primers, generally comprising a primer pair, each primer pair comprising a forward primer and a reverse primer that can be used to hybridize to an inducible target gene (for example, RNA expressed by an inducible gene may be reverse transcribed and the cDNA formed therefrom can be subject to hybridization by a primer pair of the disclosure and then subject to conditions to carry out a DNA amplification reaction to obtain an amplified inducible target gene product which may then be detected). In some embodiments, the present disclosure also describes compositions of various probe sequences that are operable to bind to an inducible target gene or a fragment thereof to enable its detection.

The present disclosure, in some embodiments, relates to isolated nucleic acid molecules having: the sequences of SEQ ID NO: 1-SEQ ID NO:1769; complementary sequences thereof; and/or nucleic acid sequences having at least 90% homology with the sequences of SEQ ID NO: 1-SEQ ID NO:1769; and/or or a labeled derivative of any of these sequences.

Some compositions of the disclosure may comprise primer pairs operable to amplify one target inducible gene and may further comprise in some embodiments a corresponding probe sequence that is operable to bind to and detect the amplified product produced by the primer pairs. Many such compositions are described. In one example, Tables 1-3 describe several primer pairs, comprising at least a first primer referred to as a forward primer and a second primer referred to as a reverse primer. In each row of Tables 1-3, each primer pair also has at least one corresponding probe sequence. Compositions comprising a primer pairs (and in some embodiments, a corresponding probe) may be used in assays for specific and efficient detection of genes identified in the Tables as well. Thus, for example, an exemplary composition of the disclosure operable for detecting Salmonella by detecting an inducible gene agsA may comprise: a forward primer having SEQ ID NO: 1, a reverse primer having SEQ ID NO: 134 and optionally a probe having SEQ ID NO: 267; complementary sequences thereof; and/or nucleic acid sequences having at least 90% homology with the sequences; and/or or a labeled derivative of any of these sequences. These and other compositions are described in Tables 1-3.

Some exemplary non-limiting compositions having primer sets of the disclosure comprise the following:

A compositions for detecting the presence of Salmonella in a sample may comprise: at least one primer pair having hybridization specificity for priming amplification of an RNA-inducing agent-responsive gene of Salmonella wherein the RNA-inducing agent-responsive gene is agsA and the at least one primer pair comprises isolated nucleic acid molecules having the sequences of SEQ ID NO:1 and SEQ ID NO:134, or SEQ ID NO:2 and SEQ ID NO:135, or SEQ ID NO:3 and SEQ ID NO:136, or SEQ ID NO: 4 and SEQ ID NO:137, or SEQ ID NO:5 and SEQ ID NO:138, or SEQ ID NO:6 and SEQ ID NO:139, or SEQ ID NO:7 and SEQ ID NO:140, or SEQ ID NO:8 and SEQ ID NO:141, or SEQ ID NO:9 and SEQ ID NO:142, or SEQ ID NO:10 and SEQ ID NO:143, or SEQ ID NO: 11 and SEQ ID NO:144, or SEQ ID NO:12 and SEQ ID NO:145, or SEQ ID NO:13 and SEQ ID NO:146, or SEQ ID NO:14 and SEQ ID NO:147, or SEQ ID NO:15 and SEQ ID NO:148, or SEQ ID NO:16 and SEQ ID NO:149, or SEQ ID NO:17 and SEQ ID NO:150, or SEQ ID NO: 18 and SEQ ID NO:151, or SEQ ID NO:19 and SEQ ID NO:152, or SEQ ID NO:20 and SEQ ID NO:153, or SEQ ID NO:21 and SEQ ID NO:154, or SEQ ID NO:22 and SEQ ID NO:155, or SEQ ID NO:23 and SEQ ID NO:156 or a nucleic acid molecule with at least 90% sequence homology to the primer pair sequences. The composition may also comprise at least one probe wherein the at least one probe has the sequence of SEQ ID: NO: 267-SEQ ID NO: 289.

A compositions for detecting the presence of Salmonella in a sample may comprise: at least one primer pair having hybridization specificity for priming amplification of an RNA-inducing agent-responsive gene of Salmonella wherein the RNA-inducing agent-responsive gene is cspH and the at least one primer pair comprises isolated nucleic acid molecules having the sequences of SEQ ID NO:24 and SEQ ID NO:157, or SEQ ID NO:25 and SEQ ID NO:158, or SEQ ID NO:26 and SEQ ID NO:159, or SEQ ID NO: 27 and SEQ ID NO:160, or SEQ ID NO:28 and SEQ ID NO:161, or SEQ ID NO:29 and SEQ ID NO:162, or SEQ ID NO:30 and SEQ ID NO:163, or SEQ ID NO:31 and SEQ ID NO:164, or SEQ ID NO:32 and SEQ ID NO:165, or SEQ ID NO:33 and SEQ ID NO:166, or SEQ ID NO: 34 and SEQ ID NO:167, or SEQ ID NO:35 and SEQ ID NO:168, or SEQ ID NO:36 and SEQ ID NO:169, or SEQ ID NO:37 and SEQ ID NO:170, or SEQ ID NO:38 and SEQ ID NO:171, or SEQ ID NO:39 and SEQ ID NO:172, or SEQ ID NO:40 and SEQ ID NO:173, or SEQ ID NO: 41 and SEQ ID NO:174, or SEQ ID NO:42 and SEQ ID NO:175, or SEQ ID NO:43 and SEQ ID NO:176, or SEQ ID NO:44 and SEQ ID NO:177 or a nucleic acid molecule with at least 90% sequence homology to the primer pair sequences. The composition may also comprise at least one probe wherein the at least one probe has the sequence of SEQ ID: NO: 290-SEQ ID NO:310.

A compositions for detecting the presence of Salmonella in a sample may comprise: at least one primer pair having hybridization specificity for priming amplification of an RNA-inducing agent-responsive gene of Salmonella wherein the RNA-inducing agent-responsive gene is dnaK and the at least one primer pair comprises isolated nucleic acid molecules having the sequences of SEQ ID NO:45 and SEQ ID NO:178, or SEQ ID NO:46 and SEQ ID NO:179, or SEQ ID NO:47 and SEQ ID NO:180, or SEQ ID NO:48 and SEQ ID NO:181, or SEQ ID NO:49 and SEQ ID NO:182, or SEQ ID NO: 50 and SEQ ID NO:183, or SEQ ID NO:51 and SEQ ID NO:184, or SEQ ID NO:52 and SEQ ID NO:185, or SEQ ID NO:53 and SEQ ID NO:186, or SEQ ID NO:54 and SEQ ID NO:187, or SEQ ID NO:55 and SEQ ID NO:188, or SEQ ID NO:56 and SEQ ID NO:189, or SEQ ID NO: 57 and SEQ ID NO:190, or SEQ ID NO:58 and SEQ ID NO:191, or SEQ ID NO:59 and SEQ ID NO:192, or SEQ ID NO:60 and SEQ ID NO:193, or SEQ ID NO:61 and SEQ ID NO:194, or SEQ ID NO:62 and SEQ ID NO:195, or SEQ ID NO:63 and SEQ ID NO:196, or SEQ ID NO: 64 and SEQ ID NO:197, or SEQ ID NO:65 and SEQ ID NO:198 or a nucleic acid molecule with at least 90% sequence homology to the primer pair sequences. The composition may also comprise at least one probe wherein the at least one probe has the sequence of SEQ ID: NO: 311-SEQ ID NO:331.

A compositions for detecting the presence of Salmonella in a sample may comprise: at least one primer pair having hybridization specificity for priming amplification of an RNA-inducing agent-responsive gene of Salmonella wherein the RNA-inducing agent-responsive gene is Hsp60 and the at least one primer pair comprises isolated nucleic acid molecules having the sequences of or SEQ ID NO:66 and SEQ ID NO:199, or SEQ ID NO:67 and SEQ ID NO:200, or SEQ ID NO:68 and SEQ ID NO:201, or SEQ ID NO:69 and SEQ ID NO:202, and SEQ ID NO:70 and SEQ ID NO:203, or SEQ ID NO:71 and SEQ ID NO:204, or SEQ ID NO:72 and SEQ ID NO:205, or SEQ ID NO:73 and SEQ ID NO:206, or SEQ ID NO:74 and SEQ ID NO:207, or SEQ ID NO:75 and SEQ ID NO:208, or SEQ ID NO:76 and SEQ ID NO:209, or SEQ ID NO:77 and SEQ ID NO:210, or SEQ ID NO:78 and SEQ ID NO:211, or SEQ ID NO:79 and SEQ ID NO:212, or SEQ ID NO:80 and SEQ ID NO:213, or SEQ ID NO:81 and SEQ ID NO:214, or SEQ ID NO:82 and SEQ ID NO:215, or SEQ ID NO:83 and SEQ ID NO:216, or SEQ ID NO:84 and SEQ ID NO:217, or SEQ ID NO:85 and SEQ ID NO:218, or SEQ ID NO:86 and SEQ ID NO:219 or a nucleic acid molecule with at least 90% sequence homology to the primer pair sequences. The composition may also comprise at least one probe wherein the at least one probe has the sequence of SEQ ID: NO: 332-SEQ ID NO:352.

A compositions for detecting the presence of Salmonella in a sample may comprise: at least one primer pair having hybridization specificity for priming amplification of an RNA-inducing agent-responsive gene of Salmonella wherein the RNA-inducing agent-responsive gene is ibpAB and the at least one primer pair comprises isolated nucleic acid molecules having the sequences of SEQ ID NO:87 and SEQ ID NO:220, and SEQ ID NO:88 and SEQ ID NO:221, or SEQ ID NO:89 and SEQ ID NO:222, or SEQ ID NO:90 and SEQ ID NO:223, or SEQ ID NO:91 and SEQ ID NO:224, or SEQ ID NO:92 and SEQ ID NO:225, or SEQ ID NO:93 and SEQ ID NO:226, or SEQ ID NO:94 and SEQ ID NO:227, or SEQ ID NO:95 and SEQ ID NO:228, or SEQ ID NO:96 and SEQ ID NO:229, or SEQ ID NO:97 and SEQ ID NO:230, or SEQ ID NO:98 and SEQ ID NO:231, or SEQ ID NO:99 and SEQ ID NO:232, or SEQ ID NO:100 and SEQ ID NO:233, or SEQ ID NO:101 and SEQ ID NO:234, or SEQ ID NO:102 and SEQ ID NO:235, or SEQ ID NO:103 and SEQ ID NO:236, or SEQ ID NO:104 and SEQ ID NO:237, or SEQ ID NO:105 and SEQ ID NO:238, or SEQ ID NO:106 and SEQ ID NO:239, or SEQ ID NO:107 and SEQ ID NO:240, or SEQ ID NO:108 and SEQ ID NO:241, or SEQ ID NO:109 and SEQ ID NO:242 or a nucleic acid molecule with at least 90% sequence homology to the primer pair sequences. The composition may also comprise at least one probe wherein the at least one probe has the sequence of SEQ ID: NO: 353-SEQ ID NO:375.

A compositions for detecting the presence of Salmonella in a sample may comprise: at least one primer pair having hybridization specificity for priming amplification of an RNA-inducing agent-responsive gene of Salmonella wherein the RNA-inducing agent-responsive gene is uspA and the at least one primer pair comprises isolated nucleic acid molecules having the sequences of or SEQ ID NO:110 and SEQ ID NO:243, or SEQ ID NO:111 and SEQ ID NO:244, or SEQ ID NO:112 and SEQ ID NO:245, or SEQ ID NO:113 and SEQ ID NO:246, or SEQ ID NO:114 and SEQ ID NO:247, or SEQ ID NO:115 and SEQ ID NO:248, or SEQ ID NO:116 and SEQ ID NO:249, or SEQ ID NO:117 and SEQ ID NO:250, or SEQ ID NO:118 and SEQ ID NO:251, or SEQ ID NO:119 and SEQ ID NO:252, or SEQ ID NO:120 and SEQ ID NO:253, or SEQ ID NO:121 and SEQ ID NO:254, or SEQ ID NO:122 and SEQ ID NO:255, or SEQ ID NO:123 and SEQ ID NO:256, or SEQ ID NO:124 and SEQ ID NO:257, or SEQ ID NO:125 and SEQ ID NO:258, or SEQ ID NO:126 and SEQ ID NO:259, or SEQ ID NO:127 and SEQ ID NO:260, or SEQ ID NO:128 and SEQ ID NO:261, or SEQ ID NO:129 and SEQ ID NO:262, or SEQ ID NO:130 and SEQ ID NO:263, or SEQ ID NO:131 and SEQ ID NO:264, or SEQ ID NO:132 and SEQ ID NO:265 or a nucleic acid molecule with at least 90% sequence homology to the primer pair sequences. The composition may also comprise at least one probe wherein the at least one probe has the sequence of SEQ ID: NO: 376-SEQ ID NO:398.

A compositions for detecting the presence of Salmonella in a sample may comprise: at least one primer pair having hybridization specificity for priming amplification of an RNA-inducing agent-responsive gene of Salmonella wherein the RNA-inducing agent-responsive gene is hilA and the at least one primer pair comprises isolated nucleic acid molecules having the sequences of or SEQ ID NO:133 and SEQ ID NO:266 or a nucleic acid molecule with at least 90% sequence homology to the primer pair sequences. The composition may also comprise at least one probe wherein the at least one probe has the sequence of SEQ ID: NO: 399.

A composition for detecting the presence of Listeria in a sample may comprise: at least one primer pair having hybridization specificity for priming amplification of an RNA-inducing agent-responsive gene of Listeria wherein the RNA-inducing agent-responsive gene is inlA and the at least one primer pair comprises isolated nucleic acid molecules having the sequences of SEQ ID NO:400 and SEQ ID NO:855, or SEQ ID NO:401 and SEQ ID NO:856, or SEQ ID NO:402 and SEQ ID NO:857, or SEQ ID NO: 403 and SEQ ID NO:858, or SEQ ID NO:404 and SEQ ID NO:859, or SEQ ID NO:405 and SEQ ID NO:860, or SEQ ID NO:406 and SEQ ID NO:861, or SEQ ID NO:407 and SEQ ID NO:862, or SEQ ID NO:408 and SEQ ID NO:863, or SEQ ID NO:409 and SEQ ID NO:864, or SEQ ID NO: 410 and SEQ ID NO:865, or SEQ ID NO:411 and SEQ ID NO:866, or SEQ ID NO:412 and SEQ ID NO:867, or SEQ ID NO:413 and SEQ ID NO:868, or SEQ ID NO:414 and SEQ ID NO:869, or SEQ ID NO:415 and SEQ ID NO:870, or SEQ ID NO:416 and SEQ ID NO:871, or SEQ ID NO:417 and SEQ ID NO:872, or SEQ ID NO:418 and SEQ ID NO:873, or SEQ ID NO:419 and SEQ ID NO:874, or SEQ ID NO: 420 and SEQ ID NO:875, or a nucleic acid molecule with at least 90% sequence homology to the primer pair sequences. The composition may also comprise at least one probe wherein the at least one probe has the sequence of SEQ ID: NO: 1310-SEQ ID NO: 1330.

A composition for detecting the presence of Listeria in a sample may comprise: at least one primer pair having hybridization specificity for priming amplification of an RNA-inducing agent-responsive gene of Listeria wherein the RNA-inducing agent-responsive gene is lmo0539 and the at least one primer pair comprises isolated nucleic acid molecules having the sequences of SEQ ID NO:421 and SEQ ID NO:876, or SEQ ID NO:422 and SEQ ID NO:877, or SEQ ID NO:423 and SEQ ID NO:878, or SEQ ID NO:424 and SEQ ID NO:879, or SEQ ID NO:425 and SEQ ID NO:880, or SEQ ID NO:426 and SEQ ID NO:881, or SEQ ID NO:427 and SEQ ID NO:882, or SEQ ID NO:428 and SEQ ID NO:883, or SEQ ID NO:429 and SEQ ID NO:884, or SEQ ID NO: 430 and SEQ ID NO:885, or SEQ ID NO:431 and SEQ ID NO:886, or SEQ ID NO:432 and SEQ ID NO:887, or SEQ ID NO:433 and SEQ ID NO:888, or SEQ ID NO:434 and SEQ ID NO:889, or SEQ ID NO:435 and SEQ ID NO:890, or SEQ ID NO:436 and SEQ ID NO:891, or SEQ ID NO:437 and SEQ ID NO:892, or SEQ ID NO:438 and SEQ ID NO:893, or SEQ ID NO:439 and SEQ ID NO:894, or SEQ ID NO: 440 and SEQ ID NO:895, or SEQ ID NO: 441 and SEQ ID NO:896, or a nucleic acid molecule with at least 90% sequence homology to the primer pair sequences. The composition may also comprise at least one probe wherein the at least one probe has the sequence of SEQ ID: NO: 1331-SEQ ID NO:1351.

A composition for detecting the presence of Listeria in a sample may comprise: at least one primer pair having hybridization specificity for priming amplification of an RNA-inducing agent-responsive gene of Listeria wherein the RNA-inducing agent-responsive gene is lmo2158 and the at least one primer pair comprises isolated nucleic acid molecules having the sequences of or SEQ ID NO:442 and SEQ ID NO:897, or SEQ ID NO:443 and SEQ ID NO:898, or SEQ ID NO:444 and SEQ ID NO:899, or SEQ ID NO:445 and SEQ ID NO:900, or SEQ ID NO:446 and SEQ ID NO:901, or SEQ ID NO:447 and SEQ ID NO:902, or SEQ ID NO:448 and SEQ ID NO:903, or SEQ ID NO:449 and SEQ ID NO:904, or SEQ ID NO: 450 and SEQ ID NO:905, or SEQ ID NO:451 and SEQ ID NO:906, or SEQ ID NO:452 and SEQ ID NO:907, or SEQ ID NO:453 and SEQ ID NO:908, or SEQ ID NO:454 and SEQ ID NO:909, or SEQ ID NO:455 and SEQ ID NO:910, or SEQ ID NO:456 and SEQ ID NO:911, or SEQ ID NO:457 and SEQ ID NO:912, or SEQ ID NO:458 and SEQ ID NO:913, or SEQ ID NO:459 and SEQ ID NO:914, or SEQ ID NO: 460 and SEQ ID NO:915, or a nucleic acid molecule with at least 90% sequence homology to the primer pair sequences. The composition may also comprise at least one probe wherein the at least one probe has the sequence of SEQ ID: NO: 1352-SEQ ID NO:1370.

A composition for detecting the presence of Listeria in a sample may comprise: at least one primer pair having hybridization specificity for priming amplification of an RNA-inducing agent-responsive gene of Listeria wherein the RNA-inducing agent-responsive gene is bsh and the at least one primer pair comprises isolated nucleic acid molecules having the sequences of SEQ ID NO:461 and SEQ ID NO:916, or SEQ ID NO:462 and SEQ ID NO:917, or SEQ ID NO:463 and SEQ ID NO:918, or SEQ ID NO:464 and SEQ ID NO:919, or SEQ ID NO:465 and SEQ ID NO:920, or SEQ ID NO:466 and SEQ ID NO:921, or SEQ ID NO:467 and SEQ ID NO:922, or SEQ ID NO:468 and SEQ ID NO:923, or SEQ ID NO:469 and SEQ ID NO:924, or SEQ ID NO: 470 and SEQ ID NO:925, SEQ ID NO:471 and SEQ ID NO:926, or SEQ ID NO:472 and SEQ ID NO:927, or SEQ ID NO:473 and SEQ ID NO:928, or SEQ ID NO:474 and SEQ ID NO:929, or SEQ ID NO:475 and SEQ ID NO:930, or SEQ ID NO:476 and SEQ ID NO:931, or SEQ ID NO:477 and SEQ ID NO:932, or SEQ ID NO:478 and SEQ ID NO:933, or SEQ ID NO:479 and SEQ ID NO:934, or SEQ ID NO: 480 and SEQ ID NO:935, or a nucleic acid molecule with at least 90% sequence homology to the primer pair sequences. The composition may also comprise at least one probe wherein the at least one probe has the sequence of SEQ ID: NO: 1371-SEQ ID NO:1390.

A composition for detecting the presence of Listeria in a sample may comprise: at least one primer pair having hybridization specificity for priming amplification of an RNA-inducing agent-responsive gene of Listeria wherein the RNA-inducing agent-responsive gene is inlB and the at least one primer pair comprises isolated nucleic acid molecules having the sequences of SEQ ID NO:481 and SEQ ID NO:936, or SEQ ID NO:482 and SEQ ID NO:937, or SEQ ID NO:483 and SEQ ID NO:938, or SEQ ID NO:484 and SEQ ID NO:939, or SEQ ID NO:485 and SEQ ID NO:940, or SEQ ID NO:486 and SEQ ID NO:941, or SEQ ID NO:487 and SEQ ID NO:942, or SEQ ID NO:488 and SEQ ID NO:943, or SEQ ID NO:489 and SEQ ID NO:944, or SEQ ID NO: 490 and SEQ ID NO:945, SEQ ID NO:491 and SEQ ID NO:946, or SEQ ID NO:492 and SEQ ID NO:947, or SEQ ID NO:493 and SEQ ID NO:948, or SEQ ID NO:494 and SEQ ID NO:949, or SEQ ID NO:495 and SEQ ID NO:950, or SEQ ID NO:496 and SEQ ID NO:951, or SEQ ID NO:497 and SEQ ID NO:952, or SEQ ID NO:498 and SEQ ID NO:953, or SEQ ID NO:499 and SEQ ID NO:954, or SEQ ID NO: 500 and SEQ ID NO:955, or SEQ ID NO: 501 and SEQ ID NO:956 or a nucleic acid molecule with at least 90% sequence homology to the primer pair sequences. The composition may also comprise at least one probe wherein the at least one probe has the sequence of SEQ ID: NO: 1391-SEQ ID NO:1411.

A composition for detecting the presence of Listeria in a sample may comprise: at least one primer pair having hybridization specificity for priming amplification of an RNA-inducing agent-responsive gene of Listeria wherein the RNA-inducing agent-responsive gene is lmo0596 and the at least one primer pair comprises isolated nucleic acid molecules having the sequences of SEQ ID NO:502 and SEQ ID NO:957, or SEQ ID NO:503 and SEQ ID NO:958, or SEQ ID NO:504 and SEQ ID NO:959, or SEQ ID NO:505 and SEQ ID NO:960, or SEQ ID NO:506 and SEQ ID NO:961, or SEQ ID NO:507 and SEQ ID NO:962, or SEQ ID NO:508 and SEQ ID NO:963, or SEQ ID NO:509 and SEQ ID NO:964, or SEQ ID NO: 510 and SEQ ID NO:965, SEQ ID NO:511 and SEQ ID NO:966, or SEQ ID NO:512 and SEQ ID NO:967, or SEQ ID NO:513 and SEQ ID NO:968, or SEQ ID NO:514 and SEQ ID NO:969, or SEQ ID NO:515 and SEQ ID NO:970, or SEQ ID NO:516 and SEQ ID NO:971, or SEQ ID NO:517 and SEQ ID NO:972, or SEQ ID NO:518 and SEQ ID NO:973, or SEQ ID NO:519 and SEQ ID NO:974, or SEQ ID NO: 520 and SEQ ID NO:975, or SEQ ID NO: 521 and SEQ ID NO:976, or SEQ ID NO: 522 and SEQ ID NO:977, or a nucleic acid molecule with at least 90% sequence homology to the primer pair sequences. The composition may also comprise at least one probe wherein the at least one probe has the sequence of SEQ ID: NO: 1412-SEQ ID NO:1432.

A composition for detecting the presence of Listeria in a sample may comprise: at least one primer pair having hybridization specificity for priming amplification of an RNA-inducing agent-responsive gene of Listeria wherein the RNA-inducing agent-responsive gene is lmo2230 and the at least one primer pair comprises isolated nucleic acid molecules having the sequences of SEQ ID NO:523 and SEQ ID NO:978, or SEQ ID NO:524 and SEQ ID NO:979, or SEQ ID NO:525 and SEQ ID NO:980, or SEQ ID NO:526 and SEQ ID NO:981, or SEQ ID NO:527 and SEQ ID NO:982, or SEQ ID NO:528 and SEQ ID NO:983, or SEQ ID NO:529 and SEQ ID NO:984, or SEQ ID NO: 530 and SEQ ID NO:985, SEQ ID NO:531 and SEQ ID NO:986, or SEQ ID NO:532 and SEQ ID NO:987, or SEQ ID NO:533 and SEQ ID NO:988, or SEQ ID NO:534 and SEQ ID NO:989, or SEQ ID NO:535 and SEQ ID NO:990, or SEQ ID NO:536 and SEQ ID NO:991, or SEQ ID NO:537 and SEQ ID NO:992, or SEQ ID NO:538 and SEQ ID NO:993, or SEQ ID NO:539 and SEQ ID NO:994, or SEQ ID NO: 540 and SEQ ID NO:995, or SEQ ID NO: 541 and SEQ ID NO:996, or SEQ ID NO: 542 and SEQ ID NO:997, or SEQ ID NO: 543 and SEQ ID NO:998, or a nucleic acid molecule with at least 90% sequence homology to the primer pair sequences. The composition may also comprise at least one probe wherein the at least one probe has the sequence of SEQ ID: NO: 1433-SEQ ID NO:1453.

A composition for detecting the presence of Listeria in a sample may comprise: at least one primer pair having hybridization specificity for priming amplification of an RNA-inducing agent-responsive gene of Listeria wherein the RNA-inducing agent-responsive gene is clpE and the at least one primer pair comprises isolated nucleic acid molecules having the sequences of SEQ ID NO:544 and SEQ ID NO:999, or SEQ ID NO:545 and SEQ ID NO:1000, or SEQ ID NO:546 and SEQ ID NO:1001, or SEQ ID NO:547 and SEQ ID NO:1002, or SEQ ID NO:548 and SEQ ID NO:1003, or SEQ ID NO:549 and SEQ ID NO:1004, or SEQ ID NO: 550 and SEQ ID NO:1005, SEQ ID NO:551 and SEQ ID NO:1006, or SEQ ID NO:552 and SEQ ID NO:1007, or SEQ ID NO:553 and SEQ ID NO:1008, or SEQ ID NO:554 and SEQ ID NO:1009, or SEQ ID NO:555 and SEQ ID NO:1010, or SEQ ID NO:556 and SEQ ID NO:1011, or SEQ ID NO:557 and SEQ ID NO:1012, or SEQ ID NO:558 and SEQ ID NO:10013, or SEQ ID NO:559 and SEQ ID NO:1014, or SEQ ID NO: 560 and SEQ ID NO:1015, SEQ ID NO:561 and SEQ ID NO:1006, or SEQ ID NO:562 and SEQ ID NO:1017, or SEQ ID NO:563 and SEQ ID NO:1018, or SEQ ID NO:564 and SEQ ID NO:1019, or a nucleic acid molecule with at least 90% sequence homology to the primer pair sequences. The composition may also comprise at least one probe wherein the at least one probe has the sequence of SEQ ID: NO: 1454-SEQ ID NO:1474.

A composition for detecting the presence of Listeria in a sample may comprise: at least one primer pair having hybridization specificity for priming amplification of an RNA-inducing agent-responsive gene of Listeria wherein the RNA-inducing agent-responsive gene is inlC and the at least one primer pair comprises isolated nucleic acid molecules having the sequences of SEQ ID NO:565 and SEQ ID NO:1020, or SEQ ID NO:566 and SEQ ID NO:1021, or SEQ ID NO:567 and SEQ ID NO:1022, or SEQ ID NO:568 and SEQ ID NO:1023, or SEQ ID NO:569 and SEQ ID NO:1024, or SEQ ID NO: 570 and SEQ ID NO:1025, SEQ ID NO:571 and SEQ ID NO:1026, or SEQ ID NO:572 and SEQ ID NO:1027, or SEQ ID NO:573 and SEQ ID NO:1028, or SEQ ID NO:574 and SEQ ID NO:1029, or SEQ ID NO:575 and SEQ ID NO:1030, or SEQ ID NO:576 and SEQ ID NO:1031, or SEQ ID NO:577 and SEQ ID NO:1032, or SEQ ID NO:578 and SEQ ID NO:1033, or SEQ ID NO:579 and SEQ ID NO:1034, or SEQ ID NO: 580 and SEQ ID NO:1035, SEQ ID NO:581 and SEQ ID NO:1036, or SEQ ID NO:582 and SEQ ID NO:1037, or SEQ ID NO:583 and SEQ ID NO:1038, or SEQ ID NO:584 and SEQ ID NO:1039, or SEQ ID NO:585 and SEQ ID NO:1040, or a nucleic acid molecule with at least 90% sequence homology to the primer pair sequences. The composition may also comprise at least one probe wherein the at least one probe has the sequence of SEQ ID: NO: 1475-SEQ ID NO:1495.

A composition for detecting the presence of Listeria in a sample may comprise: at least one primer pair having hybridization specificity for priming amplification of an RNA-inducing agent-responsive gene of Listeria wherein the RNA-inducing agent-responsive gene is lmo0670 and the at least one primer pair comprises isolated nucleic acid molecules having the sequences of or SEQ ID NO:586 and SEQ ID NO:1041, or SEQ ID NO:587 and SEQ ID NO:1042, or SEQ ID NO:588 and SEQ ID NO:1043, or SEQ ID NO:589 and SEQ ID NO:1044, or SEQ ID NO: 590 and SEQ ID NO:1045, SEQ ID NO:591 and SEQ ID NO:1046, or SEQ ID NO:592 and SEQ ID NO:1047, or SEQ ID NO:593 and SEQ ID NO:1048, or SEQ ID NO:594 and SEQ ID NO:1049, or SEQ ID NO:595 and SEQ ID NO:1050, or SEQ ID NO:596 and SEQ ID NO:1051, or SEQ ID NO:597 and SEQ ID NO:1052, or SEQ ID NO:598 and SEQ ID NO:1053, or SEQ ID NO:599 and SEQ ID NO:1054, or SEQ ID NO: 600 and SEQ ID NO:1055, SEQ ID NO:601 and SEQ ID NO:1056, or SEQ ID NO:602 and SEQ ID NO:1057, or SEQ ID NO:603 and SEQ ID NO:1058, or SEQ ID NO:604 and SEQ ID NO:1059, or SEQ ID NO:605 and SEQ ID NO:1060, or a nucleic acid molecule with at least 90% sequence homology to the primer pair sequences. The composition may also comprise at least one probe wherein the at least one probe has the sequence of SEQ ID: NO: 1496-SEQ ID NO:1515.

A composition for detecting the presence of Listeria in a sample may comprise: at least one primer pair having hybridization specificity for priming amplification of an RNA-inducing agent-responsive gene of Listeria wherein the RNA-inducing agent-responsive gene is lmo2522 and the at least one primer pair comprises isolated nucleic acid molecules having the sequences of SEQ ID NO:606 and SEQ ID NO:1061, or SEQ ID NO:607 and SEQ ID NO:1062, or SEQ ID NO:608 and SEQ ID NO:1063, or SEQ ID NO:609 and SEQ ID NO:1064, or SEQ ID NO: 610 and SEQ ID NO:1065, SEQ ID NO:611 and SEQ ID NO:1066, or SEQ ID NO:612 and SEQ ID NO:1067, or SEQ ID NO:613 and SEQ ID NO:1068, or SEQ ID NO:614 and SEQ ID NO:1069, or SEQ ID NO:615 and SEQ ID NO:1070, or SEQ ID NO:616 and SEQ ID NO:1071, or SEQ ID NO:617 and SEQ ID NO:1072, or SEQ ID NO:618 and SEQ ID NO:1073, or SEQ ID NO:619 and SEQ ID NO:1074, or SEQ ID NO: 620 and SEQ ID NO:1075, SEQ ID NO:621 and SEQ ID NO:1076, or SEQ ID NO:622 and SEQ ID NO:1077, or SEQ ID NO:623 and SEQ ID NO:1078, or SEQ ID NO:624 and SEQ ID NO:1079, or SEQ ID NO:625 and SEQ ID NO:1080, or SEQ ID NO:626 and SEQ ID NO:1081, or a nucleic acid molecule with at least 90% sequence homology to the primer pair sequences. The composition may also comprise at least one probe wherein the at least one probe has the sequence of SEQ ID: NO: 1516-SEQ ID NO:1536.

A composition for detecting the presence of Listeria in a sample may comprise: at least one primer pair having hybridization specificity for priming amplification of an RNA-inducing agent-responsive gene of Listeria wherein the RNA-inducing agent-responsive gene is cspL and the at least one primer pair comprises isolated nucleic acid molecules having the sequences of SEQ ID NO:627 and SEQ ID NO:1082, or SEQ ID NO:628 and SEQ ID NO:1083, or SEQ ID NO:629 and SEQ ID NO:1084, or SEQ ID NO: 630 and SEQ ID NO:1085, SEQ ID NO:631 and SEQ ID NO:1086, or SEQ ID NO:632 and SEQ ID NO:1087, or SEQ ID NO:633 and SEQ ID NO:1088, or SEQ ID NO:634 and SEQ ID NO:1089, or SEQ ID NO:635 and SEQ ID NO:1090, or SEQ ID NO:636 and SEQ ID NO:1091, or SEQ ID NO:637 and SEQ ID NO:1092, or SEQ ID NO:638 and SEQ ID NO:1093, or SEQ ID NO:639 and SEQ ID NO:1094, or SEQ ID NO: 640 and SEQ ID NO:1095, SEQ ID NO:641 and SEQ ID NO:1096, or SEQ ID NO:642 and SEQ ID NO:1097, or SEQ ID NO:643 and SEQ ID NO:1098, or SEQ ID NO:644 and SEQ ID NO:1099, or SEQ ID NO:645 and SEQ ID NO:1100, or SEQ ID NO:646 and SEQ ID NO:1101, or SEQ ID NO:647 and SEQ ID NO:1102, or a nucleic acid molecule with at least 90% sequence homology to the primer pair sequences. The composition may also comprise at least one probe wherein the at least one probe has the sequence of SEQ ID: NO: 1537-SEQ ID NO:1557.

A composition for detecting the presence of Listeria in a sample may comprise: at least one primer pair having hybridization specificity for priming amplification of an RNA-inducing agent-responsive gene of Listeria wherein the RNA-inducing agent-responsive gene is inlG and the at least one primer pair comprises isolated nucleic acid molecules having the sequences SEQ ID NO:648 and SEQ ID NO:1103, or SEQ ID NO:649 and SEQ ID NO:1104, or SEQ ID NO: 650 and SEQ ID NO:1105, SEQ ID NO:651 and SEQ ID NO:1106, or SEQ ID NO:652 and SEQ ID NO:1107, or SEQ ID NO:653 and SEQ ID NO:1108, or SEQ ID NO:654 and SEQ ID NO:1109, or SEQ ID NO:655 and SEQ ID NO:1110, or SEQ ID NO:656 and SEQ ID NO:1111, or SEQ ID NO:657 and SEQ ID NO:1112, or SEQ ID NO:658 and SEQ ID NO:1113, or SEQ ID NO:659 and SEQ ID NO:1114, or SEQ ID NO: 660 and SEQ ID NO:1115, SEQ ID NO:661 and SEQ ID NO:1116, or SEQ ID NO:662 and SEQ ID NO:1117, or SEQ ID NO:663 and SEQ ID NO:1118, or SEQ ID NO:664 and SEQ ID NO:1119, or SEQ ID NO:665 and SEQ ID NO:1120, or SEQ ID NO:666 and SEQ ID NO:1121, or SEQ ID NO:667 and SEQ ID NO:1122 or a nucleic acid molecule with at least 90% sequence homology to the primer pair sequences. The composition may also comprise at least one probe wherein the at least one probe has the sequence of SEQ ID: NO: 1558-SEQ ID NO:1577.

A composition for detecting the presence of Listeria in a sample may comprise: at least one primer pair having hybridization specificity for priming amplification of an RNA-inducing agent-responsive gene of Listeria wherein the RNA-inducing agent-responsive gene is Lmo0699 and the at least one primer pair comprises isolated nucleic acid molecules having the SEQ ID NO:668 and SEQ ID NO:1123, or SEQ ID NO:669 and SEQ ID NO:1124, or SEQ ID NO: 670 and SEQ ID NO:1125, SEQ ID NO:671 and SEQ ID NO:1126, or SEQ ID NO:672 and SEQ ID NO:1127, or SEQ ID NO:673 and SEQ ID NO:1128, or SEQ ID NO:674 and SEQ ID NO:1129, or SEQ ID NO:675 and SEQ ID NO:1130, or SEQ ID NO:676 and SEQ ID NO:1131, or SEQ ID NO:677 and SEQ ID NO:1132, or SEQ ID NO:678 and SEQ ID NO:1133, or SEQ ID NO:679 and SEQ ID NO:1134, or SEQ ID NO: 680 and SEQ ID NO:1135, or SEQ ID NO:681 and SEQ ID NO:1136, or SEQ ID NO:682 and SEQ ID NO:1137, or SEQ ID NO:683 and SEQ ID NO:1138, or SEQ ID NO:684 and SEQ ID NO:1139, or SEQ ID NO:685 and SEQ ID NO:1140, or SEQ ID NO:686 and SEQ ID NO:1141, or SEQ ID NO:687 and SEQ ID NO:1142, SEQ ID NO:688 and SEQ ID NO:1143, or a nucleic acid molecule with at least 90% sequence homology to the primer pair sequences. The composition may also comprise at least one probe wherein the at least one probe has the sequence of SEQ ID: NO: 1578-SEQ ID NO:1598.

A composition for detecting the presence of Listeria in a sample may comprise: at least one primer pair having hybridization specificity for priming amplification of an RNA-inducing agent-responsive gene of Listeria wherein the RNA-inducing agent-responsive gene is opuCA and the at least one primer pair comprises isolated nucleic acid molecules having the sequences of SEQ ID NO:689 and SEQ ID NO:1144, or SEQ ID NO: 690 and SEQ ID NO:1145, SEQ ID NO:691 and SEQ ID NO:1146, or SEQ ID NO:692 and SEQ ID NO:1147, or SEQ ID NO:693 and SEQ ID NO:1148, or SEQ ID NO:694 and SEQ ID NO:1149, or SEQ ID NO:695 and SEQ ID NO:1150, or SEQ ID NO:696 and SEQ ID NO:1151, or SEQ ID NO:697 and SEQ ID NO:1152, or SEQ ID NO:698 and SEQ ID NO:1153, or SEQ ID NO:699 and SEQ ID NO:1154, or SEQ ID NO: 700 and SEQ ID NO:1155, SEQ ID NO:701 and SEQ ID NO:1156, or SEQ ID NO:702 and SEQ ID NO:1157, or SEQ ID NO:703 and SEQ ID NO:1158, or SEQ ID NO:704 and SEQ ID NO:1159, or SEQ ID NO:705 and SEQ ID NO:1160, or SEQ ID NO:706 and SEQ ID NO:1161, or SEQ ID NO:707 and SEQ ID NO:1162, SEQ ID NO:708 and SEQ ID NO:1163, SEQ ID NO:709 and SEQ ID NO:1164, or a nucleic acid molecule with at least 90% sequence homology to the primer pair sequences. The composition may also comprise at least one probe wherein the at least one probe has the sequence of SEQ ID: NO: 1599-SEQ ID NO:1619.

A composition for detecting the presence of Listeria in a sample may comprise: at least one primer pair having hybridization specificity for priming amplification of an RNA-inducing agent-responsive gene of Listeria wherein the RNA-inducing agent-responsive gene is inlJ and the at least one primer pair comprises isolated nucleic acid molecules having the sequences of SEQ ID NO: 710 and SEQ ID NO:1165, SEQ ID NO:711 and SEQ ID NO:1166, or SEQ ID NO:712 and SEQ ID NO:1167, or SEQ ID NO:713 and SEQ ID NO:1168, or SEQ ID NO:714 and SEQ ID NO:1169, or SEQ ID NO:715 and SEQ ID NO:1170, or SEQ ID NO:716 and SEQ ID NO:1171, or SEQ ID NO:717 and SEQ ID NO:1172, or SEQ ID NO:718 and SEQ ID NO:1173, or SEQ ID NO:719 and SEQ ID NO:1174, or SEQ ID NO: 720 and SEQ ID NO:1175, SEQ ID NO:721 and SEQ ID NO:1176, or SEQ ID NO:722 and SEQ ID NO:1177, or SEQ ID NO:723 and SEQ ID NO:1178, or SEQ ID NO:724 and SEQ ID NO:1179, or SEQ ID NO:725 and SEQ ID NO:1180, or SEQ ID NO:726 and SEQ ID NO:1181, SEQ ID NO:727 and SEQ ID NO:1182, or SEQ ID NO:728 and SEQ ID NO:1183, or SEQ ID NO:729 and SEQ ID NO:1184, or SEQ ID NO:730 and SEQ ID NO:1185, or a nucleic acid molecule with at least 90% sequence homology to the primer pair sequences. The composition may also comprise at least one probe wherein the at least one probe has the sequence of SEQ ID: NO: 1620-SEQ ID NO:1640.

A composition for detecting the presence of Listeria in a sample may comprise: at least one primer pair having hybridization specificity for priming amplification of an RNA-inducing agent-responsive gene of Listeria wherein the RNA-inducing agent-responsive gene is Lmo0782 and the at least one primer pair comprises isolated nucleic acid molecules having the sequences of SEQ ID NO:731 and SEQ ID NO:1186, or SEQ ID NO:732 and SEQ ID NO:1187, or SEQ ID NO:733 and SEQ ID NO:1188, or SEQ ID NO:734 and SEQ ID NO:1189, or SEQ ID NO:735 and SEQ ID NO:1190, or SEQ ID NO:736 and SEQ ID NO:1191, or SEQ ID NO:737 and SEQ ID NO:1192, or SEQ ID NO:738 and SEQ ID NO:1193, or SEQ ID NO:739 and SEQ ID NO:1194, or SEQ ID NO: 740 and SEQ ID NO:1195, SEQ ID NO:741 and SEQ ID NO:1196, or SEQ ID NO:742 and SEQ ID NO:1197, or SEQ ID NO:743 and SEQ ID NO:1198, or SEQ ID NO:744 and SEQ ID NO:1199, or SEQ ID NO:745 and SEQ ID NO:1200, or SEQ ID NO:746 and SEQ ID NO:1201, or SEQ ID NO:747 and SEQ ID NO:1202, SEQ ID NO:748 and SEQ ID NO:1203, or SEQ ID NO:749 and SEQ ID NO:1204, or SEQ ID NO: 750 and SEQ ID NO:1205, SEQ ID NO:751 and SEQ ID NO:1206, or a nucleic acid molecule with at least 90% sequence homology to the primer pair sequences. The composition may also comprise at least one probe wherein the at least one probe has the sequence of SEQ ID: NO: 1641-SEQ ID NO:1661.

A composition for detecting the presence of Listeria in a sample may comprise: at least one primer pair having hybridization specificity for priming amplification of an RNA-inducing agent-responsive gene of Listeria wherein the RNA-inducing agent-responsive gene is plcA and the at least one primer pair comprises isolated nucleic acid molecules having the sequences of SEQ ID NO:752 and SEQ ID NO:1207, or SEQ ID NO:753 and SEQ ID NO:1208, or SEQ ID NO:754 and SEQ ID NO:1209, or SEQ ID NO:755 and SEQ ID NO:1210, or SEQ ID NO:756 and SEQ ID NO:1211, or SEQ ID NO:757 and SEQ ID NO:1212, or SEQ ID NO:758 and SEQ ID NO:1213, or SEQ ID NO:759 and SEQ ID NO:1214, or SEQ ID NO: 760 and SEQ ID NO:1215, SEQ ID NO:761 and SEQ ID NO:1216, or SEQ ID NO:762 and SEQ ID NO:1217, or SEQ ID NO:763 and SEQ ID NO:1218, or SEQ ID NO:764 and SEQ ID NO:1219, or SEQ ID NO:765 and SEQ ID NO:1220, or SEQ ID NO:766 and SEQ ID NO:1221, or SEQ ID NO:767 and SEQ ID NO:1222, or SEQ ID NO:768 and SEQ ID NO:1223, or SEQ ID NO:769 and SEQ ID NO:1224, or SEQ ID NO: 770 and SEQ ID NO:1225, SEQ ID NO:771 and SEQ ID NO:1226, or SEQ ID NO:772 and SEQ ID NO:1227, or a nucleic acid molecule with at least 90% sequence homology to the primer pair sequences. The composition may also comprise at least one probe wherein the at least one probe has the sequence of SEQ ID: NO: 1662-SEQ ID NO:1682.

A composition for detecting the presence of Listeria in a sample may comprise: at least one primer pair having hybridization specificity for priming amplification of an RNA-inducing agent-responsive gene of Listeria wherein the RNA-inducing agent-responsive gene is lmo0189 and the at least one primer pair comprises isolated nucleic acid molecules having the sequences of SEQ ID NO:773 and SEQ ID NO:1228, or SEQ ID NO:774 and SEQ ID NO:1229, or SEQ ID NO:775 and SEQ ID NO:1230, or SEQ ID NO:776 and SEQ ID NO:1231, or SEQ ID NO:777 and SEQ ID NO:1232, or SEQ ID NO:778 and SEQ ID NO:1233, or SEQ ID NO:779 and SEQ ID NO:1234, or SEQ ID NO: 780 and SEQ ID NO:1235, SEQ ID NO:881 and SEQ ID NO:1236, or SEQ ID NO:782 and SEQ ID NO:1237, or SEQ ID NO:783 and SEQ ID NO:1238, or SEQ ID NO:784 and SEQ ID NO:1239, or SEQ ID NO:785 and SEQ ID NO:1240, or SEQ ID NO:786 and SEQ ID NO:1241, or SEQ ID NO:787 and SEQ ID NO:1242, SEQ ID NO:788 and SEQ ID NO:1243, or SEQ ID NO:789 and SEQ ID NO:1244, or SEQ ID NO: 790 and SEQ ID NO:1245, or a nucleic acid molecule with at least 90% sequence homology to the primer pair sequences. The composition may also comprise at least one probe wherein the at least one probe has the sequence of SEQ ID: NO: 1683-SEQ ID NO:1700.

A composition for detecting the presence of Listeria in a sample may comprise: at least one primer pair having hybridization specificity for priming amplification of an RNA-inducing agent-responsive gene of Listeria wherein the RNA-inducing agent-responsive gene is lmo0880 and the at least one primer pair comprises isolated nucleic acid molecules having the sequences of SEQ ID NO:991 and SEQ ID NO:1246, or SEQ ID NO:792 and SEQ ID NO:1247, or SEQ ID NO:793 and SEQ ID NO:1248, or SEQ ID NO:794 and SEQ ID NO:1249, or SEQ ID NO:795 and SEQ ID NO:1250, or SEQ ID NO:796 and SEQ ID NO:1251, or SEQ ID NO:797 and SEQ ID NO:1252, or SEQ ID NO:798 and SEQ ID NO:1253, or SEQ ID NO:799 and SEQ ID NO:1254, or SEQ ID NO: 800 and SEQ ID NO:1255, SEQ ID NO:801 and SEQ ID NO:1256, or SEQ ID NO:802 and SEQ ID NO:1257, or SEQ ID NO:803 and SEQ ID NO:1258, or SEQ ID NO:804 and SEQ ID NO:1259, or SEQ ID NO:805 and SEQ ID NO:1260, or SEQ ID NO:806 and SEQ ID NO:1261, or SEQ ID NO:807 and SEQ ID NO:1262, SEQ ID NO:808 and SEQ ID NO:1263, SEQ ID NO:809 and SEQ ID NO:1264, or SEQ ID NO: 810 and SEQ ID NO:1265, SEQ ID NO:811 and SEQ ID NO:1266, or a nucleic acid molecule with at least 90% sequence homology to the primer pair sequences. The composition may also comprise at least one probe wherein the at least one probe has the sequence of SEQ ID: NO: 1701-SEQ ID NO:1721.

A composition for detecting the presence of Listeria in a sample may comprise: at least one primer pair having hybridization specificity for priming amplification of an RNA-inducing agent-responsive gene of Listeria wherein the RNA-inducing agent-responsive gene is lmo0514 and the at least one primer pair comprises isolated nucleic acid molecules having the sequences of SEQ ID NO:812 and SEQ ID NO:1267, or SEQ ID NO:813 and SEQ ID NO:1268, or SEQ ID NO:814 and SEQ ID NO:1269, or SEQ ID NO:815 and SEQ ID NO:1270, or SEQ ID NO:816 and SEQ ID NO:1271, or SEQ ID NO:817 and SEQ ID NO:1272, or SEQ ID NO:818 and SEQ ID NO:1273, or SEQ ID NO:819 and SEQ ID NO:1274, or SEQ ID NO: 820 and SEQ ID NO:1275, SEQ ID NO:821 and SEQ ID NO:1276, or SEQ ID NO:822 and SEQ ID NO:1277, or SEQ ID NO:823 and SEQ ID NO:1278, or SEQ ID NO:824 and SEQ ID NO:1279, or SEQ ID NO:825 and SEQ ID NO:1280, or SEQ ID NO:826 and SEQ ID NO:1281, SEQ ID NO:827 and SEQ ID NO:1282, or SEQ ID NO:828 and SEQ ID NO:1283, or SEQ ID NO:829 and SEQ ID NO:1284, or SEQ ID NO:830 and SEQ ID NO:1285, or SEQ ID NO:831 and SEQ ID NO:1286, or SEQ ID NO:832 and SEQ ID NO:1287, or a nucleic acid molecule with at least 90% sequence homology to the primer pair sequences. The composition may also comprise at least one probe wherein the at least one probe has the sequence of SEQ ID: NO: 1722-SEQ ID NO:1742.

A composition for detecting the presence of Listeria in a sample may comprise: at least one primer pair having hybridization specificity for priming amplification of an RNA-inducing agent-responsive gene of Listeria wherein the RNA-inducing agent-responsive gene is lmo0129 and the at least one primer pair comprises isolated nucleic acid molecules having the sequences of SEQ ID NO:833 and SEQ ID NO:1288, or SEQ ID NO:834 and SEQ ID NO:1289, or SEQ ID NO:835 and SEQ ID NO:1290, or SEQ ID NO:836 and SEQ ID NO:1291, or SEQ ID NO:837 and SEQ ID NO:1292, or SEQ ID NO:838 and SEQ ID NO:1293, or SEQ ID NO:839 and SEQ ID NO:1294, or SEQ ID NO: 840 and SEQ ID NO:1295, SEQ ID NO:841 and SEQ ID NO:1296, or SEQ ID NO:842 and SEQ ID NO:1297, or SEQ ID NO:843 and SEQ ID NO:1298, or SEQ ID NO:844 and SEQ ID NO:1299, or SEQ ID NO:845 and SEQ ID NO:1300, or SEQ ID NO:846 and SEQ ID NO:1301, or SEQ ID NO:847 and SEQ ID NO:1302, SEQ ID NO:848 and SEQ ID NO:1303, or SEQ ID NO:849 and SEQ ID NO:1304, or SEQ ID NO: 850 and SEQ ID NO:1305, SEQ ID NO:7851 and SEQ ID NO:1306, or SEQ ID NO:852 and SEQ ID NO:1307, or SEQ ID NO:853 and SEQ ID NO:1308, or a nucleic acid molecule with at least 90% sequence homology to the primer pair sequences. The composition may also comprise at least one probe wherein the at least one probe has the sequence of SEQ ID: NO: 1743-SEQ ID NO:1763.

A composition for detecting the presence of Listeria in a sample may comprise: at least one primer pair having hybridization specificity for priming amplification of an RNA-inducing agent-responsive gene of Listeria wherein the RNA-inducing agent-responsive gene is hylA and the at least one primer pair comprises isolated nucleic acid molecules having the sequences of SEQ ID NO:854 and SEQ ID NO:1309 or a nucleic acid molecule with at least 90% sequence homology to the primer pair sequences. The composition may also comprise at least one probe wherein the at least one probe has the sequence of SEQ ID NO:1764.

A composition for detecting the presence of Vibrio in a sample may comprise: at least one primer pair having hybridization specificity for priming amplification of an RNA-inducing agent-responsive gene of Vibrio wherein the RNA-inducing agent-responsive gene is a hsp60 gene and the at least one primer pair comprises isolated nucleic acid molecules having the sequences of SEQ ID NO:1765 and SEQ ID NO: 1766, or a nucleic acid molecule with at least 90% sequence homology to the primer pair sequences. The composition may also comprise at least one probe wherein the at least one probe has the sequence of SEQ ID: NO: 1767, SEQ ID NO: 1768 and/or SEQ ID NO:1769.

Some compositions of the disclosure may comprise a duplexed set of primer pairs and probes for detection of one or more microorganisms in a single assay. For example, a composition may comprise, at least two sets of primer pairs, a first primer set comprising a first primer (a first forward primer) and a second primer (a first reverse primer) and a second primer set comprising a first primer (a second forward primer) and a second primer (a second reverse primer), each primer set operable to amplify a different target inducible gene or target inducible gene nucleic acid fragment. The composition may also have a corresponding probe sequence that can hybridize to amplified target nucleic acids of each primer set (a first probe and a second probe). A duplexed primer set may be operable to amplify at least two different target nucleic acid sequences and their corresponding probes are operable to identify at least two different target nucleic acid sequences. Compositions of the disclosure may comprise additional primer pairs (such as three primer pairs, four primer pairs and optionally the same number of corresponding probes as well). In some embodiments, the target genes may be from the same organism, thereby providing a higher degree of confidence in detection. In some embodiments the target genes may be from different organisms, thereby providing the ability to detect multiple organisms in one reaction. Accordingly, in some embodiments, compositions of the disclosure may be operable to detect simultaneously the presence of two or more organisms that may be contaminating a sample, provided the same induction conditions are operable to induce the individual target inducible genes of those organisms.

In some embodiments, the disclosure describes methods of detecting in a sample the presence of a microorganism, comprising: exposing the sample to an RNA-inducing agent for a time to induce a gene responsive to the RNA-inducing agent; detecting presence of an RNA corresponding to the gene responsive to the RNA-inducing agent; and determining presence of the microorganism, wherein the detection of presence of the RNA corresponding to the gene responsive to the RNA-inducing agent in comparison to a control sample is indicative of the presence of the microorganism in the sample. In some embodiments, a method may also comprise the step of culturing the sample in a microorganism enrichment medium to form an enriched sample, and then exposing the enriched sample to the RNA-inducing agent.

Detection steps may be performed by a variety of methods, such as but not limited to, a nucleic acid amplification reaction using primers to amplify target inducible genes or fragments thereof. Detection in some embodiments may be performed by hybridization using probes specific to target sequences in a target inducible gene. Combinations of amplification and hybridization may be used for detection according to some embodiments.

Methods of the disclosure may further comprise steps of sample preparation and may also comprise identification steps (to identify a species/strain/serotype of contaminating organism following the initial detection). In some embodiments, sample preparation may comprise preparing a sample for PCR amplification (prior to hybridizing with a primer pair), comprising for example, but not limited to (1) microbial enrichment, (2) separation of microbial cells from the sample, (3) cell lysis, and (4) nucleic acid extraction (e.g. RNA extraction, total DNA extraction, genomic DNA extraction).

Samples may include without limitation, clinical samples, food/beverage samples, water samples, and environmental sample. Food sample may comprise raw produce, meats as well as a selectively enriched food matrix.

Microbial pathogens that may be detected using methods provided herein may be present in food samples, in samples from the environment including processing equipment, in pharmaceutical preparations, on processing equipment for making or assembling pharmaceutical preparations, or from animal or humans who are potential carriers. A sample for detection of a pathogen may be an uncooked food sample such as uncooked meats, fish, poultry vegetables, unpasteurized milk, foods made from unpasteurized milk, or dairy products, or cooked or processed foods such as hot dogs, deli meats, cheeses, poultry, ice cream, smoked fish, or seafood, for example; an environmental sample for detection of a pathogen may be soil, stream water, sewage, plants, or swabs from food or pharmaceutical processing equipment, or any surface that is involved with food or pharmaceutical processing.

Typically a portion of food or a swabbed or sponged sample is combined with an appropriate liquid, such as water, a buffer solution, or a culture medium such as a selective medium or an enrichment medium. In some embodiments, the food is chopped, macerated, liquefied, diced, or homogenized. In some embodiments, large volumes of sample, for example but not limited to, volumes of 100 mL, 250 mL, or more are processed or a portion of the food or beverage and appropriate liquid are typically combined to form a dilute suspension, for example but not limited to, ratios of about 1:3, 1:5, or 1:10 (w/vol). In some embodiments, a detergent, an emulsifying agent, or both, is added to enhance the solubility of high lipid foods, for example but not limited to butter and certain other dairy products. In certain embodiments, 25 grams of a solid or semi-solid food is combined with 225 mL of a suitable culture media. In some embodiments, 25 mL of a beverage or a liquefied or partially liquefied food is combined with 225 mL of a suitable culture media.

Samples may also be pooled to save on testing costs, e.g., instead of testing 15×25 g samples of food, a composite of 375 g, with 25 g coming from different lots of food will be tested. If any composite is tested positive, then the individual 15 samples are further evaluated. If the composite is negative, then the food testing lab has saved the cost of 15 individual tests.

Solid samples (e.g., 25 g), liquid samples (e.g., 10 ml-100 ml) or environmental samples (i.e., swabbed or sponged samples resuspended in e.g., 10 ml-100 ml) can be pre-enriched for a pathogenic species for a time in pathogen-specific enrichment media, or an equivalent thereof. Alternative enrichment media include Tryptic Soy Broth, Brain Heart Infusion Broth or Fraser Broth, for which ingredients can be found in, for example, “Compendium of Methods for the Microbiological Examination of Foods,” 4th Edition (2001) Downes and Ito, eds. American Public Health Association, or U.S. Food and Drug Administration Bacteriological Analytical Manual (BAM), Media Index on the FDA web site. For example, for enrichment of Listeria species, after 4 hours of incubation, Listeria selective agents are added (see e.g., Example 2 herein). Incubation for selective enrichment is continued for a total of 24 hours enrichment, or may be continued for up to 48 h.

In an example embodiment, a method of the disclosure may comprise, following sample preparation, steps for detecting presence of a microbe by “detecting presence of an RNA corresponding to a gene responsive to an RNA-inducing agent” which may comprise a) hybridizing at least a first pair of PCR primers, comprising a forward primer and a reverse primer, that are operable to bind to and amplify at least one gene responsive to the RNA-inducing agent or a fragment thereof; b) amplifying at least one gene responsive to the RNA-inducing agent or a fragment thereof to form at least one amplified target nucleic acid product; and d) detecting the at least one amplified target polynucleotide sequence product; wherein the detection of the at least one amplified target polynucleotide sequence product is indicative of the presence of the microorganism in the sample. The step of detecting the at least one amplified target polynucleotide sequence product may further comprise using a probe that is operable to hybridize to the at least one amplified target polynucleotide sequence product. Primer and probes used may be labeled. Nucleic acid amplification reactions may be a PCR amplification and may further comprise an end-point determination, and/or maybe quantitative. In some embodiments the quantification maybe a real-time PCR. In some embodiments, the real-time PCR maybe a SYBR® Green Assay and/or a TaqMan® Assay. In some embodiments, the amplification may be by a real-time PCR assay and the probe may be a TAQMAN® probe.

In some embodiments, sample preparation may comprise extracting RNA and detecting may comprise detecting the presence of an induced RNA by a reverse transcriptase RT-PCR assay. A method of the disclosure may further comprise detecting presence of DNA corresponding to the induced gene by an RT-PCR assay. In some embodiments, a method may further comprising subtracting a CT value of RNA and DNA detection from a CT value for DNA detection. A method for identifying an RNA inducing agent-responsive gene may comprise comparing the difference in CT that results from real time reverse transcriptase PCR assays of induced samples vs uninduced samples. A method for identifying the contribution of RNA detection may comprise subtracting CT results from assays with reverse transcriptase present from the CT results from assays without reverse transcriptase present.

In some embodiments, a method for detection of a Salmonella spp. in a sample (enriched sample) may comprise: exposing the sample to an RNA-inducing agent for a time to induce at least one Salmonella gene responsive to the RNA-inducing agent, wherein the Salmonella gene responsive to the RNA-inducing agent may be a cspH, a hilA, a hsp60, a dnaK, a ibpAB, a uspA, and/or a agsA gene; detecting presence of an RNA corresponding to the gene responsive to the RNA-inducing agent; and determining presence of the Salmonella spp, wherein the detection of presence of the RNA corresponding to the Salmonella gene responsive to the RNA-inducing agent in comparison to a control sample (an identical sample known not to have the microorganism; and/or an identical sample not subject to induction) is indicative of the presence of a Salmonella in the sample.

In some embodiments, the steps of detecting presence of an RNA corresponding to the Salmonella gene responsive to the RNA-inducing agent in the method described above may comprise a) hybridizing at least a first pair of PCR primers comprising a forward primer and a reverse primer (e.g., selected for example from a row in Table 1 of the disclosure) that are operable to bind to and amplify an inducible gene or fragment thereof corresponding to at least one of the following Salmonella inducible genes: cspH, hilA, hsp60, dnaK, ibpAB, uspA, and/or agsA; b) amplifying the at least one Salmonella inducible gene or a fragment thereof (corresponding to at least one of the following genes: cspH, hilA, hsp60, dnaK, ibpAB, uspA, and/or agsA) to form at least one amplified target nucleic acid product; and d) detecting the at least one amplified target polynucleotide sequence product; wherein the detection of the at least one amplified target polynucleotide sequence product is indicative of the presence of Salmonella spp. in the sample. The step of detecting the at least one amplified target polynucleotide sequence product may further comprise using a probe that is operable to hybridize to the at least one amplified target polynucleotide sequence product (e.g., probes may be selected for example from a row in Table 1 of the disclosure, a primer pair and probe from the same row describes the probe-primer combination for an assay). Primer and probes used may be labeled. In some embodiments the amplification may be by a real-time PCR assay and the probe may be a TAQMAN® probe.

In some embodiments, a method for detection of a Listeria spp. in a sample (enriched sample) may comprise: exposing the sample to an RNA-inducing agent for a time to induce at least one Listeria gene responsive to the RNA-inducing agent, wherein the Listeria gene responsive to the RNA-inducing agent may be a inlA, a inlB, a inlC, a inlG, a inlJ, a lmo0539, a lmo2158, a lmo0596, a lmo0189, a lmo0880, a lmo1290, a lmo0514, a lmo0670, a bsh, a plcA, a clpE, a cspL, a lmo0699, a lmo0782, a lmo2230, a lmo2522, a opuCA, a cpn60, or a hlyA gene; detecting presence of an RNA corresponding to the Listeria gene responsive to the RNA-inducing agent; and determining presence of the Listeria spp, wherein the detection of presence of the RNA corresponding to the gene responsive to the RNA-inducing agent in comparison to a control sample (an identical sample known not to have the microorganism; and/or an identical sample not subject to induction) is indicative of the presence of a Listeria in the sample.

In some embodiments, the steps of detecting presence of an RNA corresponding to the Listeria gene responsive to the RNA-inducing agent in the method described above may comprise a) hybridizing at least a first pair of PCR primers comprising a forward primer and a reverse primer (e.g., selected for example from a row in Table 2 of the disclosure) that are operable to bind to and amplify an inducible gene or fragment thereof corresponding to at least one of the following genes: a inlA, a inlB, a inlC, a inlG, a inlJ, a lmo0539, a lmo2158, a lmo0596, a lmo0189, a lmo0880, a lmo1290, a lmo0514, a lmo0670, a bsh, a plcA, a clpE, a cspL, a lmo0699, a lmo0782, a lmo2230, a lmo2522, a opuCA, a cpn60, and/or a hlyA; b) amplifying at least one inducible gene or fragment thereof to form at least one amplified target nucleic acid product; and d) detecting the at least one amplified target polynucleotide sequence product; wherein the detection of the at least one amplified target polynucleotide sequence product is indicative of the presence of Listeria spp. in the sample. The step of detecting the at least one amplified target polynucleotide sequence product may further comprise using a probe that is operable to hybridize to the at least one amplified target polynucleotide sequence product (e.g., a probe may be selected for example from a row in Table 2 of the disclosure, a primer pair and probe from the same row describes the probe-primer combination for an assay). Primer and probes used may be labeled. In some embodiments the amplification may be by a real-time PCR assay and the probe may be a TAQMAN® probe.

In some embodiments, a method for detection of a Vibrio spp. in a sample (enriched sample) may comprise: exposing the sample to an RNA-inducing agent for a time to induce at least one Vibrio gene responsive to the RNA-inducing agent, wherein an example Vibrio gene responsive to the RNA-inducing agent may be a Vibrio hsp60 gene; detecting presence of an RNA corresponding to the Vibrio gene responsive to the RNA-inducing agent; and determining presence of the Vibrio spp, wherein the detection of presence of the RNA corresponding to a Vibrio gene responsive to an RNA-inducing agent in comparison to a control sample (an identical sample known not to have the microorganism; and/or an identical sample not subject to induction) is indicative of the presence of a Vibrio in the sample.

In some embodiments, the steps of detecting presence of an RNA corresponding to a Vibrio gene responsive to the RNA-inducing agent in the method described above may comprise a) hybridizing at least a first pair of PCR primers comprising a forward primer and a reverse primer (e.g., selected for example from a row in Table 3 of the disclosure) that are operable to bind to and amplify an inducible gene or fragment thereof (for e.g., a Vibrio hsp60 gene); b) amplifying at least one inducible gene or fragment thereof (for e.g., a Vibrio hsp60 gene) to form at least one amplified target nucleic acid product; and d) detecting the at least one amplified target polynucleotide sequence product; wherein the detection of the at least one amplified target polynucleotide sequence product is indicative of the presence of Vibrio spp. in the sample. The step of detecting the at least one amplified target polynucleotide sequence product may further comprise using a probe that is operable to hybridize to the at least one amplified target polynucleotide sequence product. A probe may be selected, for example, from a row in Table 3 of the disclosure, a primer pair and probe from the same row describes the probe-primer combination for an assay. Table 3 describes three probe sequences that are all operable to function with the Vibrio specific primer pair. Primer and probes used may be labeled. In some embodiments the amplification may be by a real-time PCR assay and the probe may be a TAQMAN® probe.

Methods of the disclosure may include assays such as polymerase chain reactions, wherein hybridizing and amplifying of said first pair of polynucleotide primers occurs in a first vessel and said hybridizing and amplifying of said second pair of polynucleotide primers occurs in a second vessel, or hybridizing and amplifying of said first pair of polynucleotide primers and said hybridizing and amplifying of said second pair of polynucleotide primers occurs in a single vessel. The detection may be a real-time assay and the real-time assay may be a SYBR® Green dye assay or a TaqMan® assay.

Methods of the disclosure, in various embodiments, may comprise providing at least a first probe. Some embodiments may comprise providing at least two probes, a first probe and a second probe the probes, wherein the first and second probes are different from each other, the first probe operable to identify the first amplified target polynucleotide sequence and the second probe operable to identify the second amplified target nucleotide sequence, the first probe further comprises a first label and said second probe further comprises a second label, wherein both labels are selected from a dye, a radioactive isotope, a chemiluminescent label, and an enzyme, the dye comprises a fluorescein dye, a rhodamine dye, or a cyanine dye, the dye is a fluorescein dye and first probe is labeled with FAM™ dye and said second probe is labeled with VIC® dye; and hybridizing the first and second probes to the PCR amplified fragments to detect the presence of the first amplified target polynucleotide sequence and the second amplified target polynucleotide sequence from the sample.

Methods of the disclosure comprising using inducible RNA targets for detection of microbes may also be used for assessment of viability of microbes. Dead microbes will not respond to induction. Inducible RNA targets are synthesized by living cells upon sensing a particular stimulus, such as heat, cold, acidic pH or chemical reagents. Accordingly, the disclosure provides methods of detecting viable microbes in a sample. Use of inducible RNA targets overcomes a major disadvantage of using DNA targets which detect both live and dead bacteria. If only dead bacteria are present in a sample, a method using DNA target detection will show a false positive result by amplifying and picking up dead DNA targets that are incapable of causing disease. An additional advantage over traditional culture confirmation method is the ability to detect viable but non-culturable bacteria (VBNC) organisms that respond to induction.

Use of inducible RNA targets provided by embodiments herein for detection of pathogens has other advantages over traditional DNA targets typically used in real-time PCR based detection. For example, for RNA inducible targets, an increased copy number of the target is present upon induction. When DNA is transcribed into RNA, many copies of the target gene are generated and a greater copy number translates into more robust and potentially earlier detection of a pathogen.

Traditional culture methods as well as rapid PCR-based methods rely on an initial pre-enrichment in suitable media to revive stressed organisms in various matrices. The length of the pre-enrichment time depends on the sensitivity and limit of detection of the end assay method. Signal amplification using RNA targets, such as inducible RNA targets, allows for earlier detection of target.

The present disclosure also describes workflow methods using inducible RNA targets in assays for detection of various bacteria. A fast workflow is provided based on inducible transcription response in bacteria; the workflow includes a shortened enrichment step, a rapid induction step, an automated sample preparation step, and specific detection of induced target using reverse transcriptase RT-PCR. Example workflows provided herein allowed for detection of 1-5 cfu of Salmonella in less then 8 hr and Listeria in less than 12 hr in food samples, thereby greatly reducing the time to testing, which is very important for industries such as the food industry, where valuable shelf life can be increased, if the testing time for food safety testing is reduces by the workflow methods provided herein.

The entire volume of the enriched and induced culture, or a portion thereof, may be concentrated and processed for detection of the bacterial pathogen, for example, a one mL aliquot may be taken from 250 mL of enriched and induced culture. The medium may be clarified by filtration or low speed centrifugation prior to or after concentrating. Harvested samples are lysed using, for example, the RiboPure™ RNA extraction kit (Ambion, Austin Tex.), the PrepSEQ™ Nucleic Acid Extraction Kit (Applied Biosystems) or the PrepSEQ™ RapidSpin Kit (Applied Biosystems) or any other effective lysis system that preserves nucleic acid integrity. The lysate can be amplified directly or the nucleic acid can be extracted and amplified. Amplification products may be detected, directly or indirectly, and the presence or absence of a microbe in the test sample can be determined. Sample preparation methods used are efficient in removal of PCR inhibitors.

High quality DNA and RNA can be prepared by manual low throughput methods or by automated high throughput methods, depending on the number of samples being tested. An integrated workflow for automated high-throughput sample preparation may include enrichment for the test bacterial pathogen, lysis, binding of nucleic acids to magnetic particles, magnetic separation, followed by washings and elution of DNA and/or RNA in a PCR compatible solution. An integrated workflow for manual low-throughput sample preparation may include enrichment for the test bacterial pathogen, centrifugation to pellet bacteria, resuspension of bacteria in lysis buffer, followed by amplification using primers for RNA-inducible target specific PCR as provided herein. The integrated system may also include lyophilized reagents for the assay and data analyses software.

Embodiments of detecting DNA, RNA and/or a surrogate thereof, in a lysate or extracted nucleic acid sample includes detection means using emission by an emitter that is representative of the RNA or DNA in the test sample.

In some embodiments, a lysate or extracted nucleic acid sample is mixed with a composition comprising reverse transcriptase to form a reverse transcriptase reaction mixture. A reverse transcription reaction provides a surrogate of the RNA that can be detectable. Any reverse transcriptase known to those of ordinary skill in the art can be used such as, for example, MMLV-RT (murine maloney leukemia virus-reverse transcriptase), avian myelogenous virus reverse transcriptase (AMV-RT), human immunodeficiency virus (HIV)-RT and the Tth DNA polymerase which has reverse transcriptase activity if Mn⁺⁺ is provided.

A positive control for detection of RNA or DNA can be a non-homologous RNA random sequence such as XENO™ RNA (Applied Biosystems, Foster City, Calif.). A control for qPCR can be a β-actin probe/primer set, also available from Applied Biosystems, for example. The positive control can be mixed with the sample.

As used herein, “amplification” or “amplify” and the like refers to the production of multiple copies of the target nucleic acid, a surrogate of a target nucleic acid, or a portion thereof. Amplification can encompass a variety of chemical and enzymatic processes such as a polymerase chain reaction (PCR), a strand displacement amplification reaction, a transcription mediated amplification reaction, or a nucleic acid sequence-based amplification reaction, for example. Following at least one amplification cycle, the amplification products can be detected or can be separated from at least one other component of the amplification mixture based on their molecular weight or length or mobility prior to detection.

PCR includes introducing a molar excess of two or more extendable oligonucleotide primers to a reaction mixture where the primers hybridize to opposite strands of a DNA, RNA or RNA surrogate. The reaction mixture is subjected to a program of thermal cycling in the presence of a DNA polymerase, resulting in the amplification of the DNA or RNA surrogate sequence flanked by the primers. Reverse transcriptase PCR is a PCR reaction that uses an RNA template and a reverse transcriptase, or a polypeptide having reverse transcriptase activity, to first generate a single stranded DNA molecule prior to the multiple cycles of DNA-dependent DNA polymerase primer elongation as cited above. Methods for a wide variety of PCR applications are widely known in the art, and described in many sources, for example, Ausubel et al. (eds.), Current Protocols in Molecular Biology, Section 15, John Wiley & Sons, Inc., New York (1994).

The present disclosure describes several primers. However, in light of the present specifications, one of skill in the art may design additional primer pairs that are specific to other inducible genes in other organisms. These additional inducible genes and primers are within the scope of the present disclosure. Criteria for designing sequence-specific primers in light of this specification are well known to persons of ordinary skill in the art. Detailed descriptions of primer design that provide for sequence-specific annealing can be found, among other places, in Diffenbach and Dveksler, PCR Primer, A Laboratory Manual, Cold Spring Harbor Press, 1995, and Kwok et al. (Nucl. Acid Res. 18:999-1005, 1990). The sequence-specific portions of the primers are of sufficient length to permit specific annealing to complementary sequences, as appropriate. A primer does not need to have 100% complementarity with a primer-specific portion for primer extension to occur. Further, a primer can be detectably labeled such that the label is detected by spectroscopy. A primer pair is sometimes said to consist of a “forward primer” and a “reverse primer,” indicating that they are initiating nucleic acid polymerization in opposing directions from different strands of a duplex template.

In some embodiments, a primer as set forth herein can comprise a universal priming sequence. The term “universal primer” refers to a primer comprising a universal sequence that is able to hybridize to all, or essentially all, potential target sequences in a multiplexed reaction. The term “semi-universal primer” refers to a primer that is capable of hybridizing with more than one (e.g., a subset), but not all, of the potential target sequences in a multiplexed reaction. The terms “universal sequence,” “universal priming sequence” or “universal primer sequence” or the like refer to a sequence contained in a plurality of primers, where the universal priming sequence that is found in a target is complementary to a universal primer.

“Hybridization” refers to a process in which single-stranded nucleic acids with complementary or near-complementary base sequences interact to form hydrogen-bonded complexes called hybrids. Hybridization reactions are sensitive and selective. In vitro, the specificity of hybridization (i.e., stringency) is controlled by the concentrations of salt or formamide in prehybridization and hybridization solutions, for example, and by the hybridization temperature; such procedures are well known in the art. In particular, stringency is increased by reducing the concentration of salt, increasing the concentration of formamide, or raising the hybridization temperature. For example, high stringency conditions could occur at about 50% formamide at 37° C. to 42° C. Reduced stringency conditions could occur at about 35% to 25% formamide at 30° C. to 35° C. Examples of stringency conditions for hybridization are provided in Sambrook, J., 1989, Molecular Cloning A Laboratory Manual, Cold Spring Harbor Laboratory Press, Cold Spring Harbor, N.Y. The temperature for hybridization is about 5-10° C. less than the melting temperature (Tm) of the hybrid.

As an example of primer selection, primers can be selected by the use of any of various software programs available and known in the art for developing amplification and/or multiplex systems. Exemplary programs include, PRIMER EXPRESS® software (Applied Biosystems, Foster City, Calif.) and Primer3 software (Rozen S et al. (2000), “Primer3 on the WWW for general users and for biologist programmers,” Krawetz S et al. (eds) Bioinformatics Methods and Protocols: Methods in Molecular Biology. Humana Press, Totowa, N.J., pp 365-386). In the example of the use of software programs, sequence information can be imported into the software. The software then uses various algorithms to select primers that best meet the user's specifications.

Primer and probe sequences having at least 90% homology to those of Tables 1-3 are embodiments herein. “Homology,” as known by one of ordinary skill in the art, is the degree of sequence relatedness between nucleotide sequences as determined by matching the order and identity of nucleotides between the sequences. In one embodiment, the primer or probe sequences provided herein have 100% homology, at least 98% homology, at least 95% homology, at least 92% homology or at least 90% homology to their intended hybridization target. Computer methods for determining homology are designed to identify the greatest degree of matching of nucleotide sequences, for example, BLASTN (Altschul, S. F., et al. (1990) J. Mol. Biol. 215:403-410).

In certain embodiments, single-stranded amplification products can be generated by methods including, without limitation, asymmetric PCR, asymmetric reamplification, nuclease digestion, and chemical denaturation. For example, single-stranded sequences can be generated by combining at least one first primer or at least one second primer from a primer set, but not both, in an amplification reaction mixture, or by transcription, for example, when a promoter-primer is used in a first amplification mixture, a second amplification mixture, or both.

The term “polymerase,” as used herein, refers to a polypeptide that is able to catalyze the addition of nucleotides or analogs thereof to a nucleic acid in a template dependent manner, for example, the addition of deoxyribonucleotides to the 3′-end of a primer that is annealed to a nucleic acid template during a primer extension reaction. Nucleic acid polymerases can be thermostable or thermally degradable. Suitable thermostable polymerases include, but are not limited to, polymerases isolated from Thermus aquaticus, Thermus thermophilus, Pyrococcus woesei, Pyrococcus furiosus, Thermococcus litoralis, and Thermotoga maritima. Suitable thermodegradable polymersases include, but are not limited to, E. coli DNA polymerase I, the Klenow fragment of E. coli DNA polymerase I, T4 DNA polymerase, T5 DNA polymerase, T7 DNA polymerase, and others. Examples of other polymerizing enzymes that can be used in the methods described herein include but are not limited to T7, T3, SP6 RNA polymerases; and AMV, M-MLV and HIV reverse transcriptases.

Commercially available polymerases include, but are not limited to AMBION′S SUPERTAQ®, TAQFS®, AMPLITAQ® CS (Applied Biosystems), AMPLITAQ® FS (Applied Biosystems), KENTAQ1® (AB Peptide, St. Louis, Mo.), TAQUENASE® (Scien Tech Corp., St. Louis, Mo.), THERMOSEQUENASE® (Amersham), Bst polymerase, READER™ Taq DNA polymerase, VENT® DNA polymerase, VENT_{R}® DNA Polymerase, VENT_{R}® (exo⁻) polymerase and DEEPVENT® DNA polymerase, (all VENT® polymerases can be obtained from New England Biolabs), PFUTurbo™ DNA polymerase (Stratagene), Pwo polymerase, Tth DNA polymerase, KlenTaq-1 polymerase, SEQUENASE™ 1.0 DNA polymerase (Amersham Biosciences), SEQUENASE™ 2.0 DNA polymerase (United States Biochemicals), and an enzymatically active mutant and variant thereof.

Descriptions of DNA polymerases can be found in, among other places, Lehninger Principles of Biochemistry, 3d ed., Nelson and Cox, Worth Publishing, New York, N.Y., 2000, particularly Chapters 26 and 29; Twyman, Advanced Molecular Biology: A Concise Reference, Bios Scientific Publishers, New York, N.Y., 1999; Ausubel et al., Current Protocols in Molecular Biology, John Wiley & Sons, Inc., including supplements through May 2005 (hereinafter “Ausubel et al.”); Lin and Jaysena, J. Mol. Biol. 271:100-11, 1997; Pavlov et al., Trends in Biotechnol. 22:253-60, 2004; and Enzymatic Resource Guide: Polymerases, 1998, Promega, Madison, Wis.

In various detection embodiments, amplification is optionally followed by additional steps, for example, but not limited to, labeling, sequencing, purification, isolation, hybridization, size resolution, expression, detecting and/or cloning. In certain embodiments, one or both PCR primers can comprise a label, such as, for example, a fluorophore. A label can facilitate detection of an amplification product comprising a labeled PCR primer. In various detection embodiments, following the PCR, biotinylated strands can be captured, separated, and detected.

The term “multiplex assays” refers to PCR reactions that use more than two primers in a single reaction and at the same time so that more than one different amplified product is produced and detected. For example, more than two pair of amplification primers are contacted at the same time and/or in the same solution. Several target RNAs or DNAs can be detected simultaneously using multiplex assays. A multiplex reaction can also include a multiplicity of singleplex PCR reactions run in parallel, e.g., the TAQMAN® Low Density Array (TLDA). Sample preparation processes described herein have been demonstrated to be compatible with multiplex assays.

As used herein, “real-time PCR” refers to the detection and quantitation of a DNA, a RNA or a surrogate thereof in a sample. In some embodiments, the amplified segment or “amplicon” can be detected using a 5′-nuclease assay, particularly the TAQMAN® assay as described by e.g., Holland et al. (Proc. Natl. Acad. Sci. USA 88:7276-7280, 1991); and Heid et al. (Genome Research 6:986-994, 1996). For use herein, a TAQMAN® nucleotide sequence to which a TAQMAN® probe binds can be designed into the primer portion, or known to be present in a RNA or a DNA of a sample.

“Tₘ” refers to the melting temperature (temperature at which 50% of the oligonucleotide is a duplex) of an oligonucleotide determined experimentally or calculated using the nearest-neighbor thermodynamic values of SantaLucia J. et al. (Biochemistry 35:3555-62, 1996) for DNA or Freier et al. (Proc. Natl. Acad. Sci. USA 83:9373-9377, 1986) for RNA. In general, the Tₘ of the TAQMAN® probe is about 10 degrees above the Tₘ of amplification primer pairs. Amplification primer sequences and double dye-labeled TAQMAN® probe sequences can be designed using PRIMER EXPRESS™ (Version 1.0, Applied Biosystems, Foster City, Calif.) or mFOLD™ software (now UNIFold™) (IDT, San Jose, Calif.).

When a TAQMAN® probe is hybridized to DNA, RNA or a surrogate thereof, the 5′-exonuclease activity of a thermostable DNA-dependent DNA polymerase such as SUPERTAQ® (a Taq polymerase from Thermus aquaticus, Ambion, Austin, Tex.) digests the hybridized TAQMAN® probe during the elongation cycle, separating the fluor from the quencher. The reporter fluor dye is then free from the quenching effect of the quencher moiety resulting in a decrease in FRET and an increase in emission of fluorescence from the fluorescent reporter dye. One molecule of reporter dye is generated for each new molecule synthesized, and detection of the free reporter dye provides the basis for quantitative interpretation of the data. In real-time PCR, the amount of fluorescent signal is monitored with each cycle of PCR. Once the signal reaches a detectable level, it has reached the “threshold or cycle threshold (Ct).” A fluorogenic PCR signal of a sample can be considered to be above background if its Ct value is at least 1 cycle less than that of a no-template control sample. The term “Ct” represents the PCR cycle number when the signal is first recorded as statistically significant. Thus, the lower the Ct value, the greater the concentration of nucleic acid target. In the TAQMAN® assay, typically each cycle almost doubles the amount of PCR product and therefore, the fluorescent signal should double if there is no inhibition of the reaction and the reaction was nearly 100% efficient with purified nucleic acid. Certain systems such as the ABI 7500, 7500FAST, 7700 and 7900HT Sequence Detection Systems (Applied Biosystems, Foster City, Calif.) conduct monitoring during each thermal cycle at a pre-determined or user-defined point.

Detection method embodiments using a TAQMAN® probe sequence comprise combining the stopped mixture or the reverse transcribed mixture with PCR reagents, including a primer set having a forward primer and a reverse primer, a DNA polymerase, and a fluorescent detector oligonucleotide TAQMAN® probe, as well as dNTP's and a salt, to form an amplification reaction mixture; subjecting the amplification reaction mixture to successive cycles of amplification to generate a fluorescent signal from the detector probe; and quantitating the nucleic acid presence based on the fluorescent signal cycle threshold of the amplification reaction.

Protocols and reagents for means of carrying out further 5′-nuclease assays are well known to one of skill in the art, and are described in various sources. For example, 5′-nuclease reactions and probes are described in U.S. Pat. Nos. 6,214,979 issued Apr. 10, 2001; 5,804,375 issued Sep. 8, 1998; 5,487,972 issued Jan. 30, 1996; and 5,210,015 issued May 11, 1993, all to Gelfand et al.

In various embodiments, a detection method can utilize any probe that can detect a nucleic acid sequence. In some configurations, a detection probe can be, for example, a TAQMAN® probe described supra, a stem-loop molecular beacon, a stemless or linear beacon, a PNA MOLECULAR BEACON™, a linear PNA beacon, non-FRET probes, SUNRISE®/AMPLIFLUOR® probes, stem-loop and duplex SCORPION™ probes, bulge loop probes, pseudo knot probes, cyclicons, MGB ECLIPSE™ probe, a probe complementary to a ZIPCODE™ sequence, hairpin probes, peptide nucleic acid (PNA) light-up probes, self-assembled nanoparticle probes, and ferrocene-modified probes as known by one of ordinary skill in the art. A detection probe having a sequence complementary to a detection probe hybridization sequence, such as a ZIPCODE™ sequence, a fluorophore and a mobility modifier can be, for example, a ZIPCHUTE™ probe supplied commercially by Applied Biosystems (Foster City, Calif.).

A “label” or “reporter,” as used herein, refers to a moiety or property that allows the detection of that with which it is associated and, generally, has emission spectra at between and including 300 nm to 750 nm. In certain embodiments, the emission spectra is at less than about 499 nm such as for blue emitters such as certain Alexa Fluor emitters, Cascade Blue, and Pacific Blue; at 500 nm to 549 nm emitters such as for green emitters such as certain Alexa Fluor emitters, BODIPY FL, fluorescein (FITC), CYANINE™ 2 dye, Catskill Green, 5-FAM™ dye, 6-FAM™ dye, succinimidyl ester, JOE™ dye, MFP488, the Oregon Green emitters and TET™ dye; at 550 nm to 584 nm emitters such as yellow emitters such as certain Alexa Fluor emitters, CYANINE™ 3 dye, HEX™ dye, NED™ dye, R-Phycoerythrin (R-PE), 5-TAMRA™ dye, TRITC (Rhodamine), and VIC® dye; at 585 nm to 615 nm emitters such as orange emitters such as certain Alexa Fluor emitters, CYANINE™ 3.5 dye, Lissamine Rhodamine, ROX™ dye, and R-Phycoerythrin-TEXAS RED® dye; and at 616 nm to 700 nm emitters such as red emitters such as certain Alexa Fluor emitters, CYANINE™ 5 dye, Quantum Red, Rodamine Red-X, and TEXAS RED® dye.

The label can be attached covalently or non-covalently to a DNA product, to a RNA product, or to a surrogate thereof such as an amplicon thereof. Commonly used labels include dyes that are negatively charged, such as dyes of the fluorescein family including, e.g. FAM™ dye, HEX™ dye, TET™ dye, JOE™ dye, NAN and ZOE; or dyes that are neutral in charge, such as dyes of the rhodamine family including, e.g., TEXAS RED® dye, ROX™ dye, R110, R6G, and TAMRA™ dye; or dyes that are positively charged, such as dyes of the CYANINE™ family including e.g., Cy™2 dye, Cy™3 dye, Cy™5 dye, Cy™5.5 dye and Cy™7 dye. FAM™ dye, HEX™ dye, TET™ dye, JOE™ dye, NAN, ZOE, ROX™ dye, R110, R6G, and TAMRA™ dyes are available from, e.g., Applied Biosystems (Foster City, Calif.) or Perkin-Elmer, Inc. (Wellesley, Mass.); TEXAS RED® dye is available from, e.g., Molecular Probes, Inc. (Eugene, Oreg.); and Cy™2 dye, Cy™3 dye, Cy™5 dye, Cy™5.5 dye and Cy™7 dye, and are available from, e.g., Amersham Biosciences Corp. (Piscataway, N.J.). In certain amplification embodiments, the fluorescer molecule is a fluorescein dye and the quencher molecule is a rhodamine dye.

A label or reporter can comprise both a fluorophore and a fluorescence quencher. The fluorescence quencher can be a fluorescent fluorescence quencher, such as the fluorophore TAMRA™ dye, or a non-fluorescent fluorescence quencher (NFQ), for example, a combined NFQ-minor groove binder (MGB) such as an MGB ECLIPSE™ minor groove binder supplied by Epoch Biosciences (Bothell, Wash.) and used with TAQMAN™ probes (Applied Biosystems, Foster City, Calif.). The fluorophore can be any fluorophore that can be attached to a nucleic acid, such as, for example, FAM™ dye, HEX™ dye, TET™ dye, JOE™ dye, NAN, ZOE, TEXAS RED® dye, ROX™ dye, R110, R6G, TAMRA™ dye, Cy™2 dye, Cy™3 dye, Cy™5 dye, Cy™5.5 dye and Cy™7 dye as cited above as well as VIC® dye, NED™ dye, LIZ® dye, ALEXA, Cy™9 dye, and dR6G.

Further examples of labels include black hole quenchers (BHQ) (Biosearch), Iowa Black (IDT), QSY quencher (Molecular Probes), and Dabsyl and Dabcel sulfonate/carboxylate Quenchers (Epoch).

Labels can also comprise sulfonate derivatives of fluorescein dyes, phosphoramidite forms of fluorescein, phosphoramidite forms of CY™5 dye (available for example from Amersham), and intercalating labels such as ethidium bromide, SYBR™ Green I dye and PICOGREEN™ dye (Molecular Probes). Generally, an intercalating label is a molecule that reversibly inserts between two other molecules (or groups) such as between the bases of DNA.

In various embodiments, qPCR reactions can include master mixes such as the TAQMAN® Gene Expression Master Mix, TAQMAN® Universal PCR Master Mix, TAQMAN® Fast Universal PCR Master Mix, Power SYBR® Green PCR Master Mix, Fast SYBR® Green Master Mix, TAQMAN® RNA-to-C_{T}™ 1-Step Kit, and the Power SYBR® Green RNA-to-C_{T}™ 1-Step Kit, for example, all from Applied Biosystems.

In various embodiments, detection of emission such as fluorescence can be by any method known to skilled artisans, and can include, for example, real time detection for PCR or end point detection. Detection of fluorescence, for example, can be qualitative or quantitative. Quantitative results can be obtained, for example, with the aid of a fluorimeter, for example a fluorimeter as part of an integrated nucleic acid analysis system, such as, for example, an Applied Biosystems ABI PRISM™ 7900HT Sequence Detection System. Furthermore, quantitative results can be obtained in some configurations using a real-time PCR analysis. Some non-limiting examples of protocols for conducting fluorogenic assays such as TAQMAN® assays, including analytical methods for performing quantitative assays, can be found in publications such as, for example, “SNPLEX™ Genotyping System 48-plex”, Applied Biosystems, 2004; “User Bulletin #2 ABI PRISM™ 7700 Sequence Detection System,” Applied Biosystems 2001; “User Bulletin #5 ABI PRISM™ 7700 Sequence Detection System,” Applied Biosystems, 2001; and “Essentials of Real Time PCR,” Applied Biosystems (Foster City, Calif.). Fluorogenic PCR assays used in some configurations of the present teachings can be performed using an automated system, such as, for example, an ABI 7700 Sequence Detection System (Applied Biosystems).

For real time PCR, a passive reference dye, ROX™ dye, can be included in PCR reactions to provide an internal reference to which the reporter-dye signal can be normalized during data analysis. Normalization can be accomplished using Applied Biosystems' Sequence Detection System (SDS) software.

In general for the studies herein, the TAQMAN® probes were labeled with FAM™ dye, the TAQMAN® probe for the Internal Positive Control was labeled with VIC® dye and a Baseline Control was detected using ROX® Dye. Data analyses were carried out using the RAPIDFINDER™ Express Software (Applied Biosystems) and results are provided in an easy-to-read format with present/absent calls.

In some embodiments, detection can be achieved using microarrays or bead arrays and related software, such as the Applied Biosystems Array System with the Applied Biosystems 1700 Chemiluminescent Microarray Analyzer, and other commercially available array systems available from Affymetrix, Agilent, and Illumina, among others (see also Gerry et al., J. Mol. Biol. 292:251-62, 1999; De Bellis et al., Minerva Biotec 14:247-52, 2002; and Stears et al., Nat. Med. 9:140-45, including supplements, 2003).

A “kit,” as used herein, refers to a combination of at least some items for performing a reverse transcriptase RT-PCR assay for detection of inducible RNA targets of microbes, such as but not limited to, bacterial pathogens. Embodiments of kits may comprise at least one or more of the following reagents: at least one set of primers specific for detecting an inducible RNA target, at least one probe (e.g., a TAQMAN® probe) specific for detection of said inducible RNA target, internal positive control DNA to monitor presence of PCR inhibitors from various food and environmental sources, a baseline control, reagents for sample collection, reagents for isolating nucleic acid such as magnetic beads, spin columns, columns, particles, filters, lysis buffers, protease, reverse transcriptase, a reverse transcriptase buffer, a DNA polymerase or an enzymatically active mutant or variant thereof, a DNA polymerase buffer, deoxyribonucleotides dATP, dCTP, dGTP, or dTTP. In some kit embodiments, an enzyme comprising reverse transcriptase activity and thermostable DNA-dependent DNA polymerase activity are the same enzyme, e.g., Thermus sp. ZO5 polymerase or Thermus thermophilus polymerase. In certain kit embodiments, amplification primers are attached to a solid support such as a microarray.

The container means of the kits will generally include at least one vial, test tube, flask, bottle, syringe or other packaging means, into which a component can be placed, and in some embodiments, suitably aliquoted. Where more than one component is included in the kit (they can be packaged together), the kit also will generally contain at least one second, third or other additional container into which the additional components can be separately placed. However, various combinations of components can be packaged in a container means. The kits of the present teachings also will typically include reagent containers in close confinement for commercial sale. Such containers can include injection or blow-molded plastic containers into which the desired container means are retained.

When the components of the kit are provided in one and/or more liquid solutions, the liquid solution comprises an aqueous solution that can be a sterile aqueous solution.

In certain embodiments, at least one kit component is lyophilized and provided as dried powder(s). For example, primers and TAQMAN® probes may be lyophilized. When reagents and/or components are provided as a dry powder, the powder can be reconstituted by the addition of a suitable solvent. In certain embodiments, the solvent is provided in another container means. Kits can also comprise an additional container means for containing a sterile, pharmaceutically acceptable buffer and/or other diluent.

A kit can also include instructions for employing the kit components as well as the use of any other reagent not included in the kit. Instructions can include variations that can be implemented.

An exemplary kit may comprise one or more compositions for detecting one or more Salmonella species and may comprise at least one primer pair having hybridization specificity for amplifying an inducible gene specific to Salmonella species, or for amplifying a fragment of an inducible gene specific to Salmonella species. In some embodiments, the kit may further comprise at least one probe sequence operable to bind to an inducible gene specific to Salmonella species or a fragment thereof that may be amplified by the primer pair. A probe may be labeled for easier detection. In some embodiments, probe and primers of kits may comprise sequences selected from SEQ ID NO: 1-SEQ ID NO: 1769, including the specific primer pair combinations (and corresponding probes) described in sections above for detection of different inducible genes (also see probe primer combinations in Tables 1-3).

In one example, according to some embodiments of this disclosure a kit for detecting Salmonella may comprise: a primer pair comprising at least one Salmonella-specific primer pair having hybridization specificity for priming amplification of an RNA-inducing agent-responsive gene of Salmonella wherein the RNA-inducing agent-responsive gene is a cspH, a hilA, a hsp60, a dnaK, a ibpAB, a uspA, and/or a agsA gene.

Another exemplary kit may comprise one or more compositions for detecting one or more Listeria species and may comprise at least one primer pair having hybridization specificity for amplifying an inducible gene specific to Listeria species, or for amplifying a fragment of an inducible gene specific to Listeria species. In some embodiments, the kit may further comprise at least one probe sequence operable to bind to an inducible gene specific to Listeria species or a fragment thereof that may be amplified by the primer pair. A probe may be labeled for easier detection.

In one example, according to some embodiments of this disclosure a kit for detecting Listeria may comprise: a primer pair comprising at least one Listeria-specific primer pair having hybridization specificity for priming amplification of an RNA-inducing agent-responsive gene of Listeria wherein the RNA-inducing agent-responsive gene is a inlA, a inlB, a inlC, a inlG, a inlJ, a lmo0539, a lmo2158, a lmo0596, a lmo0189, a lmo0880, a lmo1290, a lmo0514, a lmo0670, a bsh, a plcA, a clpE, a cspL, a lmo0699, a lmo0782, a lmo2230, a lmo2522, a opuCA, a cpn60, and/or a hlyA gene.

Yet another exemplary kit may comprise one or more compositions for detecting one or more Vibrio species and may comprise at least one primer pair having hybridization specificity for amplifying an inducible gene specific to Vibrio species, or for amplifying a fragment of an inducible gene specific to Vibrio species. In some embodiments, the kit may further comprise at least one probe sequence operable to bind to an inducible gene specific to Vibrio species or a fragment thereof that may be amplified by the primer pair. A probe may be labeled for easier detection.

In one example, according to some embodiments of this disclosure a kit for detecting Vibrio may comprise: a primer pair comprising at least one Vibrio-specific primer pair having hybridization specificity for priming amplification of an RNA-inducing agent-responsive gene of Vibrio wherein the RNA-inducing agent-responsive gene is a hsp60 gene.

Aspects of the present teachings can be further understood in light of the following examples, which should not be construed as limiting the scope of the present teachings in any way.

### Example 1

### Inducible RNA Targets for Early Detection of Salmonella

The present example describes identification and evaluation of inducible RNA targets for detection of the pathogenic microbe Salmonella enterica using real-time RT-PCR.

Materials and Methods; Bacteria and Growth Conditions: Bacteria were routinely cultured on Brain Heart Infusion (BHI) Agar plates and in BHI broth at 37° C. overnight (Teknova, Hollister, Calif.). For spiking food samples, 25 g or 25 ml of the food sample was transferred into stomacher bags. Bacteria were spiked in the 1-100 cfu range. Samples were enriched according to standard procedures as described in the Bacteriological Analytical Manual (U.S. Food and Drug Administration, Bacteriological Analytical Manual, Chapter 5, Salmonella; see www.fda.gov). Enrichment media (225 ml) was added to each sample; for enrichment of Salmonella, Buffered Peptone Water or Brain Heart Infusion Broth was used (Teknova, Hollister, Calif.). Samples were enriched at 37° C. for a maximum of 16 hours.

Inducing Conditions: For Salmonella, samples were induced by heating at 45° C. for 15 minutes. A control (uninduced) sample was maintained at 37° C.

Sample Preparation: At the end of the induction period, tubes were placed in an ice bucket and cells were harvested by centrifugation at 16,000 rpm, 4° C., 3 minutes. Supernatants were quickly removed, and bacterial pellets were quick frozen in a dry-ice ethanol bath before proceeding for RNA extraction.

RNA was extracted using Nucleic Acid Extraction kits, e.g. RiboPure™ RNA extraction kit (Ambion, Austin Tex.). Briefly, a volume of 1 ml-20 ml of sample was centrifuged to harvest the bacteria at 4° C. The pellets were processed according to the extraction kit protocol.

Real-time PCR: Real-time PCR was performed on the Applied Biosystems 7500 Real-time PCR System (Applied Biosystems, Foster City Calif.). For real-time RT-PCR, ARRAYSCRIPT™ reverse transcriptase (Ambion, Austin Tex.) was included in the reactions. The Environmental Master Mix v2 (Applied Biosystems) was used for all reactions. PCR was performed under standard cycling conditions with a reverse transcription step (30 minutes at 42° C.; 10 minutes at 95° C.; 40 cycles of 15 seconds at 95° C., 1 minute at 60° C.).

Primers and Probes: TAQMAN® real-time PCR assays, which include primers and probes for each assay, were designed using a rigorous bioinformatics assay design pipeline against candidate inducible targets in Salmonella enterica (Table 1). Full length genes representing each of the candidate targets were obtained from the annotated, publicly available genome sequences of Salmonella dublin or Salmonella typhi. These gene sequences were used to query the GenBank database and obtain all other publicly available, homologous Salmonella sequences. Assays were designed to match all of the related Salmonella sequences, while not matching any other sequences in the microbial subset of GenBank. The probes were labeled with a FAM™ dye to enable detection by real-time PCR.

Assays designed for Salmonella are shown in Table 1, which has a column designated as “Gene” indicating the name of the inducible target gene; a column designated as “Assay” which indicates an assay number given to the combination of “Forward Primer” and “Reverse Primer” and “Probe” in the same row as the assay number. For example, one Salmonella assay, for detecting the inducible target gene agsA, may comprise assay agsA.0 and use the primer of SEQ ID NO: 1 (forward primer), the primer of SEQ ID NO: 134 (reverse primer) and the probe of SEQ ID NO: 267. In another example, one Salmonella assay, for detecting the inducible target gene agsA, may comprise assay agsA.1 and use the primer of SEQ ID NO: 2 (forward primer), the primer of SEQ ID NO: 135 (reverse primer) and the probe of SEQ ID NO: 268.

TABLE 1
TaqMan® assays designed against candidate inducible genes in Salmonella.

| Gene | Assay | Forward Primer | Reverse Primer | Probe |
|---|---|---|---|---|
| agsA | agsA.0 | CTGGCGTATCTCCTGTTAATTGACT | ATTCTCTTTTCCCTGACCGTTTCA | ACCGTATTGATAGACTTTTC |
| | | SEQ ID NO: 1 | SEQ ID NO: 134 | SEQ ID NO: 267 |
| | agsA.1 | TGTGTTCAATCGCCTTTGGTTTG | GGAGATCCCTGAAAGCGAGAAAC | CTTTCTATGGCAATTTTTT |
| | | SEQ ID NO: 2 | SEQ ID NO: 135 | SEQ ID NO: 268 |
| | agsA.2 | GGCGTATCTCCTGTTAATTGACTGA | TCCCGTGTTTGCTGATTCTCTTTT | CCCTGACCGTTTCAAC |
| | | SEQ ID NO: 3 | SEQ ID NO: 136 | SEQ ID NO: 269 |
| | agsA.3 | CGCGCTTTTGCAGATCGTAAG | CCTGACCGTTTCAACCGTATTG | CTGGCGTATCTCCTGTTAAT |
| | | SEQ ID NO: 4 | SEQ ID NO: 137 | SEQ ID NO: 270 |
| | agsA.4 | TGGAAATCCGCCTTACGAATCC | ACACTGAAGATACGGTAGAGGATCA | ACGCACTGGATTTATC |
| | | SEQ ID NO: 5 | SEQ ID NO: 138 | SEQ ID NO: 271 |
| | agsA.5 | AGGCCCTGTTCCAGTTTCG | GGCGGACTTCCAGTTGAGTTTT | CATGCTAAGGTGAATAAT |
| | | SEQ ID NO: 6 | SEQ ID NO: 139 | SEQ ID NO: 272 |
| | agsA.6 | GCCGCCAACCGTTTCAATT | GCTTACCGTGAGCGTTCCT | TCAAGCTCTTCCTCTTTCC |
| | | SEQ ID NO: 7 | SEQ ID NO: 140 | SEQ ID NO: 273 |
| | agsA.7 | TCCTCTACCGTATCTTCAGTGTGTT | GCTGGAAAGAGGAAGAGCTTGAAAT | TTGCCGCCAACCGTTTC |
| | | SEQ ID NO: 8 | SEQ ID NO: 141 | SEQ ID NO: 274 |
| | agsA.8 | GGAAATCCGCCTTACGAATACCA | CACTGAAGAGACGGTAGAGGATCA | ACGCACTGGATTTATC |
| | | SEQ ID NO: 9 | SEQ ID NO: 142 | SEQ ID NO: 275 |
| | agsA.9 | CGCCTTTGGTTTGCTTTCTATGG | GGCCTCTTGTTGGTCGAGATTTAC | TCGCTTTCAGGGATCTCC |
| | | SEQ ID NO: 10 | SEQ ID NO: 143 | SEQ ID NO: 276 |
| | agsA.10 | TTTCTCGCTTTCAGGGATTTCCT | TGCCTGAACATGCTAAGGTGAATAA | CCTGTTCCAGTTTTGC |
| | | SEQ ID NO: 11 | SEQ ID NO: 144 | SEQ ID NO: 277 |
| | agsA.11 | CGCCTTTGGTTTGCTTTCTATGG | GGCCTCTTGTTGGTCGAGATTTAC | TCGCTTTCAGGGATCTC |
| | | SEQ ID NO: 12 | SEQ ID NO: 145 | SEQ ID NO: 278 |
| | agsA.12 | GTCTCTTCAGTGTGTTTACCCGTAA | GCTGGAAAGAGGAAGAGCTTGAAAT | TTGGCGGCAACCTGA |
| | | SEQ ID NO: 13 | SEQ ID NO: 146 | SEQ ID NO: 279 |
| | agsA.13 | ACCGTTTCAATTTCAAGCTCTTCCT | CGATCGCGAATAACTATCTGCTTACC | TCCAGCCAGGAACGC |
| | | SEQ ID NO: 14 | SEQ ID NO: 147 | SEQ ID NO: 280 |
| | agsA.14 | GCCAACCGTTTCAATTTCAAGCT | CGATGCGAATAACTATCTGCTTACC | CCTGGCTGGAAAGAG |
| | | SEQ ID NO: 15 | SEQ ID NO: 148 | SEQ ID NO: 281 |
| | agsA.15 | CGCTCACGGTAAGCAGATAGTTATT | CAGGAGATACGCCAGTTGCT | ACGATCTGCAAAAGCG |
| | | SEQ ID NO: 16 | SEQ ID NO: 149 | SEQ ID NO: 282 |
| | agsA.16 | ACTGGAAATCTGCCTTACGAATACC | CACTGAAGAGACGGTAGAGGATCA | ACGCACTGGATTTATC |
| | | SEQ ID NO: 17 | SEQ ID NO: 150 | SEQ ID NO: 283 |
| | agsA.17 | AGAGGCCCTGTTCCAGTTTTG | CGTAAGGCAGATTTCCAGTTGAGTT | ATGCTAAGGTGAATAATG |
| | | SEQ ID NO: 18 | SEQ ID NO: 151 | SEQ ID NO: 284 |
| | agsA.18 | CGCTCACGGTAAGCAGATAGTTA | CGACGCCAGCTTACGATCT | TTCGCATCGCGCTTTT |
| | | SEQ ID NO: 19 | SEQ ID NO: 152 | SEQ ID NO: 285 |
| | agsA.19 | GTCTCTTCAGTGTGTTTACCCGTAA | GCTTACCGTGAGCGTTCCT | CCGTTTCAATTTCAAGCTCT |
| | | SEQ ID NO: 20 | SEQ ID NO: 153 | SEQ ID NO: 286 |
| | agsA.20 | CGCCTTACGAATACCACGATAAAATC | CGGCAACCTGAATATTACGGGTAAA | CTGATCCTCTACCGTCTCTT |
| | | SEQ ID NO: 21 | SEQ ID NO: 154 | SEQ ID NO: 287 |
| | agsA.21 | GACCAACAAGAGGCCCTGTT | AGGCGGATTTCCAGTTGAGTTTT | CCAGTTTCGCATTATTCAC |
| | | SEQ ID NO: 22 | SEQ ID NO: 155 | SEQ ID NO: 288 |
| | agsA.22 | ACCGTTTCAATTTCAAGCTCTTCCT | GCCAGCTTACGATCTGCAAAAG | CACGGTAAGCAGATAGTTAT |
| | | SEQ ID NO: 23 | SEQ ID NO: 156 | SEQ ID NO: 289 |

| | | | | |
|---|---|---|---|---|
| cspH | cspH.0 | TTGTCTCGTAAAATGACAGGAATTGTC | ACCTGAACATCTTTGCGTCCAT | AAGGTCTCATCACCCCCTCC |
| | | SEQ ID NO: 24 | SEQ ID NO: 157 | SEQ ID NO: 290 |
| | cspH.1 | GCGCTTATCCCCGGTATACG | GTTGGCGGCGGTAGGT | CCATTAATACGACAAAACTC |
| | | SEQ ID NO: 25 | SEQ ID NO: 158 | SEQ ID NO: 291 |
| | cspH.2 | CCCTCCGATGGACGCAAA | GTTTCGTGTTGGCTAAATGCTGAA | ATGTGGACCTGAACATC |
| | | SEQ ID NO: 26 | SEQ ID NO: 159 | SEQ ID NO: 292 |
| | cspH.3 | GTAAGAGCGGTAAAGGTCTCATCAC | CGTGTAGGCGACATGCTGAA | ATGTGGAACTGAACATCTT |
| | | SEQ ID NO: 27 | SEQ ID NO: 160 | SEQ ID NO: 293 |
| | cspH.4 | CCCCTCCGATGACGCAAA | GTGTTGGCGACATGCTGAA | ATGTGGACCTGAACATC |
| | | SEQ ID NO: 28 | SEQ ID NO: 161 | SEQ ID NO: 294 |
| | cspH.5 | TTGTCTCGTAAAATGACAGGAATTGTC | ACCTGAACATCTTTGCGTCCAT | ACCGCTCTTACAATCAA |
| | | SEQ ID NO: 29 | SEQ ID NO: 162 | SEQ ID NO: 295 |
| | cspH.6 | GAAGCGCTTATCCCCGGTAT | GTCCGCGGAGGCCATTAATA | ACGCGTTGAGTTTTATC |
| | | SEQ ID NO: 30 | SEQ ID NO: 163 | SEQ ID NO: 296 |
| | cspH.7 | GTAAGAGCGGTAAAGGTCTCATCAC | TGCTGAAATGTGGACCTGAACA | CCCCTCCGATGGACGC |
| | | SEQ ID NO: 31 | SEQ ID NO: 164 | SEQ ID NO: 297 |
| | cspH.8 | TTGTCTCGTAAAATGACAGGAATTGTC | ACCTGAACATCTTTGCGTCCAT | ACCGCTCTTACAATCAA |
| | | SEQ ID NO: 32 | SEQ ID NO: 165 | SEQ ID NO: 298 |
| | cspH.9 | GTAAGAGCGGTAAAGGTCTCATCAC | GTGTTGGCGACATGATGAGATG | CCCCTCCGATGGACGC |
| | | SEQ ID NO: 33 | SEQ ID NO: 166 | SEQ ID NO: 299 |
| | cspH.10 | TTGTCTCGTAAAATGACAGGAATTGTC | ACCTGAACATCTTTGCGTCCAT | AAGAGCGGTAAAGGTCTCA |
| | | SEQ ID NO: 34 | SEQ ID NO: 167 | SEQ ID NO: 300 |
| | cspH.11 | GTAAGAGCGGTAAAGGTCTCATCAC | GTTTCGTGTTGGCGACATGAT | ATGTGGACCTGAACATCT |
| | | SEQ ID NO: 35 | SEQ ID NO: 168 | SEQ ID NO: 301 |
| | cspH.12 | GTAAGAGCGGTAAAGGTCTCATCAC | CATGCTGAGATGTGGACCTGAA | ATGGACGCAAAGATG |
| | | SEQ ID NO: 36 | SEQ ID NO: 169 | SEQ ID NO: 302 |
| | cspH.13 | ACCTTTGATTGTAAGAGCGGTAACC | TGTTGGCGACATGCTGAAATG | ATGGACGCAAAGATG |
| | | SEQ ID NO: 37 | SEQ ID NO: 170 | SEQ ID NO: 303 |
| | cspH.14 | GATTGTAAGAGCGGTAAAGGTCTCA | TGCTGAAATGTGGACCTGAACA | ACCCCCTCCGATGACG |
| | | SEQ ID NO: 38 | SEQ ID NO: 171 | SEQ ID NO: 304 |
| | cspH.15 | CCCTCCGATGGACGCAAA | TCGTGTTGGCGACATGCT | ATGTGGACCTGAACATC |
| | | SEQ ID NO: 39 | SEQ ID NO: 172 | SEQ ID NO: 305 |
| | cspH.16 | TCCGATGGACGCAAAGATGTT | TCTGTTTCGTGTTGGCGACAT | CAGGTCCACATTTCAGC |
| | | SEQ ID NO: 40 | SEQ ID NO: 173 | SEQ ID NO: 306 |
| | cspH.17 | GACGCAAAGATGTTCAGTTCCA | GGGATAAGCGCTTCTGTTTCGT | TAGGCGACATGCTGAAATG |
| | | SEQ ID NO: 41 | SEQ ID NO: 174 | SEQ ID NO: 307 |
| | cspH.18 | GATTGTAAGAGCGGTAAAGGTCTCA | TGCTGAAATGTGGACCTGAACA | CACCCCCTCCGATGGAC |
| | | SEQ ID NO: 42 | SEQ ID NO: 175 | SEQ ID NO: 308 |
| | cspH.19 | TGTTCAGGTCCACATTTCAGCAT | CGACAAAACTCAACGCGTATACC | TCGCCAACACGAAACAG |
| | | SEQ ID NO: 43 | SEQ ID NO: 176 | SEQ ID NO: 309 |
| | cspH.20 | CACGAAACAGAAGCGCTTATCC | TCCGCGGAGGCCATTAATAC | TACGCGTTGAGTTTTGTC |
| | | SEQ ID NO: 44 | SEQ ID NO: 177 | SEQ ID NO: 310 |
| dnaK | dnaK.0 | CGGTGGTGGTACTTTCGATATCTC | GGTTGCCAGAACTTCAAAGGTTTT | TCGCCATCAACTTCG |
| | | SEQ ID NO: 45 | SEQ ID NO: 178 | SEQ ID NO: 311 |
| | dnaK.1 | GTAACCAGGGCGACCATCT | CCTGCTTCTTCGACCTGCTT | ACGGGTGCTGTGCAGC |
| | | SEQ ID NO: 46 | SEQ ID NO: 179 | SEQ ID NO: 312 |
| | dnaK.2 | AAAATCATCGGCGCTGACAAC | GCGGCGCCATTTTCTGA | TTCACATCAAGCCATGCGTC |
| | | SEQ ID NO: 47 | SEQ ID NO: 180 | SEQ ID NO: 313 |
| | dnaK.3 | CCAACTCTTGTGTAGCGATTATGGA | CGCCCTCGGCGTTCT | ACGCGTGCCTGCGTTC |
| | | SEQ ID NO: 48 | SEQ ID NO: 181 | SEQ ID NO: 314 |
| | dnaK.4 | ACCGTAAGTTTGAAGAGCTGGTT | CCTGCTTCTTCGACCTGCTTA | ATGGTCGCCCTGGTTACG |
| | | SEQ ID NO: 49 | SEQ ID NO: 182 | SEQ ID NO: 315 |
| | dnaK.5 | CTGAATCCGACCGTAAGTTCGA | GCCTGCTTCTTCAACCTGCTTA | TACGGGTCTGAACCAGC |
| | | SEQ ID NO: 50 | SEQ ID NO: 183 | SEQ ID NO: 316 |
| | dnaK.6 | CGATACCCGCCTGATCAACTAC | GATCGTTACGCAGGTCAATGC | TCGTTGACGAGTTTAAGAAA |
| | | SEQ ID NO: 51 | SEQ ID NO: 184 | SEQ ID NO: 317 |
| | dnaK.7 | GCTGCAGGGTGAGCGTAA | GATGCCATCCAGGTTGAACTGA | ACGTGCGTCTGATAACAAAT |
| | | SEQ ID NO: 52 | SEQ ID NO: 185 | SEQ ID NO: 318 |
| | dnaK.8 | GTGACCCGTGCGAAACTG | CGGCTCGATAGAACGGTTCAC | ATCTTCAACCAGGCTTTC |
| | | SEQ ID NO: 53 | SEQ ID NO: 186 | SEQ ID NO: 319 |
| | dnaK.9 | AGATCTGGTGAACCGTTCTATCGA | ACCGCCAACGAGGATCAC | CCTGCAGTGCGACTTT |
| | | SEQ ID NO: 54 | SEQ ID NO: 187 | SEQ ID NO: 320 |
| | dnaK.10 | AAATCGCTCAGCAGCAACATG | AACTCAGCGTCGACAACGT | ACGCTTCTGCAAACAA |
| | | SEQ ID NO: 55 | SEQ ID NO: 188 | SEQ ID NO: 321 |
| | dnaK.11 | GGCTTACGGTCTGGATAAAGAAGTC | TCGCCATCAACTTCGTCGATTT | CCGTACTATCGCGGTTTAC |
| | | SEQ ID NO: 56 | SEQ ID NO: 189 | SEQ ID NO: 322 |
| | dnaK.12 | CAGAAGCGAACGCTGAATCC | CCCTGGTTACGGGTCTGAAC | CAGCTCTTCGAACTTAC |
| | | SEQ ID NO: 57 | SEQ ID NO: 190 | SEQ ID NO: 323 |
| | dnaK.13 | CGGTGGTGGTACTTTCGATATCTC | GGTTGCCAGAACTTCAAAGGTTTT | CAACTTCGTCGATTTCG |
| | | SEQ ID NO: 58 | SEQ ID NO: 191 | SEQ ID NO: 324 |
| | dnaK.14 | CCGCAGATCGAAGTCACCTT | GTGATCTTCTGCTCTTTACCGCTAT | TCCTGCACGTCTCCGC |
| | | SEQ ID NO: 59 | SEQ ID NO: 192 | SEQ ID NO: 325 |
| | dnaK.15 | CACGCGTGCTGGAGAAC | CAGAGTTTCACCATCCTGGGTATAA | CTACGCCTTCTATCATTGC |
| | | SEQ ID NO: 60 | SEQ ID NO: 193 | SEQ ID NO: 326 |
| | dnaK.16 | CCGTACCGGCTTACTTTAACGAT | CAGTTGGTTCGTTGATGATACGTTT | ACGACCAGCATCTTTG |
| | | SEQ ID NO: 61 | SEQ ID NO: 194 | SEQ ID NO: 327 |
| | dnaK.17 | TTCTATCGACCGCTGAAAGTC | ACCGCCAACGAGGATCAC | CAGACACGGATAGGCC |
| | | SEQ ID NO: 62 | SEQ ID NO: 195 | SEQ ID NO: 328 |
| | dnaK.18 | CGCCTGATCAACTACCTCGTT | TTTCAGGCGCTGCATTGC | ACGCAGGTCAATGCCCT |
| | | SEQ ID NO: 63 | SEQ ID NO: 196 | SEQ ID NO: 329 |
| | dnaK.19 | AACTGGAAAGCCTGGTTGAAGAT | TCCTGCAGTGCGACTTTCAG | CTGGTGAACCGTTCTATC |
| | | SEQ ID NO: 64 | SEQ ID NO: 197 | SEQ ID NO: 330 |
| | dnaK.20 | ATCAAAGTGACTCGTGCAAAACTG | CGGCTCGATAGAACGGTTCAC | CAGATCTTCAACCAGGCTTT |
| | | SEQ ID NO: 65 | SEQ ID NO: 198 | SEQ ID NO: 331 |
| Hsp6 | hsp60.0 | CTCCGACTCTAAAGCGATTGCT | CGATCAGTTTACCTACGGTTTCGT | ACTATTTCCGCTAACTCCG |
| 0 | | SEQ ID NO: 66 | SEQ ID NO: 199 | SEQ ID NO: 332 |
| | hsp60.1 | CGACGAAACCGTAGGTAAACTGAT | AACGGTGATGACGCCTTCTTTA | CTTTATCCATCGCTTCCGCG |
| | | SEQ ID NO: 67 | SEQ ID NO: 200 | SEQ ID NO: 333 |
| | hsp60.2 | ATGGCAGCTAAAGACGTAAAATTCG | CGAGGGTTACTTTCACTGCATCTG | ACGCTCGTGTGAAAAT |
| | | SEQ ID NO: 68 | SEQ ID NO: 201 | SEQ ID NO: 334 |
| | hsp60.3 | GTAAGGCGATGCTGCAGGATAT | AGCTCCATACCGATCTCTTCAGA | CCTGACCGGCGGTACC |
| | | SEQ ID NO: 69 | SEQ ID NO: 202 | SEQ ID NO: 335 |
| | hsp60.4 | CGGCAACATGATCGATATGGGTAT | GCCACAGAAGCAGCGTACT | CAGAACGGGTAACTTTG |
| | | SEQ ID NO: 70 | SEQ ID NO: 203 | SEQ ID NO: 336 |
| | hsp60.5 | CCGTGCTGCGGTAGAAGAA | AATTTTAGAAGCAACGCGGATCAG | CCACCACCAGCAACCA |
| | | SEQ ID NO: 71 | SEQ ID NO: 204 | SEQ ID NO: 337 |
| | hsp60.6 | CCGTAGCGCGTGAAATCG | CTGCGCGCCCATGTTTT | AACTGGAAGACAAGTTTG |
| | | SEQ ID NO: 72 | SEQ ID NO: 205 | SEQ ID NO: 338 |
| | hsp60.7 | TCAACAAAGACACCACCACCAT | CCTGGATGGCAGCTTCTTCA | CCCACGCCATCAATG |
| | | SEQ ID NO: 73 | SEQ ID NO: 206 | SEQ ID NO: 339 |
| | hsp60.8 | CCAACCAAAGTTACCCGTTCTG | GTCACCATGCACTCGGTAGT | CTGTGGCTGGTCTGATGAT |
| | | SEQ ID NO: 74 | SEQ ID NO: 207 | SEQ ID NO: 340 |
| | hsp60.9 | GGAAGCCGTTGCAAAAGCA | GCGCTTCGCCTTCAACA | TCTTCAGCGATGATCAGC |
| | | SEQ ID NO: 75 | SEQ ID NO: 208 | SEQ ID NO: 341 |
| | hsp60.10 | CAGATGGTGAAAGAAGTTGCCTCTA | GCTTTCAAGCCTTCGGTAATGATG | TCGCCTGCAGCATCG |
| | | SEQ ID NO: 76 | SEQ ID NO: 209 | SEQ ID NO: 342 |
| | hsp60.11 | GAGATCGGTATGGAGCTGGAAAA | TGGTGGTGTCTTTGTTGATCACA | CTGCCCCAGGTCTTC |
| | | SEQ ID NO: 77 | SEQ ID NO: 210 | SEQ ID NO: 343 |
| | hsp60.12 | CAGATGGTGAAAGAAGTTGCCTCTA | CTTTCAGGCCTTCGGTAATAATGGA | CTGCAGCGTCGTTCGC |
| | | SEQ ID NO: 78 | SEQ ID NO: 211 | SEQ ID NO: 344 |
| | hsp60.13 | CTCCGACTCTAAAGCGATTGCT | GCGATCAGTTTACCTACAGTTTCGT | ACTATCTCCGCTAACTCC |
| | | SEQ ID NO: 79 | SEQ ID NO: 212 | SEQ ID NO: 345 |
| | hsp60.14 | GGCGAAACGTGTTGTGATCAA | CCTGGATGGCAGCTTCTTCA | ACACCACCACCATCATT |
| | | SEQ ID NO: 80 | SEQ ID NO: 213 | SEQ ID NO: 346 |
| | hsp60.15 | CCGACGAAACTGTAGGTAAACTGAT | AACGGTGATGACGCCTTCTTTA | AAGCGATGGATAAAGTC |
| | | SEQ ID NO: 81 | SEQ ID NO: 214 | SEQ ID NO: 347 |
| | hsp60.16 | GCTGCTGATCATCGCTGAAGAT | ACAGCAGCCACTTTCACGAT | CACGCATGGTGTTCAC |
| | | SEQ ID NO: 82 | SEQ ID NO: 215 | SEQ ID NO: 348 |
| | hsp60.17 | CGGTAGAAGAAGGCGTGGTT | GCCTTTCAGGTCAGCAATTTTAGAA | TCAGCGCAACGCCACC |
| | | SEQ ID NO: 83 | SEQ ID NO: 216 | SEQ ID NO: 349 |
| | hsp60.18 | GCTGCTGCGGTTGAAGAG | CTACCTGAGCAATCGCTTTAGAGT | ACGGACAGGGCTTTCA |
| | | SEQ ID NO: 84 | SEQ ID NO: 217 | SEQ ID NO: 350 |
| | hsp60.19 | GGCAGATGCAGTGAAAGTTACC | CGCACCGAAAGATTTATCCAGAAC | ACGTTACGGCCTTTCGGACCG |
| | | SEQ ID NO: 85 | SEQ ID NO: 218 | SEQ ID NO: 351 |
| | hsp60.20 | CGACCGAAGTTGAAATGAAAGAGAA | AGCAACCACGCCTTCTTCTAC | TCTTCAACGCGGGCTTT |
| | | SEQ ID NO: 86 | SEQ ID NO: 219 | SEQ ID NO: 352 |
| ibpAB | ibpAB.0 | GCTGGCTGAGAATATGGAAGTTTC | CGTTGCGGGTTAAATCAATATGCA | CCGTTGGTAAACGTCGCGCC |
| | | SEQ ID NO: 87 | SEQ ID NO: 220 | SEQ ID NO: 353 |
| | ibpAB.1 | TCCAGTTAGCGGAGAACATTCAC | CGATATACAGCAGCCCGTTGAC | TCGTGGCGCAAACC |
| | | SEQ ID NO: 88 | SEQ ID NO: 221 | SEQ ID NO: 354 |
| | ibpAB.2 | CAGTTAGCGGAAAACATTCACGTT | CGCGCTCCAGTTCGATATACAG | CCGTTGACCAGGTTTGCGC |
| | | SEQ ID NO: 89 | SEQ ID NO: 222 | SEQ ID NO: 355 |
| | ibpAB.3 | TGCGCGGCCTGTCA | TCACACTTTACGTGCGAATATGCT | TCCCATGCTCGCCGTCAG |
| | | SEQ ID NO: 90 | SEQ ID NO: 223 | SEQ ID NO: 356 |
| | ibpAB.4 | GAGCTTCCCGCCCTATAACAT | ACGCTAAGGTAATGCGATAATGGTT | ATCGTCGCTTTTTTCG |
| | | SEQ ID NO: 91 | SEQ ID NO: 224 | SEQ ID NO: 357 |
| | ibpAB.5 | TGCTGTACCGTTCTGCCATT | AGGGTGCCGCCATTACTTTG | ACAGACGGTCAAAACC |
| | | SEQ ID NO: 92 | SEQ ID NO: 225 | SEQ ID NO: 358 |
| | ibpAB.6 | GCGCCTGACGGTGAAAG | AGGCCCTGATGTAACCATTTGG | AACAGCCGGAAAACG |
| | | SEQ ID NO: 93 | SEQ ID NO: 226 | SEQ ID NO: 359 |
| | ibpAB.7 | CGCACGTAAAGTGTGAAGGTAAAA | CAGCTTGTCAAAACCGATCCATTG | CTTAGAAGGAGAAATGATTATGC |
| | | SEQ ID NO: 94 | SEQ ID NO: 227 | SEQ ID NO: 360 |
| | ibpAB.8 | CCACTGCTGCGTCAATGGA | CTGTTTTGCAGCGCATTGG | CCAGCTTGTCAAAACC |
| | | SEQ ID NO: 95 | SEQ ID NO: 228 | SEQ ID NO: 361 |
| | ibpAB.9 | CTACCGCATCGCCATTGC | GAGCGCCTTTTACCACCAGTA | TTCGCGGAAAGCGAACTG |
| | | SEQ ID NO: 96 | SEQ ID NO: 229 | SEQ ID NO: 362 |
| | ibpAB.10 | GAAAACGACCCAAATGGTTACATC | GCGTAAAGCTCAGGCTAAAAGG | CTGCATGACCAGGCCCT |
| | | SEQ ID NO: 97 | SEQ ID NO: 230 | SEQ ID NO: 363 |
| | ibpAB.11 | CGAGACAGAAAGAACGTACTTACCT | AACGTGAATGTTTTCCGCTAACTG | AAGTTACGCTCGGCAATGC |
| | | SEQ ID NO: 98 | SEQ ID NO: 231 | SEQ ID NO: 364 |
| | ibpAB.12 | GGTGGTAAAAGGCGCTCATG | AAAGTTACGCTCGGCAATGC | ACAGGTAAGTACGTTCTTTC |
| | | SEQ ID NO: 99 | SEQ ID NO: 232 | SEQ ID NO: 365 |
| | ibpAB.13 | CTTCCCGCCCTATAACATCGA | CGGCTAACGCTAAGGTAATGC | AATGGTTATCGTCGCTTTTT |
| | | SEQ ID NO: 100 | SEQ ID NO: 233 | SEQ ID NO: 366 |
| | ibpAB.14 | GGCCTGCTGCTATTGATTTAACC | CGCTGCGGCGCAAT | CAACGAGCCGGAAACC |
| | | SEQ ID NO: 101 | SEQ ID NO: 234 | SEQ ID NO: 367 |
| | ibpAB.15 | CGCACGTAAAGTGTGAAGGTAAAA | CCGATCCATTGACGCAACAG | ATGCGTAACTACGATTTATC |
| | | SEQ ID NO: 102 | SEQ ID NO: 235 | SEQ ID NO: 368 |
| | ibpAB.16 | GGCGCGACGTTTACCAA | TGGTTTCCGGCTCGTTACG | CCTGCTGCTATTGATTTA |
| | | SEQ ID NO: 103 | SEQ ID NO: 236 | SEQ ID NO: 369 |
| | ibpAB.17 | CGCACGTAAAGTGTGAAGGTAAAA | CAGCTTGTCAAAACCGATCCATTG | ATGCGTAACTACGATTTATC |
| | | SEQ ID NO: 104 | SEQ ID NO: 237 | SEQ ID NO: 370 |
| | ibpAB.18 | CGAACTGGAGCGCGTGAT | CGACCCGACGGAATCAGTTAATTT | AAACCGCGCCGTATCG |
| | | SEQ ID NO: 105 | SEQ ID NO: 238 | SEQ ID NO: 371 |
| | ibpAB.19 | CGCCGTCAGGGAGCAT | CTCCTTCTAAGAAGCGAGTTTTACCT | ATTCGCACGTAAAGTGTG |
| | | SEQ ID NO: 106 | SEQ ID NO: 239 | SEQ ID NO: 372 |
| | ibpAB.20 | CGCCGTATCGAAATTAACTGATTCC | TCACACTTTACGTGCGAATATGCT | CTGTCATCCCATGCTCG |
| | | SEQ ID NO: 107 | SEQ ID NO: 240 | SEQ ID NO: 373 |
| | ibpAB.21 | CGCCGTCAGGGAGCAT | CGTAGTTACGCATAATCATTTCTCCTTCT | ATTCGCACGTAAAGTGTG |
| | | SEQ ID NO: 108 | SEQ ID NO: 241 | SEQ ID NO: 374 |
| | ibpAB.22 | ACTGGAGATTACTGCCCAGGATAAT | TCGGCAATGCCCTGATACAG | CATGAGCGCCTTTTAC |
| | | SEQ ID NO: 109 | SEQ ID NO: 242 | SEQ ID NO: 375 |
| uspA | uspA.0 | TGGTGATGACCGCAAACGA | CGGTAGCGGCGATTTGG | CCAGATCCATATCGTATTTC |
| | | SEQ ID NO: 110 | SEQ ID NO: 243 | SEQ ID NO: 376 |
| | uspA.1 | TTTCTTCGGAGATACGTTTCTGCAT | GAAAATCTCCCTCATCCACGTTGAT | CTGTACACCGGTCTGATTG |
| | | SEQ ID NO: 111 | SEQ ID NO: 244 | SEQ ID NO: 377 |
| | uspA.2 | GCAAACGACCAGATCCATATCG | AGCGGTAGCGGCGATTT | CAGGTGCTGGTTGACGC |
| | | SEQ ID NO: 112 | SEQ ID NO: 245 | SEQ ID NO: 378 |
| | uspA.3 | CGTCAATCAGACCGGTGTACAG | CAACGCGAAAATCTCCCTCATC | ACGTTGACGTGAATTATT |
| | | SEQ ID NO: 113 | SEQ ID NO: 246 | SEQ ID NO: 379 |
| | uspA.4 | GCGGAACAATCAGCATGTCAA | TCTGGAGCAAACTGATGTCTTCTG | CTGATCAACACCGTTCACG |
| | | SEQ ID NO: 114 | SEQ ID NO: 247 | SEQ ID NO: 380 |
| | uspA.5 | GCAAACGACCAGATCCATATCG | CTGGCTACCCTATCACTGAAACC | CCCAAATCGCCGCTACC |
| | | SEQ ID NO: 115 | SEQ ID NO: 248 | SEQ ID NO: 381 |
| | uspA.6 | GTAGCCAGCGTTGGTAGACA | GCAGAAACGTATCTCCAAAGAAACC | ACCACGCGCTGACCG |
| | | SEQ ID NO: 116 | SEQ ID NO: 249 | SEQ ID NO: 382 |
| | uspA.7 | CGCAGCGGCACAATCA | CGCGCCAGCTGATCAAC | CCGTTCACGTTGACATGC |
| | | SEQ ID NO: 117 | SEQ ID NO: 250 | SEQ ID NO: 383 |
| | uspA.8 | CCGCTCAGGGTTTCAGTGATA | CGCTGACCGAGCTGTCTAC | CAACGCTGGCTACCC |
| | | SEQ ID NO: 118 | SEQ ID NO: 251 | SEQ ID NO: 384 |
| | uspA.9 | CGCTCAGGGTTTCAGTGATAGG | TGGGCGATATGCAGAAACGTAT | CAGCGTTGGTAGACAGC |
| | | SEQ ID NO: 119 | SEQ ID NO: 252 | SEQ ID NO: 385 |
| | uspA.10 | AAGACATCAGTTTGCTCCAGAAGT | GGCCAGGTGCTGGTTGAT | TCGTTTGCGGTCATCACC |
| | | SEQ ID NO: 120 | SEQ ID NO: 253 | SEQ ID NO: 386 |
| | uspA.11 | TTTCTTCGGAGATACGTTTCTGCAT | GAAAATCTCCCTCATCCACGTTGAT | CCGGTGTACAGGTCAGAAT |
| | | SEQ ID NO: 121 | SEQ ID NO: 254 | SEQ ID NO: 387 |
| | uspA.12 | GAACAATCAGCATGTCAACGTGAA | TCTGGAGCAAACTGATGTCTTCTG | CCAGCTGATCAACACCG |
| | | SEQ ID NO: 122 | SEQ ID NO: 255 | SEQ ID NO: 388 |
| | uspA.13 | CCGGTGTACAGGTCTGAATAGTTC | GCCCCTACAACGCGAAAAT | CTCCCTCATCCACGTTGAT |
| | | SEQ ID NO: 123 | SEQ ID NO: 256 | SEQ ID NO: 389 |
| | uspA.14 | GTAGCCAGCGTTGGTAGACA | TGGGCGATATGCAGAAACGTAT | CCCACCACGCGCTGAC |
| | | SEQ ID NO: 124 | SEQ ID NO: 257 | SEQ ID NO: 390 |
| | uspA.15 | TGGCGCGCAGAAGACAT | GGTCGTTTGCGGTCATCAC | CAGTTTGCTCCAGAAGTC |
| | | SEQ ID NO: 125 | SEQ ID NO: 258 | SEQ ID NO: 391 |
| | uspA.16 | CTACCGCTCAGGGTTTCAGT | CGCTGACCGAGCTGTCTAC | ACGCTGGCTACCCTATC |
| | | SEQ ID NO: 126 | SEQ ID NO: 259 | SEQ ID NO: 392 |
| | uspA.17 | CGCTCAGGGTTTCAGTGATAGG | TGGGCGATATGCAGAAACGTAT | CCGAGCTGTCTACCAACG |
| | | SEQ ID NO: 127 | SEQ ID NO: 260 | SEQ ID NO: 393 |
| | uspA.18 | ACCGGTGTACAGGTCTGAATAATTC | GCGCGCCCCTACAAC | ATGAGGGAGATTTTCG |
| | | SEQ ID NO: 128 | SEQ ID NO: 261 | SEQ ID NO: 394 |
| | uspA.19 | GTGGTGGGTTTCTTCGGAGAT | CCTGTACACCGGTCTGATTGAC | ACGTTTCTGCATATCGCC |
| | | SEQ ID NO: 129 | SEQ ID NO: 262 | SEQ ID NO: 395 |
| | uspA.20 | CGCTCAGGGTTTCAGTGATAGG | TGGGCGATATGCAGAAACGTAT | CAGCGTTGGTAGACAGC |
| | | SEQ ID NO: 130 | SEQ ID NO: 263 | SEQ ID NO: 396 |
| | uspA.21 | TGGCGCGCAGAAGACAT | GGTCGTTTGCGGTCATCAC | ACTTCTGGAGCAAACTG |
| | | SEQ ID NO: 131 | SEQ ID NO: 264 | SEQ ID NO: 397 |
| | uspA.22 | GCGCAGCGGAACAATCA | CGCGCCAGCTGATCAAC | CCGTTCACGTTGACATGC |
| | | SEQ ID NO: 132 | SEQ ID NO: 265 | SEQ ID NO: 398 |
| hilA | | GCAGTATGCGCCCTTTGG | CACTGCGGCAGTTCTTCGTA | TGCTGCCGGTGACC |
| | | SEQ ID NO: 133 | SEQ ID NO: 266 | SEQ ID NO: 399 |

The assays were evaluated under uninduced conditions as well as induced conditions (45° C., 15 minutes) using pure cultures of Salmonella enterica. Duplicate samples were maintained at 37° C. (uninduced) or 45° C. (heat-induced) for 15 minutes before processing for nucleic acids. The extracted nucleic acids were assayed for induction by real-time reverse-transcriptase-PCR (DNA+ RNA). To measure contribution due to DNA alone, control reactions were run without addition of the reverse transcriptase (DNA). Transcriptional activity (contribution to Ct value by RNA alone) was estimated by subtracting the Ct value obtained for DNA and RNA (with reverse transcriptase reaction) from the Ct value obtained for DNA alone (without reverse transcriptase reaction) for both uninduced and induced samples.

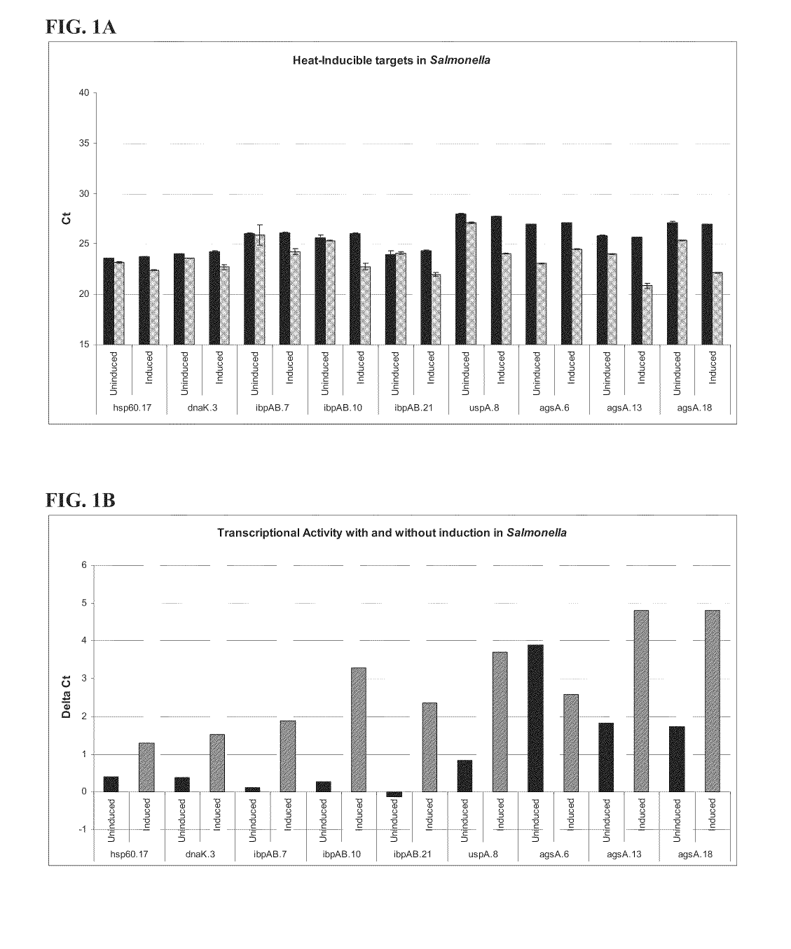

FIGS. 1A and 1B depict evaluation of heat-induced target genes for detecting Salmonella enterica. FIG. 1A depicts Ct data vs various primer/probe sets specific to some heat-inducible RNA genes for both uninduced (negative control) and induced cultures. An overnight S. enterica culture was diluted and grown at 37° C. to obtain exponential phase cells. Duplicate samples were maintained at 37° C. (uninduced) or at 45° C. (heat-induced) for 15 minutes; nucleic acids were extracted and assayed for induction by real-time reverse-transcriptase-PCR (hatched bars). To measure contribution due to DNA, control reactions were run without addition of the reverse transcriptase (solid bars). FIG. 1B depicts Delta Ct data vs. various primer/probe sets specific to some heat-inducible RNA genes for both uninduced (negative control) and induced cultures. Transcriptional activity was estimated by subtracting the CT value obtained for DNA and RNA (with reverse transcriptase reactions) from the CT value obtained for DNA alone (without reverse transcriptase reactions) for uninduced (solid bars) and induced (striped bars) samples. While inclusion of reverse transcriptase generally dropped the Ct levels in both uninduced and induced samples, a greater drop was observed upon induction, indicating higher transcript levels under these conditions. Transcriptional activity (FIG. 1B) for the uninduced samples generally ranged from 0 to <2 Ct, with the exception of one target, agsA-I, in which a 4 Ct difference was observed. For induced samples, this range was 1Ct-5Ct.

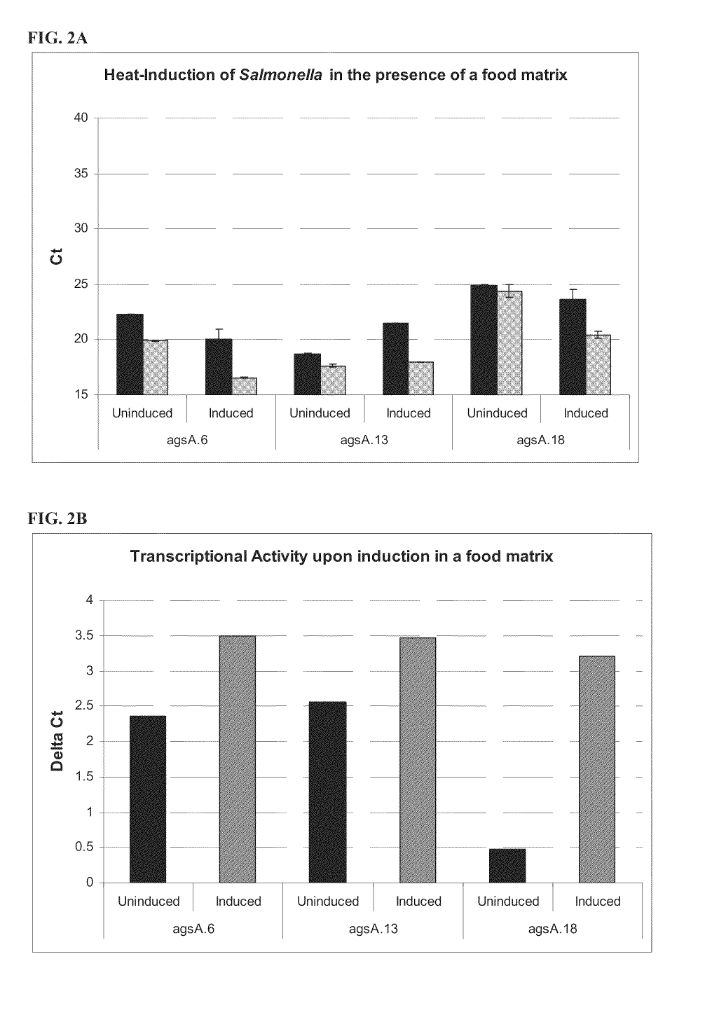

FIG. 2A-FIG. 2B show data on heat-induction of Salmonella response genes in the presence of a food matrix. The agsA assays were further verified in the context of contaminated food samples (FIG. 2A and FIG. 2B). A volume of whole milk (25 ml) was spiked with approximately 10 cfu of Salmonella. Enrichment broth (225 ml, Brian Heart Infusion Broth) was added. Samples were enriched for 16 hours at 37° C. At the end of the incubation period, duplicate samples were withdrawn. Samples were either heat-induced or uninduced. One set was maintained at 37° C. (Uninduced) and one set was incubated at 45° C. (Induced) for 15 minutes. The extracted nucleic acids were assayed for induction by real-time reverse-transcriptase-PCR (hatched bars). To measure contribution due to DNA, control reactions were run without addition of the reverse transcriptase (solid bars). Similar to the results with pure cultures, induction followed by real-time RT-PCR improved signal by 1 Ct-3 Ct (2-8 fold difference in the amount of target). FIG. 2A shows the Ct data vs various primer/probe sets specific to some heat-inducible RNA genes (see X-axis) for both uninduced (negative control) and induced cultures. FIG. 2B shows Delta Ct data vs. various primer/probe sets for specific to some heat-inducible RNA genes for both uninduced (negative control) and induced cultures. Transcriptional activity was estimated by subtracting the CT value obtained for DNA and RNA (with reverse transciptase reactions) from the CT value obtained for DNA alone (without reverse transcriptase reactions) for uninduced (solid bars) and induced (striped bars) samples.

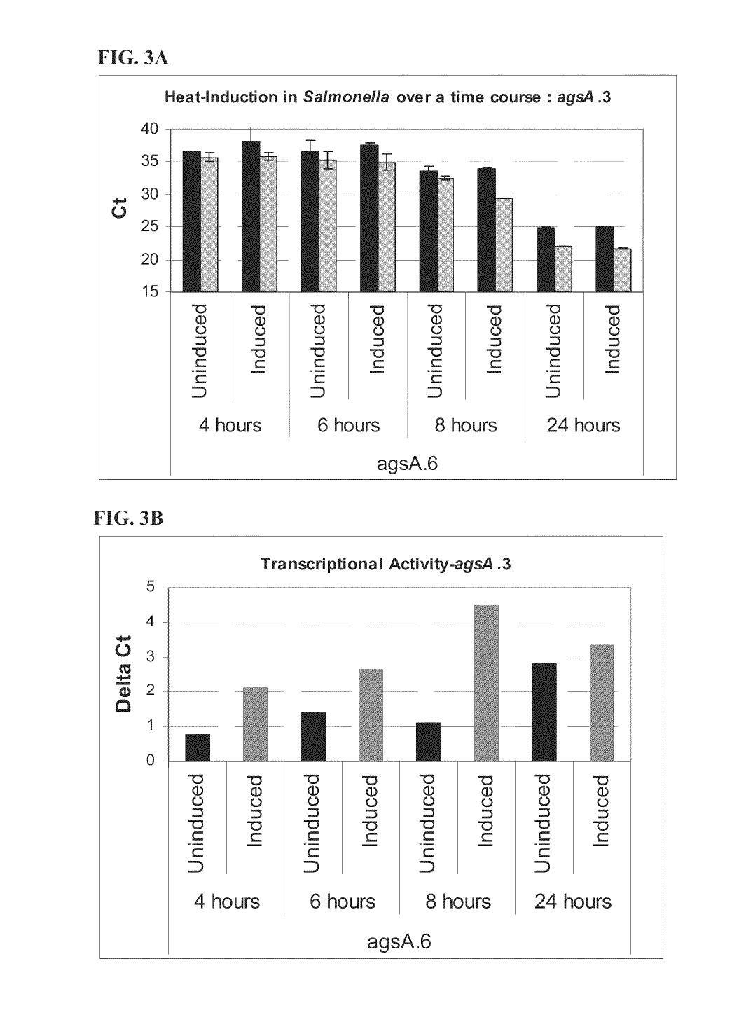

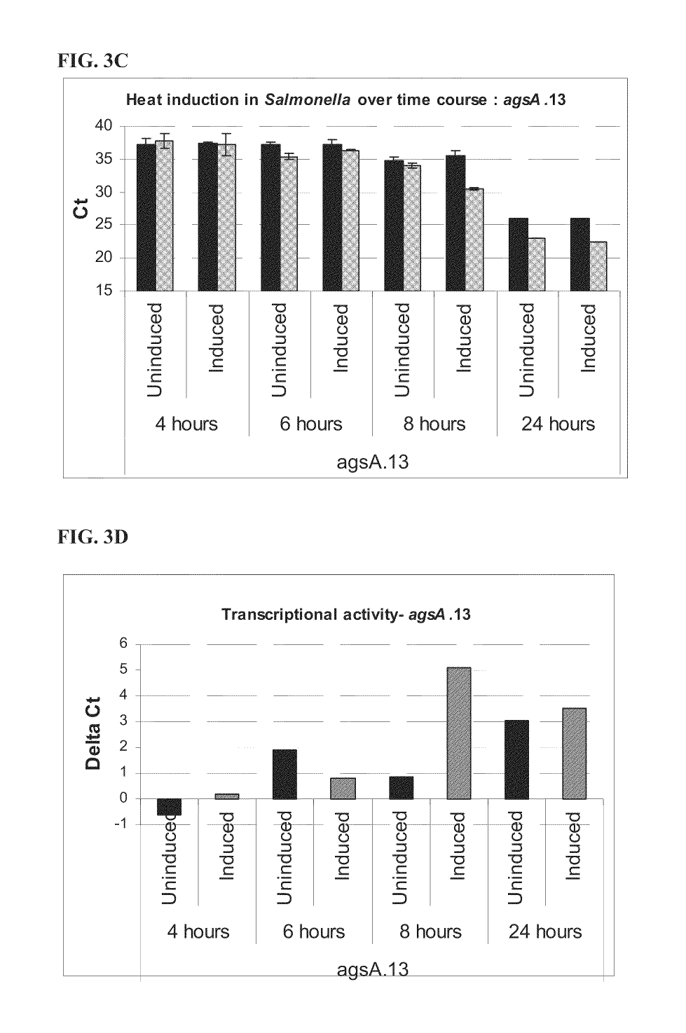

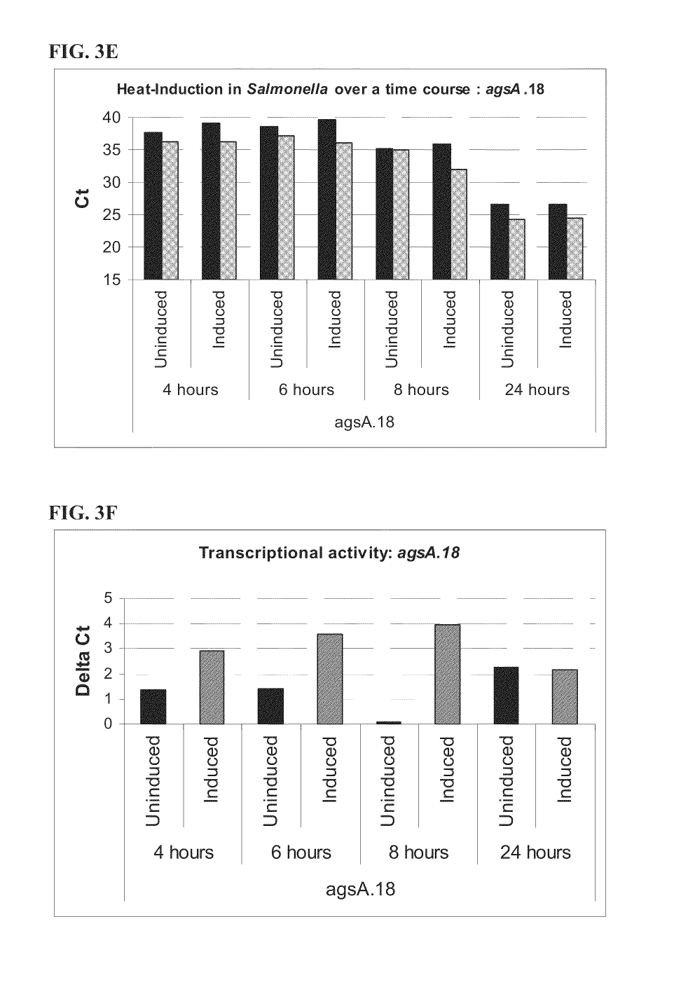

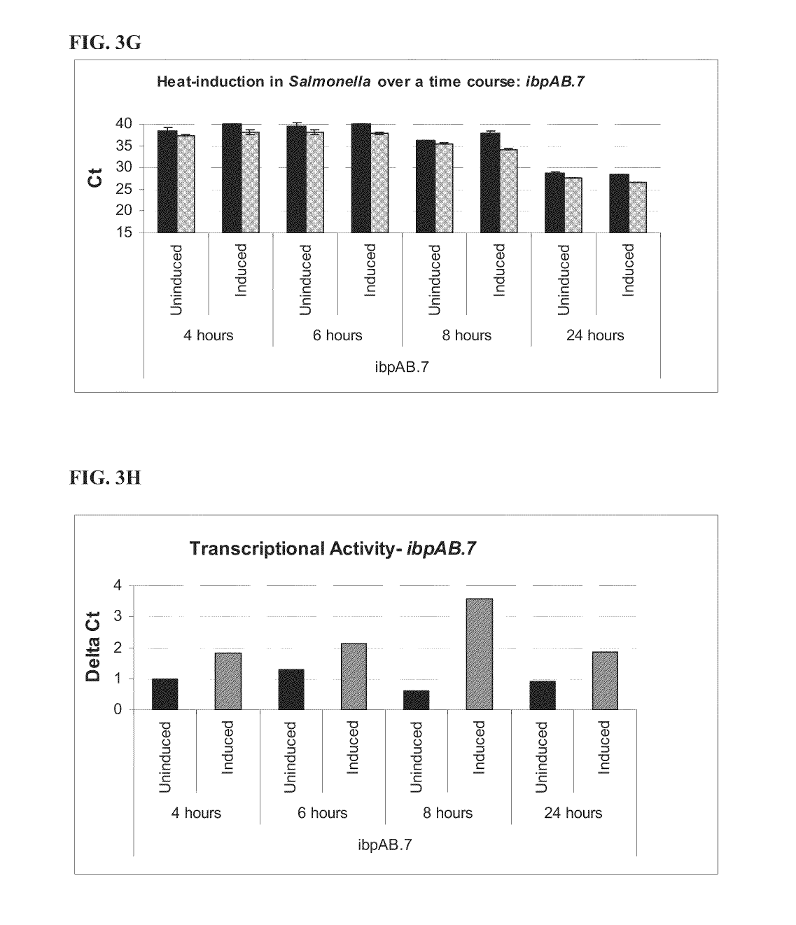

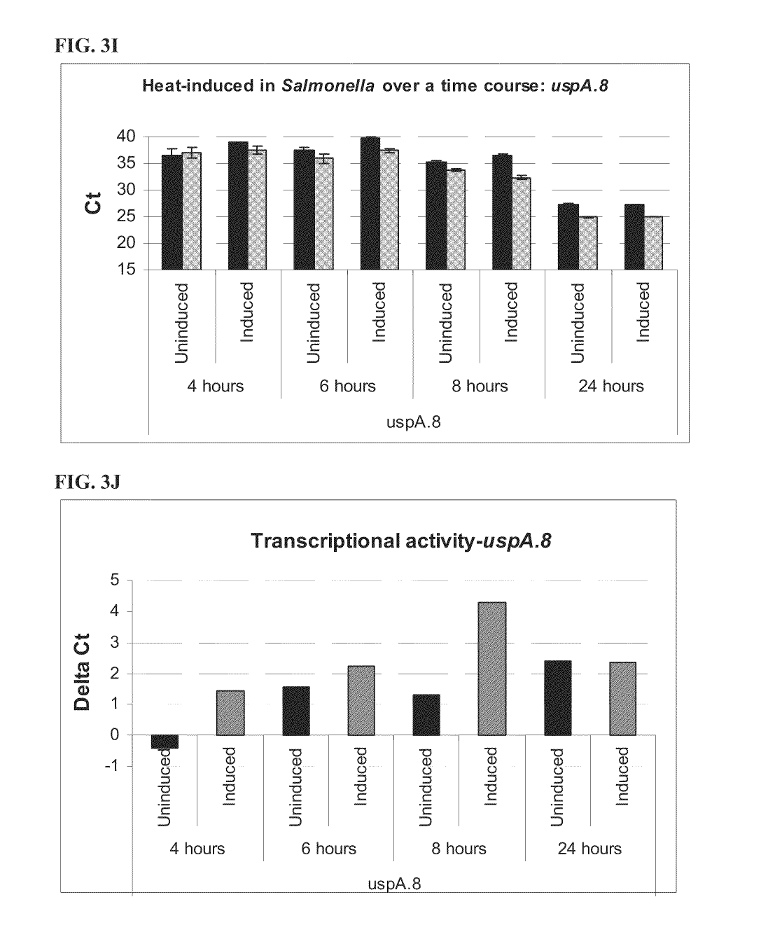

Induction was also evaluated with respect to enrichment time. FIG. 3A-FIG. 3J provide data for evaluating heat-induction of response genes in Salmonella during growth in a food matrix with respect to enrichment time. Samples (25 ml volumes) of whole milk were spiked with approximately 10 cfu of Salmonella. Enrichment broth (225 ml; Brian Heart Infusion Broth) was added. Samples were enriched at 37° C., and duplicate aliquots were withdrawn after 4 hours, 6 hours, 8 hours and 24 hours of growth. One set was maintained at 37° C. (uninduced) and one set was incubated at 45° C. (induced) for 15 minutes. For the data of FIG. 3A-FIG. 3J, duplicate 1 ml samples were withdrawn following 4, 6, 8 and 24 hours and induced or maintained as uninduced. At all times, induction followed by real time RT-PCR, gave the best signal (lowest Ct value). FIGS. 3A, 3C, 3E, 3G, and 3I show Ct data vs time for uninduced and induced cultures. The extracted nucleic acids were assayed for induction by real-time reverse-transcriptase-PCR (hatched bars). To measure contribution due to DNA, control reactions were run without addition of the reverse transcriptase (solid bars). FIGS. 3B, 3D, 3F, 3H, 3J show Delta Ct data vs. time for uninduced and induced cultures. Transcriptional activity was estimated by subtracting the CT value obtained for DNA and RNA (with reverse transciptase reactions) from the CT value obtained for DNA alone (without reverse transcriptase reactions) for uninduced (solid bars) and induced samples (striped bars). At the 8 hour time point, this difference was the highest and signal was reduced from Ct˜35 to a more robust Ct<30 for all assays tested. Additionally, the fold-differences observed during the early times were much higher, indicating the advantage of using exponential cultures for this application, versus the stationary phase cultures following 24 hours of incubation. These studies demonstrate that a shorter enrichment time combined with detection of an inducible RNA target can be used in place of longer enrichment times as a means to achieve higher signals of target.

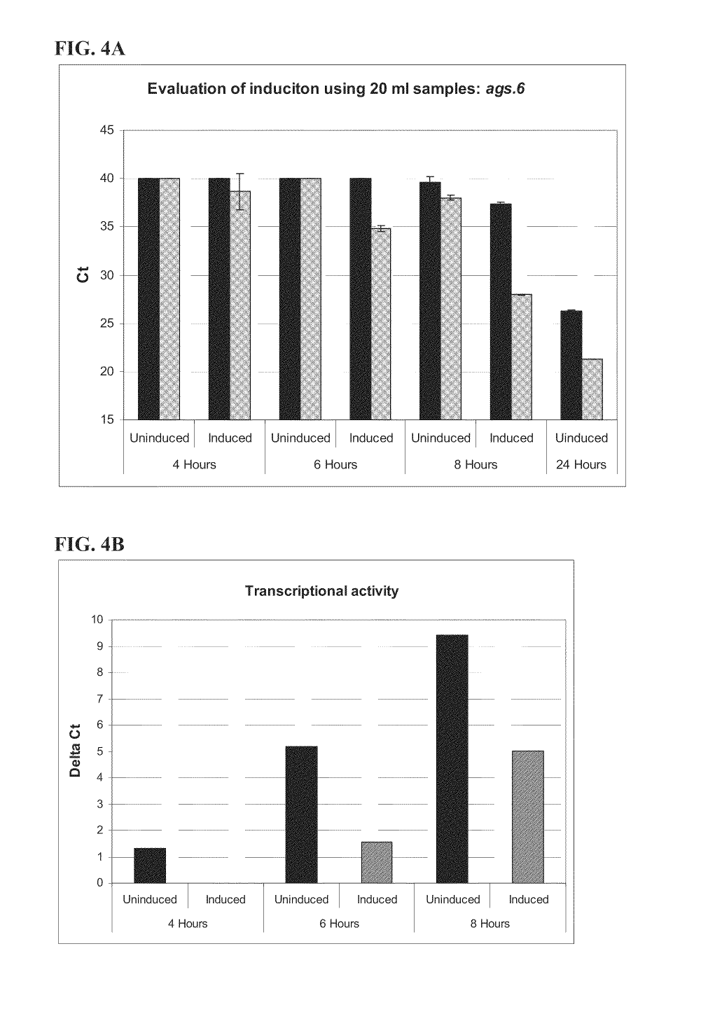

When combined with a larger volume of starting sample (e.g 20 ml), a Ct difference of as much as 9 Ct was observed with spinach rinse samples (FIG. 4A and FIG. 4B). For the data of FIGS. 1A and 4B, spinach (25 g) was spiked with approximately 30 cfu of Salmonella. Prewarmed enrichment broth (225 ml, Brian Heart Infusion Broth) was added. Samples were enriched at 37° C., and duplicate 20 ml aliquots were withdrawn after 4 hours, 6 hours, 8 hours and 24 hours of growth. One set was maintained at 37° C. (uninduced) and one set was incubated at 45° C. (induced) for 15 minutes. FIG. 4A shows Ct data vs time for uninduced and induced cultures (probe/primer set agsA.6). The extracted nucleic acids were assayed for induction by real-time reverse-transcriptase-PCR (hatched bars). To measure contribution due to DNA, control reactions were run without addition of the reverse transcriptase (solid bars). FIG. 4B shows Delta Ct data vs. time for uninduced and induced cultures. Transcriptional activity was estimated by subtracting the CT value obtained for DNA and RNA (with reverse transciptase reactions) from the CT value obtained for DNA alone (without reverse transcriptase reactions) for uninduced (solid bars) and induced samples (striped and dotted bars). The data demonstrate that use of larger volumes of starting sample combined with use of induced RNA detection provides a further improvement in signal, allowing for more robust and earlier time-to-result.

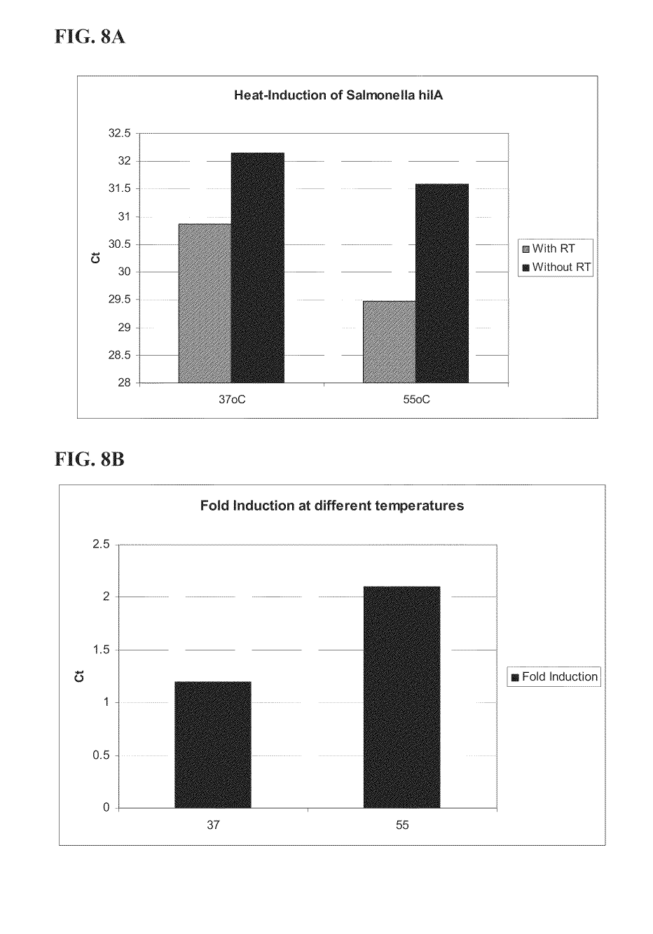

FIG. 8A and FIG. 8B provide data on the use of heat induction to detect Salmonella by measuring the target hilA. The results demonstrate that including the reverse transcriptase step in the detection improves the signal by 2 Cts.

Results for enriched samples were confirmed by plating on CHROMagar™ plates (CHROMager Microbiology, Paris France). That is, following enrichment, samples were withdrawn for RNA extraction and, in parallel, samples were also plated for culture confirmation (CHROMAgar) to verify presence of pathogen. The plate results were recorded following overnight growth at 37° C. A 100% correlation was obtained between RT-PCR results and plate confirmation.

### Example 2

### Inducible RNA Targets for Early Detection of Listeria

The present example describes identification and evaluation of inducible RNA targets for detection of the pathogenic microbe Listeria monocytogenes using real-time RT-PCR.

Materials and Methods; Bacteria and Growth Conditions: A Listeria monocytogenes overnight culture (Brain Heart Infusion broth (BHI), Teknova, Hollister, Calif.) was diluted and grown at 37° C. to obtain exponential phase cells (5 hours). For spiking food samples, 25 g or 25 ml of the food sample was transferred into stomacher bags. Bacteria were spiked in the 1-100 cfu range. Samples were enriched according to standard procedures as described in the Bacteriological Analytical Manual (U.S. Food and Drug Administration, Bacteriological Analytical Manual, Chapter 10, See www.fda.gov). Enrichment media (225 ml) was added to each food sample and sample; for enrichment of Listeria, samples were enriched in Buffered Listeria Enrichment Broth with selective supplements (Buffered Listeria Enrichment Broth, EMD Chemicals, Gibbstown, N.J.). Samples were enriched at 37° C. for a maximum of 24 hours.

Inducing Conditions: For Listeria, several different inducing conditions were evaluated as follows: a) heat induction at 48° C., 20 minutes, b) salt stress at 0.3M NaCl final concentration for 10 minutes at 37° C., c) activated charcoal induction for 5 hours, d) acid stress for 10 minutes at 37° C., and e) cold stress. For b), a 1:1 dilution of culture with brain heart infusion broth containing 0.6M NaCl was made to provide a final concentration of 0.3M NaCl. For c), the Listeria was grown in buffered Listeria enrichment broth treated with activated charcoal. The media was prepared by adding activated charcoal to enrichment broth to 0.2% final volume before autoclaving, stirred for 1 hour, then autoclaved and filtered through a 0.22 uM membrane. For d), a 1:3 dilution of culture in acidified brain heart infusion broth was made. Acidified BHI is prepared by adding HCl to BHI, and checking pH. For example, 100 μl HCl added to 5 ml BHI resulted in a pH of 2.5. For e), for food matrix experiments, the enriched sample was held on ice for 10 minutes. Uninduced controls were maintained at 37° C.

Sample Preparation and Real-time PCR: Sample preparation and real-time PCR procedures were performed as for Example 1.

Primers and Probes: TAQMAN® real-time PCR assays, which include primers and probes for each assay, were designed using a rigorous bioinformatics assay design pipeline against candidate inducible targets in Listeria (Table 2). Full length genes representing each of the candidate targets were obtained from the annotated, publicly available genome sequences of L. monocytogenes EGD-e. These gene sequences were used to query the GenBank database and obtain all other publicly available, homologous Listeria sequences. Assays were designed to match all of the related Listeria sequences, while not matching any other sequences in the microbial subset of GenBank. The probes were labeled with a FAM™ dye to enable detection by real-time PCR.

Assays designed for Listeria are shown in Table 2, which has a column designated as “Gene” indicating the name of the inducible target gene; a column designated as “Assay” which indicates an assay number given to the combination of “Forward Primer” and “Reverse Primer” and “Probe” in the same row as the assay number. For example, one Listeria assay, for detecting the inducible target gene inlA, may comprise assay inlA.0 and use the primer of SEQ ID NO: 400 (forward primer), the primer of SEQ ID NO: 855 (reverse primer) and the probe of SEQ ID NO: 1310. In another example, one Listeria assay, for detecting the inducible target gene lmo0539, may comprise assay lmno0539.1 and use the primer of SEQ ID NO: 422 (forward primer), the primer of SEQ ID NO: 877 (reverse primer) and the probe of SEQ ID NO: 1332.

TABLE 2
TaqMan® assays designed against candidate inducible genes in Listeria.

| Gene | Assay | Forward Primer | Reverse Primer | Probe |
|---|---|---|---|---|
| inLA | inlA.0 | GCGATGGTGGTAGTTATGCAGAA | GTTCCTTTTCCAATGGTGACAGATT | CCTGATATAACATGGAATTTA |
| | | SEQ ID NO: 400 | SEQ ID NO: 855 | SEQ ID NO: 1310 |
| | inlA.1 | GGCACATTGGCGAGTTTAACAAA | TGTTACTTATTTGGTTAGCTCCCA | TCGGGTCTAACAAAACTAAC |
| | | SEQ ID NO: 401 | GTTTT | SEQ ID NO: 1311 |
| | | | SEQ ID NO: 856 | |
| | inlA.2 | CGGTCTTAGGAAAAACGAATGTAACAG | GAAGCGTCGTAACTTGGTCTAGAT | CACGGTCTCACAAACAG |
| | | SEQ ID NO: 402 | SEQ ID NO: 857 | SEQ ID NO: 1312 |
| | inlA.3 | CGATAATGCGCTCTACCTGTTGATA | TGCTTTTTTAGTAAGAGCCACTGCA | TCCTACTGCTAATAACCC |
| | | SEQ ID NO: 403 | SEQ ID NO: 858 | SEQ ID NO: 1313 |
| | inlA.4 | CCAACAAAAGCCGGATATGCT | GCTGGCATTTTATCTGTCGCAAA | TTTCGTCATACCAACCTTTG |
| | | SEQ ID NO: 404 | SEQ ID NO: 859 | SEQ ID NO: 1314 |
| | inlA.5 | CGAATCTAACTGGTTTGACTTTGTTCAA | GTGTTACTGGATAGTTCTAGCCGATT | AAGCGGGTCTATATCCG |
| | | SEQ ID NO: 405 | SEQ ID NO: 860 | SEQ ID NO: 1315 |
| | inlA.6 | CCAAATAAGTGATATAACTCCACTTGGGA | ACTCGCCAATGTGCCTATATCTTTT | TTGGACGAATTATCCTTAAATGG |
| | | SEQ ID NO: 406 | SEQ ID NO: 861 | SEQ ID NO: 1316 |
| | inlA.7 | TCAGGCAGCTACAATTACACAAGAT | TCGTTTTTCCTAAGACCGTCTTCATT | CCGCTAGAGCTGTATCTG |
| | | SEQ ID NO: 407 | SEQ ID NO: 862 | SEQ ID NO: 1317 |
| | inlA.8 | CCCAATTTCTAACCTGAAAAATCTCACA | CGAGCTTACGTCACTTACCTTGTTA | AAGCCCAGTTTCTAGTTTAA |
| | | SEQ ID NO: 408 | SEQ ID NO: 863 | SEQ ID NO: 1318 |
| | inlA.9 | GGACGAATTATCCTTAAATGGTAACCAGTT | CCGACAGTGGTGCTAGATTACTAAT | ACATTGGCGAGTTTAAC |
| | | SEQ ID NO: 409 | SEQ ID NO: 864 | SEQ ID NO: 1319 |
| | inlA.10 | ACCAAATTAGTAATCTAGCACCACTGTC | GGGACTGATGTTACTTATTTGGTTAGC | AACTCAGTTAGTTTTGTTA |
| | | SEQ ID NO: 410 | SEQ ID NO: 865 | GACCC |
| | | | | SEQ ID NO: 1320 |
| | inlA.11 | CCTGTCACTATTGGAAAAGGAACGA | GCCGTCCACATGAAACTTAGCATTA | CACGGTTCCACTAAATG |
| | | SEQ ID NO: 411 | SEQ ID NO: 866 | SEQ ID NO: 1321 |
| | inlA.12 | ACGGTCTCGCAAACAGATCTAG | GTTAAATTGTTCAAGTATTCCAA | ACCAAGTTACAACGCTTCAG |
| | | SEQ ID NO: 412 | TCCATCGA | SEQ ID NO: 1322 |
| | | | SEQ ID NO: 867 | |
| | inlA.13 | CGCAGAAACAGGCGGTAAAA | AGTTACTGGATTTGCAGGCATCTT | TCTTGAGCAAAATCC |
| | | SEQ ID NO: 413 | SEQ ID NO: 868 | SEQ ID NO: 1323 |
| | inlA.14 | GCACAACAAGTACAATGAACGCTTA | CCCTAACAAAAGGTAGAGCGCATTA | CCTACAACTGGCGATAGC |
| | | SEQ ID NO: 414 | SEQ ID NO: 869 | SEQ ID NO: 1324 |
| | inlA.15 | GCAAATATACCTGGAAGCAACACAT | GCGTTCATTGTACTTGTTGTGCTA | ATCAACTGGGAATTCAGC |
| | | SEQ ID NO: 415 | SEQ ID NO: 870 | SEQ ID NO: 1325 |
| | inlA.16 | GCTTCAGGCAGATAGATTAGGGATAA | TCTTTAAGTGGCGTTATATCCGTA | CACAAATAAATTTCAGCAATAAT |
| | | SEQ ID NO: 416 | AGTTG | SEQ ID NO: 1326 |
| | | | SEQ ID NO: 871 | |
| | inlA.17 | AGCGTTAAATCGTATACAGCAACATTTG | GCTGCCGGTTCTGTCAGA | TCGTTGTTGTTCCTTCTTTG |
| | | SEQ ID NO: 417 | SEQ ID NO: 872 | SEQ ID NO: 1327 |
| | inlA.18 | CTGGTCTAACCGCACTCACTAA | GTGAGATTTTTCAAGTTAGAAATTG | CTTAGAGCTAAATGAAAATC |
| | | SEQ ID NO: 418 | GGCTAA | SEQ ID NO: 1328 |
| | | | SEQ ID NO: 873 | |
| | inlA.19 | CATGGAACTTACCTAGCTATACGAATGAA | AGTGGCTGCGTCACAGTT | AAGGAACGACAACATTTAG |
| | | SEQ ID NO: 419 | SEQ ID NO: 874 | SEQ ID NO: 1329 |
| | inlA.20 | CGACTCAAGCAGTAGACTATCAAGGA | GTTTTTTCGTCATACCAGCCTTTGA | ACGAAAGCCGGATATAC |
| | | SEQ ID NO: 420 | SEQ ID NO: 875 | SEQ ID NO: 1330 |
| lmo053 | lmo0539.0 | CGGCAAACAAGGCGATGA | GTTTTCTTTCCCTTGCGTACGAA | CCATTCGCGTAAAGCG |
| 9 | | SEQ ID NO: 421 | SEQ ID NO: 876 | SEQ ID NO: 1331 |
| | lmo0539.1 | GATCCAACAAGCAAAAGGAACAGAA | ATCGCAGAAGCATAAGGTGTAAGTT | AAAAGATGTGGAAGACTTC |
| | | SEQ ID NO: 422 | SEQ ID NO: 877 | SEQ ID NO: 1332 |
| | lmo0539.2 | GATCCAACAAGCAAAAGGAACAGAA | GCAAAATCGCAGAAGCATAAGGT | CTTGTTTCTGAAGAACTTAC |
| | | SEQ ID NO: 423 | SEQ ID NO: 878 | SEQ ID NO: 1333 |
| | lmo0539.3 | GCAACAACTCCTGGTAAACTTCCT | CAGCATCTCCGCCATTTTCTTTAAT | AAGTGCCGATAAATCTT |
| | | SEQ ID NO: 424 | SEQ ID NO: 879 | SEQ ID NO: 1334 |
| | lmo0539.4 | AATATGGTACTCCAGCAATCAAAGCT | GGAGTAGTTGCGTCATATCCTGTTT | ACGAAGGTAGCGGACTTT |
| | | SEQ ID NO: 425 | SEQ ID NO: 880 | SEQ ID NO: 1335 |
| | lmo0539.5 | TGATGCAAAAGTTACTGATTCTGGTTCT | CGAGGTTTAGAGAATTCTTTAATG | TTTGCTGGTTTTAATTTC |
| | | SEQ ID NO: 426 | GAAGCT | SEQ ID NO: 1336 |
| | | | SEQ ID NO: 881 | |
| | lmo0539.6 | TGCTTCTGCGATTTTGCTTGATTT | AGTTAACAGTCCGCTACCTTCATG | CCAGCAATCAAAGCTC |
| | | SEQ ID NO: 427 | SEQ ID NO: 882 | SEQ ID NO: 1337 |
| | lmo0539.7 | CTACACCTGGTAAACTGCCTGATT | GCATCTCCGCCGTTTTCTTTAAT | ACGAAGTGCAGATAAGTCTT |
| | | SEQ ID NO: 428 | SEQ ID NO: 883 | SEQ ID NO: 1338 |
| | lmo0539.8 | AAACCAGCAAAAGTAAAAGCTTCCATT | CTTCCGCAAAACCTTCCACATATTT | ATGGTGTAGATGTACTTAAAC |
| | | SEQ ID NO: 429 | SEQ ID NO: 884 | SEQ ID NO: 1339 |
| | lmo0539.9 | GCGAACCAACATAACGTGCAATATA | TCGATACCATCAGCCCAAGTTG | CACGACCGCAAAGTAC |
| | | SEQ ID NO: 430 | SEQ ID NO: 885 | SEQ ID NO: 1340 |
| | lmo0539.10 | ATGGTACAAATAACAAAAGGTAAATTTGATGGT | AATGGCAAGTGCTGCAATGAC | TATCGTTGGAGAGTCTTTG |
| | | SEQ ID NO: 431 | SEQ ID NO: 886 | SEQ ID NO: 1341 |
| | lmo0539.11 | AAGGATGTAGAAGACTTCAAACAACTTGT | AGCTTTGATTGCTGGAGTACCAT | TCTAAATCAAGCAAAATCG |
| | | SEQ ID NO: 432 | SEQ ID NO: 887 | SEQ ID NO: 1342 |
| | lmo0539.12 | CCTCGTTATGGTGTAGATGTACTTAAACTT | CTCGTCTTGCGTATAAGCTACTTCA | CCTTCAGCAAAACCTTC |
| | | SEQ ID NO: 433 | SEQ ID NO: 888 | SEQ ID NO: 1343 |
| | lmo0539.13 | TGCTTCTGCGATTTTGCTTGATTT | AAGTTAAAAGTCCGCTACCTTCGT | CCAGCAATCAAAGCTC |
| | | SEQ ID NO: 434 | SEQ ID NO: 889 | SEQ ID NO: 1344 |
| | lmo0539.14 | TGGCGTAACTTCTGAAATGTTCCAT | GTTGCACGACCACAAAGTACAC | AATCAACATAACGTACAATATAG |
| | | SEQ ID NO: 435 | SEQ ID NO: 890 | SEQ ID NO: 1345 |
| | lmo0539.15 | GTGAAGTCGCTTATACGCAAGAC | GGGCTTAAGTCAGAGCACTCTT | AAGCAGCTCGTCATTTTG |
| | | SEQ ID NO: 436 | SEQ ID NO: 891 | SEQ ID NO: 1346 |
| | lmo0539.16 | ACGAAGGTAGCGGACTTTTAACTT | TGCATCTCCACCATTTTCTTTAATACGA | CAGGATATGACGCAACTAC |
| | | SEQ ID NO: 437 | SEQ ID NO: 892 | SEQ ID NO: 1347 |
| | lmo0539.17 | TGGGCTGATGGTATTGAAGTGTAC | CCTTGCGTACGAAGCCATTC | CATCGCCTTGTTTGCC |
| | | SEQ ID NO: 438 | SEQ ID NO: 893 | SEQ ID NO: 1348 |
| | lmo0539.18 | CCAGCGAAAGTAAAAGCATCTATTAAAGAA | ACCTTCAGCAAAACCTTCCACATAT | CACCGTAACGAGGTTTAG |
| | | SEQ ID NO: 439 | SEQ ID NO: 894 | SEQ ID NO: 1349 |
| | lmo0539.19 | AATTGAAACCAGCAAAAGTAAAAGCTTCT | ACCTTCAGCAAAACCTTCCACATAT | ACACCATAACGAGGTTTAG |
| | | SEQ ID NO: 440 | SEQ ID NO: 895 | SEQ ID NO: 1350 |
| | lmo0539.20 | GGTGGAGATGCAGTTAAAATTCTTGTT | CGACATTCAGCACCAATTCTTTCT | ATGAGCCTGCTGAAATTA |
| | | SEQ ID NO: 441 | SEQ ID NO: 896 | SEQ ID NO: 1351 |
| lmo215 | lmo2158.0 | GTAGTAGGTGACGCAAAAGACAAGT | CTTCGCCTTTAGCTTTTTGAGCTTT | TCGGTAAAGCAACAGATGAT |
| 8 | | SEQ ID NO: 442 | SEQ ID NO: 897 | SEQ ID NO: 1352 |
| | lmo2158.1 | AGGTATGAAAGACAAAGCAAAAGGACTT | TTTGCCTTTATCATCTGTTGCTTTACC | ACGCAAAAGACAAGTTC |
| | | SEQ ID NO: 443 | SEQ ID NO: 898 | SEQ ID NO: 1353 |
| | lmo2158.2 | GGCAAACAAGTTGAAGGTAAAGCT | TTTAGCGTCACCGGTTTTATCTTCT | ACTTCGCCTTTAGCTTTT |
| | | SEQ ID NO: 444 | SEQ ID NO: 899 | SEQ ID NO: 1354 |
| | lmo2158.3 | CGAAACAAGCAGAAGGTAAAGCA | GCGTCGCCAGTTTTGTCTTC | ACTTCGCCTTTAGCTTTT |
| | | SEQ ID NO: 445 | SEQ ID NO: 900 | SEQ ID NO: 1355 |
| | lmo2158.4 | ACAAGTTCGGTAAAGCAACAGATGA | AGCGTCGCCCGTTTTATCTT | ACAAGTAGAAGGTAAAGCTC |
| | | SEQ ID NO: 446 | SEQ ID NO: 901 | SEQ ID NO: 1356 |
| | lmo2158.5 | AGGTATGAAGGACAAAGCAAAAGGAT | CTGTTGCTTTCCCGAATTTGTCTT | TTGCGTCACCTACTACTTTATC |
| | | SEQ ID NO: 447 | SEQ ID NO: 902 | SEQ ID NO: 1357 |
| | lmo2158.6 | CGCAAAAGACAAATTCGGTAAAGCA | GTCGCCAGTTTTGTCTTCTACTTC | ACCTTCTGCTTGTTTCGC |
| | | SEQ ID NO: 448 | SEQ ID NO: 903 | SEQ ID NO: 1358 |
| | lmo2158.7 | ATGAGCGAAGATAAAGGTATGAAGGA | CTGTTGCTTTCCCGAATTTGTCTT | TTGCGTCACCTACTACTTTATC |
| | | SEQ ID NO: 449 | SEQ ID NO: 904 | SEQ ID NO: 1359 |
| | lmo2158.8 | ACAAAGCAAAAGGAATGAAAGACAAAGT | GCTTTGTCGTCTGTTGCTTTACC | ACGCAAAAGACAAATTC |
| | | SEQ ID NO: 450 | SEQ ID NO: 905 | SEQ ID NO: 1360 |
| | lmo2158.9 | AGGTATGAAAGACAAAGCAAAAGGACTT | CATCTGTTGCTTTACCGAACTTGT | TTGCGTCACCTACTACTTTGTC |
| | | SEQ ID NO: 451 | SEQ ID NO: 906 | SEQ ID NO: 1361 |
| | lmo2158.10 | GTGACGCAAAAGACAAATTCGGTAA | TTTTGTCTTCTACTTCGCCTTTAGC | CAACAGACGACAAAGCG |
| | | SEQ ID NO: 452 | SEQ ID NO: 907 | SEQ ID NO: 1362 |
| | lmo2158.11 | GGGAAAGCAACAGATGACAAAGG | TLI IIII AGCGTCACCGGTTTTATCT | ACAAGTTGAAGGTAAAGCT |
| | | SEQ ID NO: 453 | SEQ ID NO: 908 | SEQ ID NO: 1363 |
| | lmo2158.12 | ACAAGTTCGGTAAAGCAACAGATGA | GCGTCGCCCGTTTTATCTTC | ACTTCGCCTTTAGCTTTT |
| | | SEQ ID NO: 454 | SEQ ID NO: 909 | SEQ ID NO: 1364 |
| | lmo2158.13 | GGTATGAAAGACAAAGCAAAAGGAATGA | TCGTCTGTTGCTTTACCGAATTTG | TTGCGTCACCTACTACTTTGTC |
| | | SEQ ID NO: 455 | SEQ ID NO: 910 | SEQ ID NO: 1365 |
| | lmo2158.14 | AGTAGTAGGTGACGCAAAAGACAAAT | GCTTTACCTTCAACTTGTTTGCCTTT | TCATCTGTTGCTTTCCC |
| | | SEQ ID NO: 456 | SEQ ID NO: 911 | SEQ ID NO: 1366 |
| | lmo2158.15 | AGGTATGAAGGACAAAGCAAAAGGAT | GCCTTTGTCATCTGTTGCTTTCC | ACGCAAAAGACAAATTC |
| | | SEQ ID NO: 457 | SEQ ID NO: 912 | SEQ ID NO: 1367 |
| | lmo2158.16 | GGCATGAAAGACAAAGCAAAAGGA | CATCTGTTGCTTTACCGAACTTGT | TTGCGTCACCTACTACTTTGT |
| | | SEQ ID NO: 458 | SEQ ID NO: 913 | SEQ ID NO: 1368 |
| | lmo2158.17 | TGAAGGACAAAGGAAAAGGATTGAAAGA | TTTGCCTTTGTCATCTGTTGCTTT | ACGCAAAAGACAAATTC |
| | | SEQ ID NO: 459 | SEQ ID NO: 914 | SEQ ID NO: 1369 |
| | lmo2158.18 | GAGCGAAGATAAAGGCATGAAAGAC | TCATCTGTTGCTTTACCGAACTTGT | AAGCAAAAGGACTTAAAGACAAA |
| | | SEQ ID NO: 460 | SEQ ID NO: 915 | SEQ ID NO: 1370 |
| bsh | bsh.0 | CGTCAATAACTTATACAACGAAGGATCACT | GGCGTAACAACCACAACTTCTTTG | TTTGGAAGGAATTTCG |
| | | SEQ ID NO: 461 | SEQ ID NO: 916 | SEQ ID NO: 1371 |
| | bsh.1 | CATTTATGATAATCCTGTTGGCGTGTT | CACTCGAAAGAACGCGATAATTGTT | CAACATTTGATTACCAACTA |
| | | SEQ ID NO: 462 | SEQ ID NO: 917 | TTTAA |
| | | | | SEQ ID NO: 1372 |
| | bsh.2 | GCCGCTATCTCCTTTACATTGGTT | TCCATCTTTCACACATTCCACTACAA | ATGGCTGATCAAAACGAATCTA |
| | | SEQ ID NO: 463 | SEQ ID NO: 918 | SEQ ID NO: 1373 |
| | bsh.3 | GCCGCTATCTCCTTTACATTGGTT | TCCATCTTTCACACATTCCACTACAA | ATGGCTGATCAAACTGAATC |
| | | SEQ ID NO: 464 | SEQ ID NO: 919 | SEQ ID NO: 1374 |
| | bsh.4 | GGATGCTTATAGTCGTGGGATGG | TGCTTTCACAAAACGAGACATAGATGA | CAGGTAAGCCAATCCCCC |
| | | SEQ ID NO: 465 | SEQ ID NO: 920 | SEQ ID NO: 1375 |
| | bsh.5 | TCTGAATCAGAGAGCATTAGCCAATTT | CACCAACATCACACAGACCTTTT | CCGAGCCTAAAATATG |
| | | SEQ ID NO: 466 | SEQ ID NO: 921 | SEQ ID NO: 1376 |
| | bsh.6 | TCTGAATCAGAGAGCATTAGCCAATTT | CCACCAACATCACAAAGACCTTTT | CCGAGCCTAAAATATG |
| | | SEQ ID NO: 467 | SEQ ID NO: 922 | SEQ ID NO: 1377 |
| | bsh.7 | GCGCTACTGTAAAAGAAGCAAGAAG | CGTTTTGATCAGCCATCAACCAA | TTGCCGCTATCTCCTTTACA |
| | | SEQ ID NO: 468 | SEQ ID NO: 923 | SEQ ID NO: 1378 |
| | bsh.8 | AGTGGAATGTGTGAAAGATGGACTTC | CACTCGAAAGAACGCGATAATTGTT | CACGCCAACAGGATTA |
| | | SEQ ID NO: 469 | SEQ ID NO: 924 | SEQ ID NO: 1379 |
| | bsh.9 | GCGCTACTGTAAAAGAAGCAAGAAG | TGGAGATAGCGGCAAATTTTCACTA | CAGAGAATCAATCTCGTAAATAT |
| | | SEQ ID NO: 470 | SEQ ID NO: 925 | SEQ ID NO: 1380 |
| | bsh.10 | GGCGATTTGTCTTCTATGTCTCGTT | TTGGCCAATGCTTTCTGATTCAG | ACCTGAAACAGAATTCA |
| | | SEQ ID NO: 471 | SEQ ID NO: 926 | SEQ ID NO: 1381 |
| | bsh.11 | GCGCTACTGTAAAAGAAGCAAGAAG | TCTGTTTGATCAGCCATCAACCA | CCGCTGTCTCCATTACAT |
| | | SEQ ID NO: 472 | SEQ ID NO: 927 | SEQ ID NO: 1382 |
| | bsh.12 | CGGCTTACCTGGTGATTTATCTTCT | TGCTTTCTGATTCAGAATCACCTGAAA | CTTTTACCAAATTGAATTCTG |
| | | SEQ ID NO: 473 | SEQ ID NO: 928 | SEQ ID NO: 1383 |
| | bsh.13 | TGGGATGGGCGGAATTGG | AAAAGTTGCTTTCACAAAACGAGACAT | CTTACCTGGTGATTTATCTTC |
| | | SEQ ID NO: 474 | SEQ ID NO: 929 | SEQ ID NO: 1384 |
| | bsh.14 | CGCGTTCTTTCGAGTGAAACTC | TCGCCAGGTAAGCCGATTC | CCCACGACTATAAGCATCC |
| | | SEQ ID NO: 475 | SEQ ID NO: 930 | SEQ ID NO: 1385 |
| | bsh.15 | CAATTATCGCGTTCTTTCGAGTGAA | GTAAGCCGATTCCGCCCAT | CCCACGACTATAAGCATCC |
| | | SEQ ID NO: 476 | SEQ ID NO: 931 | SEQ ID NO: 1386 |
| | bsh.16 | CGGTGGAACAACAAAAAGGTCTTT | CCACACCAGTAATTTGACTGTTTCC | CTCGTGTTGCAATATC |
| | | SEQ ID NO: 477 | SEQ ID NO: 932 | SEQ ID NO: 1387 |
| | bsh.17 | TCAGAGAGCATTAGCCAATTTTTCCA | TCCCCACCAACATCACAAAGAC | CTTTTTGTTGTTCCACCGAGCCT |
| | | SEQ ID NO: 478 | SEQ ID NO: 933 | SEQ ID NO: 1388 |
| | bsh.18 | CATTTATGATAATCCTGTTGGCGTGTTAA | TCGCTCGAAAGAACACGATAATTGT | CAAATAATCCAACATTTGATTACC |
| | | SEQ ID NO: 479 | SEQ ID NO: 934 | SEQ ID NO: 1389 |
| | bsh.19 | AGAGATTGATTTGGATGCTTATAGTCGTG | GCTTTCACAAAACGAGACATAGAAGAC | AAGCCGATTCCGCCCATC |
| | | SEQ ID NO: 480 | SEQ ID NO: 935 | SEQ ID NO: 1390 |
| inlB | inlB.0 | TGTTGATAGAGAAGCCCGAAATGG | TCCGCTTTAGTCCAGCCAATATTTT | CCTGTACCATAATTTCC |
| | | SEQ ID NO: 481 | SEQ ID NO: 936 | SEQ ID NO: 1391 |
| | inlB.1 | GGATGGCTTTTTTTAGACGAAAATAAAA | ACTTATACCATTATGCTCCAAAGA | CCTTGAGCGAACTTAG |
| | | TTAAAGAC | AAGTG | SEQ ID NO: 1392 |
| | | SEQ ID NO: 482 | SEQ ID NO: 937 | |
| | inlB.2 | TGAAAGAAAAGTACAACCCAAGAAGGAA | CGGTGATAGTCTCCGCTTGT | CTTTCGCCCCGTTTCC |
| | | SEQ ID NO: 483 | SEQ ID NO: 938 | SEQ ID NO: 1393 |
| | inlB.3 | GCGCACTTGATACGTTCTATAAGCA | GCTTCGTTTACTTTGTTCGCAATCA | AAGCATGGAGAAAGATACTAAT |
| | | SEQ ID NO: 484 | SEQ ID NO: 939 | SEQ ID NO: 1394 |
| | inlB.4 | ACAGCCCAAGAAGGAAGTATTGTTT | GCTTGATTGGCGTTGGCA | CAAGCGGAGACTATCAC |
| | | SEQ ID NO: 485 | SEQ ID NO: 940 | SEQ ID NO: 1395 |
| | inlB.5 | GTACGGATTATATGTCCGGAAACGA | GCGTGTTAAGTTCACCG | CAGTCGTTTCCACTTTAAAC |
| | | SEQ ID NO: 486 | SEQ ID NO: 941 | SEQ ID NO: 1396 |
| | inlB.6 | AGACCTAAGTTCGCTCAAGGATCTA | CAAATACAAACTTTCCAGCTGTGGTA | ATGAACAAGTCCGTTTATATCAC |
| | | SEQ ID NO: 487 | SEQ ID NO: 942 | SEQ ID NO: 1397 |
| | inlB.7 | TGTTGTGGATGCGGATGTTAGAT | GTTGCTTGTTTATCCACGGTTAGTT | TTTATAAGCAGCTAAAGTGCCCC |
| | | SEQ ID NO: 488 | SEQ ID NO: 943 | SEQ ID NO: 1398 |
| | inlB.8 | TCCGGTAGTAGATAGCCCAATCAAA | ACCTTCAACGGTGGCTGTT | CGATCGACAATTAATGTTT |
| | | SEQ ID NO: 489 | SEQ ID NO: 944 | GATTTT |
| | | | | SEQ ID NO: 1399 |
| | inlB.9 | CAATCTAATTTGGTCGTTCCGAATACAG | CGGTTTTTCATAATCGCCATCATCA | AACGACCCATCAGTGTTTT |
| | | SEQ ID NO: 490 | SEQ ID NO: 945 | SEQ ID NO: 1400 |
| | inlB.10 | AGTGATGATGGCGATTATGAAAAACCT | GCTTTTCCAATAGTGACTGGCTGAT | ATGGCATTTACCAGAATTTA |
| | | SEQ ID NO: 491 | SEQ ID NO: 946 | SEQ ID NO: 1401 |
| | inlB.11 | GGCATGAGTGGAATTTTAATACGGATT | AGCGGGTTAAGTTGACTGCTT | TTTCGGTCGTTTCCGCTTTA |
| | | SEQ ID NO: 492 | SEQ ID NO: 947 | SEQ ID NO: 1402 |
| | inlB.12 | AGTAAGTTATGATGTTGATGGAACGGT | CATAGCCTTGTTTAGTCGGAGGT | ACGCGGATAACTGCACCTAA |
| | | SEQ ID NO: 493 | SEQ ID NO: 948 | SEQ ID NO: 1403 |
| | inlB.13 | CGAAGCCAAAACACCAATTACCA | TGTTAAGTGCACGCGTATCGA | TTAGCATTGATGGTAAAGTAATT |
| | | SEQ ID NO: 494 | SEQ ID NO: 949 | SEQ ID NO: 1404 |
| | inlB.14 | CCTCCGACCAAACAAGGCTAT | CGTACAAAGTAAAATCATTTCCGGACAT | CTCATGCCCACCATTTT |
| | | SEQ ID NO: 495 | SEQ ID NO: 950 | SEQ ID NO: 1405 |
| | inlB.15 | ACAGATATAAAGCCCTTAGCAAACTTGAA | CTTGAGCGAACTTAGGTCCTTAACT | ATGGCTTTTTTTAGACGAAAAT |
| | | SEQ ID NO: 496 | SEQ ID NO: 951 | SEQ ID NO: 1406 |
| | inlB.16 | TGCCAACACCAATCAAGCAAATTTT | ATTTTGTGTCACTAGATCCGTCACA | CAGAAACTATCAAAGACAATTT |
| | | SEQ ID NO: 497 | SEQ ID NO: 952 | SEQ ID NO: 1407 |
| | inlB.17 | GAAACGATTTTACTTTGTACGCGATGT | GCGGGTTAAGTTCACTGCTTTT | TCAGTCGTTTCCGCTTTAA |
| | | SEQ ID NO: 498 | SEQ ID NO: 953 | SEQ ID NO: 1408 |
| | inlB.18 | GCGATTTAAGAGCATTAGCAGGACTT | TCACTGTATTCGGAACAACCAAATTAGA | AAGACATTCTTGGCTAAATAA |
| | | SEQ ID NO: 499 | SEQ ID NO: 954 | SEQ ID NO: 1409 |
| | inlB.19 | GTACGCGATGTTTAAAGCGGAAA | AGTTTGTAGATCCCCGCATTCC | ATAGCGTGTTAAATTCAC |
| | | SEQ ID NO: 500 | SEQ ID NO: 955 | SEQ ID NO: 1410 |
| | inlB.20 | CCTCCGACCAAACAAGGCTATG | CGTTTCCGGACATATAATCCGTACT | ATGCCCACCATTTTTT |
| | | SEQ ID NO: 501 | SEQ ID NO: 956 | SEQ ID NO: 1411 |
| lmo059 | lmo0596.0 | TGAATTTGACGGAACTTTAACACTCGTA | CGCCAATTGTACGCATAATACCA | CCCACATACCGAAAAGT |
| 6 | | SEQ ID NO: 502 | SEQ ID NO: 957 | SEQ ID NO: 1412 |
| | lmo0596.1 | GGGCTGGATTTTAACAATCGGTATT | CGATACCAATTGCGGAAACAACTG | ATAGCGCGATAAAACC |
| | | SEQ ID NO: 503 | SEQ ID NO: 958 | SEQ ID NO: 1413 |
| | lmo0596.2 | CACAAAATGTTTCTGGCTGGGTAC | CCACATACCGAAAAGTAATACGAGTGT | TAGCTGACGGAATTTT |
| | | SEQ ID NO: 504 | SEQ ID NO: 959 | SEQ ID NO: 1414 |
| | lmo0596.3 | CCACACAATCTCTTACTTTTCAGACAGA | GATGGATAAAATCCCGTCAGCTAGT | ACCCAGCCAGAAACA |
| | | SEQ ID NO: 505 | SEQ ID NO: 960 | SEQ ID NO: 1415 |
| | lmo0596.4 | CGCAATTGGTATCGTCCTTGTC | GTTGCGATTGCGCCGAT | ACCTTGAACGATGAAGAAA |
| | | SEQ ID NO: 506 | SEQ ID NO: 961 | SEQ ID NO: 1416 |
| | lmo0596.5 | AAAATCACAAAATGTTTCTGGTTGGGT | CAAGTGTTAACGTTCCCTCAAATTCA | TAGCTGACGGAATTTT |
| | | SEQ ID NO: 507 | SEQ ID NO: 962 | SEQ ID NO: 1417 |
| | lmo0596.6 | AGACTTTTACACGAATTCTTGTTTTATTAGCG | GGAGGTTAATAATGAAATTCCTGGATGGA | CCAAACCCCTAGTATAATCAT |
| | | SEQ ID NO: 508 | SEQ ID NO: 963 | SEQ ID NO: 1418 |
| | lmo0596.7 | GGCGCATTCACTGCTAAACAAAATA | GCGGAGACAACTGGATTGAATAGAG | ACCAACAATGATACCAATAAT |
| | | SEQ ID NO: 509 | SEQ ID NO: 964 | SEQ ID NO: 1419 |
| | lo0596.8 | GCGCATTCACTGCTAAACAAAACA | GCGGAAACAACTGGATTGAATAGAG | CAATAATGCCGATAATACC |
| | | SEQ ID NO: 510 | SEQ ID NO: 965 | SEQ ID NO: 1420 |
| | lmo0596.9 | ATCCAGGAATTTCATTATTAACCTCGACAT | CCCAGCCAGAAACATTTTGTGAT | ACTGATTTCTGGTATTTTC |
| | | SEQ ID NO: 511 | SEQ ID NO: 966 | SEQ ID NO: 1421 |
| | lmo0596.10 | CTCGTATTACTTTTCGGTATGTGGGTAT | GTTGTTTTGTTTGGCAGTGAATGC | CTGGTATTATGCGTACAATTG |
| | | SEQ ID NO: 512 | SEQ ID NO: 967 | SEQ ID NO: 1422 |
| | lmo0596.11 | TGTATTGCTGTTTGGTATGTGGGT | CAGTGAATGCGCCGATTGTAC | TTGTTTGCTGGTATTATGC |
| | | SEQ ID NO: 513 | SEQ ID NO: 968 | SEQ ID NO: 1423 |
| | lmo0596.12 | CCACACAATCTCTTATTTCTCAGACAGA | GTAAGATGGATAAAATTCCGTCAGCTAGT | CCCAACCAGAAACATTT |
| | | SEQ ID NO: 514 | SEQ ID NO: 969 | SEQ ID NO: 1424 |
| | lmo0596.13 | ACCTCAACATTGATGATCGGTTTCTT | ACCCACCCAGAAACATTTTGTGAT | ATTTTCCACACAATTTCT |
| | | SEQ ID NO: 515 | SEQ ID NO: 970 | SEQ ID NO: 1425 |
| | lmo0596.14 | ACTGCCAAACAAAACAACGTACAAG | GAGACAACTGGATTGAATAGAGCGATA | CAATAATGCCGATAATACC |
| | | SEQ ID NO: 516 | SEQ ID NO: 971 | SEQ ID NO: 1426 |
| | lmo0596.15 | GGCATTATTGTTGGTTTTATCGCTCTA | TCGCAACGACAAGGACGATAC | CAATTGCGGAAACAACT |
| | | SEQ ID NO: 517 | SEQ ID NO: 972 | SEQ ID NO: 1427 |
| | lmo0596.16 | GCGCTATTCAATCCAGTTGTTTCC | GTTGCGATTGCGCCGATAC | CAACGACAAGGACGATACC |
| | | SEQ ID NO: 518 | SEQ ID NO: 973 | SEQ ID NO: 1428 |
| | lmo0596.17 | ATCATTGTTGGATTTATCGCTCTCTTCA | GCAATTGCTCCGATACCTTGAAC | TTGCAACGATAAGTACTATTC |
| | | SEQ ID NO: 519 | SEQ ID NO: 974 | SEQ ID NO: 1429 |
| | lmo0596.18 | GGTGCTAGCTGACGGGATTTTATC | CCACATACCGAAAAGTAATACGAGTGT | CCGTCAAATTCATTAAATAAT |
| | | SEQ ID NO: 520 | SEQ ID NO: 975 | SEQ ID NO: 1430 |
| | lmo0596.19 | GGGATTGCAATGATTATACTTGGGATT | ACCAGAAATCAGTAATAAGAAACCGAACAT | CCTGGATGGAATAAGAACC |
| | | SEQ ID NO: 521 | SEQ ID NO: 976 | SEQ ID NO: 1431 |
| | lmo0596.20 | GGGTTGGATCCTAACAGTTGGTATT | TGCAACGATAAGTACTATTCCAATTGCT | AAGAGAGCGATAAATCC |
| | | SEQ ID NO: 522 | SEQ ID NO: 977 | SEQ ID NO: 1432 |
| Lmo223 | lmo2230.0 | CTTGGGCGAAGAAACTTTCACTT | TTAACGCTTCAGCAATAAATGGAGTTG | CCTGGATGGAATAAGAACC |
| 0 | | SEQ ID NO: 523 | SEQ ID NO: 978 | SEQ ID NO: 1433 |
| | lmo2230.1 | CGAGCCACCTGAAAGTTTGTCTTAT | GTGCAGTTTCATGTGCAGAATCATA | CCCTAGCTCAGAACTACT |
| | | SEQ ID NO: 524 | SEQ ID NO: 979 | SEQ ID NO: 1434 |
| | lmo2230.2 | TTTATGATTCTGCACATGAAACTGCAC | TTGTTCTGGATCATCAATGTCCCAAT | TTAGCTGGAAATTTTG |
| | | SEQ ID NO: 525 | SEQ ID NO: 980 | SEQ ID NO: 1435 |
| | lmo2230.3 | CACCACGTTTCCCAGCTAATATACA | AAGCCCATTTTTTTGGTAACGCTATT | CTGGATCATCAATGTCCC |
| | | SEQ ID NO: 526 | SEQ ID NO: 981 | SEQ ID NO: 1436 |
| | lmo2230.4 | CGAGCCACCTGAAAGTTTGTCTTAT | GTGCAGTTTCATGTGCAGAATCATA | CAGATGCTGATCTCATTGTAAC |
| | | SEQ ID NO: 527 | SEQ ID NO: 982 | SEQ ID NO: 1437 |
| | lmo2230.5 | CATATTCGAAGTGCCATTGCTGAAG | CCAAGAACCGCTAATGAATTTGACA | TTGGGCGAAAAGACTTT |
| | | SEQ ID NO: 528 | SEQ ID NO: 983 | SEQ ID NO: 1438 |
| | lmo2230.6 | GGCATAAATCCAAAGCAACTCCATT | GCTTTCAGGTGGCTCAATCG | ACTCATTCAACGCTTCTGC |
| | | SEQ ID NO: 529 | SEQ ID NO: 984 | SEQ ID NO: 1439 |
| | lmo2230.7 | AGCGTTGAATGAGTTTGCAATTGAG | CATCTGCCAGTAGTTCTGAGCTA | CTGAAAGCTTGTCTTATTCTC |
| | | SEQ ID NO: 530 | SEQ ID NO: 985 | SEQ ID NO: 1440 |
| | lmo2230.8 | AGAACAAGAAATAGCGTTACCCCAAA | CTAAGCCTCTATCAATACGTGCTCTAA | CAAGAAGTATGTGATAACATTG |
| | | SEQ ID NO: 531 | SEQ ID NO: 986 | SEQ ID NO: 1441 |
| | lmo2230.9 | CTGCACCAAAATTTCCAGCTAACAT | ACATACTTCTTGATAACTCGCCCATT | CTGGATCATCAATGTCCC |
| | | SEQ ID NO: 532 | SEQ ID NO: 987 | SEQ ID NO: 1442 |
| | lmo2230.10 | GGCAGATGCTGATCTCATTGTAACA | GTTCCGGATCATCAATGTCCCAATA | CTGCACCAAAATTTC |
| | | SEQ ID NO: 533 | SEQ ID NO: 988 | SEQ ID NO: 1443 |
| | lmo2230.11 | CAGAACAAGAAATAGCGTTACCCAAA | CTAAGCCTCTATCAATACGTGCTCTAA | CAATGTTATCACAGACTTCTT |
| | | SEQ ID NO: 534 | SEQ ID NO: 989 | SEQ ID NO: 1444 |
| | lmo2230.12 | GCCACCTGAAAGCTTGTCTTATTC | GTGCAGTTTCATGTGCAGAATCATA | CAGATGCTGATCTCATTGTAAC |
| | | SEQ ID NO: 535 | SEQ ID NO: 990 | SEQ ID NO: 1445 |
| | lmo2230.13 | TGCACATGAAACTGCACCAAAATT | GTTCCGGATCATCAATGTCCCAATA | CCAGCTAACATACAAGAAAA |
| | | SEQ ID NO: 536 | SEQ ID NO: 991 | SEQ ID NO: 1446 |
| | lmo2230.14 | ATGACGCAAAAGCTGATCTACT | CAAGCTTCAGCAATGGCACTT | TTTTATCACAAACGCATATTCG |
| | | SEQ ID NO: 537 | SEQ ID NO: 992 | SEQ ID NO: 1447 |
| | lmo2230.15 | GCACATGAAACTGCACCACAATTT | TTGTTCTGGATCATCAATGTCCCAAT | CTGCTAACATACAAGAAAAAA |
| | | SEQ ID NO: 538 | SEQ ID NO: 993 | SEQ ID NO: 1448 |
| | lmo2230.16 | CAGAACAAGAAATAGCGTTACCCAAA | CTAAGCCTCTATCAATACGTGCTCTAA | AAATGGGCGAGTTATCA |
| | | SEQ ID NO: 539 | SEQ ID NO: 994 | SEQ ID NO: 1449 |
| | lmo2230.17 | GCACCACAATTTCCTGCTAACATAC | AGACTTCTTGATAACTCGCCCATTT | TCCAGAACAAGAAATAGC |
| | | SEQ ID NO: 540 | SEQ ID NO: 995 | SEQ ID NO: 1450 |
| | lmo2230.18 | GCAATTGAACCACCTGAAAGCTTAT | CTGCTTCGTGTGCTGAATCATAAAT | CAGAGGCTGATCTCATTGTTA |
| | | SEQ ID NO: 541 | SEQ ID NO: 996 | SEQ ID NO: 1451 |
| | lmo2230.19 | TGTCTTATTCCCCTAGCTCTGAACT | GCAGGAAATTGTGGTGCAGTTT | CAGATGCTGATCTCATTGTAAC |
| | | SEQ ID NO: 542 | SEQ ID NO: 997 | SEQ ID NO: 1452 |
| | lmo2230.20 | GCGTTGAATGAGTTTGCGATTGAG | ATCAGCATCTGCCAGTAGTTCAG | AAAGCTTGTCTTATTCCCC |
| | | SEQ ID NO: 543 | SEQ ID NO: 998 | SEQ ID NO: 1453 |
| clpE | clpE.0 | AGAAAGCACATCCAGATGTTCAACA | TGCACCAGCATTACTTGTCATGATA | ACGCCCTTGTGAATCG |
| | | SEQ ID NO: 544 | SEQ ID NO: 999 | SEQ ID NO: 1454 |
| | clpE.1 | GCTAGCTCGTGAATTGTTTGGTACT | GGAACCGATTAATTTAGAGATGCTGTGT | CTCATGTCTAAACGAATCAT |
| | | SEQ ID NO: 545 | SEQ ID NO: 1000 | SEQ ID NO: 1455 |
| | clpE.2 | TGTGCCTCTTGTTATGCAGAAGTTA | CGCTTGCTCCTGGGAACT | CATTCGCCCCAAAGTT |
| | | SEQ ID NO: 546 | SEQ ID NO: 1001 | SEQ ID NO: 1456 |
| | clpE.3 | TGCCCGCGGAGAACTG | ACGTCTTTCTAGTGCAGCATCTTTT | TCGGTGCAACTACATTGG |
| | | SEQ ID NO: 547 | SEQ ID NO: 1002 | SEQ ID NO: 1457 |
| | clpE.4 | AACTAGGCGCATACTTTAAACCAGAA | GTCAACGAGCATTAAGTCGATAATTTGT | CCGTCTAGATAGCGTTATTG |
| | | SEQ ID NO: 548 | SEQ ID NO: 1003 | SEQ ID NO: 1458 |
| | clpE.5 | ACCAACCACTAAAGAAACACTCACA | GCTGCTGTTAAAGCTTCAGGAGAAT | ATGGTCTGAAAACAAAGTATG |
| | | SEQ ID NO: 549 | SEQ ID NO: 1004 | SEQ ID NO: 1459 |
| | clpE.6 | CAAGAAGACGAGCAATCCAAAATGA | CTTTAGCTACTTTTTTCACGGCATCT | TCTTGACCAATTACTTTTCC |
| | | SEQ ID NO: 550 | SEQ ID NO: 1005 | SEQ ID NO: 1460 |
| | clpE.7 | CATTCCAGTTGGACGTTTACAAGAA | CGCTACTTTTTTCACGGCATCTT | ACTTAACAGGTAAAGTTAT |
| | | SEQ ID NO: 551 | SEQ ID NO: 1006 | TGGCC |
| | | | | SEQ ID NO: 1461 |
| | clpE.8 | GGTTCTCCGTTTGACGATATTTTCC | TTCTTTGTTCACGGTTTGCTTGAC | TAGCAGCACCACTCAATT |
| | | SEQ ID NO: 552 | SEQ ID NO: 1007 | SEQ ID NO: 1462 |
| | clpE.9 | TCGCTTCGCTTGTTTCTGGAA | GAATCGTGTTTTTACGTTCTTGTAGTTCTT | CCGCGGTCAATTTG |
| | | SEQ ID NO: 553 | SEQ ID NO: 1008 | SEQ ID NO: 1463 |
| | clpE.10 | ACAATCGATGTTTCCAAGGAAGTGA | CTTGGATAGTCCGGCGTAGTG | CTCCGAATTTAGGGTCATAAC |
| | | SEQ ID NO: 554 | SEQ ID NO: 1009 | SEQ ID NO: 1464 |
| | clpE.11 | GGTCAATTTGAAGAACGCATGAAAC | CCCAACGATAGTATGCACTTCATCA | ACTTCAAGAACGTAAAAAC |
| | | SEQ ID NO: 555 | SEQ ID NO: 1010 | SEQ ID NO: 1465 |
| | clpE.12 | CGTCCGTGTCATCGGTGAA | CCTGCGACAATTGCATTTGCA | CAGGTGTTGGTAAAACT |
| | | SEQ ID NO: 556 | SEQ ID NO: 1011 | SEQ ID NO: 1466 |
| | clpE.13 | GCGCTGGATCCGCAGAA | GCGAGCTAAGGCTGGTTTTAAAATA | CTGCGTCCATTGAACC |
| | | SEQ ID NO: 557 | SEQ ID NO: 1012 | SEQ ID NO: 1467 |
| | clpE.14 | CAATTCGTCGTAGCCGTGTT | GGTCCAACAAATAGGAAGGAACCA | ATTGGGCGATTTTTTGATTTG |
| | | SEQ ID NO: 558 | SEQ ID NO: 1013 | SEQ ID NO: 1468 |
| | clpE.15 | TCCATTGAAAAACTAGACGAAAATACGGT | GCTGCCTTTTCGTAATCTTCCATTT | AAGCGAACGTGTTGCCCG |
| | | SEQ ID NO: 559 | SEQ ID NO: 1014 | SEQ ID NO: 1469 |
| | clpE.16 | CGCACAAGAAGGCGTAACTATAGA | CGGCGAAGTGGTCTAGCA | CTCGATTAAATGTTCTTT |
| | | SEQ ID NO: 560 | SEQ ID NO: 1015 | AACTTC |
| | | | | SEQ ID NO: 1470 |
| | clpE.17 | ACAGGGATCCGTGGTCAATTTG | CCAACAATCGTATGCACTTCATCA | AAGAACGCATGAAACAAC |
| | | SEQ ID NO: 561 | SEQ ID NO: 1016 | SEQ ID NO: 1471 |
| | clpE.18 | CAGCTAAAGTTCGTGACGAGATTAC | GCTTGGATATCAGATGCTTGGATGA | CAATTCCTTCTCAGAACGC |
| | | SEQ ID NO: 562 | SEQ ID NO: 1017 | SEQ ID NO: 1472 |
| | clpE.19 | AGCGCTCGTTACATCCAAGAC | CAGAAAGATTGTATTTTGAGCCAACTTCA | CATTTGCCAGATAAAGCA |
| | | SEQ ID NO: 563 | SEQ ID NO: 1018 | SEQ ID NO: 1473 |
| | clpE.20 | CGCTTGCTGAGTCTATGTTTGG | GAGGCGCTCCAACTAAACGA | CATATCAATCCGAATCATCG |
| | | SEQ ID NO: 564 | SEQ ID NO: 1019 | SEQ ID NO: 1474 |
| inlC | inlC.0 | GGCCTAGCGAATGCAGTGAA | GATAGTTCCATTTGTGATACAAGGTCTGT | TTGCTTCCCTAAATTTTG |
| | | SEQ ID NO: 565 | SEQ ID NO: 1020 | SEQ ID NO: 1475 |
| | inlC.1 | AGAAGAGCTATCTGTGAATAGAAACAGACT | GAGTCAGTATCTCTGAGTTCGTTGT | AAACAAGCGAGATAAAC |
| | | SEQ ID NO: 566 | SEQ ID NO: 1021 | SEQ ID NO: 1476 |
| | inlC.2 | CTGTCAAAGACCCCGATGGAA | CCGTCTACATAATTCCCGCCATT | ATGGATTTCCCCTTATTA |
| | | SEQ ID NO: 567 | SEQ ID NO: 1022 | TATCAG |
| | | | | SEQ ID NO: 1477 |
| | inlC.3 | GCAGTGAAACAAAATTTAGGGAAGCA | CCGCAAGAGATTGAATGTTGCTATT | CAGACCTTGTATCACAAAAG |
| | | SEQ ID NO: 568 | AEEQ ID NO: 1023 | SEQ ID NO: 1478 |
| | inlC.4 | AAACTAGAGGTATTAGATTTGCATGGTAATGAAA | GTTCATTCACACATTTCTGACCAGTT | CAGGTGGACTAACTAGATTGA |
| | | SEQ ID NO: 569 | SEQ ID NO: 1024 | SEQ ID NO: 1479 |
| | inlC.5 | GAACAAAAGTACAAGCTGAGAGCAT | CTAGGCCGGGATCTGGAAAA | CAACAACCAACGCCTATTAA |
| | | SEQ ID NO: 570 | SEQ ID NO: 1025 | SEQ ID NO: 1480 |
| | inlC.6 | GGCACAAAATTTCAATGGAGATAATAGCA | AGGTCACTTATTTGATTGTGGGATAGATG | CCGGAATGCAATTTT |
| | | SEQ ID NO: 571 | SEQ ID NO: 1026 | SEQ ID NO: 1481 |
| | inlC.7 | GAAAGCAAAGTGTTACAGACCTTGT | TGCATTCCGGCAAGAGATTGAA | ACTCCAGATAGTTCCTTTTGTG |
| | | SEQ ID NO: 572 | SEQ ID NO: 1027 | SEQ ID NO: 1482 |
| | inlC.8 | TGACCTTGGTCCTTTGAAGGATTT | GCGTGATAAACAAGCACTTGGAATT | ACAGATAGCTCTTCTAACTTAG |
| | | SEQ ID NO: 573 | SEQ ID NO: 1028 | SEQ ID NO: 1483 |
| | inlC.9 | CAGGTGGACTAACTAGATTGAAGAAAGT | AGCATTTGCTATATACAATTCTGGTTGGT | CAGAAATGTGTGAATGAACC |
| | | SEQ ID NO: 574 | SEQ ID NO: 1029 | SEQ ID NO: 1484 |
| | inlC.10 | CTAGTGTTAATTGTGGGTTTGTGCAT | GGTTAATAGGCGTTGGTTGTTGAAT | TCGCAGCTTGTACTTTT |
| | | SEQ ID NO: 575 | SEQ ID NO: 1030 | SEQ ID NO: 1485 |
| | inlC.11 | TGGAGTACAAAATTTCAATGGAGATAATAGCA | GGACTAAGGTCACTTATTTGATTA | TTCCCGCAAGAGATTG |
| | | SEQ ID NO: 576 | TGGGAT | SEQ ID NO: 1486 |
| | | | SEQ ID NO: 1031 | |
| | inlC.12 | CATTAAATCTCTTGCCGGAATGCA | AGTTAGATCATTTAAAGGACTAAGGTC | TTGTGGGATAGATGAAGTTC |
| | | SEQ ID NO: 577 | ACTTATTTG | SEQ ID NO: 1487 |
| | | | SEQ ID NO: 1032 | |
| | inlC.13 | CTGTACATTAATACGGGCTTCGGAA | CTAGGCCGGGATCTGGAAAA | CTCTCAGCTTGTACTTTTG |
| | | SEQ ID NO: 578 | SEQ ID NO: 1033 | SEQ ID NO: 1488 |
| | inlC.14 | CTGTCAAAGACCCTGATGGAAGAT | CCACAGGACACAACCATCTACATAA | ACTGATATAATATGGAGATATCC |
| | | SEQ ID NO: 579 | SEQ ID NO: 1034 | SEQ ID NO: 1489 |
| | inlC.15 | CAGGTGGACTAACTAGATTGAAGAAAGT | ACAGTGTTTGTTATATCAATTCTG | CTGGTTCATTCACACATTTC |
| | | SEQ ID NO: 580 | GTTGGT | SEQ ID NO: 1490 |
| | | | SEQ ID NO: 1035 | |
| | inlC.16 | TGGAACAAAAGTACAAGCTGAGAGT | CTAGGCCGGGATCTGGAAAA | TCAACGACCAACCCC |
| | | SEQ ID NO: 581 | SEQ ID NO: 1036 | SEQ ID NO: 1491 |
| | inlC.17 | GAAAGCAAAGTGTTACAGACCTTGT | TCCGGCAAGAGATTTAATGTTGCTA | CCCCAGATAGTTCCTTTTGTG |
| | | SEQ ID NO: 582 | SEQ ID NO: 1037 | SEQ ID NO: 1492 |
| | inlC.18 | GTGGGAGTTATGTAGATGGTTGTGT | CCTCAGTCTCCCCAACGTTTATAT | CTGTATAAACTGGCAATTCC |
| | | SEQ ID NO: 583 | SEQ ID NO: 1038 | SEQ ID NO: 1493 |
| | inlC.19 | CTGTCAAAGACCCAGATGGAAGAT | CAACCGTCTACATAATTCCCACCAT | ACTGATATAATATGGAGATATCC |
| | | SEQ ID NO: 584 | SEQ ID NO: 1039 | SEQ ID NO: 1494 |
| | inlC.20 | GTCTGTGCATTAATACGGGTTCTG | GTTAATAGGCGTTGGTCGTTGAAT | CTCTCAGCTTGTACTTTTG |
| | | SEQ ID NO: 585 | SEQ ID NO: 1040 | SEQ ID NO: 1495 |
| lmo067 | lmo0670.0 | GCCATGGACTAAAGACGATTATCCAA | CCGGATATCCATCTTTGAGTAAAGCA | CCAATCTCAATAGCTTTTTC |
| 0 | | SEQ ID NO: 586 | SEQ ID NO: 1041 | SEQ ID NO: 1496 |
| | lmo0670.1 | ATCGGCAATGCGCTATTAAAAGATG | CGCTTTCGAAGTGGCAATTGG | TCAGAAAGCCGCGCCATC |
| | | SEQ ID NO: 587 | SEQ ID NO: 1042 | SEQ ID NO: 1497 |
| | lmo0670.2 | CGCGAAAAAGCTATTGAGATTGGT | TCTGCTTTCGAGGTGGCAAT | CCGGAAGATCGAGCTATAC |
| | | SEQ ID NO: 588 | SEQ ID NO: 1043 | SEQ ID NO: 1498 |
| | lmo0670.3 | TGCCGTGGACAAAAAATGATTATCC | CATCTTTTAATAGGGCATTGCCGATT | TCGGTCTTTCTTAGATTTT |
| | | SEQ ID NO: 589 | SEQ ID NO: 1044 | SEQ ID NO: 1499 |
| | lmo0670.4 | TCGGCAACGCGTTACTAAAAGA | CCATTCTTCTGCTTTTGAAGTAGCAA | CAGAAAGCCGCGCCATC |
| | | SEQ ID NO: 590 | SEQ ID NO: 1045 | SEQ ID NO: 1500 |
| | lmo0670.5 | GGAAAAATCTAAGAAAGACCGAACGC | AGCGCGGCTTTCTGAGTAG | CCGATTTCAATAGCTTTTTC |
| | | SEQ ID NO: 591 | SEQ ID NO: 1046 | SEQ ID NO: 1501 |
| | lmo0670.6 | ACGATTATCCGGATTCGTGGAAAA | GCGCGGCTTTCTGAATAGC | TTGCCGATTTCAATAGCTTT |
| | | SEQ ID NO: 592 | SEQ ID NO: 1047 | SEQ ID NO: 1502 |
| | lmo0670.7 | CGGCAACGCGCTATTAAAAGATG | TCTGCTTTTGAAGTGGCAATTGG | TCAGAAAGCCGCGCCATC |
| | | SEQ ID NO: 593 | SEQ ID NO: 1048 | SEQ ID NO: 1503 |
| | lmo0670.8 | CGCGAAAAGGCAATTGAAATCG | GCGCGGCTTTCTGAGTAG | CAACGCGCTATTAAAAG |
| | | SEQ ID NO: 594 | SEQ ID NO: 1049 | SEQ ID NO: 1504 |
| | lmo0670.9 | GCCGTGGACAAAAAATGAATATCCT | CGTTGCCGATTTCAATTGCCTTT | TCGCGTTCGGTCTTTCT |
| | | SEQ ID NO: 595 | SEQ ID NO: 1050 | SEQ ID NO: 1505 |
| | lmo0670.10 | TGCCATGGACTAAAGACGATTATCC | TGAGTAAAGCATTACCAATCTCA | TCGCGTTCTGATTTTT |
| | | SEQ ID NO: 596 | ATAGCTT | SEQ ID NO: 1506 |
| | | | SEQ ID NO: 1051 | |
| | lmo0670.11 | CGCGAGAAGGCAATTGAAATCG | GCGCGGCTTTCTGAGTAG | ACGCGTTACTAAAAGAAG |
| | | SEQ ID NO: 597 | SEQ ID NO: 1052 | SEQ ID NO: 1507 |
| | lmo0670.12 | CGCGAAAAAGCTATTGAGATTGGT | GTGGCAATAGGTATAGCTCGATCTT | ATCCATCTTTGAGTAAAGCATT |
| | | SEQ ID NO: 598 | SEQ ID NO: 1053 | SEQ ID NO: 1508 |
| | lmo0670.13 | AGAAAGACCGAACGCGAGAAG | GCGCGGCTTTCTGAGTAG | TTGCCGATTTCAATTGC |
| | | SEQ ID NO: 599 | SEQ ID NO: 1054 | SEQ ID NO: 1509 |
| | lmo0670.14 | CGCGCTATTAAAAGATGGCTACTCA | CGATTCCTTATGATTTTTATACCAT | TCCCAATTGCCACTTCAA |
| | | SEQ ID NO: 600 | TCTTCTGC | SEQ ID NO: 1510 |
| | | | SEQ ID NO: 1055 | |
| | lmo0670.15 | GGAAAAATCTAAGAAAGACCGAACGC | GCGCGGCTTTCTGAGTAG | CCGATTTCAATTGCCTTTTC |
| | | SEQ ID NO: 601 | SEQ ID NO: 1056 | SEQ ID NO: 1511 |
| | lmo0670.16 | GCCGTGGACAAAAAACGATTATCC | TTTTAATAGCGCATTGCCGATTTCA | CTGTTTTCTTTAGATTTTTCC |
| | | SEQ ID NO: 602 | SEQ ID NO: 1057 | SEQ ID NO: 1512 |
| | lmo0670.17 | CGCGTTACTAAAAGAAGGCTACTCA | CCATTCTTCTGCTTTTGAAGTAGCAA | CCGCGCCATCCCGA |
| | | SEQ ID NO: 603 | SEQ ID NO: 1058 | SEQ ID NO: 1513 |
| | lmo0670.18 | TCTAAGAAAGACCGAACGCGAAAA | AGCGCGGCTTTCTGAGTAG | ATCGGCAATGCCCTATTAA |
| | | SEQ ID NO: 604 | SEQ ID NO: 1059 | SEQ ID NO: 1514 |
| | lmo0670.19 | ACGATTATCCGGATTCGTGGAAAA | GCGCGGCTTTCTGAATAGC | CAATGCGCTATTAAAAGAT |
| | | SEQ ID NO: 605 | SEQ ID NO: 1060 | SEQ ID NO: 1515 |
| lmo252 | lmo2522.0 | TGTCTGTAGGTGAAAATGCTAAAGCT | TGCTGTGCTGGTTGTTCTGTAG | CACCTTCTGAAAACAAC |
| 2 | | SEQ ID NO: 606 | SEQ ID NO: 1061 | SEQ ID NO: 1516 |
| | lmo2522.1 | AATAAAGCAGCATCTTCAAACAAATCGT | CCCATTCCTGGTTCTTCTTTACTGT | CTGCATCTCAAGGTAATGTAT |
| | | SEQ ID NO: 607 | SEQ ID NO: 1062 | SEQ ID NO: 1517 |
| | lmo2522.2 | TGCAAGATGGTGATTCACTATGGAA | ACTTGCAATGTTTGGTTCGGAAAAA | CAATTGAAAGAAGATAACAA |
| | | SEQ ID NO: 608 | SEQ ID NO: 1063 | TTTG |
| | | | | SEQ ID NO: 1518 |
| | lmo2522.3 | CGGTGACTCACTTTGGAAAATCTCA | GTTTGGTTTGGAAAAATTAAGTTTGAGCTT | AAGTTGTTATCTTCTTTC |
| | | SEQ ID NO: 609 | SEQ ID NO: 1064 | AATTGTT |
| | | | | SEQ ID NO: 1519 |
| | lmo2522.4 | ATCATAGTTGGTCAAAAATTGTCTGTAGGT | ATTTGTTGTTTTCAGAAGGTGTGCTT | TCACTAGCTTTAGCATTTTC |
| | | SEQ ID NO: 610 | SEQ ID NO: 1065 | SEQ ID NO: 1520 |
| | lmo2522.5 | ACAAATCGTCTGCATCTCAAGGTAA | TCTACAGCAATAACGCGTGGATT | CTGCATACAGTAAAGAAGAAC |
| | | SEQ ID NO: 611 | SEQ ID NO: 1066 | SEQ ID NO: 1521 |
| | lmo2522.6 | GCACAAGGGAATGTTTCAAAAGAGT | CATTTAAATCAATTCCAGTCGCTGTCA | ACAGCATACAGTAAATCT |
| | | SEQ ID NO: 612 | SEQ ID NO: 1067 | SEQ ID NO: 1522 |
| | lmo2522.7 | GCAGCTCCAACAACTCAAAAATCAA | CTGTTGCAGTTACCGTTAACTCTTT | CCTTGTGCTGAAGAACT |
| | | SEQ ID NO: 613 | SEQ ID NO: 1068 | SEQ ID NO: 1523 |
| | lmo2522.8 | AGTAACAGAGCAACCGAAAGAAGAAA | CTTGTGCTGATGAAGAACTGTTTGA | CAGCTCCAGCAACTCA |
| | | SEQ ID NO: 614 | SEQ ID NO: 1069 | SEQ ID NO: 1524 |
| | lmo2522.9 | GCTCAAACTTAATTTTTCCAAACCAAACG | GTGTGTCACCAGCTACAACTGTATA | TTACTAGTAGCAGCTTTTTTC |
| | | SEQ ID NO: 615 | SEQ ID NO: 1070 | SEQ ID NO: 1525 |
| | lmo2522.10 | TTAACGGTAACTGCAACAGCATATAGT | TCCCTGTCGCTGTCATGTG | ATGCCAGGTTCAGCTTT |
| | | SEQ ID NO: 616 | SEQ ID NO: 1071 | SEQ ID NO: 1526 |
| | lmo2522.11 | TTTTCCGAACCAAACATTGCAAGT | TGTTACACCATTATCAACGGCGAT | ATGTCCAAGGGTATCTCC |
| | | SEQ ID NO: 617 | SEQ ID NO: 1072 | SEQ ID NO: 1527 |
| | lmo2522.12 | GGTAACTGCAACAGCATATAGTAAAGC | TGGATCAACTGCAATTACTCGTGAA | TCGCTGTCATGTGGCCCA |
| | | SEQ ID NO: 618 | SEQ ID NO: 1073 | SEQ ID NO: 1528 |
| | lmo2522.13 | ATCTTTCATCTGATTTAATCGTAGTTGGTCAA | GTAGCCGGGCTCTCATTAGATT | TCTATCGGAAGTAAAGCTG |
| | | SEQ ID NO: 619 | SEQ ID NO: 1074 | SEQ ID NO: 1529 |
| | lmo2522.14 | AGCCATCTACAAATAATGCGGAACA | ACGATTTGTTTGAAGATGCTGCTTT | TTGGAGCAGCTTTTTCTT |
| | | SEQ ID NO: 620 | SEQ ID NO: 1075 | SEQ ID NO: 1530 |
| | lmo2522.15 | AGGATACGGACAAGCAATTGCA | CCCCAGTTGTTAGCTTCTTGTACT | TTGCACCACCAGTATCAG |
| | | SEQ ID NO: 621 | SEQ ID NO: 1076 | SEQ ID NO: 1531 |
| | lmo2522.16 | ACAATTCAACAGCAAATAATTCAGCCAAT | GCTGAAGAACTGTTTGAAGATGCTT | CCGCCTTTTCTTCTTTCG |
| | | SEQ ID NO: 622 | SEQ ID NO: 1077 | SEQ ID NO: 1532 |
| | lmo2522.17 | CATCAGCAAATAAAGCAGCATCTTCA | TCTTTACTGTATGCAGTTGCTGTTACA | AACAAATCGTCTGCATCTCA |
| | | SEQ ID NO: 623 | SEQ ID NO: 1078 | SEQ ID NO: 1533 |
| | lmo2522.18 | GGTCAAAAGTTATCTATCGGAAGTAAAGCT | TGTTCCGCATTATTTGTAGATGGCTTA | CCGGGCTCTCATTAGAT |
| | | SEQ ID NO: 624 | SEQ ID NO: 1079 | SEQ ID NO: 1534 |
| | lmo2522.19 | CGGTGTGACAGTTAACCAATTAAAAGAA | GGTGTGCTTTCACTAGCTTTAGC | CACCTACAGACAATTTT |
| | | SEQ ID NO: 625 | SEQ ID NO: 1080 | SEQ ID NO: 1535 |
| | lmo2522.20 | AGGATACGGACAAGCAATTGCA | CCCCAGTTGTTAGCTTCTTGTACT | TTGCACCGCCAGTATCA |
| | | SEQ ID NO: 626 | SEQ ID NO: 1081 | SEQ ID NO: 1536 |
| cspL | cspL.0 | GACGTATTCGTTCACTTCTCAGCTA | ACGTCGAAAGTCACTGCTTGA | CCAAGGTGACGGATTC |
| | | SEQ ID NO: 627 | SEQ ID NO: 1082 | SEQ ID NO: 1537 |
| | cspL.1 | AGAAGGCCAAGCGGTAACATT | CTTTTTCTACATTAGCTGCCTGAGC | ATTACCTTCGACAACTTCA |
| | | SEQ ID NO: 628 | SEQ ID NO: 1083 | SEQ ID NO: 1538 |
| | cspL.2 | TCGTACATTTCAGCGCTATCCAA | TCTTCAACGTCGAAAGTTACTGCTT | ACCTTCGTCTAAAGATTTG |
| | | SEQ ID NO: 629 | SEQ ID NO: 1084 | SEQ ID NO: 1539 |
| | cspL.3 | GAAGGCGGAGATGATGTATTCGT | CGCTTTGACCTTCGTCTAAAGTTTT | ACACTTCAGCGCTATCG |
| | | SEQ ID NO: 630 | SEQ ID NO: 1085 | SEQ ID NO: 1540 |
| | cspL.4 | CAAGGTGACGGCTACAAATCTTTAG | GCGATTACCTTCGACAACTTCAAAT | CCGCTTGGCCTTCTT |
| | | SEQ ID NO: 631 | SEQ ID NO: 1086 | SEQ ID NO: 1541 |
| | cspL.5 | ATGCAAACAGGTACAGTTAAATGGT | CGATAGCGCTGAAGTGTACGAATAT | AAGGCTTCGGTTTCATCG |
| | | SEQ ID NO: 632 | SEQ ID NO: 1087 | SEQ ID NO: 1542 |
| | cspL.6 | CAAGGTGACGGCTACAAATCTTTAG | TGAGCGCCGCGGTTA | CCTTCAACTACTTCAAATGTT |
| | | SEQ ID NO: 633 | SEQ ID NO: 1088 | SEQ ID NO: 1543 |
| | cspL.7 | GCGCTATCGAAGGTGAAGGATT | CTTGTGGGCCACGTTGAC | CAACGCTTTGACCTTCG |
| | | SEQ ID NO: 634 | SEQ ID NO: 1089 | SEQ ID NO: 1544 |
| | cspL.8 | GGATTTGGTTTTATCGAACGCGAAA | TTTGAATCCGTCGCCTTGGA | ACGATGTATTCGTACATTTC |
| | | SEQ ID NO: 635 | SEQ ID NO: 1090 | SEQ ID NO: 1545 |
| | cspL.9 | GCGCTATCGAAGGTGAAGGATT | CTTGTGGGCCACGTTGAC | AAAGCGTTGAATTTGAAATC |
| | | SEQ ID NO: 636 | SEQ ID NO: 1091 | SEQ ID NO: 1546 |
| | cspL.10 | ATGCAAACAGGTACAGTTAAATGGT | CGATAGCGCTGAAGTGTACGAATAC | CTCCGCCTTCTACTTCG |
| | | SEQ ID NO: 637 | SEQ ID NO: 1092 | SEQ ID NO: 1547 |
| | cspL.11 | ATGCAAACAGGTACAGTTAAATGGT | CATCGTCTCCGCCTTCTACTTC | AAAGGCTTCGGTTTCATC |
| | | SEQ ID NO: 638 | SEQ ID NO: 1093 | SEQ ID NO: 1548 |
| | cspL.12 | CAAGGTGACGGCTACAAATCTTTAG | TTTTTCCACATTGGCTGCTTGAG | ACCTTCAACTACTTCAAATGT |
| | | SEQ ID NO: 639 | SEQ ID NO: 1094 | SEQ ID NO: 1549 |
| | cspL.13 | GCGAAAACGGTGACGATGTATTC | CGTCGAAAGTTACTGCTTGACCTT | TCGCCTTGGATAGCGC |
| | | SEQ ID NO: 640 | SEQ ID NO: 1095 | SEQ ID NO: 1550 |
| | cspL.14 | GGCGGAGATGATATATTCGTACACTT | GTTGGCCTTCAACGATTTCAAATTC | TCGAAGGTGAAGGATTCA |
| | | SEQ ID NO: 641 | SEQ ID NO: 1096 | SEQ ID NO: 1551 |
| | cspL.15 | TCGAAGTAGAAGGCGGAGATGATAT | CGCTTTGACCTTCGTCTAAAGTTTT | ACACTTCAGCGCTATCG |
| | | SEQ ID NO: 642 | SEQ ID NO: 1097 | SEQ ID NO: 1552 |
| | cspL.16 | CAAGGTGACGGCTACAAATCTTTAG | CCGCGATTACCTTCAACTACTTCAA | CCGCTTGGCCTTCTT |
| | | SEQ ID NO: 643 | SEQ ID NO: 1098 | SEQ ID NO: 1553 |
| | cspL.17 | GACGGCGGCGAAGATATTTTC | ACCGCTTGGCCTTCTTCTAAAG | CACTTCACAGCGATCCAA |
| | | SEQ ID NO: 644 | SEQ ID NO: 1099 | SEQ ID NO: 1554 |
| | cspL.18 | AGACGGCGGCGAAGATATTTT | ACCGCTTGGCCTTCTTCTAAAG | TCGCTGTGAAGTGAACG |
| | | SEQ ID NO: 645 | SEQ ID NO: 1100 | SEQ ID NO: 1555 |
| | cspL.19 | ATGCAAAATGGGAAAGTAAAATGGTTT | TTCGCCGCCGTCTGATT | AAGGGTTACGGTTTTATCG |
| | | SEQ ID NO: 646 | SEQ ID NO: 1101 | SEQ ID NO: 1556 |
| | cspL.20 | GAAGGCGGAGATGATGTATTCGTA | CTTCAACGATTTCAAATTCAACGCTTT | TCGAAGGTGAAGGATTCA |
| | | SEQ ID NO: 647 | SEQ ID NO: 1102 | SEQ ID NO: 1557 |
| inlG | inlG.0 | TCACTGATGAAGTTACGCAAACAGA | CTGATTGGAAGATACACCCAACATATTCA | ACGACAAAGGAATAAATTC |
| | | SEQ ID NO: 648 | SEQ ID NO: 1103 | SEQ ID NO: 1558 |
| | inlG.1 | GCCACTTTCAGGATTGACACAGTTA | CGTTAGATTAGCAAGAGGTGTCACA | CATTCGTACAATTATCTATCA |
| | | SEQ ID NO: 649 | SEQ ID NO: 1104 | ATCAA |
| | | | | SEQ ID NO: 1559 |
| | inlG.2 | CCCGCTGACGAACCTTACTAAATTA | TGGTCAATCTTGAAAGCGGAGTTAA | CTCTTTATATTTAGGGAAAAATC |
| | | SEQ ID NO: 650 | SEQ ID NO: 1105 | SEQ ID NO: 1560 |
| | inlG.3 | GCGGAAACTGGTGGGAATGAA | TGTCATATTCGTAGCCGGCATTTTA | CTACTGCAAAATCCC |
| | | SEQ ID NO: 651 | SEQ ID NO: 1106 | SEQ ID NO: 1561 |
| | inlG.4 | CCGACGAAAGATGGCTACACATTT | AATCCCATTCATTCCCACCTGTTT | TTGCATCGTACCATCCTAC |
| | | SEQ ID NO: 652 | SEQ ID NO: 1107 | SEQ ID NO: 1562 |
| | inlG.5 | CGCAACTAGCAAAATGCCAACTA | GCGCTGTCGTTAGCTTTAATTGTTA | CAATTCTCAAACAGCTCCC |
| | | SEQ ID NO: 653 | SEQ ID NO: 1108 | SEQ ID NO: 1563 |
| | inlG.6 | GTGACACCTCTTGCTAATCTAACGA | GCGCGGCTGTTATTTGTTGTTT | TCAAATAAGTGATGCAAGTCC |
| | | SEQ ID NO: 654 | SEQ ID NO: 1109 | SEQ ID NO: 1564 |
| | inlG.7 | AGTTTCACGAGTGAGGTTAGTTATGATTTT | GCTTCTACTATCGGTTGAACAACAG | ATGGGAAGGTAACTTTTG |
| | | SEQ ID NO: 655 | SEQ ID NO: 1110 | SEQ ID NO: 1656 |
| | inlG.8 | GTGAAGTAGTACCACCAACAACGAT | CTCACTCGTGAAACTATCTAAGTTCCA | TTGGACTAGCAAAGGTTC |
| | | SEQ ID NO: 656 | SEQ ID NO: 1111 | SEQ ID NO: 1566 |
| | inlG.9 | GACAGAAAACGTGGTAGTGGATACA | TCCGCATCATACCAACCAGAAAA | ACCGACAAAAGAAGGTTATAC |
| | | SEQ ID NO: 657 | SEQ ID NO: 1112 | SEQ ID NO: 1567 |
| | inlG.10 | CCCCCGGAAATGATGATAAAAACGA | TTTTGGAAGTTTAGTACTCGTCGTATCTG | CTGTCGTTAGCTTTAATTGT |
| | | SEQ ID NO: 658 | SEQ ID NO: 1113 | SEQ ID NO: 1568 |
| | inlG.11 | GCCACTTTCAGGATTGACACAGTTA | GACTTGCATCACTTATTTGATTTTCTCGTA | ATGTGACACCTCTTGCTAATC |
| | | SEQ ID NO: 659 | SEQ ID NO: 1114 | SEQ ID NO: 1569 |
| | inlG.12 | TGACACCACTTGCTAATCTAACGAA | AGCGGCCGTTATTTGTTGTTTT | AAGGACTTGCATCACTTATT |
| | | SEQ ID NO: 660 | SEQ ID NO: 1115 | SEQ ID NO: 1570 |
| | inlG.13 | CGCTCTACGCCCAGTTCA | GTTTCTCCGTCCACATCAAATGC | CCGTGTAGCTATTTATTG |
| | | SEQ ID NO: 661 | SEQ ID NO: 1116 | SEQ ID NO: 1571 |
| | inlG.14 | CTTGTAGTGACAGCTATCTTGGGAAT | AATACTCTCTGCCTGAGCTTTCATC | CTTGCATTTACCCATAAACT |
| | | SEQ ID NO: 662 | SEQ ID NO: 1117 | SEQ ID NO: 1572 |
| | inlG.15 | AAGCTCAGGCAGAGAGTATTGC | GCTAATGCTGGATCCGTGAAAAT | CAGCGCCAATTAACGAA |
| | | SEQ ID NO: 663 | SEQ ID NO: 1118 | SEQ ID NO: 1573 |
| | inlG.16 | TGCTGAACCGACAAAAGAAGGT | GCCGGCATTTTATCTACTGCAAA | CCCACCAGTTTCCG |
| | | SEQ ID NO: 664 | SEQ ID NO: 1119 | SEQ ID NO: 1574 |
| | inlG.17 | GACAACAACAGAAAACGTGACAGTA | ACCAGTTTTTGCATCATACCATCCA | CTGAGCCAACAAAAGAA |
| | | SEQ ID NO: 665 | SEQ ID NO: 1120 | SEQ ID NO: 1575 |
| | inlG.18 | GCGCCCGTGAATTACATTACTACAT | CGGTTCAGCAGGTTCGGTTATTAA | TCGTCGTCGTTCCATCCAC |
| | | SEQ ID NO: 666 | SEQ ID NO: 1121 | SEQ ID NO: 1576 |
| | inlG.19 | ACAGCTCCCGGAAAAGATGATAAAA | CATCACTTGTTTTTGGAAGTTTAGTACTCG | TCGCATCTGCGCTGTC |
| | | SEQ ID NO: 667 | SEQ ID NO: 1122 | SEQ ID NO: 1577 |
| Lmo069 | lmo0699.0 | GCGAAGCACTCAAAGACAGTTTTAA | CACGTAGAAGTGCTGGGTTAGTTT | CCAGAAATCGTTAATATCG |
| 9 | | SEQ ID NO: 668 | SEQ ID NO: 1123 | SEQ ID NO: 1578 |
| | lmo0699.1 | TGGGCGCTTTCATCCCTATG | CGTCTTTAAGCTTTCGAGGTGTTC | CATAAACCAGAGAAATTTG |
| | | SEQ ID NO: 669 | SEQ ID NO: 1124 | SEQ ID NO: 1579 |
| | lmo0699.2 | TGTTTTTGAAGAGCTGACGTTGAAA | TGGTGAGATTGGATGCACTGATTT | AAGCACTCAAAGACAGTTTT |
| | | SEQ ID NO: 670 | SEQ ID NO: 1125 | SEQ ID NO: 1580 |
| | lmo0699.3 | GCGACGTGCTTCTGACAGA | CGGCTTTTTGGTTACCACTTTTTCC | CTCATATACGTGACACACTCAA |
| | | SEQ ID NO: 671 | SEQ ID NO: 1126 | SEQ ID NO: 1581 |
| | lmo0699.4 | GAAACTAACCCAGCACTTCTACGT | ATGCGCATTGTACTAATCCAAAACT | CACATCACCGAACGACATG |
| | | SEQ ID NO: 672 | SEQ ID NO: 1127 | SEQ ID NO: 1582 |
| | lmo0699.5 | AGAAAAATTCTGCGAAGCACTCAAAG | GTAGAAGTGCTGGGTTAGTTTCGAT | ATGCACCGATTTAAAACTGT |
| | | SEQ ID NO: 673 | SEQ ID NO: 1128 | SEQ ID NO: 1583 |
| | lmo0699.6 | CAACGTGGAAAGAGCTCAACAAG | CCTTTGAGTGTATCGCGTATGTG | ACGTCCTTCTAACAGAAACA |
| | | SEQ ID NO: 674 | SEQ ID NO: 1129 | SEQ ID NO: 1584 |
| | lmo0699.7 | TGAGCGAGCGCAGACTTTTAT | GTCTTTGAGTGCTTCGCAGAATTTT | CAGCTCTTCAAAAACAG |
| | | SEQ ID NO: 675 | SEQ ID NO: 1130 | SEQ ID NO: 1585 |
| | lmo0699.8 | GGTTACGTCACCGAAAAATGGAAAT | GCTCTGTTCGCCCTGTATGAC | AACGGCTTTTTGGTTACCAC |
| | | SEQ ID NO: 676 | SEQ ID NO: 1131 | SEQ ID NO: 1586 |
| | lmo0699.9 | GCGCATACGTGTAGGTGAAATTAAA | CAGAAGCACGTCGCCTACTT | CAAGCTTATTCAGTTCTTTCC |
| | | SEQ ID NO: 677 | SEQ ID NO: 1132 | SEQ ID NO: 1587 |
| | lmo0699.10 | TGATGCGGAAGTCGAGCAA | CACGAATGCGCGCTTCT | TTTTCGACTTGATGCAATTC |
| | | SEQ ID NO: 678 | SEQ ID NO: 1133 | SEQ ID NO: 1588 |
| | lmo0699.11 | AGAGAAATTTGGCGCAGAACAC | CTGGGAAACAAACTCCATGCTTTT | ATGCAATCGTCTTTAAGCTT |
| | | SEQ ID NO: 679 | SEQ ID NO: 1134 | SEQ ID NO: 1589 |
| | lmo0699.12 | TGAGTGAAGGTAAGGTAGTCGATGAA | GTGTTCCGCACCAAATTTCTCT | CATAGGGATGAAAGCGCC |
| | | SEQ ID NO: 680 | SEQ ID NO: 1135 | SEQ ID NO: 1590 |
| | lmo0699.13 | GTTGAGAAAGAAGCGCGTATTCG | CACGTATATGAGTTTCTGTCAGAAGCA | CCAACTTCTAGCATATTAAG |
| | | SEQ ID NO: 681 | SEQ ID NO: 1136 | SEQ ID NO: 1591 |
| | lmo0699.14 | CGCATCGGCGTCCCATT | CGCATCAAAATTCCGTCGTTTATCA | TTTCTCAGTAGAAGAGATTATG |
| | | SEQ ID NO: 682 | SEQ ID NO: 1137 | SEQ ID NO: 1592 |
| | lmo0699.15 | CCGAACGATAGTTACATTTTCTGCAT | TGGATTTCCACCTGATAAACATTCGT | CCGGATTACCGAGATCAA |
| | | SEQ ID NO: 683 | SEQ ID NO: 1138 | SEQ ID NO: 1593 |
| | lmo0699.16 | CTTAACATGCTAGAAGTTGGAGATGTACT | TTTCTCGGTGACATAGCCTTTGAG | TCGCGTATATGGGTTTCT |
| | | SEQ ID NO: 684 | SEQ ID NO: 1139 | SEQ ID NO: 1594 |
| | lmo0699.17 | AGCGAGCGCAGACTTTTATCT | GTCTTTGAGTGCTTCGCAGAATTTT | ATTTCAACGTCAGCTCTTC |
| | | SEQ ID NO: 685 | SEQ ID NO: 1140 | SEQ ID NO: 1595 |
| | lmo0699.18 | CGCATCGGCGTCCCATT | CGCATCAAAATTCCGTCGTTTATCA | ACGACATTCTCAAGTTTATTCA |
| | | SEQ ID NO: 686 | SEQ ID NO: 1141 | SEQ ID NO: 1596 |
| | lmo0699.19 | GGAGTTTGTTTCCCAGCGTATTC | CGCAAAAGACACTTGATCTGCAA | TTCGGTATGAATTGGAATCC |
| | | SEQ ID NO: 687 | SEQ ID NO: 1142 | SEQ ID NO: 1597 |
| | lmo0699.20 | GGCGACGTCCTTCTAACAGA | CATTTTTCGGTGACATAGCCTTTGA | CACACATACGCGATACAC |
| | | SEQ ID NO: 688 | SEQ ID NO: 1143 | SEQ ID NO: 1598 |
| opuCA | opuCA | GGATGTAACGCAGGTAGCTCAAA | CTTGAAGTGATTTGTCGGCTGTAAT | CCGGGTTGGTATTCAT |
| | | SEQ ID NO: 689 | SEQ ID NO: 1144 | SEQ ID NO: 1599 |
| | opuCA.1 | GGGCAAAAAAGCCGTTAATGATCTA | GTAGTTTTCCCACAACCACTTGGA | CCGATAAAACAAACAAATTC |
| | | SEQ ID NO: 690 | SEQ ID NO: 1145 | SEQ ID NO: 1600 |
| | opuCA.2 | CGACTTATTGAGCCAACAGAAGGAA | GTTGAATGACATATCCAATAGAGCGTCTT | CCGCCATGATGTCTTTATCA |
| | | SEQ ID NO: 691 | SEQ ID NO: 1146 | SEQ ID NO: 1601 |
| | opuCA.3 | ACGAATCCAGTGTCTATTACAGCAG | ACCTTCATCCACTACAAGTAACGTATCT | CAGTCATGAAAGAAAAACG |
| | | SEQ ID NO: 692 | SEQ ID NO: 1147 | SEQ ID NO: 1602 |
| | opuCA.4 | GGCTTTATCGACGTAGAGCAAATTG | CAGTGTCACGAAGCAAGGTATCTT | TCGTACAGCAACTTCTG |
| | | SEQ ID NO: 693 | SEQ ID NO: 1148 | SEQ ID NO: 1603 |
| | opuCA.5 | AGAAAAAACAAGAACGGGCAAAAGAA | TGCTGTCCACCACTTAATTCGTAA | AAAACTCTTCTGGTAAATCT |
| | | SEQ ID NO: 694 | SEQ ID NO: 1149 | SEQ ID NO: 1604 |
| | opuCA.6 | ATACGTTACTTGTTGTGGATGAAGGT | ACAGAAGTTGCTGTACGACGATTTA | TCGATAAAGCCTTTTAAAACAT |
| | | SEQ ID NO: 695 | SEQ ID NO: 1150 | SEQ ID NO: 1605 |
| | opuCA.7 | TTGTGGGAAAACGACAACAATGAAG | GGATCTTCCGCCATGATGTCTTTAT | CAATAAGCCGGTTAATCAT |
| | | SEQ ID NO: 696 | SEQ ID NO: 1151 | SEQ ID NO: 1606 |
| | opuCA.8 | TCGAACACGTAACAAAGACTTATAAAGGG | CCACTTGGACCGATAAAACAAACAA | AATGATCTAACACTAAACATCG |
| | | SEQ ID NO: 697 | SEQ ID NO: 1152 | SEQ ID NO: 1607 |
| | opuCA.9 | GTGTTAAAATTCGAACACGTAACAAAGAC | CCACTTGGACCGATAAAACAAACAA | ACTGCTTTTTTGCCCCCTTT |
| | | SEQ ID NO: 698 | SEQ ID NO: 1153 | SEQ ID NO: 1608 |
| | opuCA.10 | CCACATATGACGATTCGCGAAAATA | CTTTCTTGTTTTTTCTCTTCGGACCATT | CCTTGTACCAAAATTAC |
| | | SEQ ID NO: 699 | SEQ ID NO: 1154 | SEQ ID NO: 1609 |
| | opuCA.11 | TGGTTATGTTATTCAGCAAATTGGCTTG | TCTCTTCGGACCATTTAAGTAATTTTGGT | ATGCCACATATGACGATTC |
| | | SEQ ID NO: 700 | SEQ ID NO: 1155 | SEQ ID NO: 1610 |
| | opuCA.12 | CCAATTACTCGTGATTCCCTACAAGA | GCCAGTTTAATCGCTTCATCCAT | CTTGCCAAGTTCTTTTTGCA |
| | | SEQ ID NO: 701 | SEQ ID NO: 1156 | SEQ ID NO: 1611 |
| | opuCA.13 | CGTGCCAGCTTAGTTGATATCGTAT | CGTTTTGGAATCCGCCTGTTC | ATGCGACAGAAAACCAG |
| | | SEQ ID NO: 702 | SEQ ID NO: 1157 | SEQ ID NO: 1612 |
| | opuCA.14 | GGATTTATCGACGTAGAGCAAATCGA | ACGGAGTAAGGTATCTTCATACACGTA | TTTAAATCGCCGCACAGCAA |
| | | SEQ ID NO: 703 | SEQ ID NO: 1158 | SEQ ID NO: 1613 |
| | opuCA.15 | CTTGTAGTCGACGAAGGGAATGTAT | ACAGAAGTTGCAGTACGACGATTT | TTTGTTCTACGTCAATAAAG |
| | | SEQ ID NO: 704 | SEQ ID NO: 1159 | SEQ ID NO: 1614 |
| | opuCA.16 | GATGAAGCAATAAAACTTGCAGATCGT | CGCTGGATTACGCAAAATTTCATCT | ATGAAAGCTGGTGAAATC |
| | | SEQ ID NO: 705 | SEQ ID NO: 1160 | SEQ ID NO: 1615 |
| | opuCA.17 | GGGCAAAAAAGCTGTTAATGATCTAAC | TCGTCGTTTTCCCACAACCA | CCGATAAAACAAACAAATTC |
| | | SEQ ID NO: 706 | SEQ ID NO: 1161 | SEQ ID NO: 1616 |
| | opuCA.18 | CAAGTGGTTGTGGGAAAACGA | GGATCTTCCGCCATGATGTCTTTAT | AAGCCGGTTAATCATCTTC |
| | | SEQ ID NO: 707 | SEQ ID NO: 1162 | SEQ ID NO: 1617 |
| | opuCA.19 | ACGGATTCTAAAACGCGGCTATAAA | ACTAAGCTGGCACGAGTAACAATT | CCGACAAGTCGTTTATC |
| | | SEQ ID NO: 708 | SEQ ID NO: 1163 | SEQ ID NO: 1618 |
| | opuCA.20 | TGGGAAAACGACAACAATGAAGATG | GGATCTTCCGCCATGATGTCTTTAT | ACCGGCTTATTGAACCTACA |
| | | SEQ ID NO: 709 | SEQ ID NO: 1164 | SEQ ID NO: 1619 |
| inlJ | inlJ.0 | CGACACGAACAAACTCACAAAGA | TTGCGTGCGCAGTTTAAATAAGTTA | ACAATGGATTTTGACTTA |
| | | SEQ ID NO: 710 | SEQ ID NO: 1165 | CATCTA |
| | | | | SEQ ID NO: 1620 |
| | inlJ.1 | CAGCGAAGAACAACTAGCTACTCT | GGTGGTAATGTTGTTACTTGTGCAA | TCGGTTATGGATGAATTAT |
| | | SEQ ID NO: 711 | SEQ ID NO: 1166 | SEQ ID NO: 1621 |
| | inlJ.2 | GAGTTGTAGAAATTCACTATGTTGACGAA | CAGTTGTGTAATTATCTCCTACTGTTCCA | ACAACTGAGCTCCGCCACT |
| | | SEQ ID NO: 712 | SEQ ID NO: 1167 | SEQ ID NO: 1622 |
| | inlJ.3 | ACACAAGTAGCCAAACAGTGACAT | GTTTTGCCATTAGCATCCACGTAA | TACAGGCTCTGCTGCTACG |
| | | SEQ ID NO: 713 | SEQ ID NO: 1168 | SEQ ID NO: 1623 |
| | inlJ.4 | CCAATGCAACTGGACAATTCACA | GTAAAGGTTGGTCGATGTTTGTGTT | AACCGTCAACTACATTTAC |
| | | SEQ ID NO: 714 | SEQ ID NO: 1169 | SEQ ID NO: 1624 |
| | inlJ.5 | CGACACGAACAAACTCACAAAGT | GGCTGACATCTATTTCGGTTAAGGT | CCACTGTTAACTTATTTAA |
| | | SEQ ID NO: 715 | SEQ ID NO: 1170 | ACTGC |
| | | | | SEQ ID NO: 1625 |
| | inlJ.6 | CCGCCGAACCAACCAATG | GGTTTGCGCGCTGCTT | CAACTGGACAATTCAC |
| | | SEQ ID NO: 716 | SEQ ID NO: 1171 | SEQ ID NO: 1626 |
| | inlJ.7 | CAGCAGAGCCTGTAACAGTGAATTA | GCCAATAGTTCCGTTTAGGATTTCG | ATGGAGCGAGCATTTT |
| | | SEQ ID NO: 717 | SEQ ID NO: 1172 | SEQ ID NO: 1627 |
| | inlJ.8 | CGACACGAACAAACTCACAAAGT | GGTTAATTGTGTATTGTGGCTGACA | CAACACCTTAACCGAAATAG |
| | | SEQ ID NO: 718 | SEQ ID NO: 1173 | SEQ ID NO: 1628 |
| | inlJ.9 | ACCGTTAATTACGTGGACGATACTG | GTGTCGCCAACATTTCCGTTTA | CTCTCCATCCGAAATAT |
| | | SEQ ID NO: 719 | SEQ ID NO: 1174 | SEQ ID NO: 1629 |
| | inlJ.10 | ACCCCGCTTACACAGTTAACATATTT | TTGTGTGTTGTGTGTTAGGTCTATTTCT | CTACGCTTTCAAAATTAAC |
| | | SEQ ID NO: 720 | SEQ ID NO: 1175 | SEQ ID NO: 1630 |
| | inlJ.11 | CGCGCAAACCGTCAACT | CGTCAACATAGTGAATTTCTACAA | CTGGATTTTTTGTGTAAATAT |
| | | SEQ ID NO: 721 | CTCCTT | SEQ ID NO: 1631 |
| | | | SEQ ID NO: 1176 | |
| | inlJ.12 | AGGAGAAAAATTGGCGGCTGAT | CGCGCTAGAAGTATAAGGATCGT | AATTACCGCTTAAAACTTC |
| | | SEQ ID NO: 722 | | SEQ ID NO: 1632 |
| | inlJ.13 | AGTAGGAACCGTAACAACTCCATTTG | CGCCAATTTTTCTCCTTTGTCATCA | AAGCCCCTCAACCCATC |
| | | SEQ ID NO: 723 | SEQ ID NO: 1178 | SEQ ID NO: 1633 |
| | inlJ.14 | GTAGCAGCAGAGCCTGTAACA | GGAGCGAGCATTTTGCCATTAG | CATCCACGTAATTCAC |
| | | SEQ ID NO: 724 | SEQ ID NO: 1179 | SEQ ID NO: 1634 |
| | inlJ.15 | AGCTAACTTTCCTAAATTGCTCCAGTAA | CGCTACAATCAAAATATGTTAAC | ACCGAAATAGATGTAACCCC |
| | | SEQ ID NO: 725 | TGTGTAAGC | SEQ ID NO: 1635 |
| | | | SEQ ID NO: 1180 | |
| | inlJ.16 | AAGCAGCAGAGCCTGTAACA | AGCGAGTGTTTTGCCGGTAT | CATCCACGTAATTCAC |
| | | SEQ ID NO: 726 | SEQ ID NO: 1181 | SEQ ID NO: 1636 |
| | inlJ.17 | ACACAAGTAGCCAAACAGTGACAT | GTTTTGCCATTAGCATCCACGTAA | AAGCAGCAGAGCCTG |
| | | SEQ ID NO: 727 | SEQ ID NO: 1182 | SEQ ID NO: 1637 |
| | inlJ.18 | CAATGCGACTGGACAATTCACAAG | TGGTAAAGGTTGATCTGTGTTTGTGT | TAGCGCACAAACTGTC |
| | | SEQ ID NO: 728 | SEQ ID NO: 1183 | SEQ ID NO: 1638 |
| | inlJ.19 | GATCTTTCACAAAACCCCAAATTAGTCT | CGCAAGACAAACTTTTCAGCTTTG | ATGAGAAACGTCTAATTTC |
| | | SEQ ID NO: 729 | SEQ ID NO: 1184 | SEQ ID NO: 1639 |
| | inlJ.20 | AACAGGCGATTCCACACCAT | CTATTTTTTCTTTTTCCAGATAAC | AAGACCCCAAGTAGAGCTG |
| | | SEQ ID NO: 730 | TAGAGCT | SEQ ID NO: 1640 |
| | | | SEQ ID NO: 1185 | |
| Lmo078 | lmo0782.0 | GCGCTTGGATGGATGAATATCG | GCTGATATCTTGTCCCCCAGTAAT | CAGCATCTGGAGCAACC |
| 2 | | SEQ ID NO: 731 | SEQ ID NO: 1186 | SEQ ID NO: 1641 |
| | lmo0782.1 | GCGCTTGGATGGATGAATATTGG | GCTGATATCTTGTCCCCCTGTAAT | CAGTGGCACCCGATGCT |
| | | SEQ ID NO: 732 | SEQ ID NO: 1187 | SEQ ID NO: 1642 |
| | lmo0782.2 | GCAGCATTTACTGCGTTTAACTTAGT | GCCGTATTGTTGGATAGCTTCTTTC | CTGCAACTAGTCCTAAAACA |
| | | SEQ ID NO: 733 | SEQ ID NO: 1188 | SEQ ID NO: 1643 |
| | lmo0782.3 | CAATGGTTATTAACATGATGTCCGCTAA | GCTGCAACTAGTCCTAAAACACCTA | CTGCGACAACAAAACCTAAGA |
| | | SEQ ID NO: 734 | SEQ ID NO: 1189 | SEQ ID NO: 1644 |
| | lmo0782.4 | AACGTAGCTGGTGGATTTATCGTT | CTGCGACAACAAAACCTAAGAAGAA | CATTGCGTAACCAACAAC |
| | | SEQ ID NO: 735 | SEQ ID NO: 1190 | SEQ ID NO: 1645 |
| | lmo0782.5 | GTTGGTACACAAGCAGTCGAAAATT | ACGATAAATCCACCAGCTACGTTT | CCGGACGTTATTGTTAATG |
| | | SEQ ID NO: 736 | SEQ ID NO: 1191 | SEQ ID NO: 1646 |
| | lmo0782.6 | CTTGAAATGATCGCGCTTGGAT | GGGCAGCATCTGGAGCAA | CAGCTCCGATATTCATCC |
| | | SEQ ID NO: 737 | SEQ ID NO: 1192 | SEQ ID NO: 1647 |
| | lmo0782.7 | AGTTGGTACTAGCGCCGTAGA | CCACCAGCTACGTTTAGACCATTAA | ATGGCATTGAGTAAATTT |
| | | SEQ ID NO: 738 | SEQ ID NO: 1193 | SEQ ID NO: 1648 |
| | lmo0782.8 | GGTGGATTTATCGTTGTTGTTGGTT | CTGCGACAACAAAACCTAAGAAGAA | TTAGCGGACATCATGTTAATAA |
| | | SEQ ID NO: 739 | SEQ ID NO: 1194 | SEQ ID NO: 1649 |
| | lmo0782.9 | GACTGGATTCATGTTTCAGCACTAC | GGTGACTGCCACGATAATTGCT | ATGCGTATCGCAATTCC |
| | | SEQ ID NO: 740 | SEQ ID NO: 1195 | SEQ ID NO: 1650 |
| | lmo0782.10 | AGTCTCGACTGGATTCATGTTTCAG | ACTGCCACGATAATTGCTGGAAT | ACGCATTGCTTGAAGTAGT |
| | | SEQ ID NO: 741 | SEQ ID NO: 1196 | SEQ ID NO: 1651 |
| | lmo0782.11 | CCTTTCTTCTTCTTAGGCTTTGTTGT | GCAACGAGTCCTATAACACCTAGTG | CAGCATTCACTCAATTCA |
| | | SEQ ID NO: 742 | SEQ ID NO: 1197 | SEQ ID NO: 1652 |
| | lmo0782.12 | AGCCTGTACATTAATTGGTCTTGTTCTT | CGCAGCTCCGATATTCATCCAT | CAAGCGCGATCATTTC |
| | | SEQ ID NO: 743 | SEQ ID NO: 1198 | SEQ ID NO: 1653 |
| | lmo0782.13 | GGGAAAGAAGGAAATTTGCGAAGTC | CGATACGCATTGCTTGAAGTAGTAG | TCGACTGGATTCATGTTTCA |
| | | SEQ ID NO: 744 | SEQ ID NO: 1199 | SEQ ID NO: 1654 |
| | lmo0782.14 | GCTGCTCTTGGTCAAGTACTTACAA | CTTACCAGCTTTATCAGCCATGTG | TTGGAATGCAACTGTAATTG |
| | | SEQ ID NO: 745 | SEQ ID NO: 1200 | SEQ ID NO: 1655 |
| | lmo0782.15 | GCGCTTGGATGGATGAATATCG | TGATATCTTGTCCCCCCGTAATAACT | CAGCATCTGGAGCAACC |
| | | SEQ ID NO: 746 | SEQ ID NO: 1201 | SEQ ID NO: 1656 |
| | lmo0782.16 | GGTAAGGAAGGTAATCTGCGAAGT | GCGATACGCATAGCTTGAAGTAGTA | CTCGACTGGATACATGTTAC |
| | | SEQ ID NO: 747 | SEQ ID NO: 1202 | SEQ ID NO: 1657 |
| | lmo0782.17 | GCAGCATTCACTCAATTCAACTTAGT | CCGTATTGCTCGATAGCTTCTTTCA | CTGCAACGAGTCCTATAAC |
| | | SEQ ID NO: 748 | SEQ ID NO: 1203 | SEQ ID NO: 1658 |
| | lmo0782.18 | GCTGCTCTTGGTCAGGTACTTACTA | TCCCAGCTTTATCAGCCATGTG | TTGAAATGCAACTGTAATTGT |
| | | SEQ ID NO: 749 | SEQ ID NO: 1204 | SEQ ID NO: 1659 |
| | lmo0782.19 | GTAGTATTTTGGATGAGTGGCAAACG | CCCCCAATAATGATACCGGTTGTAA | CACCGTCCATTAATTG |
| | | SEQ ID NO: 750 | SEQ ID NO: 1205 | SEQ ID NO: 1660 |
| | lmo0782.20 | GAAATGATCGCGCTTGGCT | GCAGCATCAGGTGCAACAG | CAGCTCCGATGTTCATC |
| | | SEQ ID NO: 751 | SEQ ID NO: 1206 | SEQ ID NO: 1661 |
| plcA | plcA.0 | CGCAAACGATGTCATTGTACCAA | GGCCCATGGTAAATTTTGAGATTGT | CAACTAGAAGCAGGAATAC |
| | | SEQ ID NO: 752 | SEQ ID NO: 1207 | SEQ ID NO: 1662 |
| | plcA.1 | GAACCACACAAAAAAGCCATTAGTCA | CTCGGACCATTGTAGTCATCTTGAA | ACTGCATGCCGAATTT |
| | | SEQ ID NO: 753 | SEQ ID NO: 1208 | SEQ ID NO: 1663 |
| | plcA.2 | GAAGCAGGAATACGGTACATCGA | CGTTTCTAATACACCTGAAAGTGATGCA | CATGGGCCAATTTATT |
| | | SEQ ID NO: 754 | SEQ ID NO: 1209 | SEQ ID NO: 1664 |
| | plcA.3 | GCGGGAAAGCATATTCGCTTAATAA | GCGCTGCTAGGTTTGTTGTG | TCAGGTAGAGCGGACATC |
| | | SEQ ID NO: 755 | SEQ ID NO: 1210 | SEQ ID NO: 1665 |
| | plcA.4 | CCAGAACTGACACGAGCAATAAAAT | GGCTTTTTTGTGTGGTTCTCTGAAA | CCGCGGAAAAATAT |
| | | SEQ ID NO: 756 | SEQ ID NO: 1211 | SEQ ID NO: 1666 |
| | plcA.5 | AGACGAGCAAAATAGCAACGATAGT | AGGGATTTTATTGCTCGTGTCATTTCT | CAAAGATTATTTTTACACCA |
| | | SEQ ID NO: 757 | SEQ ID NO: 1212 | CTCCC |
| | | | | SEQ ID NO: 1667 |
| | plcA.6 | ACCAACAACTAGAAGCAGGAATACG | TGGCCCATGGTAAATGTTGAGATT | TATCGATATTAGAGCAAAAGAC |
| | | SEQ ID NO: 758 | SEQ ID NO: 1213 | SEQ ID NO: 1668 |
| | plcA.7 | CAATGGTCCGAGTGTGAAAACAAAA | TGTTGTCCGCTTTAGAAGCTTGATA | CAGTCTGGACAATCTCTTT |
| | | SEQ ID NO: 759 | SEQ ID NO: 1214 | SEQ ID NO: 1669 |
| | plcA.8 | CGAGCAAAACAGCAACGATAGTT | CGTGTCAGTTCTGGGAGTAGTGTA | CCAACCACTAATCAACATTT |
| | | SEQ ID NO: 760 | SEQ ID NO: 1215 | ATAAA |
| | | | | SEQ ID NO: 1670 |
| | plcA.9 | CGGATCCAACCATTAATCAACATTTACAA | CGCGGACATCTTTTAATGTAGGGAT | CTGACACGAGCAATAAA |
| | | SEQ ID NO: 761 | SEQ ID NO: 1216 | SEQ ID NO: 1671 |
| | plcA.10 | GCATCACTTTCAGGTGTATTAGAAACG | TGGTTGGGTCCGATAATCAAAACT | TCGTTGCTGTTTTGCTCG |
| | | SEQ ID NO: 762 | SEQ ID NO: 1217 | SEQ ID NO: 1672 |
| | plcA.11 | CCAGAACTGACACGAGCAATAAAAT | GGCTTTTTTGTGTGGTTCTCTGAAA | CCGCGGACAT |
| | | SEQ ID NO: 763 | SEQ ID NO: 1218 | SEQ ID NO: 1673 |
| | plcA.12 | CAGCACTCTCTATACCAGGTACAC | TGTGTTTGAGCTAGTGGTTTGGTTA | CCGTTATAGCTCATCGTATCAT |
| | | SEQ ID NO: 764 | SEQ ID NO: 1219 | SEQ ID NO: 1674 |
| | plcA.13 | TGATACGATGAGCTATAACGGAGACA | GTTTGTGTTTGAGCCAGTGGTT | ACGCGGACATTAACCA |
| | | SEQ ID NO: 765 | SEQ ID NO: 1220 | SEQ ID NO: 1675 |
| | plcA.14 | CAATCTCAAAATTTACCATGGGCCAA | GTTCTGCTCGTCTTTTAAACGCATA | CCTGAAAGTGATGCATTTAA |
| | | SEQ ID NO: 766 | SEQ ID NO: 1221 | SEQ ID NO: 1676 |
| | plcA.15 | TTGTATAAGAATTATTTGCAACGCACATT | GCGAATATGCTTTTCCGCCTAAT | AACAAAACAGAGCAATAAAA |
| | | SEQ ID NO: 767 | SEQ ID NO: 1222 | SEQ ID NO: 1677 |
| | plcA.16 | GATACGATGAGTTACAACGGAGACA | CGTATTCCTGCTTCTAGTTGTTGGT | ACCACTAGCTCAAACACA |
| | | SEQ ID NO: 768 | SEQ ID NO: 1223 | SEQ ID NO: 1678 |
| | plcA.17 | AAACCACTAGCTCAAACACAAACG | TGCTCTAATATCGATGTACCGTATTCCT | ATGTCATTGTACCAACAACTAG |
| | | SEQ ID NO: 769 | SEQ ID NO: 1224 | SEQ ID NO: 1679 |
| | plcA.18 | GAAGCAGGAATACGGTACATCGA | ATCGTTCCTAATACACCTGAAAGTGATG | ATGGGCCAATTTTTTTAAATG |
| | | SEQ ID NO: 770 | SEQ ID NO: 1225 | SEQ ID NO: 1680 |
| | plcA.19 | ATTCAAAGAGATTGTCCAGACTGCTT | GTGAAAGTTAATGAAGTGGCGCTAA | CCGCTTTGGAAGCTTG |
| | | SEQ ID NO: 771 | SEQ ID NO: 1226 | SEQ ID NO: 1581 |
| | plcA.20 | ACGCAAACGATGTCATTGTATCAAC | GGCCCATGGTAAATTTTGAGATTGT | ACCGTATTCCTGCTTCTAATT |
| | | SEQ ID NO: 772 | SEQ ID NO: 1227 | SEQ ID NO: 1682 |
| lmo018 | lo0189.0 | CGGTATCCTAGCTGAAACATATCCA | TCTACTGCGTCTGTTAAGATATCCGTAT | TTTTCATTGTGGAGCTTGATCA |
| 9 | | SEQ ID NO: 773 | SEQ ID NO: 1228 | SEQ ID NO: 1683 |
| | lmo0189.1 | AAAACCATTGCAAGCATTAAGACGAA | ACGACCACCGTTTGCTTTCA | ATGTCAATGCTTTACCTAATCTA |
| | | SEQ ID NO: 774 | SEQ ID NO: 1229 | SEQ ID NO: 1684 |
| | lmo0189.2 | CCGTTTTCATTGTGGAGCTTGATC | TCTACTGCGTCTGTTAAGATATCCGTAT | CTATAGGATACTCTTTCAAAGTTG |
| | | SEQ ID NO: 775 | SEQ ID NO: 1230 | SEQ ID NO: 1685 |
| | lmo0189.3 | CCGTTTTCATTGTGGAGCTTGATC | TGTAAATACAAGTTCTACTGCGTCTGTT | CCGTGTAACTATAGGATACTC |
| | | SEQ ID NO: 776 | SEQ ID NO: 1231 | SEQ ID NO: 1686 |
| | lmo0189.4 | GCAAACGGTGGTCGTAAGAAAAC | TTGATCAAGCTCCACAATGAAAACG | ACGTTGCGGTATCCTAG |
| | | SEQ ID NO: 777 | SEQ ID NO: 1232 | SEQ ID NO: 1687 |
| | lmo0189.5 | AAAACCATTGCAAGCATTAAGACGAA | GCAACGTTCAATCGTTTTCTTACG | ACCACCGTTTGCTTTC |
| | | SEQ ID NO: 778 | SEQ ID NO: 1233 | SEQ ID NO: 1688 |
| | lmo0189.6 | AAAACCATTGCAAGCATTAAGACGAA | GCAACGTTCAATCGTTTTCTTACGA | CCGCCGTTTGCTTTC |
| | | SEQ ID NO: 779 | SEQ ID NO: 1234 | SEQ ID NO: 1689 |
| | lmo0189.7 | AGCAAACGGCGGTCGTA | CGGATGGATATGTTTCAGCTAGGAT | CAACGTTCAATCGTTTTCT |
| | | SEQ ID NO: 780 | SEQ ID NO: 1235 | SEQ ID NO: 1690 |
| | lmo0189.8 | CAAACGGCGGTCGTAAGAAAAC | TTGATCAAGCTCCACAATGAAAACG | ACGTTGCGGTATCCTAG |
| | | SEQ ID NO: 781 | SEQ ID NO: 1236 | SEQ ID NO: 1691 |
| | lmo0189.9 | CCGTTTTCATTGTGGAGCTTGATC | TGTAAATACAAGTTCTACTGCGTCTGTT | ATCCTATAGTTATACGGAT |
| | | SEQ ID NO: 782 | SEQ ID NO: 1237 | ATCTT |
| | | | | SEQ ID NO: 1692 |
| | lmo0189.10 | AAAACCATTGCAAGCATTAAGACGAA | GCAACGTTCAATCGTTTTCTTACGA | AAGCATTGACATTGAAAGCA |
| | | SEQ ID NO: 783 | SEQ ID NO: 1238 | SEQ ID NO: 1693 |
| | lmo0189.11 | CCGTTTTCATTGTGGAGCTTGATC | CTGCGTCTGTTAAGATATCCGTGTA | CTATAGGATACTCTTTC |
| | | SEQ ID NO: 784 | SEQ ID NO: 1239 | AAAGTTG |
| | | | | SEQ ID NO: 1694 |
| | lmo0189.12 | CGGTATCCTAGCTGAAACATATCCA | CTGCGTCTGTTAAGATATCCGTGTA | TTTTCATTGTGGAGCTTGATCA |
| | | SEQ ID NO: 785 | SEQ ID NO: 1240 | SEQ ID NO: 1695 |
| | lmo0189.13 | CGATTGAACGTTGCGGTATCCTA | CTGCGTCTGTTAAGATATCCGTGTA | AACGGATGGATATGTTTCAGC |
| | | SEQ ID NO: 786 | SEQ ID NO: 1240 | SEQ ID NO: 1696 |
| | lmo0189.14 | TGAAAGCAAACGGTGGTCGTA | CGGATGGATATGTTTCAGCTAGGAT | CAACGTTCAATCGTTTTCT |
| | | SEQ ID NO: 787 | SEQ ID NO: 1242 | SEQ ID NO: 1697 |
| | lmo0189.15 | AAAACCATTGCAAGCATTAAGACGAA | GACCGCCGTTTGCTTTCA | ATGTCAATGCTTTACCTAATCTA |
| | | SEQ ID NO: 788 | SEQ ID NO: 1243 | SEQ ID NO: 1698 |
| | lmo0189.16 | CGATTGAACGTTGCGGTATCCTA | CTGCGTCTGTTAAGATATCCGTGTA | AACGGATGGATATGTTTCAGC |
| | | SEQ ID NO: 789 | SEQ ID NO: 1244 | SEQ ID NO: 1699 |
| | lmo0189.17 | AAAACCATTGCAAGCATTAAGACGAA | CCACCGTTTGCTTTCAATGTCA | ATGCTTTACCTAATCTAGAATCT |
| | | SEQ ID NO: 790 | SEQ ID NO: 1245 | SEQ ID NO: 1700 |
| lmo088 | lmo0880.0 | GCGATACTTTACCAGGAGTAGCT | GTCTGATGTAAGGTTGTTCCAGTCT | CTGCAACGGTAACATC |
| 0 | | SEQ ID NO: 791 | SEQ ID NO: 1246 | SEQ ID NO: 1701 |
| | lmo0880.1 | CGAAACAGATTTTGGTCCACTTGAA | CTCCAGGTAATGTATCGCCATCTTT | CACTTTATAGATGACTTTAA |
| | | SEQ ID NO: 792 | SEQ ID NO: 1247 | TTTT |
| | | | | SEQ ID NO: 1702 |
| | lmo0880.2 | GCGGACCGCAATTTTGAATTATCTA | AGCTTTTTTGTTTCGTCGTAATTTTCGT | ATGACAACAGATTGATTAT |
| | | SEQ ID NO: 793 | SEQ ID NO: 1248 | TTAAC |
| | | | | SEQ ID NO: 1703 |
| | lmo0880.3 | CAAACAGACCAAGTAAATCCAATTGCT | CCGCCACCAGTATTAACTTTGCTAA | TCGTTTGGGTCGTATTTT |
| | | SEQ ID NO: 794 | SEQ ID NO: 1249 | SEQ ID NO: 1704 |
| | lmo0880.4 | CAACAGATTACGGTAGCAG | CTACCCGCACTTTGCTATTTTCTTT | AATGTAGCTTTGCAAAATC |
| | | SEQ ID NO: 795 | SEQ ID NO: 1250 | SEQ ID NO: 1705 |
| | lmo0880.5 | CGAAACAGATTTTGGTCCACTTGAA | CAGGTAATGTATCGCCATCTTTCAC | CCATTTTGTTCGGAATCTT |
| | | SEQ ID NO: 796 | SEQ ID NO: 1251 | SEQ ID NO: 1706 |
| | lmo0880.6 | CGGTGTCTAGAAATGCCACTGTTAA | GTGGTGTGGTTCCTGATCCTT | CCCGCCACTCCCGTAAT |
| | | SEQ ID NO: 797 | SEQ ID NO: 1252 | SEQ ID NO: 1707 |
| | lmo0880.7 | CCATTGATCAAACAACAGGAACGAT | GGCTGCCACTTAAATTTGTATGTGT | ACGTAGTCGGTAAAAGTC |
| | | SEQ ID NO: 798 | SEQ ID NO: 1253 | SEQ ID NO: 1708 |
| | lmo0880.8 | GTTGACACCTATTGATAATGCCGTTT | CGGTATTTGATTGTTGCACTTCCAA | TTGGCTCAGGGCAAAAT |
| | | SEQ ID NO: 799 | SEQ ID NO: 1254 | SEQ ID NO: 1709 |
| | lmo0880.9 | GACGAAACAAAAAAGCTATACGAATAAAGC | CTTGTCCACCACTACCGTAATCATT | CACTGTCGCGTTTCTC |
| | | SEQ ID NO: 800 | SEQ ID NO: 1255 | SEQ ID NO: 1710 |
| | lmo0880.10 | CAACAGATTACGGCAGCA | CGTTGTTTGCTCCCCGTTT | ACTAATGTATCATTACAAAATCA |
| | | SEQ ID NO: 801 | SEQ ID NO: 1256 | SEQ ID NO: 1711 |
| | lmo0880.11 | CATAACCTCCTTGGAAATGCTACCA | TGCGGTTACTATCTAGCGCAA | TTGGTTGTACACTTGTAGAT |
| | | SEQ ID NO: 802 | SEQ ID NO: 1257 | ATTAC |
| | | | | SEQ ID NO: 1712 |
| | lmo0880.12 | ATGAAAAAAAGATGGTTGGTATTTGCAAT | CCCGTAATCTGTTGCAGCTTCT | TCACTGGATTTTTAAGCCC |
| | | SEQ ID NO: 803 | SEQ ID NO: 1258 | SEQ ID NO: 1713 |
| | lmo0880.13 | ACTTACGATACTAAAATCACCACGAAACAA | CGGTTGCATTTCTTGAAACTGCAT | AAGCCACTTTATCAGGTGACAAT |
| | | SEQ ID NO: 804 | SEQ ID NO: 1259 | SEQ ID NO: 1714 |
| | lmo0880.14 | GCCGGTCAAAAATTACAACTAACGA | CGCTTGTTGTGCTAGTCACTTTT | CAGGTGGAATGGTGATTTT |
| | | SEQ ID NO: 805 | SEQ ID NO: 1260 | SEQ ID NO: 1715 |
| | lmo0880.15 | CAGACTTTGGACCAATTGAAGTTGTC | GCAACGCCTGGTAAAGTATCG | ATCTTGTTCGGAATCTTT |
| | | SEQ ID NO: 806 | SEQ ID NO: 1261 | SEQ ID NO: 1716 |
| | lmo0880.16 | CGGATACTTTACAAGCCGGTCAAAA | GCACAGGTGGCACGGTAAT | TTTGATAAGAGCGTTTTTTC |
| | | SEQ ID NO: 807 | SEQ ID NO: 1262 | SEQ ID NO: 1717 |
| | lmo0880.17 | CTGTGAAAAATACGACCCAAACGAT | CCTGTACCGCCACCAGTAT | ACGCCTTTATTAGCAAAGTT |
| | | SEQ ID NO: 808 | SEQ ID NO: 1263 | SEQ ID NO: 1718 |
| | lmo0880.18 | CCACCAATATCCAACCAATTACGCT | TGTTTCGTCGTAATTTTCGTATCATACGT | ATGCGAATCGAAATTT |
| | | SEQ ID NO: 809 | SEQ ID NO: 1264 | SEQ ID NO: 1719 |
| | lmo0880.19 | GGCGAATGGATGATAAAGACTTGAG | CATTATCAATAGGTGTCAGCGCATT | ACATTGGACCGTTACTTTAA |
| | | SEQ ID NO: 810 | SEQ ID NO: 1265 | SEQ ID NO: 1720 |
| | lmo0880.20 | AGTTGCAAGTGGAGAAACAATGACT | GTCATTCACTGGAAAACCACCAAAA | CCGGAACAGCTGAAATTAATTA |
| | | SEQ ID NO: 811 | SEQ ID NO: 1266 | SEQ ID NO: 1721 |
| lmo051 | lmo0514.0 | GATGGTATTACAGTCAGCGGTGTA | AGTCGGAAAATCATAGCGTGCATTA | AAA GAAGAATTCGACAATCTT |
| 4 | | SEQ ID NO: 812 | SEQ ID NO: 1267 | SEQ ID NO: 1722 |
| | lmo0514.1 | GCCAGCTTTAAATACGCTTTTCTTACA | CGAATGCAGCAAGTGCTTTTAAATT | ATGGTCTAAAATCATCAATTCC |
| | | SEQ ID NO: 813 | SEQ ID NO: 1268 | SEQ ID NO: 1723 |
| | lmo0514.2 | GGAAGCTGGAAATCTGGTAACACTA | CCGCCCACTGTTAAGTAAGTCAAA | CCGATTAAAAACCAAACTTC |
| | | SEQ ID NO: 814 | SEQ ID NO: 1269 | SEQ ID NO: 1724 |
| | lmo0514.3 | CGCGACACTTACAAGCGATTTTAAT | CCGCGCTCATTTTCAGCATTTAAT | CCACACCAGGAAAATA |
| | | SEQ ID NO: 815 | SEQ ID NO: 1270 | SEQ ID NO: 1725 |
| | lmo0514.4 | TTAAGCTTGATGTACCAGGCGAAT | CTTTGAGTCCTGCAGCATTTTCT | ACCGTGACATTAAACGC |
| | | SEQ ID NO: 816 | SEQ ID NO: 1271 | SEQ ID NO: 1726 |
| | lmo0514.5 | ACACGTTAGATTTAACAGCGGATGA | CCATCGTCCGTTTTTGCTTGAAC | CATTTCTGAAGAACAATTTT |
| | | SEQ ID NO: 817 | SEQ ID NO: 1272 | SEQ ID NO: 1727 |
| | lmo0514.6 | GGGCGGAGACAATGTAAAAGATAGT | TCATGGAGTTTTGCGCATAGAGAT | AAGACATTATGCGTCACTTCA |
| | | SEQ ID NO: 818 | SEQ ID NO: 1273 | SEQ ID NO: 1728 |
| | lmo0514.7 | CAGGTGCGAATTATAGCGATTTAACAG | GCTAGTGTTTGGATGCTCGTGTTAT | AAGCAGCATCAAACCTT |
| | | SEQ ID NO: 819 | SEQ ID NO: 1274 | SEQ ID NO: 1729 |
| | lmo0514.8 | GCTGGTCTAAAAGCGGATCCAATAA | TGTCGGATCTGCTGTGATAACTG | TTTTTCATGAACAACTACT |
| | | SEQ ID NO: 820 | SEQ ID NO: 1275 | TTTAC |
| | | | | SEQ ID NO: 1730 |
| | lmo0514.9 | CCAAACACTTGAACCGATTAAAAACCA | ACAAGTCCATTTAAATCTACGAAAA | TCCGCCCACTGTTAAGT |
| | | SEQ ID NO: 821 | GACTATCTT | SEQ ID NO: 1731 |
| | | | SEQ ID NO: 1276 | |
| | lmo0514.10 | TTGCCAGCATTAACAACGCTTTT | TTTGTCCAAATGCTGCTAAAGCTTTT | ATGGAATTGATGATTTTCG |
| | | SEQ ID NO: 822 | SEQ ID NO: 1277 | SEQ ID NO: 1732 |
| | lmo0514.11 | GGTTGGTGGAGACAATGTAACAGAT | GACTAACATTTTTACTGCTAATACTT | AAGCATGTAAATCAACAAACG |
| | | SEQ ID NO: 823 | ATACTTTTCAAGGTT | SEQ ID NO: 1733 |
| | | | SEQ ID NO: 1278 | |
| | lmo0514.12 | CCAGCCTTAACAACACTTTTCTTACAA | GAGGGCTTTCAAGTTGCCATTTT | CAAAGTCATTTAAAGGTCT |
| | | SEQ ID NO: 824 | SEQ ID NO: 1279 | AAAATC |
| | | | | SEQ ID NO: 1734 |
| | lmo0514.13 | GAAGAAACCGACCCAACAATCATC | TTGCATTATTTTCTTTCACTTGGT | CCTCAAAGCGAAAATT |
| | | SEQ ID NO: 825 | CTTCTG | SEQ ID NO: 1735 |
| | | | SEQ ID NO: 1280 | |
| | lmo0514.14 | AGCCTCTTTGCCAGCATTAACA | TTTGTCCAAATGCTGCTAAAG | ACGCTTTTTGTGCAATTTG |
| | | SEQ ID NO: 826 | SEQ ID NO: 1281 | SEQ ID NO: 1736 |
| | lmo0514.15 | CCCAGGCGAATATACCGTAACTTTA | GTCGCTTGTAGTCACTGTTACGA | ACCGCGGCCAAAAA |
| | | SEQ ID NO: 827 | SEQ ID NO: 1282 | SEQ ID NO: 1737 |
| | lmo0514.16 | CGACTTCCGCAAGTAATACTTATTTAGGA | CACCGCTGACTGTAATACCATCAT | TTGGGAGTGCCACATCAT |
| | | SEQ ID NO: 828 | SEQ ID NO: 1283 | SEQ ID NO: 1738 |
| | lmo0514.17 | CACGGTCACATTAAACGCAGAAAAT | GTAGGATCTGCTGTGATAACAGGTT | CACAGGAGTTGCTTTTAG |
| | | SEQ ID NO: 829 | SEQ ID NO: 1284 | SEQ ID NO: 1739 |
| | lmo0514.18 | TGGCACGGTTGCTCCTTTT | CGGTAATGGAAAGACGTGAGCTT | CCACATCATTAAATCCTA |
| | | SEQ ID NO: 830 | SEQ ID NO: 1285 | AATAAG |
| | | | | SEQ ID NO: 1740 |
| | lmo0514.19 | CTGGAAATGCCGTTTTAACAAGTGA | GCCGCGGTCATTTATTGCATTTAA | ACCCCAGGCGAATAT |
| | | SEQ ID NO: 831 | SEQ ID NO: 1286 | SEQ ID NO: 1741 |
| | lmo0514.20 | AGACCCTGTAACCGTAGTTGTGA | GTTGGTGGAACCGGATTAACATG | CCGTCGCTAGTAGTCACT |
| | | SEQ ID NO: 832 | SEQ ID NO: 1287 | SEQ ID NO: 1742 |
| lmo129 | lmo1290.0 | CGACTGTTGTAGATCTTTCCAAACC | ACTTGCACTGGCAAAGCTTTT | AAACGCAGAAAATGATTTAC |
| 0 | | SEQ ID NO: 833 | SEQ ID NO: 1288 | SEQ ID NO: 1743 |
| | lmo1290.1 | AGGACTAAAAGCTAGCCAAGATGTC | GCATCAGCAGATTTGCCAGTTAAAA | CCGGATCTGGAATATT |
| | | SEQ ID NO: 834 | SEQ ID NO: 1289 | SEQ ID NO: 1744 |
| | lmo1290.2 | CGGGCTTAGTAATCTTGAACGCTTA | CCAGTAGAGTTAAATTAGTCAACCCACTT | CAGCTCCCATAATGCG |
| | | SEQ ID NO: 835 | SEQ ID NO: 1290 | SEQ ID NO: 1745 |
| | lmo1290.3 | GGAACCGCGATTACAAGTGATTTTG | GCTTTTTGCAAATCATTTTCCGCATT | TTCCTGGTTTAGAAAGATCT |
| | | SEQ ID NO: 836 | SEQ ID NO: 1291 | SEQ ID NO: 1746 |
| | lmo1290.4 | CAACCAGTGACAGAAGCAGAATTTT | CGTTCCAGGAGTCTTGAAATCTACT | CAACTGTTACAAGTGATTTC |
| | | SEQ ID NO: 837 | SEQ ID NO: 1292 | SEQ ID NO: 1747 |
| | lmo1290.5 | TGCTTTACCTAAAACAGGAGATTCTTTACC | TCTCCTTGAAACAATAAAGCTTAATCCGA | ATCCTACAGAAATCCC |
| | | SEQ ID NO: 838 | SEQ ID NO: 1293 | SEQ ID NO: 1748 |
| | lmo1290.6 | CAGTGCAAGTGATGGTTATTGTTGA | GGATTAGGTGTTGGACTTGGATCTG | TCGGGTCTGGTATTGGTG |
| | | SEQ ID NO: 839 | SEQ ID NO: 1294 | SEQ ID NO: 1749 |
| | lmo1290.7 | TGTGCATGATTATCGAGGGATTGAA | TTTTACCGCCTATGGTTTGGCTAT | AAGCAGTGTATTCAATTTAG |
| | | SEQ ID NO: 840 | SEQ ID NO: 1295 | SEQ ID NO: 1750 |
| | lmo1290.8 | TGGGACGTACACAGTTACTCTACAA | GCAGTAATCGTTGTCTTCGCTTTAA | CACCTGTTCAAGTAAACG |
| | | SEQ ID NO: 841 | SEQ ID NO: 1296 | SEQ ID NO: 1751 |
| | lmo1290.9 | GAATCAACTGTATGCATTTTCCCAAACT | GTCATCAAGCTGAATGGGACGTA | ATGCAGAAAAGCAAACATTA |
| | | SEQ ID NO: 842 | SEQ ID NO: 1297 | SEQ ID NO: 1752 |
| | lmo1290.10 | AACATCTCAGCTGACAGTGGAAAA | GTTGATCCGTCATCTGTTTTTGCAT | ACCAGTGACAGAAGCAG |
| | | SEQ ID NO: 843 | SEQ ID NO: 1298 | SEQ ID NO: 1753 |
| | lmo1290.11 | GCACAGTGCACATGATGATTCTATT | TTTTAAGTGGCATGATATCCGTTATTGC | ACCATTATAACTAAGGTCA |
| | | SEQ ID NO: 844 | SEQ ID NO: 1299 | ATACT |
| | | | | SEQ ID NO: 1754 |
| | lmo1290.12 | CGCTGCCAGAACTTAAGAGCTT | TGGCCATATGCATACAGTTGATTCA | CATGCACTCCATCGTATTGA |
| | | SEQ ID NO: 845 | SEQ ID NO: 1300 | SEQ ID NO: 1755 |
| | lmo1290.13 | CGCATTATGGGAGCTGATGTTACAT | ATGAGCACTGTGTGAAAGATCCA | AACCCACTTAAATTCG |
| | | SEQ ID NO: 846 | SEQ ID NO: 1301 | SEQ ID NO: 1756 |
| | lmo1290.14 | ACTCCTGGAACGTACACAGTTACT | GCCGTAATCGCTGTCTTCTCTTTAA | TTACAATCTGAGAATAATTCC |
| | | SEQ ID NO: 847 | SEQ ID NO: 1302 | SEQ ID NO: 1757 |
| | lmo1290.15 | TTCACACAGTGCTCATGATGATTCT | GGCATAATATCTGTTATTGCACCATTGT | CTAAGGTCGATGCTAGTAACT |
| | | SEQ ID NO: 848 | SEQ ID NO: 1303 | SEQ ID NO: 1758 |
| | lmo1290.16 | CCAGAAGTGCCAAGTCACAAAAT | CTCTTTGCTTGTTTCTGCTTTAGCT | TCCATCTTTAACTGTAAATGAG |
| | | SEQ ID NO: 849 | SEQ ID NO: 1304 | SEQ ID NO: 1759 |
| | lmo1290.17 | GTGCCAAGTCACAAAATTCCATCTT | AGGCAAGGAATCCCCTGTTTT | AAGCTAAAGCAGAAACAAG |
| | | SEQ ID NO: 850 | SEQ ID NO: 1305 | SEQ ID NO: 1760 |
| | lmo1290.18 | TGGAGTACAACGCGAAAATCGA | CAGATATATTGTACGCCCCACCATT | TTGAGTTCTCAAACTTATAA |
| | | SEQ ID NO: 851 | SEQ ID NO: 1306 | TATTC |
| | | | | SEQ ID NO: 1761 |
| | lmo1290.19 | AATCTTACAATATTCCGGAACAGTTCCA | GATGTTCAACGAATGGTCTACAGTGA | ATGGTGGCTCTTACACTATAT |
| | | SEQ ID NO: 852 | SEQ ID NO: 1307 | SEQ ID NO: 1762 |
| | lmo1290.20 | GTAACATTAAATGCGGAAAATGATTTGCA | TCGGATCTGGTATTGGTGTTTCTTT | CATTGGTGCAGCTTTT |
| | | SEQ ID NO: 853 | SEQ ID NO: 1308 | SEQ ID NO: 1763 |
| hlyA | hlyA | CCAGATTTTTCGGCAAAGCT | TGCAGGAGGATTTTCTGCATT | AGAGCAGTTGCAAGCG |
| | | SEQ ID NO: 854 | SEQ ID NO: 1309 | SEQ ID NO: 1764 |

The assays were evaluated under uninduced conditions as well as under induced conditions using pure cultures of Listeria monocytogenes. The extracted nucleic acids were assayed for induction by real-time reverse-transcriptase-PCR (DNA+RNA). To measure contribution due to DNA alone, control reactions were run without addition of the reverse transcriptase (DNA). Transcriptional activity (contribution to Ct value by RNA alone) was estimated by subtracting the Ct value obtained for DNA and RNA (with reverse transcriptase reaction) from the Ct value obtained for DNA alone (without reverse transcriptase reaction) for both uninduced and induced samples.

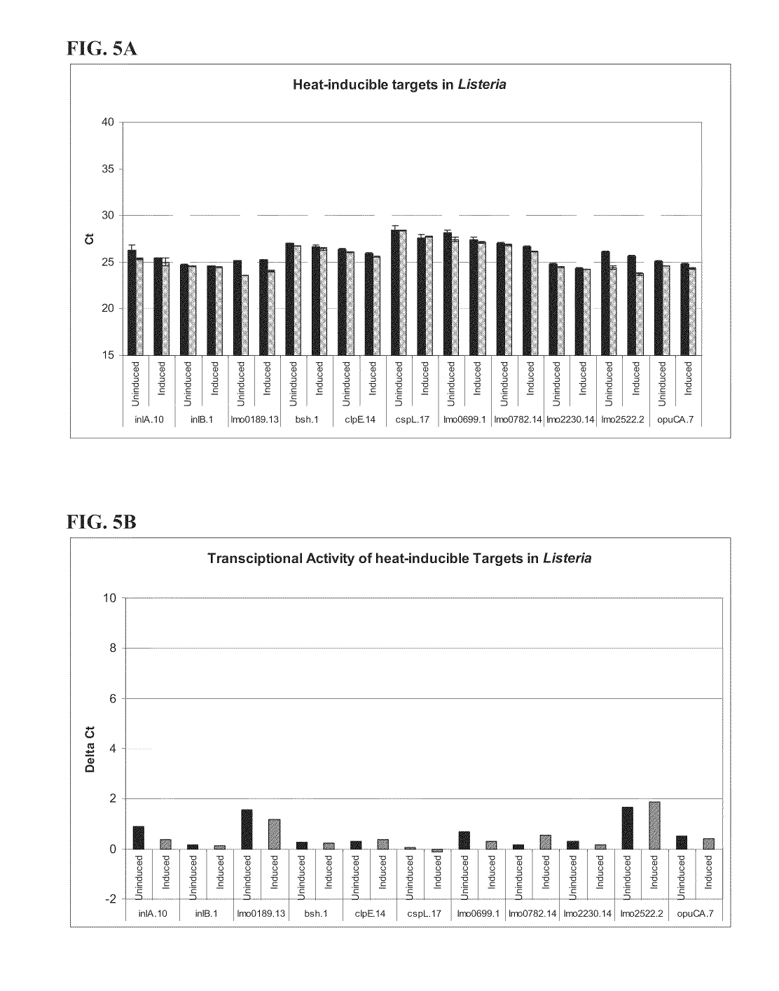

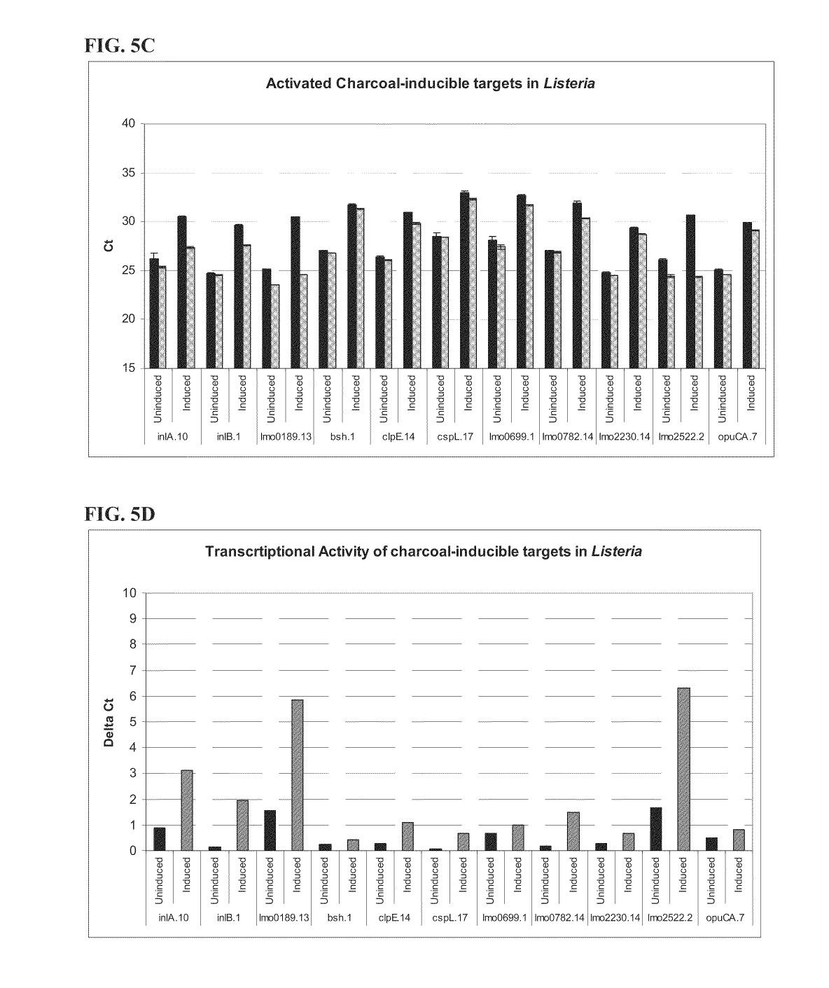

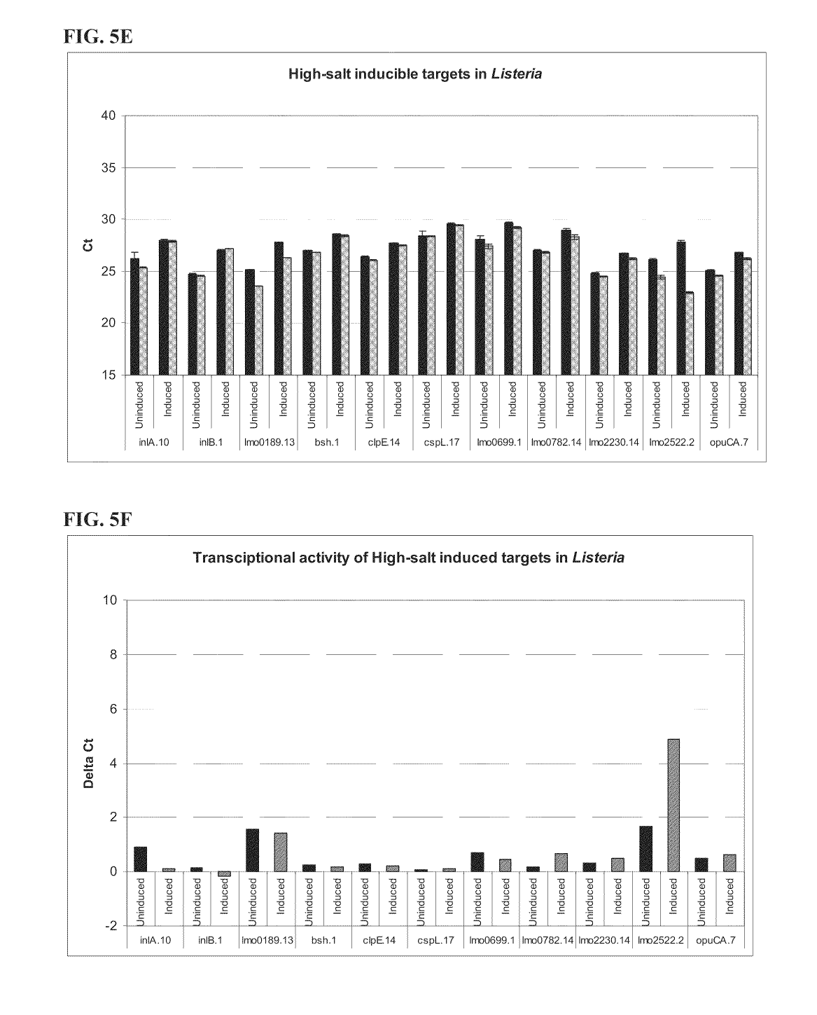

Heat-induction: Duplicate samples were maintained at 37° C. (uninduced) or 48° C. (heat-induced) for 20 minutes before processing for nucleic acids. Targets were initially evaluated using a pure culture of Listeria (FIG. 5A-FIG. 5F). Overall, addition of the reverse transcription step improved signal for both uninduced and induced samples. Heat-induction proved to be one of the milder inducers for Listeria, improving the signal by ˜2 Ct (FIG. 5A and FIG. 5B).

FIG. 5A-FIG. 5F show data for evaluation of targets under various inducible conditions for Listeria. FIG. 5A and FIG. 5B provide results using heat-induction. Ct (FIG. 5A) or Delta Ct (FIG. 5B) values are provided for various targets for uninduced (37° C.) and induced (48° C.) samples before processing for nucleic acids. FIG. 5C and FIG. 5D provide results using activated charcoal induction. Samples were grown in enrichment media treated with 0.2% activated charcoal for 5 hours, along with parallel uninduced samples. FIG. 5E and FIG. 5F provide results using salt induction. An aliquot of an exponentially grown culture was treated to enrichment media containing 0.3M NaCl (final concentration) for 10 minutes. Uninduced samples were processed the same way with media alone. FIG. 5A, FIG. 5C and FIG. 5E—The extracted nucleic acids were assayed for induction by real-time reverse-transcriptase-PCR (hatched bars). To measure contribution due to DNA, control reactions were run without addition of the reverse transcriptase (solid bars). FIG. 5B, FIG. 5D and FIG. 5F show transcriptional activity was estimated by subtracting the Ct value obtained for DNA and RNA (with reverse transciptase reactions) from the Ct value obtained for DNA alone (without reverse transcriptase reactions) for uninduced (solid bars) and induced (striped bars) samples.

Charcoal-induction: A parallel sample was grown in enrichment media treated with 0.2% activated charcoal for 5 hours, along with parallel uninduced samples. Growth in media treated with activated charcoal (FIG. 5C and FIG. 5D) proved to be a strong inducer of a transcriptional response. While for most genes, transcriptional activity was in the 0.5-1.5Ct range, multiple genes were strongly induced (Delta Ct=2-6; 4-64 fold differences).

Salt-Induction: One aliquot of an exponentially grown culture was treated to enrichment media containing 0.3M NaCl (final concentration) for 10 minutes. Uninduced samples were processed the same way with media alone. Brief exposure to high salt (FIG. 5E and FIG. 5F) proved also to be a strong inducer of lmo2522 (transcriptional activity=5 Ct), a gene that was also strongly induced by growth in media treated with activated charcoal.

Acid Induction: For acid conditions, a bacterial culture (2 mL) was diluted in 6 mL acidified BHI (acidified BHI prepared by adding HCl to BHI, and checking pH: such as 100 ul HCl to 5 ml BHI resulted in pH=2.5); incubation at 37° C., 10 minutes, with shaking. The moderately inducible RNA targets were: lmo0189 (encoding a sequence highly similar to B. subtilis Veg protein) and lmo2522 (encoding a sequence similar to hypothetical cell wall binding protein from B. subtilis). For both transcripts, acid conditions produced ˜3 fold induction. At the same time, the amount of an additional lmo0699 transcript (encoding a sequence similar to flagellar switch protein FliM) was ˜3 fold reduced under the acidic conditions.

Cold Induction: For cold conditions, a culture was placed on ice for 16, 20, or 24 minutes. The moderately inducible RNA targets were: lmo0189 (encoding a sequence highly similar to B. subtilis Veg protein) and lmo2522 (encoding a sequence similar to hypothetical cell wall binding protein from B. subtilis). For both transcripts, cold conditions produced ˜4 fold induction.

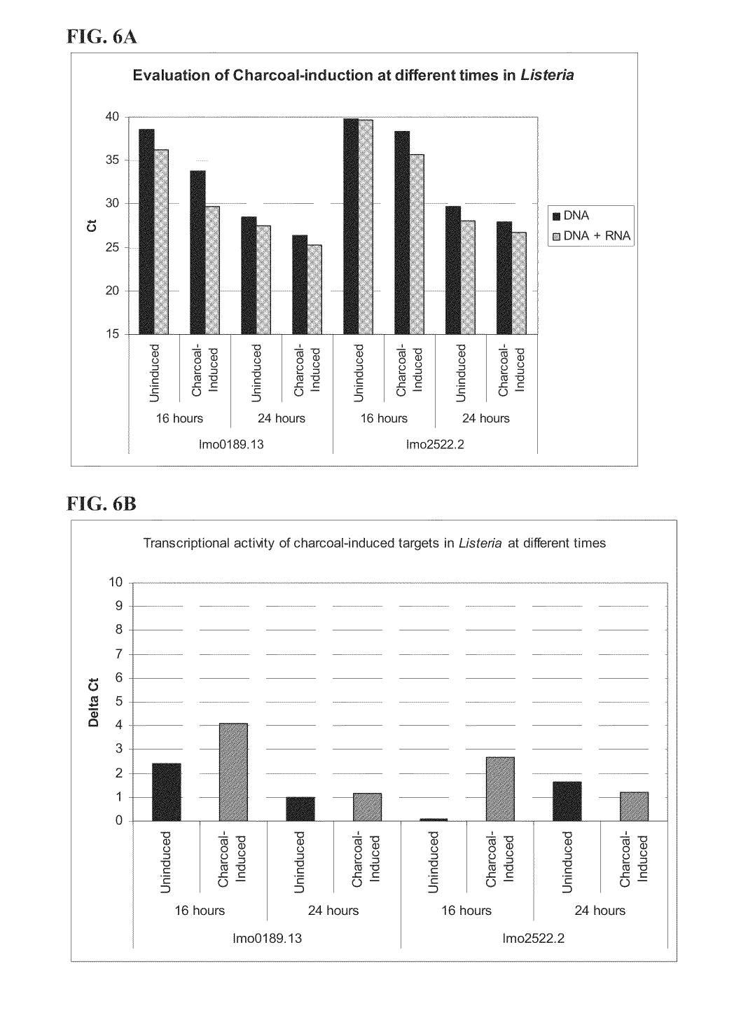

The Listeria targets were also evaluated in the context of a time-course study for activated charcoal induction in a milk sample. Raw milk (50 ml) was spiked with approximately 60 cfu of L. monocytogenes. The sample was split equally into 2×25 ml samples. One 25 ml sample was enriched in 225 ml of BLEB with supplements (Buffered Listeria Enrichment Broth, EMD Chemicals, Gibbstown, N.J.). The other 25 ml aliquot was enriched in BLEB treated with 0.2% activated charcoal (supplements added at the beginning of enrichment). Samples were incubated at 37° C. and withdrawn at 16 and 24 hours post-enrichment. FIG. 6A shows that extracted nucleic acids were assayed for induction by real-time reverse-transcriptase-PCR (hatched bars). To measure contribution due to DNA, control reactions were run without addition of the reverse transcriptase (solid bars). FIG. 6B. shows transcriptional activity was estimated by subtracting the Ct value obtained for DNA and RNA (with reverse transciptase reactions) from the Ct value obtained for DNA alone (without reverse transcriptase reactions) for uninduced (solid bars) and induced (striped bars) samples. As shown by the data of FIGS. 6A and 6B, greater effects of induction were observed during the earlier times of enrichment (16 hours versus 24 hours). The combination of induction and reverse transcription to detect RNA induced target dropped the signal to a more robust detection range for both targets tested; the data for target lmo0189 are particularly noted (FIG. 6A and FIG. 6B).

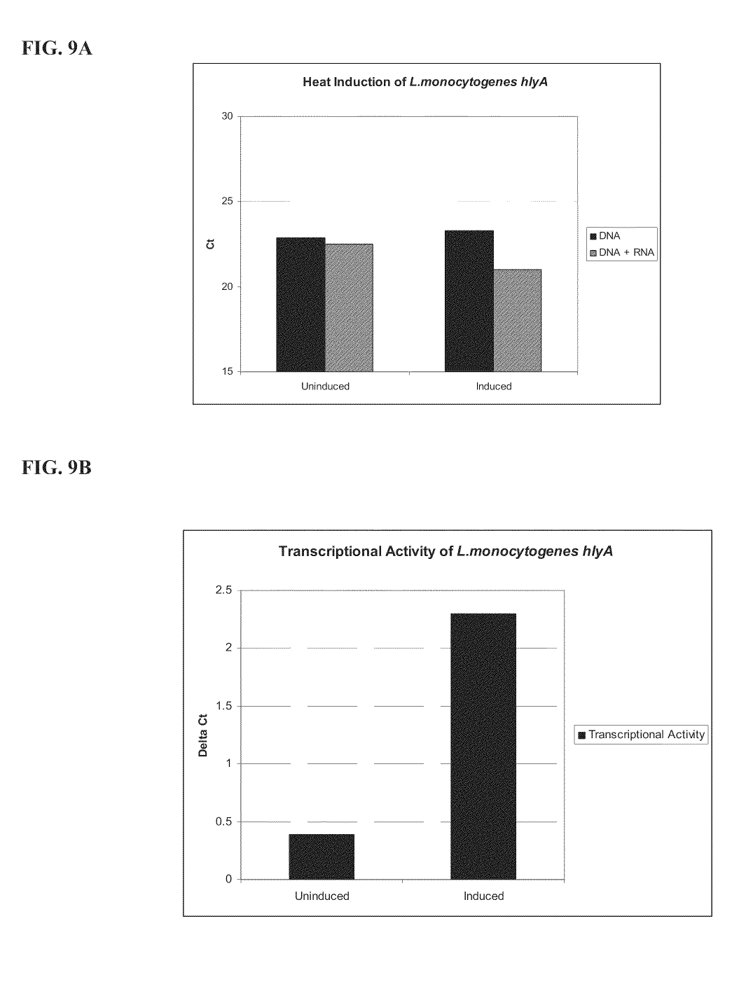

FIG. 9A and FIG. 9B provide data on the use of heat induction to detect Listeria monocytogenes by measuring the target hlyA. The data demonstrate that including the reverse transcriptase step in detection allows for more sensitive detection by 2 Cts.

Considering the long generation times for Listeria growth, an improvement of 0.5-1Ct in a detection assay can translate into reducing the enrichment times by 1-1.5 hours and a difference of 4 Ct will allow for at least a 4 hour reduction in enrichment time.

Results for enriched samples were confirmed by plating on CHROMagar™ plates (CHROMager Microbiology, Paris France). That is, following enrichment, samples were withdrawn for RNA extraction and, in parallel, samples were also plated for culture confirmation (CHROMAgar) to verify presence of pathogen. The plate results were recorded following overnight growth at 37° C. A 100% correlation was obtained between RT-PCR results and plate confirmation.

### Example 3

### Inducible RNA Targets for Early Detection of Vibrio

The present example provides identification and evaluation of inducible RNA targets for detection of the pathogenic microbe Vibrio cholerae using real-time RT-PCR. The normal growth temperature of Vibrio species is at 30° C.; it was discovered that growing Vibrio at 37° C. induced RNA such that reverse transcription RT-PCR detection increased sensitivity of the assay.

Sample Preparation and Real-time PCR: A single Vibrio cholerae bacterial colony was inoculated into 5 ml of nutrient enrichment broth containing 3% NaCl and grown overnight at 37° C. Nucleic acids were prepared for real-time PCR and real-time RT-PCR using PrepSEQ™ nucleic acids extraction protocol. Samples were assayed with and without the inclusion of the reverse transcriptase step.

Primers and Probes: TAQMAN® real-time PCR assay primers and probes are listed in Table 3. The hsp60 gene contains several polymorphic sequences and thus, a combination of three probes is provided for detecting all Vibrio cholerae strains. The probes were labeled with a FAM™ dye to enable detection by real-time PCR.

TABLE 3
TaqMan® assays designed against the hsp60 gene of Vibrio

| Gene | Forward Primer | Reverse Primer | Probe |
|---|---|---|---|
| hsp60 | GGTAGAAGAGTTGAAAGCACTGTC | ATGGTACCTACTTGGGCAATCG | CCTTGTGCCGATACTAAA |
| | (SEQ ID NO: 1765) | (SEQ ID NO: 1766) | (SEQ ID NO: 1767) |
| | | | CCTTGTGCTGATACTAAA |
| | | | (SEQ ID NO: 1768) |
| | | | ACCTGTGCAGATACTAAA |
| | | | (SEQ ID NO: 1769) |

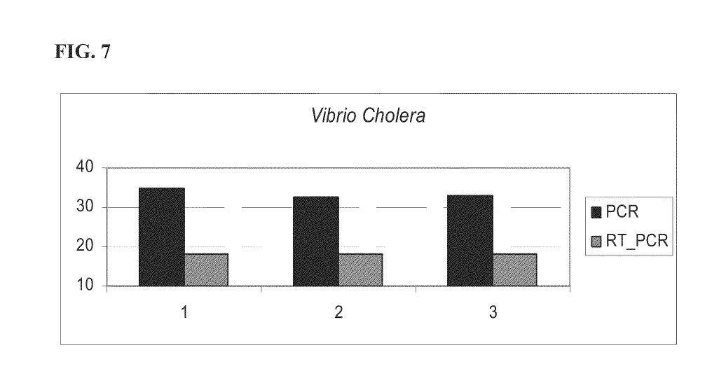

Results from PCR and reverse transcriptase RT-PCR assays for the hsp60 target demonstrate a significant difference in detection levels as shown by the data of FIG. 7. More than 10 Ct difference was detected when the value of Ct for DNA targets was compared to the value of Ct for RNA and DNA targets, which corresponds to more than 1000 fold difference in target concentration.

### Example 4

### Inducible RNA Targets for Early Detection: Evaluation on Food Testing Work Flows

The present disclosure has evaluated food testing workflow methods using inducible RNA targets in assays for detection of various bacteria. A fast workflow method is provided based on inducible transcription response in bacteria.

A typical workflow, according to this disclosure, includes a shortened enrichment step, a rapid induction step, an automated sample preparation step, and specific detection of induced target using reverse transcriptase RT-PCR.

In one embodiment, a workflow of the disclosure for Salmonella is as follows:

Enrichment (6 hours) [symbol] Induction+Sample Prep (45 min) [symbol] Real-Time RT-PCR (1 hour)

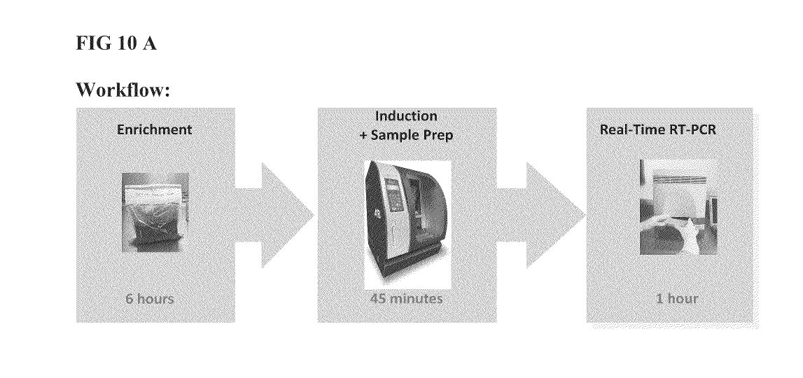

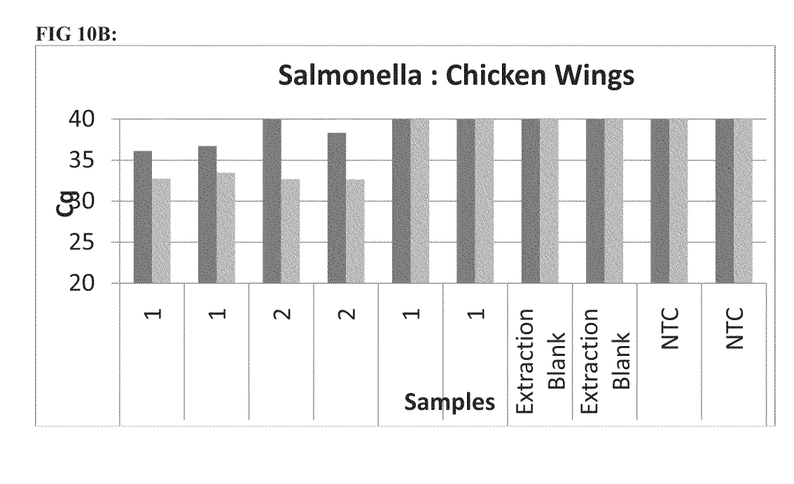

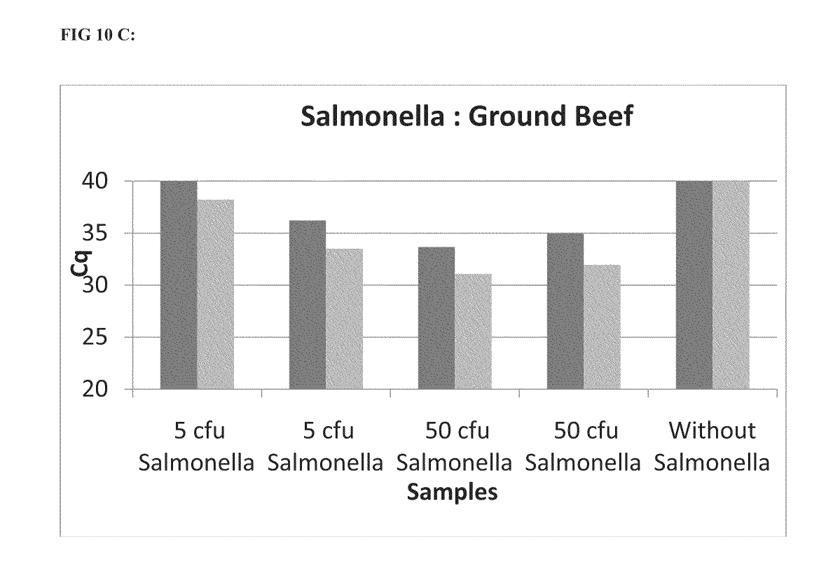

An example workflow is shown in FIG. 10A. As depicted in FIGS. 10B-10C, 25 g/25 ml food samples (chicken wings in FIG. 10 B and ground beef in FIG. 10C respectively) were spiked with known number of Salmonella and enriched in 225 ml media for 6 hours at 37° C. A 250 μl enriched sample was processed through AutoMate Express® System for induction and sample preparation. Eluted nucleic acid samples were evaluated by real-time reverse transcriptase PCR.

As shown in FIGS. 10 B and 10C, DNA (dark gray bars) and DNA plus induced RNA (light gray bars) were evaluated using real-time PCR in the absence or presence of ArrayScript®M-MLV reverse transcriptase. 100% detection was observed with induced RNA in all samples containing Salmonella, whereas few samples were not detected with DNA alone. All samples that were determined as positive by induced RNA were also confirmed to have Salmonella by a traditional culture method.

Additionally, FIG. 10B depicts detection of induced RNA in chicken wing samples spiked with Salmonella and shows an enrichment time of 6 hours with a Detection=100%. Similarly, FIG. 10C shows detection of induced RNA in ground beef samples spiked with Salmonella having an enrichment time of 6 hours and a Detection=100%.

Example workflows provided herein allowed for detection of 1-5 cfu of Salmonella in less then 8 hr (in 6 hours for the workflows shown in FIGS. 10B and 10C) and Listeria in less than 12 hr in food samples (results described in Example 2), thereby greatly reducing the time-to-result for testing, which is very important for industries such as the food industry, where valuable shelf life can be increased, if the testing time for food safety testing is reduces by the workflow methods provided herein.

### Example 5

### Use of Present Methods in Viability Testing

The present disclosure describes methods of detecting viable bacteria as induced RNA gene targets are only expressed in live organisms thus, detecting only live organisms and not dead organisms (which cannot cause diseases in samples). Accordingly, methods of the disclosure also reduce wastage of food attributed to amplification of dead microbes by traditional testing methods.

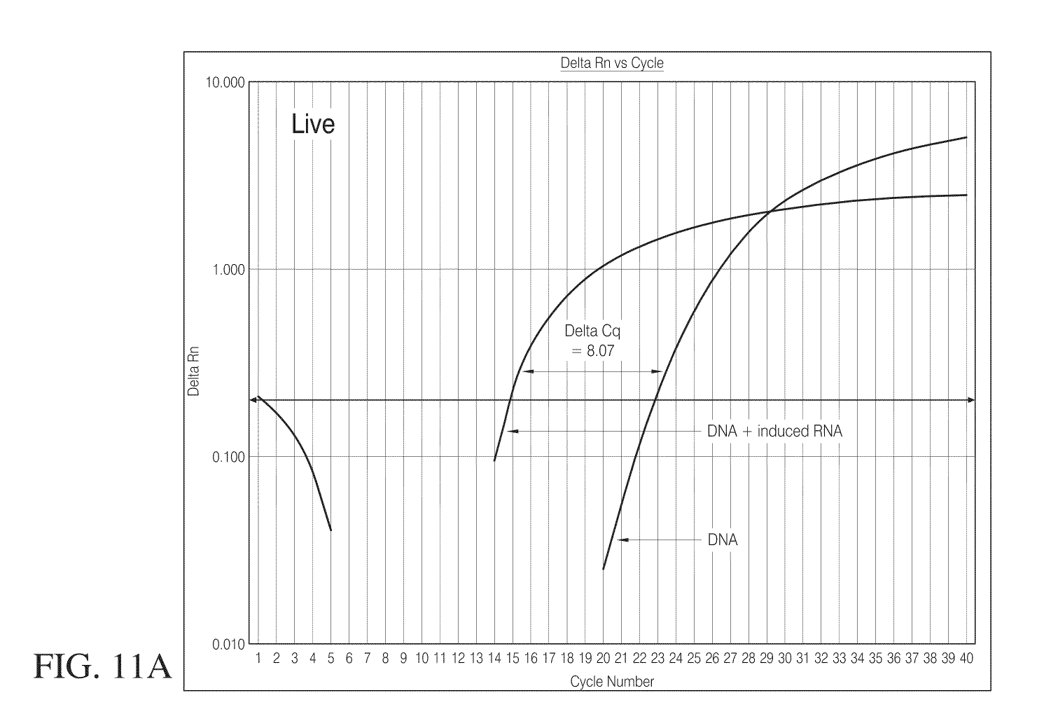

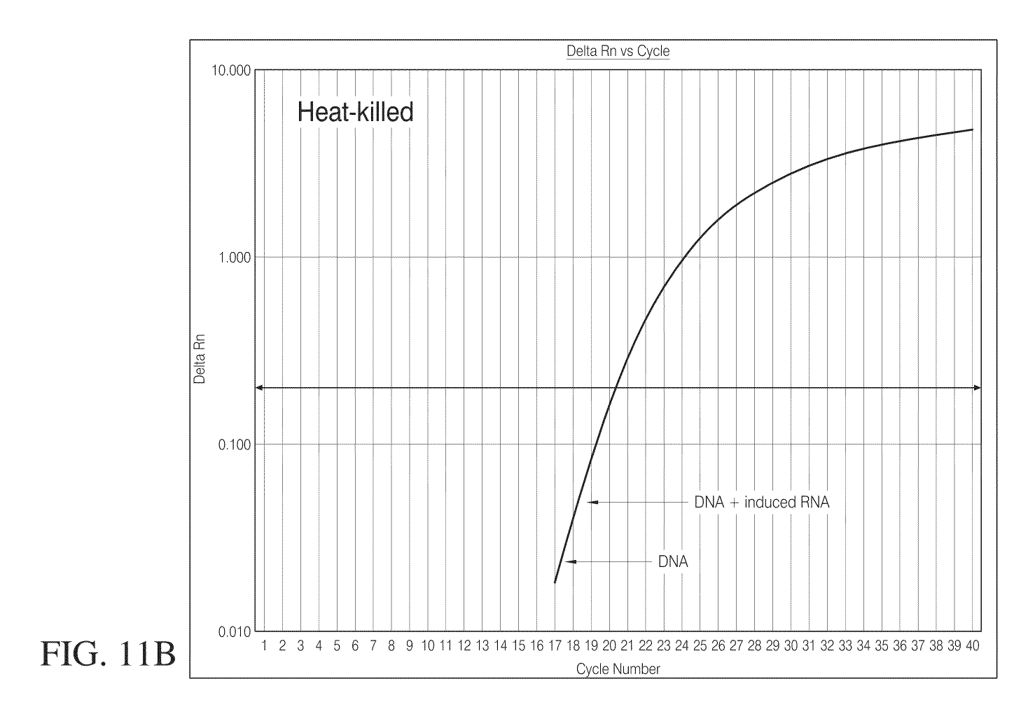

To test viability of detected bacteria, duplicate samples of Salmonella culture were prepared. One set was heat-treated at 95° C. for 15 minutes to kill the bacteria. A control set was maintained at 37° C. Both sets were induced at 45° C. for 15 minutes, followed by sample extraction. Assays were evaluated by real-time PCR in the presence and absence of ArrayScript® M-MLV reverse transcriptase. Live and viable cells responded to the heat-induction by increase in target mRNA production, while the heat-killed cells did not. These results are shown in FIG. 11A and FIG. 11B, where FIG. 11A shows live and viable Salmonella cells responding to the heat-induction by increase in target mRNA production, while in FIG. 11B heat-killed Salmonella cells do not increase target mRNA production.

The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed in part by preferred embodiments, exemplary embodiments and optional features, modification and variation of the concepts herein disclosed may be resorted to by those skilled in the art, and such modifications and variations are considered to be within the scope of this invention as defined by the appended claims. The specific embodiments provided herein are examples of useful embodiments of the present invention and it will be apparent to one skilled in the art that the present invention may be carried out using a large number of variations of the compositions, kits and methods steps set forth in the present description.

When a group of materials, compositions, components or compounds is disclosed herein, it is understood that all individual members of those groups and all subgroups thereof are disclosed separately. When a Markush group or other grouping is used herein, all individual members of the group and all combinations and subcombinations possible of the group are intended to be individually included in the disclosure. Every formulation or combination of components described or exemplified herein can be used to practice the invention, unless otherwise stated. Whenever a range is given in the specification, for example, a temperature range, a time range, or a composition range, all intermediate ranges and subranges, as well as all individual values included in the ranges given are intended to be included in the disclosure.

1. A method of detecting presence of a microorganism in a sample, comprising:
exposing the sample to an RNA-inducing agent for a time to induce a gene responsive to the RNA-inducing agent;
detecting presence of an RNA corresponding to the gene responsive to the RNA-inducing agent; and
determining presence of the microorganism, wherein the detection of presence of the RNA corresponding to the gene responsive to the RNA-inducing agent in comparison to a control sample is indicative of the presence of the microorganism in the sample.

2. The method of claim 1, further comprising the step of culturing the sample in a microorganism enrichment medium to form an enriched sample, and wherein the exposing the sample comprises exposing the enriched sample to the RNA-inducing agent.

3. The method of claim 1 wherein the sample is a food sample, an environmental sample, or a clinical sample.

4. The method of claim 1 wherein exposing the sample to an RNA-inducing agent comprises exposing the sample to a thermal condition, a chemical agent, a high salt concentration, a pH change, a stress, activated charcoal, or a nutritive deficiency.

5. The method of claim 1, wherein the microorganism is a pathogen.

6. The method of claim 5 wherein the pathogen is Salmonella.

7. The method of claim 6 wherein the gene responsive to the RNA-inducing agent is a cspH, a hilA, a hsp60, a dnaK, a ibpAB, a uspA, or a agsA gene.

8. The method of claim 5 wherein the pathogen is Listeria.

9. The method of claim 8 wherein the gene responsive to the RNA-inducing agent is a inlA, a inlB, a inlC, a inlG, a inlJ, a lmo0539, a lmo2158, a lmo0596, a lmo0189, a lmo0880, a lmo1290, a lmo0514, a lmo0670, a bsh, a plcA, a clpE, a cspL, a lmo0699, a lmo0782, a lmo2230, a lmo2522, a opuCA, a cpn60, or a hlyA gene

10. The method of claim 5 wherein the pathogen is Vibrio.

11. The method of claim 10 wherein and the gene responsive to the RNA-inducing agent is a hsp60 gene.

12. The method of claim 1 wherein detecting presence of RNA is by a reverse transcriptase RT-PCR assay.

13. The method of claim 12 further comprising detecting presence of DNA corresponding to the gene by an RT-PCR assay.

14. The method of claim 13 further comprising subtracting a CT value of RNA and DNA detection from a CT value for DNA detection.

15. A kit comprising:
at least one primer pair selected from:
a primer pair operable to detect a Salmonella spp in a sample comprising at least one Salmonella-specific primer pair having hybridization specificity for priming amplification of an RNA-inducing agent-responsive gene of Salmonella or a fragment thereof wherein the RNA-inducing agent-responsive gene is a cspH, a hilA, a hsp60, a dnaK, a ibpAB, a uspA, and/or a agsA gene;
a primer pair operable to detect a Listeria spp in a sample comprising at least one Listeria-specific primer pair having hybridization specificity for priming amplification of an RNA-inducing agent-responsive gene of Listeria or a fragment thereof wherein the RNA-inducing agent-responsive gene is a inlA, a inlB, a inlC, a inlG, a inlJ, a lmo0539, a lmo2158, a lmo0596, a lmo0189, a lmo0880, a lmo1290, a lmo0514, a lmo0670, a bsh, a plcA, a clpE, a cspL, a lmo0699, a lmo0782, a lmo2230, a lmo2522, a opuCA, a cpn60, or a hlyA gene; and
a primer pair operable to detect a Vibrio spp in a sample comprising at least one Vibrio-specific primer pair having hybridization specificity for priming amplification of an RNA-inducing agent-responsive gene of Vibrio or a fragment thereof wherein the RNA-inducing agent-responsive gene is a hsp60 gene.

16. The kit of claim 15 further comprising a probe having hybridization specificity for said RNA-inducing agent-responsive gene.

17. The kit of claim 15 further comprising at least one of a reverse transcriptase, a DNA polymerase, dNTP's, a filtration medium, a surfactant, magnetic beads, a spin column and combinations thereof.

18. The kits of claim 15, further comprising at least three primer pairs comprising at least one Salmonella-specific primer pair, at least one Listeria-specific primer pair and at least one Vibrio-specific primer pair.

19. A compositions for detecting the presence of Salmonella in a sample comprising:
at least one primer pair having hybridization specificity for priming amplification of an RNA-inducing agent-responsive gene of Salmonella wherein the RNA-inducing agent-responsive gene is a cspH, a hilA, a hsp60, a dnaK, a ibpAB, a uspA, or a agsA gene; and
optionally comprising at least one probe having hybridization specificity for said RNA-inducing agent-responsive gene.

20.-33. (canceled)

34. A composition for detecting the presence of Listeria in a sample comprising:
at least one primer pair having hybridization specificity for priming amplification of an RNA-inducing agent-responsive gene of Listeria wherein the RNA-inducing agent-responsive gene is a inlA, a inlB, a inlC, a inlG, a inlJ, a lmo0539, a lmo2158, a lmo0596, a lmo0189, a lmo0880, a lmo1290, a lmo0514, a lmo0670, a bsh, a plcA, a clpE, a cspL, a lmo0699, a lmo0782, a lmo2230, a lmo2522, a opuCA, a cpn60, or a hlyA gene; and
optionally comprising at least one probe having hybridization specificity for said RNA-inducing agent-responsive gene.

35-80. (canceled)

81. A compositions for detecting the presence of a Vibrio spp in a sample comprising at least one primer pair having hybridization specificity for priming amplification of an RNA-inducing agent-responsive gene of Vibrio wherein the RNA-inducing agent-responsive gene is a hsp60 gene.

82.-83. (canceled)


SEQUENCE LISTING (1769 sequences)

SEQ ID NO: 1 - DNA, 25 bp, Artificial Sequence
  Description of Artificial Sequence Synthetic primer
ctggcgtatctcctgttaattgact

SEQ ID NO: 2 - DNA, 23 bp, Artificial Sequence
  Description of Artificial Sequence Synthetic primer
tgtgttcaatcgcctttggtttg

SEQ ID NO: 3 - DNA, 25 bp, Artificial Sequence
  Description of Artificial Sequence Synthetic primer
ggcgtatctcctgttaattgactga

SEQ ID NO: 4 - DNA, 21 bp, Artificial Sequence
  Description of Artificial Sequence Synthetic primer
cgcgcttttgcagatcgtaag

SEQ ID NO: 5 - DNA, 22 bp, Artificial Sequence
  Description of Artificial Sequence Synthetic primer
tggaaatccgccttacgaatcc

SEQ ID NO: 6 - DNA, 19 bp, Artificial Sequence
  Description of Artificial Sequence Synthetic primer
aggccctgttccagtttcg

SEQ ID NO: 7 - DNA, 19 bp, Artificial Sequence
  Description of Artificial Sequence Synthetic primer
gccgccaaccgtttcaatt

SEQ ID NO: 8 - DNA, 25 bp, Artificial Sequence
  Description of Artificial Sequence Synthetic primer
tcctctaccgtatcttcagtgtgtt

SEQ ID NO: 9 - DNA, 23 bp, Artificial Sequence
  Description of Artificial Sequence Synthetic primer
ggaaatccgccttacgaatacca

SEQ ID NO: 10 - DNA, 23 bp, Artificial Sequence
  Description of Artificial Sequence Synthetic primer
cgcctttggtttgctttctatgg

SEQ ID NO: 11 - DNA, 23 bp, Artificial Sequence
  Description of Artificial Sequence Synthetic primer
tttctcgctttcagggatttcct

SEQ ID NO: 12 - DNA, 23 bp, Artificial Sequence
  Description of Artificial Sequence Synthetic primer
cgcctttggtttgctttctatgg

SEQ ID NO: 13 - DNA, 25 bp, Artificial Sequence
  Description of Artificial Sequence Synthetic primer
gtctcttcagtgtgtttacccgtaa

SEQ ID NO: 14 - DNA, 25 bp, Artificial Sequence
  Description of Artificial Sequence Synthetic primer
accgtttcaatttcaagctcttcct

SEQ ID NO: 15 - DNA, 23 bp, Artificial Sequence
  Description of Artificial Sequence Synthetic primer
gccaaccgtttcaatttcaagct

SEQ ID NO: 16 - DNA, 25 bp, Artificial Sequence
  Description of Artificial Sequence Synthetic primer
cgctcacggtaagcagatagttatt

SEQ ID NO: 17 - DNA, 25 bp, Artificial Sequence
  Description of Artificial Sequence Synthetic primer
actggaaatctgccttacgaatacc

SEQ ID NO: 18 - DNA, 21 bp, Artificial Sequence
  Description of Artificial Sequence Synthetic primer
agaggccctgttccagttttg

SEQ ID NO: 19 - DNA, 23 bp, Artificial Sequence
  Description of Artificial Sequence Synthetic primer
cgctcacggtaagcagatagtta

SEQ ID NO: 20 - DNA, 25 bp, Artificial Sequence
  Description of Artificial Sequence Synthetic primer
gtctcttcagtgtgtttacccgtaa

SEQ ID NO: 21 - DNA, 25 bp, Artificial Sequence
  Description of Artificial Sequence Synthetic primer
cgccttacgaataccacgataaatc

SEQ ID NO: 22 - DNA, 20 bp, Artificial Sequence
  Description of Artificial Sequence Synthetic primer
gaccaacaagaggccctgtt

SEQ ID NO: 23 - DNA, 25 bp, Artificial Sequence
  Description of Artificial Sequence Synthetic primer
accgtttcaatttcaagctcttcct

SEQ ID NO: 24 - DNA, 27 bp, Artificial Sequence
  Description of Artificial Sequence Synthetic primer
ttgtctcgtaaaatgacaggaattgtc

SEQ ID NO: 25 - DNA, 20 bp, Artificial Sequence
  Description of Artificial Sequence Synthetic primer
gcgcttatccccggtatacg

SEQ ID NO: 26 - DNA, 18 bp, Artificial Sequence
  Description of Artificial Sequence Synthetic primer
ccctccgatggacgcaaa

SEQ ID NO: 27 - DNA, 25 bp, Artificial Sequence
  Description of Artificial Sequence Synthetic primer
gtaagagcggtaaaggtctcatcac

SEQ ID NO: 28 - DNA, 18 bp, Artificial Sequence
  Description of Artificial Sequence Synthetic primer
cccctccgatgacgcaaa

SEQ ID NO: 29 - DNA, 27 bp, Artificial Sequence
  Description of Artificial Sequence Synthetic primer
ttgtctcgtaaaatgacaggaattgtc

SEQ ID NO: 30 - DNA, 20 bp, Artificial Sequence
  Description of Artificial Sequence Synthetic primer
gaagcgcttatccccggtat

SEQ ID NO: 31 - DNA, 25 bp, Artificial Sequence
  Description of Artificial Sequence Synthetic primer
gtaagagcggtaaaggtctcatcac

SEQ ID NO: 32 - DNA, 27 bp, Artificial Sequence
  Description of Artificial Sequence Synthetic primer
ttgtctcgtaaaatgacaggaattgtc

SEQ ID NO: 33 - DNA, 25 bp, Artificial Sequence
  Description of Artificial Sequence Synthetic primer
gtaagagcggtaaaggtctcatcac

SEQ ID NO: 34 - DNA, 27 bp, Artificial Sequence
  Description of Artificial Sequence Synthetic primer
ttgtctcgtaaaatgacaggaattgtc

SEQ ID NO: 35 - DNA, 25 bp, Artificial Sequence
  Description of Artificial Sequence Synthetic primer
gtaagagcggtaaaggtctcatcac

SEQ ID NO: 36 - DNA, 25 bp, Artificial Sequence
  Description of Artificial Sequence Synthetic primer
gtaagagcggtaaaggtctcatcac

SEQ ID NO: 37 - DNA, 25 bp, Artificial Sequence
  Description of Artificial Sequence Synthetic primer
acctttgattgtaagagcggtaacc

SEQ ID NO: 38 - DNA, 25 bp, Artificial Sequence
  Description of Artificial Sequence Synthetic primer
gattgtaagagcggtaaaggtctca

SEQ ID NO: 39 - DNA, 18 bp, Artificial Sequence
  Description of Artificial Sequence Synthetic primer
ccctccgatggacgcaaa

SEQ ID NO: 40 - DNA, 21 bp, Artificial Sequence
  Description of Artificial Sequence Synthetic primer
tccgatggacgcaaagatgtt

SEQ ID NO: 41 - DNA, 22 bp, Artificial Sequence
  Description of Artificial Sequence Synthetic primer
gacgcaaagatgttcagttcca

SEQ ID NO: 42 - DNA, 25 bp, Artificial Sequence
  Description of Artificial Sequence Synthetic primer
gattgtaagagcggtaaaggtctca

SEQ ID NO: 43 - DNA, 23 bp, Artificial Sequence
  Description of Artificial Sequence Synthetic primer
tgttcaggtccacatttcagcat

SEQ ID NO: 44 - DNA, 22 bp, Artificial Sequence
  Description of Artificial Sequence Synthetic primer
cacgaaacagaagcgcttatcc

SEQ ID NO: 45 - DNA, 24 bp, Artificial Sequence
  Description of Artificial Sequence Synthetic primer
cggtggtggtactttcgatatctc

SEQ ID NO: 46 - DNA, 19 bp, Artificial Sequence
  Description of Artificial Sequence Synthetic primer
gtaaccagggcgaccatct

SEQ ID NO: 47 - DNA, 21 bp, Artificial Sequence
  Description of Artificial Sequence Synthetic primer
aaaatcatcggcgctgacaac

SEQ ID NO: 48 - DNA, 25 bp, Artificial Sequence
  Description of Artificial Sequence Synthetic primer
ccaactcttgtgtagcgattatgga

SEQ ID NO: 49 - DNA, 23 bp, Artificial Sequence
  Description of Artificial Sequence Synthetic primer
accgtaagtttgaagagctggtt

SEQ ID NO: 50 - DNA, 22 bp, Artificial Sequence
  Description of Artificial Sequence Synthetic primer
ctgaatccgaccgtaagttcga

SEQ ID NO: 51 - DNA, 22 bp, Artificial Sequence
  Description of Artificial Sequence Synthetic primer
cgatacccgcctgatcaactac

SEQ ID NO: 52 - DNA, 18 bp, Artificial Sequence
  Description of Artificial Sequence Synthetic primer
gctgcagggtgagcgtaa

SEQ ID NO: 53 - DNA, 18 bp, Artificial Sequence
  Description of Artificial Sequence Synthetic primer
gtgacccgtgcgaaactg

SEQ ID NO: 54 - DNA, 24 bp, Artificial Sequence
  Description of Artificial Sequence Synthetic primer
agatctggtgaaccgttctatcga

SEQ ID NO: 55 - DNA, 21 bp, Artificial Sequence
  Description of Artificial Sequence Synthetic primer
aaatcgctcagcagcaacatg

SEQ ID NO: 56 - DNA, 25 bp, Artificial Sequence
  Description of Artificial Sequence Synthetic primer
ggcttacggtctggataaagaagtc

SEQ ID NO: 57 - DNA, 20 bp, Artificial Sequence
  Description of Artificial Sequence Synthetic primer
cagaagcgaacgctgaatcc

SEQ ID NO: 58 - DNA, 24 bp, Artificial Sequence
  Description of Artificial Sequence Synthetic primer
cggtggtggtactttcgatatctc

SEQ ID NO: 59 - DNA, 20 bp, Artificial Sequence
  Description of Artificial Sequence Synthetic primer
ccgcagatcgaagtcacctt

SEQ ID NO: 60 - DNA, 17 bp, Artificial Sequence
  Description of Artificial Sequence Synthetic primer
cacgcgtgctggagaac

SEQ ID NO: 61 - DNA, 23 bp, Artificial Sequence
  Description of Artificial Sequence Synthetic primer
ccgtaccggcttactttaacgat

SEQ ID NO: 62 - DNA, 22 bp, Artificial Sequence
  Description of Artificial Sequence Synthetic primer
ttctatcgagccgctgaaagtc

SEQ ID NO: 63 - DNA, 21 bp, Artificial Sequence
  Description of Artificial Sequence Synthetic primer
cgcctgatcaactacctcgtt

SEQ ID NO: 64 - DNA, 23 bp, Artificial Sequence
  Description of Artificial Sequence Synthetic primer
aactggaaagcctggttgaagat

SEQ ID NO: 65 - DNA, 24 bp, Artificial Sequence
  Description of Artificial Sequence Synthetic primer
atcaaagtgactcgtgcaaaactg

SEQ ID NO: 66 - DNA, 22 bp, Artificial Sequence
  Description of Artificial Sequence Synthetic primer
ctccgactctaaagcgattgct

SEQ ID NO: 67 - DNA, 24 bp, Artificial Sequence
  Description of Artificial Sequence Synthetic primer
cgacgaaaccgtaggtaaactgat

SEQ ID NO: 68 - DNA, 25 bp, Artificial Sequence
  Description of Artificial Sequence Synthetic primer
atggcagctaaagacgtaaaattcg

SEQ ID NO: 69 - DNA, 22 bp, Artificial Sequence
  Description of Artificial Sequence Synthetic primer
gtaaggcgatgctgcaggatat

SEQ ID NO: 70 - DNA, 24 bp, Artificial Sequence
  Description of Artificial Sequence Synthetic primer
cggcaacatgatcgatatgggtat

SEQ ID NO: 71 - DNA, 19 bp, Artificial Sequence
  Description of Artificial Sequence Synthetic primer
ccgtgctgcggtagaagaa

SEQ ID NO: 72 - DNA, 18 bp, Artificial Sequence
  Description of Artificial Sequence Synthetic primer
ccgtagcgcgtgaaatcg

SEQ ID NO: 73 - DNA, 22 bp, Artificial Sequence
  Description of Artificial Sequence Synthetic primer
tcaacaaagacaccaccaccat

SEQ ID NO: 74 - DNA, 22 bp, Artificial Sequence
  Description of Artificial Sequence Synthetic primer
ccaaccaaagttacccgttctg

SEQ ID NO: 75 - DNA, 19 bp, Artificial Sequence
  Description of Artificial Sequence Synthetic primer
ggaagccgttgcaaaagca

SEQ ID NO: 76 - DNA, 25 bp, Artificial Sequence
  Description of Artificial Sequence Synthetic primer
cagatggtgaaagaagttgcctcta

SEQ ID NO: 77 - DNA, 23 bp, Artificial Sequence
  Description of Artificial Sequence Synthetic primer
gagatcggtatggagctggaaaa

SEQ ID NO: 78 - DNA, 25 bp, Artificial Sequence
  Description of Artificial Sequence Synthetic primer
cagatggtgaaagaagttgcctcta

SEQ ID NO: 79 - DNA, 22 bp, Artificial Sequence
  Description of Artificial Sequence Synthetic primer
ctccgactctaaagcgattgct

SEQ ID NO: 80 - DNA, 21 bp, Artificial Sequence
  Description of Artificial Sequence Synthetic primer
ggcgaaacgtgttgtgatcaa

SEQ ID NO: 81 - DNA, 25 bp, Artificial Sequence
  Description of Artificial Sequence Synthetic primer
ccgacgaaactgtaggtaaactgat

SEQ ID NO: 82 - DNA, 22 bp, Artificial Sequence
  Description of Artificial Sequence Synthetic primer
gctgctgatcatcgctgaagat

SEQ ID NO: 83 - DNA, 20 bp, Artificial Sequence
  Description of Artificial Sequence Synthetic primer
cggtagaagaaggcgtggtt

SEQ ID NO: 84 - DNA, 18 bp, Artificial Sequence
  Description of Artificial Sequence Synthetic primer
gctgctgcggttgaagag

SEQ ID NO: 85 - DNA, 22 bp, Artificial Sequence
  Description of Artificial Sequence Synthetic primer
ggcagatgcagtgaaagttacc

SEQ ID NO: 86 - DNA, 25 bp, Artificial Sequence
  Description of Artificial Sequence Synthetic primer
cgaccgaagttgaaatgaaagagaa

SEQ ID NO: 87 - DNA, 24 bp, Artificial Sequence
  Description of Artificial Sequence Synthetic primer
gctggctgagaatatggaagtttc

SEQ ID NO: 88 - DNA, 23 bp, Artificial Sequence
  Description of Artificial Sequence Synthetic primer
tccagttagcggagaacattcac

SEQ ID NO: 89 - DNA, 24 bp, Artificial Sequence
  Description of Artificial Sequence Synthetic primer
cagttagcggaaaacattcacgtt

SEQ ID NO: 90 - DNA, 14 bp, Artificial Sequence
  Description of Artificial Sequence Synthetic primer
tgcgcggcctgtca

SEQ ID NO: 91 - DNA, 21 bp, Artificial Sequence
  Description of Artificial Sequence Synthetic primer
gagcttcccgccctataacat

SEQ ID NO: 92 - DNA, 20 bp, Artificial Sequence
  Description of Artificial Sequence Synthetic primer
tgctgtaccgttctgccatt

SEQ ID NO: 93 - DNA, 17 bp, Artificial Sequence
  Description of Artificial Sequence Synthetic primer
gcgcctgacggtgaaag

SEQ ID NO: 94 - DNA, 24 bp, Artificial Sequence
  Description of Artificial Sequence Synthetic primer
cgcacgtaaagtgtgaaggtaaaa

SEQ ID NO: 95 - DNA, 19 bp, Artificial Sequence
  Description of Artificial Sequence Synthetic primer
ccactgctgcgtcaatgga

SEQ ID NO: 96 - DNA, 18 bp, Artificial Sequence
  Description of Artificial Sequence Synthetic primer
ctaccgcatcgccattgc

SEQ ID NO: 97 - DNA, 25 bp, Artificial Sequence
  Description of Artificial Sequence Synthetic primer
gaaaacgagcccaaatggttacatc

SEQ ID NO: 98 - DNA, 24 bp, Artificial Sequence
  Description of Artificial Sequence Synthetic primer
cgagcagaaagaacgtacttacct

SEQ ID NO: 99 - DNA, 20 bp, Artificial Sequence
  Description of Artificial Sequence Synthetic primer
ggtggtaaaaggcgctcatg

SEQ ID NO: 100 - DNA, 21 bp, Artificial Sequence
  Description of Artificial Sequence Synthetic primer
cttcccgccctataacatcga

SEQ ID NO: 101 - DNA, 24 bp, Artificial Sequence
  Description of Artificial Sequence Synthetic primer
ggcctgctgcatattgatttaacc

SEQ ID NO: 102 - DNA, 24 bp, Artificial Sequence
  Description of Artificial Sequence Synthetic primer
cgcacgtaaagtgtgaaggtaaaa

SEQ ID NO: 103 - DNA, 17 bp, Artificial Sequence
  Description of Artificial Sequence Synthetic primer
ggcgcgacgtttaccaa

SEQ ID NO: 104 - DNA, 24 bp, Artificial Sequence
  Description of Artificial Sequence Synthetic primer
cgcacgtaaagtgtgaaggtaaaa

SEQ ID NO: 105 - DNA, 18 bp, Artificial Sequence
  Description of Artificial Sequence Synthetic primer
cgaactggagcgcgtgat

SEQ ID NO: 106 - DNA, 16 bp, Artificial Sequence
  Description of Artificial Sequence Synthetic primer
cgccgtcagggagcat

SEQ ID NO: 107 - DNA, 25 bp, Artificial Sequence
  Description of Artificial Sequence Synthetic primer
cgccgtatcgaaattaactgattcc

SEQ ID NO: 108 - DNA, 16 bp, Artificial Sequence
  Description of Artificial Sequence Synthetic primer
cgccgtcagggagcat

SEQ ID NO: 109 - DNA, 25 bp, Artificial Sequence
  Description of Artificial Sequence Synthetic primer
actggagattactgcccaggataat

SEQ ID NO: 110 - DNA, 19 bp, Artificial Sequence
  Description of Artificial Sequence Synthetic primer
tggtgatgaccgcaaacga

SEQ ID NO: 111 - DNA, 25 bp, Artificial Sequence
  Description of Artificial Sequence Synthetic primer
tttcttcggagatacgtttctgcat

SEQ ID NO: 112 - DNA, 22 bp, Artificial Sequence
  Description of Artificial Sequence Synthetic primer
gcaaacgaccagatccatatcg

SEQ ID NO: 113 - DNA, 22 bp, Artificial Sequence
  Description of Artificial Sequence Synthetic primer
cgtcaatcagaccggtgtacag

SEQ ID NO: 114 - DNA, 21 bp, Artificial Sequence
  Description of Artificial Sequence Synthetic primer
gcggaacaatcagcatgtcaa

SEQ ID NO: 115 - DNA, 22 bp, Artificial Sequence
  Description of Artificial Sequence Synthetic primer
gcaaacgaccagatccatatcg

SEQ ID NO: 116 - DNA, 20 bp, Artificial Sequence
  Description of Artificial Sequence Synthetic primer
gtagccagcgttggtagaca

SEQ ID NO: 117 - DNA, 16 bp, Artificial Sequence
  Description of Artificial Sequence Synthetic primer
cgcagcggcacaatca

SEQ ID NO: 118 - DNA, 21 bp, Artificial Sequence
  Description of Artificial Sequence Synthetic primer
ccgctcagggtttcagtgata

SEQ ID NO: 119 - DNA, 22 bp, Artificial Sequence
  Description of Artificial Sequence Synthetic primer
cgctcagggtttcagtgatagg

SEQ ID NO: 120 - DNA, 24 bp, Artificial Sequence
  Description of Artificial Sequence Synthetic primer
aagacatcagtttgctccagaagt

SEQ ID NO: 121 - DNA, 25 bp, Artificial Sequence
  Description of Artificial Sequence Synthetic primer
tttcttcggagatacgtttctgcat

SEQ ID NO: 122 - DNA, 24 bp, Artificial Sequence
  Description of Artificial Sequence Synthetic primer
gaacaatcagcatgtcaacgtgaa

SEQ ID NO: 123 - DNA, 24 bp, Artificial Sequence
  Description of Artificial Sequence Synthetic primer
ccggtgtacaggtctgaatagttc

SEQ ID NO: 124 - DNA, 20 bp, Artificial Sequence
  Description of Artificial Sequence Synthetic primer
gtagccagcgttggtagaca

SEQ ID NO: 125 - DNA, 17 bp, Artificial Sequence
  Description of Artificial Sequence Synthetic primer
tggcgcgcagaagacat

SEQ ID NO: 126 - DNA, 20 bp, Artificial Sequence
  Description of Artificial Sequence Synthetic primer
ctaccgctcagggtttcagt

SEQ ID NO: 127 - DNA, 22 bp, Artificial Sequence
  Description of Artificial Sequence Synthetic primer
cgctcagggtttcagtgatagg

SEQ ID NO: 128 - DNA, 25 bp, Artificial Sequence
  Description of Artificial Sequence Synthetic primer
accggtgtacaggtctgaataattc

SEQ ID NO: 129 - DNA, 21 bp, Artificial Sequence
  Description of Artificial Sequence Synthetic primer
gtggtgggtttcttcggagat

SEQ ID NO: 130 - DNA, 22 bp, Artificial Sequence
  Description of Artificial Sequence Synthetic primer
cgctcagggtttcagtgatagg

SEQ ID NO: 131 - DNA, 17 bp, Artificial Sequence
  Description of Artificial Sequence Synthetic primer
tggcgcgcagaagacat

SEQ ID NO: 132 - DNA, 17 bp, Artificial Sequence
  Description of Artificial Sequence Synthetic primer
gcgcagcggaacaatca

SEQ ID NO: 133 - DNA, 18 bp, Artificial Sequence
  Description of Artificial Sequence Synthetic primer
gcagtatgcgccctttgg

SEQ ID NO: 134 - DNA, 24 bp, Artificial Sequence
  Description of Artificial Sequence Synthetic primer
attctcttttccctgaccgtttca

SEQ ID NO: 135 - DNA, 23 bp, Artificial Sequence
  Description of Artificial Sequence Synthetic primer
ggagatccctgaaagcgagaaac

SEQ ID NO: 136 - DNA, 24 bp, Artificial Sequence
  Description of Artificial Sequence Synthetic primer
tcccgtgtttgctgattctctttt

SEQ ID NO: 137 - DNA, 22 bp, Artificial Sequence
  Description of Artificial Sequence Synthetic primer
cctgaccgtttcaaccgtattg

SEQ ID NO: 138 - DNA, 25 bp, Artificial Sequence
  Description of Artificial Sequence Synthetic primer
acactgaagatacggtagaggatca

SEQ ID NO: 139 - DNA, 22 bp, Artificial Sequence
  Description of Artificial Sequence Synthetic primer
ggcggacttccagttgagtttt

SEQ ID NO: 140 - DNA, 19 bp, Artificial Sequence
  Description of Artificial Sequence Synthetic primer
gcttaccgtgagcgttcct

SEQ ID NO: 141 - DNA, 25 bp, Artificial Sequence
  Description of Artificial Sequence Synthetic primer
gctggaaagaggaagagcttgaaat

SEQ ID NO: 142 - DNA, 24 bp, Artificial Sequence
  Description of Artificial Sequence Synthetic primer
cactgaagagacggtagaggatca

SEQ ID NO: 143 - DNA, 24 bp, Artificial Sequence
  Description of Artificial Sequence Synthetic primer
ggcctcttgttggtcgagatttac

SEQ ID NO: 144 - DNA, 25 bp, Artificial Sequence
  Description of Artificial Sequence Synthetic primer
tgcctgaacatgctaaggtgaataa

SEQ ID NO: 145 - DNA, 24 bp, Artificial Sequence
  Description of Artificial Sequence Synthetic primer
ggcctcttgttggtcgagatttac

SEQ ID NO: 146 - DNA, 25 bp, Artificial Sequence
  Description of Artificial Sequence Synthetic primer
gctggaaagaggaagagcttgaaat

SEQ ID NO: 147 - DNA, 25 bp, Artificial Sequence
  Description of Artificial Sequence Synthetic primer
cgatgcgaataactatctgcttacc

SEQ ID NO: 148 - DNA, 25 bp, Artificial Sequence
  Description of Artificial Sequence Synthetic primer
cgatgcgaataactatctgcttacc

SEQ ID NO: 149 - DNA, 20 bp, Artificial Sequence
  Description of Artificial Sequence Synthetic primer
caggagatacgccagttgct

SEQ ID NO: 150 - DNA, 24 bp, Artificial Sequence
  Description of Artificial Sequence Synthetic primer
cactgaagagacggtagaggatca

SEQ ID NO: 151 - DNA, 25 bp, Artificial Sequence
  Description of Artificial Sequence Synthetic primer
cgtaaggcagatttccagttgagtt

SEQ ID NO: 152 - DNA, 19 bp, Artificial Sequence
  Description of Artificial Sequence Synthetic primer
cgacgccagcttacgatct

SEQ ID NO: 153 - DNA, 19 bp, Artificial Sequence
  Description of Artificial Sequence Synthetic primer
gcttaccgtgagcgttcct

SEQ ID NO: 154 - DNA, 25 bp, Artificial Sequence
  Description of Artificial Sequence Synthetic primer
cggcaacctgaatattacgggtaaa

SEQ ID NO: 155 - DNA, 23 bp, Artificial Sequence
  Description of Artificial Sequence Synthetic primer
aggcggatttccagttgagtttt

SEQ ID NO: 156 - DNA, 22 bp, Artificial Sequence
  Description of Artificial Sequence Synthetic primer
gccagcttacgatctgcaaaag

SEQ ID NO: 157 - DNA, 22 bp, Artificial Sequence
  Description of Artificial Sequence Synthetic primer
acctgaacatctttgcgtccat

SEQ ID NO: 158 - DNA, 16 bp, Artificial Sequence
  Description of Artificial Sequence Synthetic primer
gttggcggcggtaggt

SEQ ID NO: 159 - DNA, 24 bp, Artificial Sequence
  Description of Artificial Sequence Synthetic primer
gtttcgtgttggctaaatgctgaa

SEQ ID NO: 160 - DNA, 20 bp, Artificial Sequence
  Description of Artificial Sequence Synthetic primer
cgtgtaggcgacatgctgaa

SEQ ID NO: 161 - DNA, 19 bp, Artificial Sequence
  Description of Artificial Sequence Synthetic primer
gtgttggcgacatgctgaa

SEQ ID NO: 162 - DNA, 22 bp, Artificial Sequence
  Description of Artificial Sequence Synthetic primer
acctgaacatctttgcgtccat

SEQ ID NO: 163 - DNA, 20 bp, Artificial Sequence
  Description of Artificial Sequence Synthetic primer
gtccgcggaggccattaata

SEQ ID NO: 164 - DNA, 22 bp, Artificial Sequence
  Description of Artificial Sequence Synthetic primer
tgctgaaatgtggacctgaaca

SEQ ID NO: 165 - DNA, 22 bp, Artificial Sequence
  Description of Artificial Sequence Synthetic primer
acctgaacatctttgcgtccat

SEQ ID NO: 166 - DNA, 22 bp, Artificial Sequence
  Description of Artificial Sequence Synthetic primer
gtgttggcgacatgatgagatg

SEQ ID NO: 167 - DNA, 22 bp, Artificial Sequence
  Description of Artificial Sequence Synthetic primer
acctgaacatctttgcgtccat

SEQ ID NO: 168 - DNA, 21 bp, Artificial Sequence
  Description of Artificial Sequence Synthetic primer
gtttcgtgttggcgacatgat

SEQ ID NO: 169 - DNA, 22 bp, Artificial Sequence
  Description of Artificial Sequence Synthetic primer
catgctgagatgtggacctgaa

SEQ ID NO: 170 - DNA, 21 bp, Artificial Sequence
  Description of Artificial Sequence Synthetic primer
tgttggcgacatgctgaaatg

SEQ ID NO: 171 - DNA, 22 bp, Artificial Sequence
  Description of Artificial Sequence Synthetic primer
tgctgaaatgtggacctgaaca

SEQ ID NO: 172 - DNA, 18 bp, Artificial Sequence
  Description of Artificial Sequence Synthetic primer
tcgtgttggcgacatgct

SEQ ID NO: 173 - DNA, 21 bp, Artificial Sequence
  Description of Artificial Sequence Synthetic primer
tctgtttcgtgttggcgacat

SEQ ID NO: 174 - DNA, 22 bp, Artificial Sequence
  Description of Artificial Sequence Synthetic primer
gggataagcgcttctgtttcgt

SEQ ID NO: 175 - DNA, 22 bp, Artificial Sequence
  Description of Artificial Sequence Synthetic primer
tgctgaaatgtggacctgaaca

SEQ ID NO: 176 - DNA, 23 bp, Artificial Sequence
  Description of Artificial Sequence Synthetic primer
cgacaaaactcaacgcgtatacc

SEQ ID NO: 177 - DNA, 20 bp, Artificial Sequence
  Description of Artificial Sequence Synthetic primer
tccgcggaggccattaatac

SEQ ID NO: 178 - DNA, 24 bp, Artificial Sequence
  Description of Artificial Sequence Synthetic primer
ggttgccagaacttcaaaggtttt

SEQ ID NO: 179 - DNA, 20 bp, Artificial Sequence
  Description of Artificial Sequence Synthetic primer
cctgcttcttcgacctgctt

SEQ ID NO: 180 - DNA, 17 bp, Artificial Sequence
  Description of Artificial Sequence Synthetic primer
gcggcgccattttctga

SEQ ID NO: 181 - DNA, 15 bp, Artificial Sequence
  Description of Artificial Sequence Synthetic primer
cgccctcggcgttct

SEQ ID NO: 182 - DNA, 21 bp, Artificial Sequence
  Description of Artificial Sequence Synthetic primer
cctgcttcttcgacctgctta

SEQ ID NO: 183 - DNA, 22 bp, Artificial Sequence
  Description of Artificial Sequence Synthetic primer
gcctgcttcttcaacctgctta

SEQ ID NO: 184 - DNA, 21 bp, Artificial Sequence
  Description of Artificial Sequence Synthetic primer
gatcgttacgcaggtcaatgc

SEQ ID NO: 185 - DNA, 22 bp, Artificial Sequence
  Description of Artificial Sequence Synthetic primer
gatgccatccaggttgaactga

SEQ ID NO: 186 - DNA, 21 bp, Artificial Sequence
  Description of Artificial Sequence Synthetic primer
cggctcgatagaacggttcac

SEQ ID NO: 187 - DNA, 18 bp, Artificial Sequence
  Description of Artificial Sequence Synthetic primer
accgccaacgaggatcac

SEQ ID NO: 188 - DNA, 19 bp, Artificial Sequence
  Description of Artificial Sequence Synthetic primer
aactcagcgtcgacaacgt

SEQ ID NO: 189 - DNA, 22 bp, Artificial Sequence
  Description of Artificial Sequence Synthetic primer
tcgccatcaacttcgtcgattt

SEQ ID NO: 190 - DNA, 20 bp, Artificial Sequence
  Description of Artificial Sequence Synthetic primer
ccctggttacgggtctgaac

SEQ ID NO: 191 - DNA, 24 bp, Artificial Sequence
  Description of Artificial Sequence Synthetic primer
ggttgccagaacttcaaaggtttt

SEQ ID NO: 192 - DNA, 25 bp, Artificial Sequence
  Description of Artificial Sequence Synthetic primer
gtgatcttctgctctttaccgctat

SEQ ID NO: 193 - DNA, 25 bp, Artificial Sequence
  Description of Artificial Sequence Synthetic primer
cagagtttcaccatcctgggtataa

SEQ ID NO: 194 - DNA, 25 bp, Artificial Sequence
  Description of Artificial Sequence Synthetic primer
cagttggttcgttgatgatacgttt

SEQ ID NO: 195 - DNA, 18 bp, Artificial Sequence
  Description of Artificial Sequence Synthetic primer
accgccaacgaggatcac

SEQ ID NO: 196 - DNA, 18 bp, Artificial Sequence
  Description of Artificial Sequence Synthetic primer
tttcaggcgctgcattgc

SEQ ID NO: 197 - DNA, 20 bp, Artificial Sequence
  Description of Artificial Sequence Synthetic primer
tcctgcagtgcgactttcag

SEQ ID NO: 198 - DNA, 21 bp, Artificial Sequence
  Description of Artificial Sequence Synthetic primer
cggctcgatagaacggttcac

SEQ ID NO: 199 - DNA, 24 bp, Artificial Sequence
  Description of Artificial Sequence Synthetic primer
cgatcagtttacctacggtttcgt

SEQ ID NO: 200 - DNA, 22 bp, Artificial Sequence
  Description of Artificial Sequence Synthetic primer
aacggtgatgacgccttcttta

SEQ ID NO: 201 - DNA, 24 bp, Artificial Sequence
  Description of Artificial Sequence Synthetic primer
cgagggttactttcactgcatctg

SEQ ID NO: 202 - DNA, 23 bp, Artificial Sequence
  Description of Artificial Sequence Synthetic primer
agctccataccgatctcttcaga

SEQ ID NO: 203 - DNA, 19 bp, Artificial Sequence
  Description of Artificial Sequence Synthetic primer
gccacagaagcagcgtact

SEQ ID NO: 204 - DNA, 24 bp, Artificial Sequence
  Description of Artificial Sequence Synthetic primer
aattttagaagcaacgcggatcag

SEQ ID NO: 205 - DNA, 17 bp, Artificial Sequence
  Description of Artificial Sequence Synthetic primer
ctgcgcgcccatgtttt

SEQ ID NO: 206 - DNA, 20 bp, Artificial Sequence
  Description of Artificial Sequence Synthetic primer
cctggatggcagcttcttca

SEQ ID NO: 207 - DNA, 20 bp, Artificial Sequence
  Description of Artificial Sequence Synthetic primer
gtcaccatgcactcggtagt

SEQ ID NO: 208 - DNA, 17 bp, Artificial Sequence
  Description of Artificial Sequence Synthetic primer
gcgcttcgccttcaaca

SEQ ID NO: 209 - DNA, 24 bp, Artificial Sequence
  Description of Artificial Sequence Synthetic primer
gctttcaagccttcggtaatgatg

SEQ ID NO: 210 - DNA, 23 bp, Artificial Sequence
  Description of Artificial Sequence Synthetic primer
tggtggtgtctttgttgatcaca

SEQ ID NO: 211 - DNA, 25 bp, Artificial Sequence
  Description of Artificial Sequence Synthetic primer
ctttcaggccttcggtaataatgga

SEQ ID NO: 212 - DNA, 25 bp, Artificial Sequence
  Description of Artificial Sequence Synthetic primer
gcgatcagtttacctacagtttcgt

SEQ ID NO: 213 - DNA, 20 bp, Artificial Sequence
  Description of Artificial Sequence Synthetic primer
cctggatggcagcttcttca

SEQ ID NO: 214 - DNA, 22 bp, Artificial Sequence
  Description of Artificial Sequence Synthetic primer
aacggtgatgacgccttcttta

SEQ ID NO: 215 - DNA, 20 bp, Artificial Sequence
  Description of Artificial Sequence Synthetic primer
acagcagccactttcacgat

SEQ ID NO: 216 - DNA, 25 bp, Artificial Sequence
  Description of Artificial Sequence Synthetic primer
gcctttcaggtcagcaattttagaa

SEQ ID NO: 217 - DNA, 24 bp, Artificial Sequence
  Description of Artificial Sequence Synthetic primer
ctacctgagcaatcgctttagagt

SEQ ID NO: 218 - DNA, 24 bp, Artificial Sequence
  Description of Artificial Sequence Synthetic primer
cgcaccgaaagatttatccagaac

SEQ ID NO: 219 - DNA, 21 bp, Artificial Sequence
  Description of Artificial Sequence Synthetic primer
agcaaccacgccttcttctac

SEQ ID NO: 220 - DNA, 24 bp, Artificial Sequence
  Description of Artificial Sequence Synthetic primer
cgttgcgggttaaatcaatatgca

SEQ ID NO: 221 - DNA, 22 bp, Artificial Sequence
  Description of Artificial Sequence Synthetic primer
cgatatacagcagcccgttgac

SEQ ID NO: 222 - DNA, 22 bp, Artificial Sequence
  Description of Artificial Sequence Synthetic primer
cgcgctccagttcgatatacag

SEQ ID NO: 223 - DNA, 24 bp, Artificial Sequence
  Description of Artificial Sequence Synthetic primer
tcacactttacgtgcgaatatgct

SEQ ID NO: 224 - DNA, 25 bp, Artificial Sequence
  Description of Artificial Sequence Synthetic primer
acgctaaggtaatgcgataatggtt

SEQ ID NO: 225 - DNA, 21 bp, Artificial Sequence
  Description of Artificial Sequence Synthetic primer
agggtagccgccattactttg

SEQ ID NO: 226 - DNA, 22 bp, Artificial Sequence
  Description of Artificial Sequence Synthetic primer
aggccctgatgtaaccatttgg

SEQ ID NO: 227 - DNA, 24 bp, Artificial Sequence
  Description of Artificial Sequence Synthetic primer
cagcttgtcaaaaccgatccattg

SEQ ID NO: 228 - DNA, 19 bp, Artificial Sequence
  Description of Artificial Sequence Synthetic primer
ctgttttgcagcgcattgg

SEQ ID NO: 229 - DNA, 21 bp, Artificial Sequence
  Description of Artificial Sequence Synthetic primer
gagcgccttttaccaccagta

SEQ ID NO: 230 - DNA, 22 bp, Artificial Sequence
  Description of Artificial Sequence Synthetic primer
gcgtaaagctcaggctaaaagg

SEQ ID NO: 231 - DNA, 24 bp, Artificial Sequence
  Description of Artificial Sequence Synthetic primer
aacgtgaatgttttccgctaactg

SEQ ID NO: 232 - DNA, 20 bp, Artificial Sequence
  Description of Artificial Sequence Synthetic primer
aaagttacgctcggcaatgc

SEQ ID NO: 233 - DNA, 21 bp, Artificial Sequence
  Description of Artificial Sequence Synthetic primer
cggctaacgctaaggtaatgc

SEQ ID NO: 234 - DNA, 14 bp, Artificial Sequence
  Description of Artificial Sequence Synthetic primer
cgctgcggcgcaat

SEQ ID NO: 235 - DNA, 20 bp, Artificial Sequence
  Description of Artificial Sequence Synthetic primer
ccgatccattgacgcaacag

SEQ ID NO: 236 - DNA, 19 bp, Artificial Sequence
  Description of Artificial Sequence Synthetic primer
tggtttccggctcgttacg

SEQ ID NO: 237 - DNA, 24 bp, Artificial Sequence
  Description of Artificial Sequence Synthetic primer
cagcttgtcaaaaccgatccattg

SEQ ID NO: 238 - DNA, 24 bp, Artificial Sequence
  Description of Artificial Sequence Synthetic primer
cgacccgacggaatcagttaattt

SEQ ID NO: 239 - DNA, 26 bp, Artificial Sequence
  Description of Artificial Sequence Synthetic primer
ctccttctaagaagcgagttttacct

SEQ ID NO: 240 - DNA, 24 bp, Artificial Sequence
  Description of Artificial Sequence Synthetic primer
tcacactttacgtgcgaatatgct

SEQ ID NO: 241 - DNA, 29 bp, Artificial Sequence
  Description of Artificial Sequence Synthetic primer
cgtagttacgcataatcatttctccttct

SEQ ID NO: 242 - DNA, 20 bp, Artificial Sequence
  Description of Artificial Sequence Synthetic primer
tcggcaatgccctgatacag

SEQ ID NO: 243 - DNA, 17 bp, Artificial Sequence
  Description of Artificial Sequence Synthetic primer
cggtagcggcgatttgg

SEQ ID NO: 244 - DNA, 25 bp, Artificial Sequence
  Description of Artificial Sequence Synthetic primer
gaaaatctccctcatccacgttgat

SEQ ID NO: 245 - DNA, 17 bp, Artificial Sequence
  Description of Artificial Sequence Synthetic primer
agcggtagcggcgattt

SEQ ID NO: 246 - DNA, 22 bp, Artificial Sequence
  Description of Artificial Sequence Synthetic primer
caacgcgaaaatctccctcatc

SEQ ID NO: 247 - DNA, 24 bp, Artificial Sequence
  Description of Artificial Sequence Synthetic primer
tctggagcaaactgatgtcttctg

SEQ ID NO: 248 - DNA, 23 bp, Artificial Sequence
  Description of Artificial Sequence Synthetic primer
ctggctaccctatcactgaaacc

SEQ ID NO: 249 - DNA, 25 bp, Artificial Sequence
  Description of Artificial Sequence Synthetic primer
gcagaaacgtatctccaaagaaacc

SEQ ID NO: 250 - DNA, 17 bp, Artificial Sequence
  Description of Artificial Sequence Synthetic primer
cgcgccagctgatcaac

SEQ ID NO: 251 - DNA, 19 bp, Artificial Sequence
  Description of Artificial Sequence Synthetic primer
cgctgaccgagctgtctac

SEQ ID NO: 252 - DNA, 22 bp, Artificial Sequence
  Description of Artificial Sequence Synthetic primer
tgggcgatatgcagaaacgtat

SEQ ID NO: 253 - DNA, 18 bp, Artificial Sequence
  Description of Artificial Sequence Synthetic primer
ggccaggtgctggttgat

SEQ ID NO: 254 - DNA, 25 bp, Artificial Sequence
  Description of Artificial Sequence Synthetic primer
gaaaatctccctcatccacgttgat

SEQ ID NO: 255 - DNA, 24 bp, Artificial Sequence
  Description of Artificial Sequence Synthetic primer
tctggagcaaactgatgtcttctg

SEQ ID NO: 256 - DNA, 19 bp, Artificial Sequence
  Description of Artificial Sequence Synthetic primer
gcccctacaacgcgaaaat

SEQ ID NO: 257 - DNA, 22 bp, Artificial Sequence
  Description of Artificial Sequence Synthetic primer
tgggcgatatgcagaaacgtat

SEQ ID NO: 258 - DNA, 19 bp, Artificial Sequence
  Description of Artificial Sequence Synthetic primer
ggtcgtttgcggtcatcac

SEQ ID NO: 259 - DNA, 19 bp, Artificial Sequence
  Description of Artificial Sequence Synthetic primer
cgctgaccgagctgtctac

SEQ ID NO: 260 - DNA, 22 bp, Artificial Sequence
  Description of Artificial Sequence Synthetic primer
tgggcgatatgcagaaacgtat

SEQ ID NO: 261 - DNA, 15 bp, Artificial Sequence
  Description of Artificial Sequence Synthetic primer
gcgcgcccctacaac

SEQ ID NO: 262 - DNA, 22 bp, Artificial Sequence
  Description of Artificial Sequence Synthetic primer
cctgtacaccggtctgattgac

SEQ ID NO: 263 - DNA, 22 bp, Artificial Sequence
  Description of Artificial Sequence Synthetic primer
tgggcgatatgcagaaacgtat

SEQ ID NO: 264 - DNA, 19 bp, Artificial Sequence
  Description of Artificial Sequence Synthetic primer
ggtcgtttgcggtcatcac

SEQ ID NO: 265 - DNA, 17 bp, Artificial Sequence
  Description of Artificial Sequence Synthetic primer
cgcgccagctgatcaac

SEQ ID NO: 266 - DNA, 20 bp, Artificial Sequence
  Description of Artificial Sequence Synthetic primer
cactgcggcagttcttcgta

SEQ ID NO: 267 - DNA, 20 bp, Artificial Sequence
  Description of Artificial Sequence Synthetic probe
accgtattgatagacttttc

SEQ ID NO: 268 - DNA, 19 bp, Artificial Sequence
  Description of Artificial Sequence Synthetic probe
ctttctatggcaatttttt

SEQ ID NO: 269 - DNA, 16 bp, Artificial Sequence
  Description of Artificial Sequence Synthetic probe
ccctgaccgtttcaac

SEQ ID NO: 270 - DNA, 20 bp, Artificial Sequence
  Description of Artificial Sequence Synthetic probe
ctggcgtatctcctgttaat

SEQ ID NO: 271 - DNA, 16 bp, Artificial Sequence
  Description of Artificial Sequence Synthetic probe
acgcactggatttatc

SEQ ID NO: 272 - DNA, 18 bp, Artificial Sequence
  Description of Artificial Sequence Synthetic probe
catgctaaggtgaataat

SEQ ID NO: 273 - DNA, 19 bp, Artificial Sequence
  Description of Artificial Sequence Synthetic probe
tcaagctcttcctctttcc

SEQ ID NO: 274 - DNA, 17 bp, Artificial Sequence
  Description of Artificial Sequence Synthetic probe
ttgccgccaaccgtttc

SEQ ID NO: 275 - DNA, 16 bp, Artificial Sequence
  Description of Artificial Sequence Synthetic probe
acgcactggatttatc

SEQ ID NO: 276 - DNA, 18 bp, Artificial Sequence
  Description of Artificial Sequence Synthetic probe
tcgctttcagggatctcc

SEQ ID NO: 277 - DNA, 16 bp, Artificial Sequence
  Description of Artificial Sequence Synthetic probe
cctgttccagttttgc

SEQ ID NO: 278 - DNA, 17 bp, Artificial Sequence
  Description of Artificial Sequence Synthetic probe
tcgctttcagggatctc

SEQ ID NO: 279 - DNA, 15 bp, Artificial Sequence
  Description of Artificial Sequence Synthetic probe
ttggcggcaacctga

SEQ ID NO: 280 - DNA, 15 bp, Artificial Sequence
  Description of Artificial Sequence Synthetic probe
tccagccaggaacgc

SEQ ID NO: 281 - DNA, 15 bp, Artificial Sequence
  Description of Artificial Sequence Synthetic probe
cctggctggaaagag

SEQ ID NO: 282 - DNA, 16 bp, Artificial Sequence
  Description of Artificial Sequence Synthetic probe
acgatctgcaaaagcg

SEQ ID NO: 283 - DNA, 16 bp, Artificial Sequence
  Description of Artificial Sequence Synthetic probe
acgcactggatttatc

SEQ ID NO: 284 - DNA, 18 bp, Artificial Sequence
  Description of Artificial Sequence Synthetic probe
atgctaaggtgaataatg

SEQ ID NO: 285 - DNA, 16 bp, Artificial Sequence
  Description of Artificial Sequence Synthetic probe
ttcgcatcgcgctttt

SEQ ID NO: 286 - DNA, 20 bp, Artificial Sequence
  Description of Artificial Sequence Synthetic probe
ccgtttcaatttcaagctct

SEQ ID NO: 287 - DNA, 20 bp, Artificial Sequence
  Description of Artificial Sequence Synthetic probe
ctgatcctctaccgtctctt

SEQ ID NO: 288 - DNA, 19 bp, Artificial Sequence
  Description of Artificial Sequence Synthetic probe
ccagtttcgcattattcac

SEQ ID NO: 289 - DNA, 20 bp, Artificial Sequence
  Description of Artificial Sequence Synthetic probe
cacggtaagcagatagttat

SEQ ID NO: 290 - DNA, 20 bp, Artificial Sequence
  Description of Artificial Sequence Synthetic probe
aaggtctcatcaccccctcc

SEQ ID NO: 291 - DNA, 20 bp, Artificial Sequence
  Description of Artificial Sequence Synthetic probe
ccattaatacgacaaaactc

SEQ ID NO: 292 - DNA, 17 bp, Artificial Sequence
  Description of Artificial Sequence Synthetic probe
atgtggacctgaacatc

SEQ ID NO: 293 - DNA, 19 bp, Artificial Sequence
  Description of Artificial Sequence Synthetic probe
atgtggaactgaacatctt

SEQ ID NO: 294 - DNA, 17 bp, Artificial Sequence
  Description of Artificial Sequence Synthetic probe
atgtggacctgaacatc

SEQ ID NO: 295 - DNA, 17 bp, Artificial Sequence
  Description of Artificial Sequence Synthetic probe
accgctcttacaatcaa

SEQ ID NO: 296 - DNA, 17 bp, Artificial Sequence
  Description of Artificial Sequence Synthetic probe
acgcgttgagttttatc

SEQ ID NO: 297 - DNA, 16 bp, Artificial Sequence
  Description of Artificial Sequence Synthetic probe
cccctccgatggacgc

SEQ ID NO: 298 - DNA, 17 bp, Artificial Sequence
  Description of Artificial Sequence Synthetic probe
accgctcttacaatcaa

SEQ ID NO: 299 - DNA, 16 bp, Artificial Sequence
  Description of Artificial Sequence Synthetic probe
cccctccgatggacgc

SEQ ID NO: 300 - DNA, 19 bp, Artificial Sequence
  Description of Artificial Sequence Synthetic probe
aagagcggtaaaggtctca

SEQ ID NO: 301 - DNA, 18 bp, Artificial Sequence
  Description of Artificial Sequence Synthetic probe
atgtggacctgaacatct

SEQ ID NO: 302 - DNA, 15 bp, Artificial Sequence
  Description of Artificial Sequence Synthetic probe
atggacgcaaagatg

SEQ ID NO: 303 - DNA, 15 bp, Artificial Sequence
  Description of Artificial Sequence Synthetic probe
atggacgcaaagatg

SEQ ID NO: 304 - DNA, 16 bp, Artificial Sequence
  Description of Artificial Sequence Synthetic probe
accccctccgatgacg

SEQ ID NO: 305 - DNA, 17 bp, Artificial Sequence
  Description of Artificial Sequence Synthetic probe
atgtggacctgaacatc

SEQ ID NO: 306 - DNA, 17 bp, Artificial Sequence
  Description of Artificial Sequence Synthetic probe
caggtccacatttcagc

SEQ ID NO: 307 - DNA, 19 bp, Artificial Sequence
  Description of Artificial Sequence Synthetic probe
taggcgacatgctgaaatg

SEQ ID NO: 308 - DNA, 17 bp, Artificial Sequence
  Description of Artificial Sequence Synthetic probe
caccccctccgatggac

SEQ ID NO: 309 - DNA, 17 bp, Artificial Sequence
  Description of Artificial Sequence Synthetic probe
tcgccaacacgaaacag

SEQ ID NO: 310 - DNA, 18 bp, Artificial Sequence
  Description of Artificial Sequence Synthetic probe
tacgcgttgagttttgtc

SEQ ID NO: 311 - DNA, 15 bp, Artificial Sequence
  Description of Artificial Sequence Synthetic probe
tcgccatcaacttcg

SEQ ID NO: 312 - DNA, 16 bp, Artificial Sequence
  Description of Artificial Sequence Synthetic probe
acgggtgctgtgcagc

SEQ ID NO: 313 - DNA, 20 bp, Artificial Sequence
  Description of Artificial Sequence Synthetic probe
ttcacatcaagccatgcgtc

SEQ ID NO: 314 - DNA, 16 bp, Artificial Sequence
  Description of Artificial Sequence Synthetic probe
acgcgtgcctgcgttc

SEQ ID NO: 315 - DNA, 18 bp, Artificial Sequence
  Description of Artificial Sequence Synthetic probe
atggtcgccctggttacg

SEQ ID NO: 316 - DNA, 17 bp, Artificial Sequence
  Description of Artificial Sequence Synthetic probe
tacgggtctgaaccagc

SEQ ID NO: 317 - DNA, 20 bp, Artificial Sequence
  Description of Artificial Sequence Synthetic probe
tcgttgacgagtttaagaaa

SEQ ID NO: 318 - DNA, 20 bp, Artificial Sequence
  Description of Artificial Sequence Synthetic probe
acgtgcgtctgataacaaat

SEQ ID NO: 319 - DNA, 18 bp, Artificial Sequence
  Description of Artificial Sequence Synthetic probe
atcttcaaccaggctttc

SEQ ID NO: 320 - DNA, 16 bp, Artificial Sequence
  Description of Artificial Sequence Synthetic probe
cctgcagtgcgacttt

SEQ ID NO: 321 - DNA, 16 bp, Artificial Sequence
  Description of Artificial Sequence Synthetic probe
acgcttctgcaaacaa

SEQ ID NO: 322 - DNA, 19 bp, Artificial Sequence
  Description of Artificial Sequence Synthetic probe
ccgtactatcgcggtttac

SEQ ID NO: 323 - DNA, 17 bp, Artificial Sequence
  Description of Artificial Sequence Synthetic probe
cagctcttcgaacttac

SEQ ID NO: 324 - DNA, 17 bp, Artificial Sequence
  Description of Artificial Sequence Synthetic probe
caacttcgtcgatttcg

SEQ ID NO: 325 - DNA, 16 bp, Artificial Sequence
  Description of Artificial Sequence Synthetic probe
tcctgcacgtctccgc

SEQ ID NO: 326 - DNA, 19 bp, Artificial Sequence
  Description of Artificial Sequence Synthetic probe
ctacgccttctatcattgc

SEQ ID NO: 327 - DNA, 16 bp, Artificial Sequence
  Description of Artificial Sequence Synthetic probe
acgaccagcatctttg

SEQ ID NO: 328 - DNA, 16 bp, Artificial Sequence
  Description of Artificial Sequence Synthetic probe
cagacacggataggcc

SEQ ID NO: 329 - DNA, 17 bp, Artificial Sequence
  Description of Artificial Sequence Synthetic probe
acgcaggtcaatgccct

SEQ ID NO: 330 - DNA, 18 bp, Artificial Sequence
  Description of Artificial Sequence Synthetic probe
ctggtgaaccgttctatc

SEQ ID NO: 331 - DNA, 20 bp, Artificial Sequence
  Description of Artificial Sequence Synthetic probe
cagatcttcaaccaggcttt

SEQ ID NO: 332 - DNA, 19 bp, Artificial Sequence
  Description of Artificial Sequence Synthetic probe
actatttccgctaactccg

SEQ ID NO: 333 - DNA, 20 bp, Artificial Sequence
  Description of Artificial Sequence Synthetic probe
ctttatccatcgcttccgcg

SEQ ID NO: 334 - DNA, 16 bp, Artificial Sequence
  Description of Artificial Sequence Synthetic probe
acgctcgtgtgaaaat

SEQ ID NO: 335 - DNA, 16 bp, Artificial Sequence
  Description of Artificial Sequence Synthetic probe
cctgaccggcggtacc

SEQ ID NO: 336 - DNA, 17 bp, Artificial Sequence
  Description of Artificial Sequence Synthetic probe
cagaacgggtaactttg

SEQ ID NO: 337 - DNA, 16 bp, Artificial Sequence
  Description of Artificial Sequence Synthetic probe
ccaccaccagcaacca

SEQ ID NO: 338 - DNA, 18 bp, Artificial Sequence
  Description of Artificial Sequence Synthetic probe
aactggaagacaagtttg

SEQ ID NO: 339 - DNA, 15 bp, Artificial Sequence
  Description of Artificial Sequence Synthetic probe
cccacgccatcaatg

SEQ ID NO: 340 - DNA, 19 bp, Artificial Sequence
  Description of Artificial Sequence Synthetic probe
ctgtggctggtctgatgat

SEQ ID NO: 341 - DNA, 18 bp, Artificial Sequence
  Description of Artificial Sequence Synthetic probe
tcttcagcgatgatcagc

SEQ ID NO: 342 - DNA, 15 bp, Artificial Sequence
  Description of Artificial Sequence Synthetic probe
tcgcctgcagcatcg

SEQ ID NO: 343 - DNA, 15 bp, Artificial Sequence
  Description of Artificial Sequence Synthetic probe
ctgccccaggtcttc

SEQ ID NO: 344 - DNA, 16 bp, Artificial Sequence
  Description of Artificial Sequence Synthetic probe
ctgcagcgtcgttcgc

SEQ ID NO: 345 - DNA, 18 bp, Artificial Sequence
  Description of Artificial Sequence Synthetic probe
actatctccgctaactcc

SEQ ID NO: 346 - DNA, 17 bp, Artificial Sequence
  Description of Artificial Sequence Synthetic probe
acaccaccaccatcatt

SEQ ID NO: 347 - DNA, 17 bp, Artificial Sequence
  Description of Artificial Sequence Synthetic probe
aagcgatggataaagtc

SEQ ID NO: 348 - DNA, 16 bp, Artificial Sequence
  Description of Artificial Sequence Synthetic probe
cacgcatggtgttcac

SEQ ID NO: 349 - DNA, 16 bp, Artificial Sequence
  Description of Artificial Sequence Synthetic probe
tcagcgcaacgccacc

SEQ ID NO: 350 - DNA, 16 bp, Artificial Sequence
  Description of Artificial Sequence Synthetic probe
acggacagggctttca

SEQ ID NO: 351 - DNA, 21 bp, Artificial Sequence
  Description of Artificial Sequence Synthetic probe
acgttacggcctttcggaccg

SEQ ID NO: 352 - DNA, 17 bp, Artificial Sequence
  Description of Artificial Sequence Synthetic probe
tcttcaacgcgggcttt

SEQ ID NO: 353 - DNA, 20 bp, Artificial Sequence
  Description of Artificial Sequence Synthetic probe
ccgttggtaaacgtcgcgcc

SEQ ID NO: 354 - DNA, 14 bp, Artificial Sequence
  Description of Artificial Sequence Synthetic probe
tcgtggcgcaaacc

SEQ ID NO: 355 - DNA, 19 bp, Artificial Sequence
  Description of Artificial Sequence Synthetic probe
ccgttgaccaggtttgcgc

SEQ ID NO: 356 - DNA, 18 bp, Artificial Sequence
  Description of Artificial Sequence Synthetic probe
tcccatgctcgccgtcag

SEQ ID NO: 357 - DNA, 16 bp, Artificial Sequence
  Description of Artificial Sequence Synthetic probe
atcgtcgcttttttcg

SEQ ID NO: 358 - DNA, 16 bp, Artificial Sequence
  Description of Artificial Sequence Synthetic probe
acagacggtcaaaacc

SEQ ID NO: 359 - DNA, 15 bp, Artificial Sequence
  Description of Artificial Sequence Synthetic probe
aacagccggaaaacg

SEQ ID NO: 360 - DNA, 23 bp, Artificial Sequence
  Description of Artificial Sequence Synthetic probe
cttagaaggagaaatgattatgc

SEQ ID NO: 361 - DNA, 16 bp, Artificial Sequence
  Description of Artificial Sequence Synthetic probe
ccagcttgtcaaaacc

SEQ ID NO: 362 - DNA, 18 bp, Artificial Sequence
  Description of Artificial Sequence Synthetic probe
ttcgcggaaagcgaactg

SEQ ID NO: 363 - DNA, 17 bp, Artificial Sequence
  Description of Artificial Sequence Synthetic probe
ctgcatgaccaggccct

SEQ ID NO: 364 - DNA, 19 bp, Artificial Sequence
  Description of Artificial Sequence Synthetic probe
aagttacgctcggcaatgc

SEQ ID NO: 365 - DNA, 20 bp, Artificial Sequence
  Description of Artificial Sequence Synthetic probe
acaggtaagtacgttctttc

SEQ ID NO: 366 - DNA, 20 bp, Artificial Sequence
  Description of Artificial Sequence Synthetic probe
aatggttatcgtcgcttttt

SEQ ID NO: 367 - DNA, 16 bp, Artificial Sequence
  Description of Artificial Sequence Synthetic probe
caacgagccggaaacc

SEQ ID NO: 368 - DNA, 20 bp, Artificial Sequence
  Description of Artificial Sequence Synthetic probe
atgcgtaactacgatttatc

SEQ ID NO: 369 - DNA, 19 bp, Artificial Sequence
  Description of Artificial Sequence Synthetic probe
cctgctgcatattgattta

SEQ ID NO: 370 - DNA, 20 bp, Artificial Sequence
  Description of Artificial Sequence Synthetic probe
atgcgtaactacgatttatc

SEQ ID NO: 371 - DNA, 16 bp, Artificial Sequence
  Description of Artificial Sequence Synthetic probe
aaaccgcgccgtatcg

SEQ ID NO: 372 - DNA, 18 bp, Artificial Sequence
  Description of Artificial Sequence Synthetic probe
attcgcacgtaaagtgtg

SEQ ID NO: 373 - DNA, 17 bp, Artificial Sequence
  Description of Artificial Sequence Synthetic probe
ctgtcatcccatgctcg

SEQ ID NO: 374 - DNA, 18 bp, Artificial Sequence
  Description of Artificial Sequence Synthetic probe
attcgcacgtaaagtgtg

SEQ ID NO: 375 - DNA, 16 bp, Artificial Sequence
  Description of Artificial Sequence Synthetic probe
catgagcgccttttac

SEQ ID NO: 376 - DNA, 20 bp, Artificial Sequence
  Description of Artificial Sequence Synthetic probe
ccagatccatatcgtatttc

SEQ ID NO: 377 - DNA, 19 bp, Artificial Sequence
  Description of Artificial Sequence Synthetic probe
ctgtacaccggtctgattg

SEQ ID NO: 378 - DNA, 17 bp, Artificial Sequence
  Description of Artificial Sequence Synthetic probe
caggtgctggttgacgc

SEQ ID NO: 379 - DNA, 18 bp, Artificial Sequence
  Description of Artificial Sequence Synthetic probe
acgttgacgtgaattatt

SEQ ID NO: 380 - DNA, 19 bp, Artificial Sequence
  Description of Artificial Sequence Synthetic probe
ctgatcaacaccgttcacg

SEQ ID NO: 381 - DNA, 17 bp, Artificial Sequence
  Description of Artificial Sequence Synthetic probe
cccaaatcgccgctacc

SEQ ID NO: 382 - DNA, 15 bp, Artificial Sequence
  Description of Artificial Sequence Synthetic probe
accacgcgctgaccg

SEQ ID NO: 383 - DNA, 18 bp, Artificial Sequence
  Description of Artificial Sequence Synthetic probe
ccgttcacgttgacatgc

SEQ ID NO: 384 - DNA, 15 bp, Artificial Sequence
  Description of Artificial Sequence Synthetic probe
caacgctggctaccc

SEQ ID NO: 385 - DNA, 17 bp, Artificial Sequence
  Description of Artificial Sequence Synthetic probe
cagcgttggtagacagc

SEQ ID NO: 386 - DNA, 18 bp, Artificial Sequence
  Description of Artificial Sequence Synthetic probe
tcgtttgcggtcatcacc

SEQ ID NO: 387 - DNA, 19 bp, Artificial Sequence
  Description of Artificial Sequence Synthetic probe
ccggtgtacaggtcagaat

SEQ ID NO: 388 - DNA, 17 bp, Artificial Sequence
  Description of Artificial Sequence Synthetic probe
ccagctgatcaacaccg

SEQ ID NO: 389 - DNA, 19 bp, Artificial Sequence
  Description of Artificial Sequence Synthetic probe
ctccctcatccacgttgat

SEQ ID NO: 390 - DNA, 16 bp, Artificial Sequence
  Description of Artificial Sequence Synthetic probe
cccaccacgcgctgac

SEQ ID NO: 391 - DNA, 18 bp, Artificial Sequence
  Description of Artificial Sequence Synthetic probe
cagtttgctccagaagtc

SEQ ID NO: 392 - DNA, 17 bp, Artificial Sequence
  Description of Artificial Sequence Synthetic probe
acgctggctaccctatc

SEQ ID NO: 393 - DNA, 18 bp, Artificial Sequence
  Description of Artificial Sequence Synthetic probe
ccgagctgtctaccaacg

SEQ ID NO: 394 - DNA, 16 bp, Artificial Sequence
  Description of Artificial Sequence Synthetic probe
atgagggagattttcg

SEQ ID NO: 395 - DNA, 18 bp, Artificial Sequence
  Description of Artificial Sequence Synthetic probe
acgtttctgcatatcgcc

SEQ ID NO: 396 - DNA, 17 bp, Artificial Sequence
  Description of Artificial Sequence Synthetic probe
cagcgttggtagacagc

SEQ ID NO: 397 - DNA, 17 bp, Artificial Sequence
  Description of Artificial Sequence Synthetic probe
acttctggagcaaactg

SEQ ID NO: 398 - DNA, 18 bp, Artificial Sequence
  Description of Artificial Sequence Synthetic probe
ccgttcacgttgacatgc

SEQ ID NO: 399 - DNA, 14 bp, Artificial Sequence
  Description of Artificial Sequence Synthetic probe
tgctgccggtgacc

SEQ ID NO: 400 - DNA, 23 bp, Artificial Sequence
  Description of Artificial Sequence Synthetic primer
gcgatggtggtagttatgcagaa

SEQ ID NO: 401 - DNA, 23 bp, Artificial Sequence
  Description of Artificial Sequence Synthetic primer
ggcacattggcgagtttaacaaa

SEQ ID NO: 402 - DNA, 27 bp, Artificial Sequence
  Description of Artificial Sequence Synthetic primer
cggtcttaggaaaaacgaatgtaacag

SEQ ID NO: 403 - DNA, 25 bp, Artificial Sequence
  Description of Artificial Sequence Synthetic primer
cgataatgcgctctacctgttgata

SEQ ID NO: 404 - DNA, 21 bp, Artificial Sequence
  Description of Artificial Sequence Synthetic primer
ccaacaaaagccggatatgct

SEQ ID NO: 405 - DNA, 28 bp, Artificial Sequence
  Description of Artificial Sequence Synthetic primer
cgaatctaactggtttgactttgttcaa

SEQ ID NO: 406 - DNA, 29 bp, Artificial Sequence
  Description of Artificial Sequence Synthetic primer
ccaaataagtgatataactccacttggga

SEQ ID NO: 407 - DNA, 25 bp, Artificial Sequence
  Description of Artificial Sequence Synthetic primer
tcaggcagctacaattacacaagat

SEQ ID NO: 408 - DNA, 28 bp, Artificial Sequence
  Description of Artificial Sequence Synthetic primer
cccaatttctaacctgaaaaatctcaca

SEQ ID NO: 409 - DNA, 30 bp, Artificial Sequence
  Description of Artificial Sequence Synthetic primer
ggacgaattatccttaaatggtaaccagtt

SEQ ID NO: 410 - DNA, 28 bp, Artificial Sequence
  Description of Artificial Sequence Synthetic primer
accaaattagtaatctagcaccactgtc

SEQ ID NO: 411 - DNA, 25 bp, Artificial Sequence
  Description of Artificial Sequence Synthetic primer
cctgtcactattggaaaaggaacga

SEQ ID NO: 412 - DNA, 22 bp, Artificial Sequence
  Description of Artificial Sequence Synthetic primer
acggtctcgcaaacagatctag

SEQ ID NO: 413 - DNA, 20 bp, Artificial Sequence
  Description of Artificial Sequence Synthetic primer
cgcagaaacaggcggtaaaa

SEQ ID NO: 414 - DNA, 25 bp, Artificial Sequence
  Description of Artificial Sequence Synthetic primer
gcacaacaagtacaatgaacgctta

SEQ ID NO: 415 - DNA, 25 bp, Artificial Sequence
  Description of Artificial Sequence Synthetic primer
gcaaatatacctggaagcaacacat

SEQ ID NO: 416 - DNA, 26 bp, Artificial Sequence
  Description of Artificial Sequence Synthetic primer
gcttcaggcagatagattagggataa

SEQ ID NO: 417 - DNA, 28 bp, Artificial Sequence
  Description of Artificial Sequence Synthetic primer
agcgttaaatcgtatacagcaacatttg

SEQ ID NO: 418 - DNA, 22 bp, Artificial Sequence
  Description of Artificial Sequence Synthetic primer
ctggtctaaccgcactcactaa

SEQ ID NO: 419 - DNA, 29 bp, Artificial Sequence
  Description of Artificial Sequence Synthetic primer
catggaacttacctagctatacgaatgaa

SEQ ID NO: 420 - DNA, 26 bp, Artificial Sequence
  Description of Artificial Sequence Synthetic primer
cgactcaagcagtagactatcaagga

SEQ ID NO: 421 - DNA, 18 bp, Artificial Sequence
  Description of Artificial Sequence Synthetic primer
cggcaaacaaggcgatga

SEQ ID NO: 422 - DNA, 25 bp, Artificial Sequence
  Description of Artificial Sequence Synthetic primer
gatccaacaagcaaaaggaacagaa

SEQ ID NO: 423 - DNA, 25 bp, Artificial Sequence
  Description of Artificial Sequence Synthetic primer
gatccaacaagcaaaaggaacagaa

SEQ ID NO: 424 - DNA, 24 bp, Artificial Sequence
  Description of Artificial Sequence Synthetic primer
gcaacaactcctggtaaacttcct

SEQ ID NO: 425 - DNA, 26 bp, Artificial Sequence
  Description of Artificial Sequence Synthetic primer
aatatggtactccagcaatcaaagct

SEQ ID NO: 426 - DNA, 28 bp, Artificial Sequence
  Description of Artificial Sequence Synthetic primer
tgatgcaaaagttactgattctggttct

SEQ ID NO: 427 - DNA, 24 bp, Artificial Sequence
  Description of Artificial Sequence Synthetic primer
tgcttctgcgattttgcttgattt

SEQ ID NO: 428 - DNA, 24 bp, Artificial Sequence
  Description of Artificial Sequence Synthetic primer
ctacacctggtaaactgcctgatt

SEQ ID NO: 429 - DNA, 27 bp, Artificial Sequence
  Description of Artificial Sequence Synthetic primer
aaaccagcaaaagtaaaagcttccatt

SEQ ID NO: 430 - DNA, 25 bp, Artificial Sequence
  Description of Artificial Sequence Synthetic primer
gcgaaccaacataacgtgcaatata

SEQ ID NO: 431 - DNA, 33 bp, Artificial Sequence
  Description of Artificial Sequence Synthetic primer
atggtacaaataacaaaaggtaaatttgatggt

SEQ ID NO: 432 - DNA, 29 bp, Artificial Sequence
  Description of Artificial Sequence Synthetic primer
aaggatgtagaagacttcaaacaacttgt

SEQ ID NO: 433 - DNA, 30 bp, Artificial Sequence
  Description of Artificial Sequence Synthetic primer
cctcgttatggtgtagatgtacttaaactt

SEQ ID NO: 434 - DNA, 24 bp, Artificial Sequence
  Description of Artificial Sequence Synthetic primer
tgcttctgcgattttgcttgattt

SEQ ID NO: 435 - DNA, 25 bp, Artificial Sequence
  Description of Artificial Sequence Synthetic primer
tggcgtaacttctgaaatgttccat

SEQ ID NO: 436 - DNA, 23 bp, Artificial Sequence
  Description of Artificial Sequence Synthetic primer
gtgaagtcgcttatacgcaagac

SEQ ID NO: 437 - DNA, 24 bp, Artificial Sequence
  Description of Artificial Sequence Synthetic primer
acgaaggtagcggacttttaactt

SEQ ID NO: 438 - DNA, 24 bp, Artificial Sequence
  Description of Artificial Sequence Synthetic primer
tgggctgatggtattgaagtgtac

SEQ ID NO: 439 - DNA, 30 bp, Artificial Sequence
  Description of Artificial Sequence Synthetic primer
ccagcgaaagtaaaagcatctattaaagaa

SEQ ID NO: 440 - DNA, 29 bp, Artificial Sequence
  Description of Artificial Sequence Synthetic primer
aattgaaaccagcaaaagtaaaagcttct

SEQ ID NO: 441 - DNA, 27 bp, Artificial Sequence
  Description of Artificial Sequence Synthetic primer
ggtggagatgcagttaaaattcttgtt

SEQ ID NO: 442 - DNA, 25 bp, Artificial Sequence
  Description of Artificial Sequence Synthetic primer
gtagtaggtgacgcaaaagacaagt

SEQ ID NO: 443 - DNA, 28 bp, Artificial Sequence
  Description of Artificial Sequence Synthetic primer
aggtatgaaagacaaagcaaaaggactt

SEQ ID NO: 444 - DNA, 24 bp, Artificial Sequence
  Description of Artificial Sequence Synthetic primer
ggcaaacaagttgaaggtaaagct

SEQ ID NO: 445 - DNA, 23 bp, Artificial Sequence
  Description of Artificial Sequence Synthetic primer
cgaaacaagcagaaggtaaagca

SEQ ID NO: 446 - DNA, 25 bp, Artificial Sequence
  Description of Artificial Sequence Synthetic primer
acaagttcggtaaagcaacagatga

SEQ ID NO: 447 - DNA, 26 bp, Artificial Sequence
  Description of Artificial Sequence Synthetic primer
aggtatgaaggacaaagcaaaaggat

SEQ ID NO: 448 - DNA, 25 bp, Artificial Sequence
  Description of Artificial Sequence Synthetic primer
cgcaaaagacaaattcggtaaagca

SEQ ID NO: 449 - DNA, 26 bp, Artificial Sequence
  Description of Artificial Sequence Synthetic primer
atgagcgaagataaaggtatgaagga

SEQ ID NO: 450 - DNA, 28 bp, Artificial Sequence
  Description of Artificial Sequence Synthetic primer
acaaagcaaaaggaatgaaagacaaagt

SEQ ID NO: 451 - DNA, 28 bp, Artificial Sequence
  Description of Artificial Sequence Synthetic primer
aggtatgaaagacaaagcaaaaggactt

SEQ ID NO: 452 - DNA, 25 bp, Artificial Sequence
  Description of Artificial Sequence Synthetic primer
gtgacgcaaaagacaaattcggtaa

SEQ ID NO: 453 - DNA, 23 bp, Artificial Sequence
  Description of Artificial Sequence Synthetic primer
gggaaagcaacagatgacaaagg

SEQ ID NO: 454 - DNA, 25 bp, Artificial Sequence
  Description of Artificial Sequence Synthetic primer
acaagttcggtaaagcaacagatga

SEQ ID NO: 455 - DNA, 28 bp, Artificial Sequence
  Description of Artificial Sequence Synthetic primer
ggtatgaaagacaaagcaaaaggaatga

SEQ ID NO: 456 - DNA, 26 bp, Artificial Sequence
  Description of Artificial Sequence Synthetic primer
agtagtaggtgacgcaaaagacaaat

SEQ ID NO: 457 - DNA, 26 bp, Artificial Sequence
  Description of Artificial Sequence Synthetic primer
aggtatgaaggacaaagcaaaaggat

SEQ ID NO: 458 - DNA, 24 bp, Artificial Sequence
  Description of Artificial Sequence Synthetic primer
ggcatgaaagacaaagcaaaagga

SEQ ID NO: 459 - DNA, 28 bp, Artificial Sequence
  Description of Artificial Sequence Synthetic primer
tgaaggacaaaggaaaaggattgaaaga

SEQ ID NO: 460 - DNA, 25 bp, Artificial Sequence
  Description of Artificial Sequence Synthetic primer
gagcgaagataaaggcatgaaagac

SEQ ID NO: 461 - DNA, 30 bp, Artificial Sequence
  Description of Artificial Sequence Synthetic primer
cgtcaataacttatacaacgaaggatcact

SEQ ID NO: 462 - DNA, 27 bp, Artificial Sequence
  Description of Artificial Sequence Synthetic primer
catttatgataatcctgttggcgtgtt

SEQ ID NO: 463 - DNA, 24 bp, Artificial Sequence
  Description of Artificial Sequence Synthetic primer
gccgctatctcctttacattggtt

SEQ ID NO: 464 - DNA, 24 bp, Artificial Sequence
  Description of Artificial Sequence Synthetic primer
gccgctatctcctttacattggtt

SEQ ID NO: 465 - DNA, 23 bp, Artificial Sequence
  Description of Artificial Sequence Synthetic primer
ggatgcttatagtcgtgggatgg

SEQ ID NO: 466 - DNA, 27 bp, Artificial Sequence
  Description of Artificial Sequence Synthetic primer
tctgaatcagagagcattagccaattt

SEQ ID NO: 467 - DNA, 27 bp, Artificial Sequence
  Description of Artificial Sequence Synthetic primer
tctgaatcagagagcattagccaattt

SEQ ID NO: 468 - DNA, 25 bp, Artificial Sequence
  Description of Artificial Sequence Synthetic primer
gcgctactgtaaaagaagcaagaag

SEQ ID NO: 469 - DNA, 26 bp, Artificial Sequence
  Description of Artificial Sequence Synthetic primer
agtggaatgtgtgaaagatggacttc

SEQ ID NO: 470 - DNA, 25 bp, Artificial Sequence
  Description of Artificial Sequence Synthetic primer
gcgctactgtaaaagaagcaagaag

SEQ ID NO: 471 - DNA, 25 bp, Artificial Sequence
  Description of Artificial Sequence Synthetic primer
ggcgatttgtcttctatgtctcgtt

SEQ ID NO: 472 - DNA, 25 bp, Artificial Sequence
  Description of Artificial Sequence Synthetic primer
gcgctactgtaaaagaagcaagaag

SEQ ID NO: 473 - DNA, 25 bp, Artificial Sequence
  Description of Artificial Sequence Synthetic primer
cggcttacctggtgatttatcttct

SEQ ID NO: 474 - DNA, 18 bp, Artificial Sequence
  Description of Artificial Sequence Synthetic primer
tgggatgggcggaattgg

SEQ ID NO: 475 - DNA, 22 bp, Artificial Sequence
  Description of Artificial Sequence Synthetic primer
cgcgttctttcgagtgaaactc

SEQ ID NO: 476 - DNA, 25 bp, Artificial Sequence
  Description of Artificial Sequence Synthetic primer
caattatcgcgttctttcgagtgaa

SEQ ID NO: 477 - DNA, 24 bp, Artificial Sequence
  Description of Artificial Sequence Synthetic primer
cggtggaacaacaaaaaggtcttt

SEQ ID NO: 478 - DNA, 26 bp, Artificial Sequence
  Description of Artificial Sequence Synthetic primer
tcagagagcattagccaatttttcca

SEQ ID NO: 479 - DNA, 29 bp, Artificial Sequence
  Description of Artificial Sequence Synthetic primer
catttatgataatcctgttggcgtgttaa

SEQ ID NO: 480 - DNA, 29 bp, Artificial Sequence
  Description of Artificial Sequence Synthetic primer
agagattgatttggatgcttatagtcgtg

SEQ ID NO: 481 - DNA, 24 bp, Artificial Sequence
  Description of Artificial Sequence Synthetic primer
tgttgatagagaagcccgaaatgg

SEQ ID NO: 482 - DNA, 36 bp, Artificial Sequence
  Description of Artificial Sequence Synthetic primer
ggatggctttttttagacgaaaataaaattaaagac

SEQ ID NO: 483 - DNA, 28 bp, Artificial Sequence
  Description of Artificial Sequence Synthetic primer
tgaaagaaaagtacaacccaagaaggaa

SEQ ID NO: 484 - DNA, 25 bp, Artificial Sequence
  Description of Artificial Sequence Synthetic primer
gcgcacttgatacgttctataagca

SEQ ID NO: 485 - DNA, 25 bp, Artificial Sequence
  Description of Artificial Sequence Synthetic primer
acagcccaagaaggaagtattgttt

SEQ ID NO: 486 - DNA, 25 bp, Artificial Sequence
  Description of Artificial Sequence Synthetic primer
gtacggattatatgtccggaaacga

SEQ ID NO: 487 - DNA, 25 bp, Artificial Sequence
  Description of Artificial Sequence Synthetic primer
agacctaagttcgctcaaggatcta

SEQ ID NO: 488 - DNA, 23 bp, Artificial Sequence
  Description of Artificial Sequence Synthetic primer
tgttgtggatgcggatgttagat

SEQ ID NO: 489 - DNA, 25 bp, Artificial Sequence
  Description of Artificial Sequence Synthetic primer
tccggtagtagatagcccaatcaaa

SEQ ID NO: 490 - DNA, 28 bp, Artificial Sequence
  Description of Artificial Sequence Synthetic primer
caatctaatttggtcgttccgaatacag

SEQ ID NO: 491 - DNA, 27 bp, Artificial Sequence
  Description of Artificial Sequence Synthetic primer
agtgatgatggcgattatgaaaaacct

SEQ ID NO: 492 - DNA, 27 bp, Artificial Sequence
  Description of Artificial Sequence Synthetic primer
ggcatgagtggaattttaatacggatt

SEQ ID NO: 493 - DNA, 27 bp, Artificial Sequence
  Description of Artificial Sequence Synthetic primer
agtaagttatgatgttgatggaacggt

SEQ ID NO: 494 - DNA, 23 bp, Artificial Sequence
  Description of Artificial Sequence Synthetic primer
cgaagccaaaacaccaattacca

SEQ ID NO: 495 - DNA, 21 bp, Artificial Sequence
  Description of Artificial Sequence Synthetic primer
cctccgaccaaacaaggctat

SEQ ID NO: 496 - DNA, 29 bp, Artificial Sequence
  Description of Artificial Sequence Synthetic primer
acagatataaagcccttagcaaacttgaa

SEQ ID NO: 497 - DNA, 25 bp, Artificial Sequence
  Description of Artificial Sequence Synthetic primer
tgccaacaccaatcaagcaaatttt

SEQ ID NO: 498 - DNA, 27 bp, Artificial Sequence
  Description of Artificial Sequence Synthetic primer
gaaacgattttactttgtacgcgatgt

SEQ ID NO: 499 - DNA, 26 bp, Artificial Sequence
  Description of Artificial Sequence Synthetic primer
gcgatttaagagcattagcaggactt

SEQ ID NO: 500 - DNA, 23 bp, Artificial Sequence
  Description of Artificial Sequence Synthetic primer
gtacgcgatgtttaaagcggaaa

SEQ ID NO: 501 - DNA, 22 bp, Artificial Sequence
  Description of Artificial Sequence Synthetic primer
cctccgaccaaacaaggctatg

SEQ ID NO: 502 - DNA, 28 bp, Artificial Sequence
  Description of Artificial Sequence Synthetic primer
tgaatttgacggaactttaacactcgta

SEQ ID NO: 503 - DNA, 25 bp, Artificial Sequence
  Description of Artificial Sequence Synthetic primer
gggctggattttaacaatcggtatt

SEQ ID NO: 504 - DNA, 24 bp, Artificial Sequence
  Description of Artificial Sequence Synthetic primer
cacaaaatgtttctggctgggtac

SEQ ID NO: 505 - DNA, 28 bp, Artificial Sequence
  Description of Artificial Sequence Synthetic primer
ccacacaatctcttacttttcagacaga

SEQ ID NO: 506 - DNA, 22 bp, Artificial Sequence
  Description of Artificial Sequence Synthetic primer
cgcaattggtatcgtccttgtc

SEQ ID NO: 507 - DNA, 27 bp, Artificial Sequence
  Description of Artificial Sequence Synthetic primer
aaaatcacaaaatgtttctggttgggt

SEQ ID NO: 508 - DNA, 32 bp, Artificial Sequence
  Description of Artificial Sequence Synthetic primer
agacttttacacgaattcttgttttattagcg

SEQ ID NO: 509 - DNA, 25 bp, Artificial Sequence
  Description of Artificial Sequence Synthetic primer
ggcgcattcactgctaaacaaaata

SEQ ID NO: 510 - DNA, 24 bp, Artificial Sequence
  Description of Artificial Sequence Synthetic primer
gcgcattcactgctaaacaaaaca

SEQ ID NO: 511 - DNA, 30 bp, Artificial Sequence
  Description of Artificial Sequence Synthetic primer
atccaggaatttcattattaacctcgacat

SEQ ID NO: 512 - DNA, 28 bp, Artificial Sequence
  Description of Artificial Sequence Synthetic primer
ctcgtattacttttcggtatgtgggtat

SEQ ID NO: 513 - DNA, 24 bp, Artificial Sequence
  Description of Artificial Sequence Synthetic primer
tgtattgctgtttggtatgtgggt

SEQ ID NO: 514 - DNA, 28 bp, Artificial Sequence
  Description of Artificial Sequence Synthetic primer
ccacacaatctcttatttctcagacaga

SEQ ID NO: 515 - DNA, 26 bp, Artificial Sequence
  Description of Artificial Sequence Synthetic primer
acctcaacattgatgatcggtttctt

SEQ ID NO: 516 - DNA, 25 bp, Artificial Sequence
  Description of Artificial Sequence Synthetic primer
actgccaaacaaaacaacgtacaag

SEQ ID NO: 517 - DNA, 27 bp, Artificial Sequence
  Description of Artificial Sequence Synthetic primer
ggcattattgttggttttatcgctcta

SEQ ID NO: 518 - DNA, 24 bp, Artificial Sequence
  Description of Artificial Sequence Synthetic primer
gcgctattcaatccagttgtttcc

SEQ ID NO: 519 - DNA, 28 bp, Artificial Sequence
  Description of Artificial Sequence Synthetic primer
atcattgttggatttatcgctctcttca

SEQ ID NO: 520 - DNA, 24 bp, Artificial Sequence
  Description of Artificial Sequence Synthetic primer
ggtgctagctgacgggattttatc

SEQ ID NO: 521 - DNA, 27 bp, Artificial Sequence
  Description of Artificial Sequence Synthetic primer
gggattgcaatgattatacttgggatt

SEQ ID NO: 522 - DNA, 25 bp, Artificial Sequence
  Description of Artificial Sequence Synthetic primer
gggttggatcctaacagttggtatt

SEQ ID NO: 523 - DNA, 23 bp, Artificial Sequence
  Description of Artificial Sequence Synthetic primer
cttgggcgaagaaactttcactt

SEQ ID NO: 524 - DNA, 25 bp, Artificial Sequence
  Description of Artificial Sequence Synthetic primer
cgagccacctgaaagtttgtcttat

SEQ ID NO: 525 - DNA, 27 bp, Artificial Sequence
  Description of Artificial Sequence Synthetic primer
tttatgattctgcacatgaaactgcac

SEQ ID NO: 526 - DNA, 25 bp, Artificial Sequence
  Description of Artificial Sequence Synthetic primer
caccacgtttcccagctaatataca

SEQ ID NO: 527 - DNA, 25 bp, Artificial Sequence
  Description of Artificial Sequence Synthetic primer
cgagccacctgaaagtttgtcttat

SEQ ID NO: 528 - DNA, 25 bp, Artificial Sequence
  Description of Artificial Sequence Synthetic primer
catattcgaagtgccattgctgaag

SEQ ID NO: 529 - DNA, 25 bp, Artificial Sequence
  Description of Artificial Sequence Synthetic primer
ggcataaatccaaagcaactccatt

SEQ ID NO: 530 - DNA, 25 bp, Artificial Sequence
  Description of Artificial Sequence Synthetic primer
agcgttgaatgagtttgcaattgag

SEQ ID NO: 531 - DNA, 26 bp, Artificial Sequence
  Description of Artificial Sequence Synthetic primer
agaacaagaaatagcgttaccccaaa

SEQ ID NO: 532 - DNA, 25 bp, Artificial Sequence
  Description of Artificial Sequence Synthetic primer
ctgcaccaaaatttccagctaacat

SEQ ID NO: 533 - DNA, 25 bp, Artificial Sequence
  Description of Artificial Sequence Synthetic primer
ggcagatgctgatctcattgtaaca

SEQ ID NO: 534 - DNA, 26 bp, Artificial Sequence
  Description of Artificial Sequence Synthetic primer
cagaacaagaaatagcgttacccaaa

SEQ ID NO: 535 - DNA, 24 bp, Artificial Sequence
  Description of Artificial Sequence Synthetic primer
gccacctgaaagcttgtcttattc

SEQ ID NO: 536 - DNA, 24 bp, Artificial Sequence
  Description of Artificial Sequence Synthetic primer
tgcacatgaaactgcaccaaaatt

SEQ ID NO: 537 - DNA, 22 bp, Artificial Sequence
  Description of Artificial Sequence Synthetic primer
atgacgcaaaagctgatctact

SEQ ID NO: 538 - DNA, 24 bp, Artificial Sequence
  Description of Artificial Sequence Synthetic primer
gcacatgaaactgcaccacaattt

SEQ ID NO: 539 - DNA, 26 bp, Artificial Sequence
  Description of Artificial Sequence Synthetic primer
cagaacaagaaatagcgttacccaaa

SEQ ID NO: 540 - DNA, 25 bp, Artificial Sequence
  Description of Artificial Sequence Synthetic primer
gcaccacaatttcctgctaacatac

SEQ ID NO: 541 - DNA, 25 bp, Artificial Sequence
  Description of Artificial Sequence Synthetic primer
gcaattgaaccacctgaaagcttat

SEQ ID NO: 542 - DNA, 25 bp, Artificial Sequence
  Description of Artificial Sequence Synthetic primer
tgtcttattcccctagctctgaact

SEQ ID NO: 543 - DNA, 24 bp, Artificial Sequence
  Description of Artificial Sequence Synthetic primer
gcgttgaatgagtttgcgattgag

SEQ ID NO: 544 - DNA, 25 bp, Artificial Sequence
  Description of Artificial Sequence Synthetic primer
agaaagcacatccagatgttcaaca

SEQ ID NO: 545 - DNA, 25 bp, Artificial Sequence
  Description of Artificial Sequence Synthetic primer
gctagctcgtgaattgtttggtact

SEQ ID NO: 546 - DNA, 25 bp, Artificial Sequence
  Description of Artificial Sequence Synthetic primer
tgtgcctcttgttatgcagaagtta

SEQ ID NO: 547 - DNA, 16 bp, Artificial Sequence
  Description of Artificial Sequence Synthetic primer
tgcccgcggagaactg

SEQ ID NO: 548 - DNA, 26 bp, Artificial Sequence
  Description of Artificial Sequence Synthetic primer
aactaggcgcatactttaaaccagaa

SEQ ID NO: 549 - DNA, 25 bp, Artificial Sequence
  Description of Artificial Sequence Synthetic primer
accaaccactaaagaaacactcaca

SEQ ID NO: 550 - DNA, 25 bp, Artificial Sequence
  Description of Artificial Sequence Synthetic primer
caagaagacgagcaatccaaaatga

SEQ ID NO: 551 - DNA, 25 bp, Artificial Sequence
  Description of Artificial Sequence Synthetic primer
cattccagttggacgtttacaagaa

SEQ ID NO: 552 - DNA, 25 bp, Artificial Sequence
  Description of Artificial Sequence Synthetic primer
ggttctccgtttgacgatattttcc

SEQ ID NO: 553 - DNA, 21 bp, Artificial Sequence
  Description of Artificial Sequence Synthetic primer
tcgcttcgcttgtttctggaa

SEQ ID NO: 554 - DNA, 25 bp, Artificial Sequence
  Description of Artificial Sequence Synthetic primer
acaatcgatgtttccaaggaagtga

SEQ ID NO: 555 - DNA, 25 bp, Artificial Sequence
  Description of Artificial Sequence Synthetic primer
ggtcaatttgaagaacgcatgaaac

SEQ ID NO: 556 - DNA, 19 bp, Artificial Sequence
  Description of Artificial Sequence Synthetic primer
cgtccgtgtcatcggtgaa

SEQ ID NO: 557 - DNA, 17 bp, Artificial Sequence
  Description of Artificial Sequence Synthetic primer
gcgctggatccgcagaa

SEQ ID NO: 558 - DNA, 20 bp, Artificial Sequence
  Description of Artificial Sequence Synthetic primer
caattcgtcgtagccgtgtt

SEQ ID NO: 559 - DNA, 29 bp, Artificial Sequence
  Description of Artificial Sequence Synthetic primer
tccattgaaaaactagacgaaaatacggt

SEQ ID NO: 560 - DNA, 24 bp, Artificial Sequence
  Description of Artificial Sequence Synthetic primer
cgcacaagaaggcgtaactataga

SEQ ID NO: 561 - DNA, 22 bp, Artificial Sequence
  Description of Artificial Sequence Synthetic primer
acagggatccgtggtcaatttg

SEQ ID NO: 562 - DNA, 25 bp, Artificial Sequence
  Description of Artificial Sequence Synthetic primer
cagctaaagttcgtgacgagattac

SEQ ID NO: 563 - DNA, 21 bp, Artificial Sequence
  Description of Artificial Sequence Synthetic primer
agcgctcgttacatccaagac

SEQ ID NO: 564 - DNA, 22 bp, Artificial Sequence
  Description of Artificial Sequence Synthetic primer
cgcttgctgagtctatgtttgg

SEQ ID NO: 565 - DNA, 20 bp, Artificial Sequence
  Description of Artificial Sequence Synthetic primer
ggcctagcgaatgcagtgaa

SEQ ID NO: 566 - DNA, 30 bp, Artificial Sequence
  Description of Artificial Sequence Synthetic primer
agaagagctatctgtgaatagaaacagact

SEQ ID NO: 567 - DNA, 21 bp, Artificial Sequence
  Description of Artificial Sequence Synthetic primer
ctgtcaaagaccccgatggaa

SEQ ID NO: 568 - DNA, 26 bp, Artificial Sequence
  Description of Artificial Sequence Synthetic primer
gcagtgaaacaaaatttagggaagca

SEQ ID NO: 569 - DNA, 34 bp, Artificial Sequence
  Description of Artificial Sequence Synthetic primer
aaactagaggtattagatttgcatggtaatgaaa

SEQ ID NO: 570 - DNA, 25 bp, Artificial Sequence
  Description of Artificial Sequence Synthetic primer
gaacaaaagtacaagctgagagcat

SEQ ID NO: 571 - DNA, 29 bp, Artificial Sequence
  Description of Artificial Sequence Synthetic primer
ggcacaaaatttcaatggagataatagca

SEQ ID NO: 572 - DNA, 25 bp, Artificial Sequence
  Description of Artificial Sequence Synthetic primer
gaaagcaaagtgttacagaccttgt

SEQ ID NO: 573 - DNA, 24 bp, Artificial Sequence
  Description of Artificial Sequence Synthetic primer
tgaccttggtcctttgaaggattt

SEQ ID NO: 574 - DNA, 28 bp, Artificial Sequence
  Description of Artificial Sequence Synthetic primer
caggtggactaactagattgaagaaagt

SEQ ID NO: 575 - DNA, 26 bp, Artificial Sequence
  Description of Artificial Sequence Synthetic primer
ctagtgttaattgtgggtttgtgcat

SEQ ID NO: 576 - DNA, 32 bp, Artificial Sequence
  Description of Artificial Sequence Synthetic primer
tggagtacaaaatttcaatggagataatagca

SEQ ID NO: 577 - DNA, 24 bp, Artificial Sequence
  Description of Artificial Sequence Synthetic primer
cattaaatctcttgccggaatgca

SEQ ID NO: 578 - DNA, 25 bp, Artificial Sequence
  Description of Artificial Sequence Synthetic primer
ctgtacattaatacgggcttcggaa

SEQ ID NO: 579 - DNA, 24 bp, Artificial Sequence
  Description of Artificial Sequence Synthetic primer
ctgtcaaagaccctgatggaagat

SEQ ID NO: 580 - DNA, 28 bp, Artificial Sequence
  Description of Artificial Sequence Synthetic primer
caggtggactaactagattgaagaaagt

SEQ ID NO: 581 - DNA, 25 bp, Artificial Sequence
  Description of Artificial Sequence Synthetic primer
tggaacaaaagtacaagctgagagt

SEQ ID NO: 582 - DNA, 25 bp, Artificial Sequence
  Description of Artificial Sequence Synthetic primer
gaaagcaaagtgttacagaccttgt

SEQ ID NO: 583 - DNA, 25 bp, Artificial Sequence
  Description of Artificial Sequence Synthetic primer
gtgggagttatgtagatggttgtgt

SEQ ID NO: 584 - DNA, 24 bp, Artificial Sequence
  Description of Artificial Sequence Synthetic primer
ctgtcaaagacccagatggaagat

SEQ ID NO: 585 - DNA, 24 bp, Artificial Sequence
  Description of Artificial Sequence Synthetic primer
gtctgtgcattaatacgggttctg

SEQ ID NO: 586 - DNA, 26 bp, Artificial Sequence
  Description of Artificial Sequence Synthetic primer
gccatggactaaagacgattatccaa

SEQ ID NO: 587 - DNA, 25 bp, Artificial Sequence
  Description of Artificial Sequence Synthetic primer
atcggcaatgcgctattaaaagatg

SEQ ID NO: 588 - DNA, 24 bp, Artificial Sequence
  Description of Artificial Sequence Synthetic primer
cgcgaaaaagctattgagattggt

SEQ ID NO: 589 - DNA, 25 bp, Artificial Sequence
  Description of Artificial Sequence Synthetic primer
tgccgtggacaaaaaatgattatcc

SEQ ID NO: 590 - DNA, 22 bp, Artificial Sequence
  Description of Artificial Sequence Synthetic primer
tcggcaacgcgttactaaaaga

SEQ ID NO: 591 - DNA, 26 bp, Artificial Sequence
  Description of Artificial Sequence Synthetic primer
ggaaaaatctaagaaagaccgaacgc

SEQ ID NO: 592 - DNA, 24 bp, Artificial Sequence
  Description of Artificial Sequence Synthetic primer
acgattatccggattcgtggaaaa

SEQ ID NO: 593 - DNA, 23 bp, Artificial Sequence
  Description of Artificial Sequence Synthetic primer
cggcaacgcgctattaaaagatg

SEQ ID NO: 594 - DNA, 22 bp, Artificial Sequence
  Description of Artificial Sequence Synthetic primer
cgcgaaaaggcaattgaaatcg

SEQ ID NO: 595 - DNA, 25 bp, Artificial Sequence
  Description of Artificial Sequence Synthetic primer
gccgtggacaaaaaatgaatatcct

SEQ ID NO: 596 - DNA, 25 bp, Artificial Sequence
  Description of Artificial Sequence Synthetic primer
tgccatggactaaagacgattatcc

SEQ ID NO: 597 - DNA, 22 bp, Artificial Sequence
  Description of Artificial Sequence Synthetic primer
cgcgagaaggcaattgaaatcg

SEQ ID NO: 598 - DNA, 24 bp, Artificial Sequence
  Description of Artificial Sequence Synthetic primer
cgcgaaaaagctattgagattggt

SEQ ID NO: 599 - DNA, 21 bp, Artificial Sequence
  Description of Artificial Sequence Synthetic primer
agaaagaccgaacgcgagaag

SEQ ID NO: 600 - DNA, 25 bp, Artificial Sequence
  Description of Artificial Sequence Synthetic primer
cgcgctattaaaagatggctactca

SEQ ID NO: 601 - DNA, 26 bp, Artificial Sequence
  Description of Artificial Sequence Synthetic primer
ggaaaaatctaagaaagaccgaacgc

SEQ ID NO: 602 - DNA, 24 bp, Artificial Sequence
  Description of Artificial Sequence Synthetic primer
gccgtggacaaaaaacgattatcc

SEQ ID NO: 603 - DNA, 25 bp, Artificial Sequence
  Description of Artificial Sequence Synthetic primer
cgcgttactaaaagaaggctactca

SEQ ID NO: 604 - DNA, 24 bp, Artificial Sequence
  Description of Artificial Sequence Synthetic primer
tctaagaaagaccgaacgcgaaaa

SEQ ID NO: 605 - DNA, 24 bp, Artificial Sequence
  Description of Artificial Sequence Synthetic primer
acgattatccggattcgtggaaaa

SEQ ID NO: 606 - DNA, 26 bp, Artificial Sequence
  Description of Artificial Sequence Synthetic primer
tgtctgtaggtgaaaatgctaaagct

SEQ ID NO: 607 - DNA, 28 bp, Artificial Sequence
  Description of Artificial Sequence Synthetic primer
aataaagcagcatcttcaaacaaatcgt

SEQ ID NO: 608 - DNA, 25 bp, Artificial Sequence
  Description of Artificial Sequence Synthetic primer
tgcaagatggtgattcactatggaa

SEQ ID NO: 609 - DNA, 25 bp, Artificial Sequence
  Description of Artificial Sequence Synthetic primer
cggtgactcactttggaaaatctca

SEQ ID NO: 610 - DNA, 30 bp, Artificial Sequence
  Description of Artificial Sequence Synthetic primer
atcatagttggtcaaaaattgtctgtaggt

SEQ ID NO: 611 - DNA, 25 bp, Artificial Sequence
  Description of Artificial Sequence Synthetic primer
acaaatcgtctgcatctcaaggtaa

SEQ ID NO: 612 - DNA, 25 bp, Artificial Sequence
  Description of Artificial Sequence Synthetic primer
gcacaagggaatgtttcaaaagagt

SEQ ID NO: 613 - DNA, 25 bp, Artificial Sequence
  Description of Artificial Sequence Synthetic primer
gcagctccaacaactcaaaaatcaa

SEQ ID NO: 614 - DNA, 26 bp, Artificial Sequence
  Description of Artificial Sequence Synthetic primer
agtaacagagcaaccgaaagaagaaa

SEQ ID NO: 615 - DNA, 29 bp, Artificial Sequence
  Description of Artificial Sequence Synthetic primer
gctcaaacttaatttttccaaaccaaacg

SEQ ID NO: 616 - DNA, 27 bp, Artificial Sequence
  Description of Artificial Sequence Synthetic primer
ttaacggtaactgcaacagcatatagt

SEQ ID NO: 617 - DNA, 24 bp, Artificial Sequence
  Description of Artificial Sequence Synthetic primer
ttttccgaaccaaacattgcaagt

SEQ ID NO: 618 - DNA, 27 bp, Artificial Sequence
  Description of Artificial Sequence Synthetic primer
ggtaactgcaacagcatatagtaaagc

SEQ ID NO: 619 - DNA, 32 bp, Artificial Sequence
  Description of Artificial Sequence Synthetic primer
atctttcatctgatttaatcgtagttggtcaa

SEQ ID NO: 620 - DNA, 25 bp, Artificial Sequence
  Description of Artificial Sequence Synthetic primer
agccatctacaaataatgcggaaca

SEQ ID NO: 621 - DNA, 22 bp, Artificial Sequence
  Description of Artificial Sequence Synthetic primer
aggatacggacaagcaattgca

SEQ ID NO: 622 - DNA, 29 bp, Artificial Sequence
  Description of Artificial Sequence Synthetic primer
acaattcaacagcaaataattcagccaat

SEQ ID NO: 623 - DNA, 26 bp, Artificial Sequence
  Description of Artificial Sequence Synthetic primer
catcagcaaataaagcagcatcttca

SEQ ID NO: 624 - DNA, 30 bp, Artificial Sequence
  Description of Artificial Sequence Synthetic primer
ggtcaaaagttatctatcggaagtaaagct

SEQ ID NO: 625 - DNA, 28 bp, Artificial Sequence
  Description of Artificial Sequence Synthetic primer
cggtgtgacagttaaccaattaaaagaa

SEQ ID NO: 626 - DNA, 22 bp, Artificial Sequence
  Description of Artificial Sequence Synthetic primer
aggatacggacaagcaattgca

SEQ ID NO: 627 - DNA, 25 bp, Artificial Sequence
  Description of Artificial Sequence Synthetic primer
gacgtattcgttcacttctcagcta

SEQ ID NO: 628 - DNA, 21 bp, Artificial Sequence
  Description of Artificial Sequence Synthetic primer
agaaggccaagcggtaacatt

SEQ ID NO: 629 - DNA, 23 bp, Artificial Sequence
  Description of Artificial Sequence Synthetic primer
tcgtacatttcagcgctatccaa

SEQ ID NO: 630 - DNA, 23 bp, Artificial Sequence
  Description of Artificial Sequence Synthetic primer
gaaggcggagatgatgtattcgt

SEQ ID NO: 631 - DNA, 25 bp, Artificial Sequence
  Description of Artificial Sequence Synthetic primer
caaggtgacggctacaaatctttag

SEQ ID NO: 632 - DNA, 25 bp, Artificial Sequence
  Description of Artificial Sequence Synthetic primer
atgcaaacaggtacagttaaatggt

SEQ ID NO: 633 - DNA, 25 bp, Artificial Sequence
  Description of Artificial Sequence Synthetic primer
caaggtgacggctacaaatctttag

SEQ ID NO: 634 - DNA, 22 bp, Artificial Sequence
  Description of Artificial Sequence Synthetic primer
gcgctatcgaaggtgaaggatt

SEQ ID NO: 635 - DNA, 25 bp, Artificial Sequence
  Description of Artificial Sequence Synthetic primer
ggatttggttttatcgaacgcgaaa

SEQ ID NO: 636 - DNA, 22 bp, Artificial Sequence
  Description of Artificial Sequence Synthetic primer
gcgctatcgaaggtgaaggatt

SEQ ID NO: 637 - DNA, 25 bp, Artificial Sequence
  Description of Artificial Sequence Synthetic primer
atgcaaacaggtacagttaaatggt

SEQ ID NO: 638 - DNA, 25 bp, Artificial Sequence
  Description of Artificial Sequence Synthetic primer
atgcaaacaggtacagttaaatggt

SEQ ID NO: 639 - DNA, 25 bp, Artificial Sequence
  Description of Artificial Sequence Synthetic primer
caaggtgacggctacaaatctttag

SEQ ID NO: 640 - DNA, 23 bp, Artificial Sequence
  Description of Artificial Sequence Synthetic primer
gcgaaaacggtgacgatgtattc

SEQ ID NO: 641 - DNA, 26 bp, Artificial Sequence
  Description of Artificial Sequence Synthetic primer
ggcggagatgatatattcgtacactt

SEQ ID NO: 642 - DNA, 25 bp, Artificial Sequence
  Description of Artificial Sequence Synthetic primer
tcgaagtagaaggcggagatgatat

SEQ ID NO: 643 - DNA, 25 bp, Artificial Sequence
  Description of Artificial Sequence Synthetic primer
caaggtgacggctacaaatctttag

SEQ ID NO: 644 - DNA, 21 bp, Artificial Sequence
  Description of Artificial Sequence Synthetic primer
gacggcggcgaagatattttc

SEQ ID NO: 645 - DNA, 21 bp, Artificial Sequence
  Description of Artificial Sequence Synthetic primer
agacggcggcgaagatatttt

SEQ ID NO: 646 - DNA, 27 bp, Artificial Sequence
  Description of Artificial Sequence Synthetic primer
atgcaaaatgggaaagtaaaatggttt

SEQ ID NO: 647 - DNA, 24 bp, Artificial Sequence
  Description of Artificial Sequence Synthetic primer
gaaggcggagatgatgtattcgta

SEQ ID NO: 648 - DNA, 25 bp, Artificial Sequence
  Description of Artificial Sequence Synthetic primer
tcactgatgaagttacgcaaacaga

SEQ ID NO: 649 - DNA, 25 bp, Artificial Sequence
  Description of Artificial Sequence Synthetic primer
gccactttcaggattgacacagtta

SEQ ID NO: 650 - DNA, 25 bp, Artificial Sequence
  Description of Artificial Sequence Synthetic primer
cccgctgacgaaccttactaaatta

SEQ ID NO: 651 - DNA, 21 bp, Artificial Sequence
  Description of Artificial Sequence Synthetic primer
gcggaaactggtgggaatgaa

SEQ ID NO: 652 - DNA, 24 bp, Artificial Sequence
  Description of Artificial Sequence Synthetic primer
ccgacgaaagatggctacacattt

SEQ ID NO: 653 - DNA, 23 bp, Artificial Sequence
  Description of Artificial Sequence Synthetic primer
cgcaactagcaaaatgccaacta

SEQ ID NO: 654 - DNA, 25 bp, Artificial Sequence
  Description of Artificial Sequence Synthetic primer
gtgacacctcttgctaatctaacga

SEQ ID NO: 655 - DNA, 30 bp, Artificial Sequence
  Description of Artificial Sequence Synthetic primer
agtttcacgagtgaggttagttatgatttt

SEQ ID NO: 656 - DNA, 25 bp, Artificial Sequence
  Description of Artificial Sequence Synthetic primer
gtgaagtagtaccaccaacaacgat

SEQ ID NO: 657 - DNA, 25 bp, Artificial Sequence
  Description of Artificial Sequence Synthetic primer
gacagaaaacgtggtagtggataca

SEQ ID NO: 658 - DNA, 25 bp, Artificial Sequence
  Description of Artificial Sequence Synthetic primer
cccccggaaatgatgataaaaacga

SEQ ID NO: 659 - DNA, 25 bp, Artificial Sequence
  Description of Artificial Sequence Synthetic primer
gccactttcaggattgacacagtta

SEQ ID NO: 660 - DNA, 25 bp, Artificial Sequence
  Description of Artificial Sequence Synthetic primer
tgacaccacttgctaatctaacgaa

SEQ ID NO: 661 - DNA, 18 bp, Artificial Sequence
  Description of Artificial Sequence Synthetic primer
cgctctacgcccagttca

SEQ ID NO: 662 - DNA, 26 bp, Artificial Sequence
  Description of Artificial Sequence Synthetic primer
cttgtagtgacagctatcttgggaat

SEQ ID NO: 663 - DNA, 22 bp, Artificial Sequence
  Description of Artificial Sequence Synthetic primer
aagctcaggcagagagtattgc

SEQ ID NO: 664 - DNA, 22 bp, Artificial Sequence
  Description of Artificial Sequence Synthetic primer
tgctgaaccgacaaaagaaggt

SEQ ID NO: 665 - DNA, 25 bp, Artificial Sequence
  Description of Artificial Sequence Synthetic primer
gacaacaacagaaaacgtgacagta

SEQ ID NO: 666 - DNA, 25 bp, Artificial Sequence
  Description of Artificial Sequence Synthetic primer
gcgcccgtgaattacattactacat

SEQ ID NO: 667 - DNA, 25 bp, Artificial Sequence
  Description of Artificial Sequence Synthetic primer
acagctcccggaaaagatgataaaa

SEQ ID NO: 668 - DNA, 25 bp, Artificial Sequence
  Description of Artificial Sequence Synthetic primer
gcgaagcactcaaagacagttttaa

SEQ ID NO: 669 - DNA, 20 bp, Artificial Sequence
  Description of Artificial Sequence Synthetic primer
tgggcgctttcatccctatg

SEQ ID NO: 670 - DNA, 25 bp, Artificial Sequence
  Description of Artificial Sequence Synthetic primer
tgtttttgaagagctgacgttgaaa

SEQ ID NO: 671 - DNA, 19 bp, Artificial Sequence
  Description of Artificial Sequence Synthetic primer
gcgacgtgcttctgacaga

SEQ ID NO: 672 - DNA, 24 bp, Artificial Sequence
  Description of Artificial Sequence Synthetic primer
gaaactaacccagcacttctacgt

SEQ ID NO: 673 - DNA, 26 bp, Artificial Sequence
  Description of Artificial Sequence Synthetic primer
agaaaaattctgcgaagcactcaaag

SEQ ID NO: 674 - DNA, 23 bp, Artificial Sequence
  Description of Artificial Sequence Synthetic primer
caacgtggaaagagctcaacaag

SEQ ID NO: 675 - DNA, 21 bp, Artificial Sequence
  Description of Artificial Sequence Synthetic primer
tgagcgagcgcagacttttat

SEQ ID NO: 676 - DNA, 25 bp, Artificial Sequence
  Description of Artificial Sequence Synthetic primer
ggttacgtcaccgaaaaatggaaat

SEQ ID NO: 677 - DNA, 25 bp, Artificial Sequence
  Description of Artificial Sequence Synthetic primer
gcgcatacgtgtaggtgaaattaaa

SEQ ID NO: 678 - DNA, 19 bp, Artificial Sequence
  Description of Artificial Sequence Synthetic primer
tgatgcggaagtcgagcaa

SEQ ID NO: 679 - DNA, 22 bp, Artificial Sequence
  Description of Artificial Sequence Synthetic primer
agagaaatttggcgcagaacac

SEQ ID NO: 680 - DNA, 26 bp, Artificial Sequence
  Description of Artificial Sequence Synthetic primer
tgagtgaaggtaaggtagtcgatgaa

SEQ ID NO: 681 - DNA, 23 bp, Artificial Sequence
  Description of Artificial Sequence Synthetic primer
gttgagaaagaagcgcgtattcg

SEQ ID NO: 682 - DNA, 17 bp, Artificial Sequence
  Description of Artificial Sequence Synthetic primer
cgcatcggcgtcccatt

SEQ ID NO: 683 - DNA, 26 bp, Artificial Sequence
  Description of Artificial Sequence Synthetic primer
ccgaacgatagttacattttctgcat

SEQ ID NO: 684 - DNA, 29 bp, Artificial Sequence
  Description of Artificial Sequence Synthetic primer
cttaacatgctagaagttggagatgtact

SEQ ID NO: 685 - DNA, 21 bp, Artificial Sequence
  Description of Artificial Sequence Synthetic primer
agcgagcgcagacttttatct

SEQ ID NO: 686 - DNA, 17 bp, Artificial Sequence
  Description of Artificial Sequence Synthetic primer
cgcatcggcgtcccatt

SEQ ID NO: 687 - DNA, 23 bp, Artificial Sequence
  Description of Artificial Sequence Synthetic primer
ggagtttgtttcccagcgtattc

SEQ ID NO: 688 - DNA, 20 bp, Artificial Sequence
  Description of Artificial Sequence Synthetic primer
ggcgacgtccttctaacaga

SEQ ID NO: 689 - DNA, 23 bp, Artificial Sequence
  Description of Artificial Sequence Synthetic primer
ggatgtaacgcaggtagctcaaa

SEQ ID NO: 690 - DNA, 25 bp, Artificial Sequence
  Description of Artificial Sequence Synthetic primer
gggcaaaaaagccgttaatgatcta

SEQ ID NO: 691 - DNA, 25 bp, Artificial Sequence
  Description of Artificial Sequence Synthetic primer
cgacttattgagccaacagaaggaa

SEQ ID NO: 692 - DNA, 25 bp, Artificial Sequence
  Description of Artificial Sequence Synthetic primer
acgaatccagtgtctattacagcag

SEQ ID NO: 693 - DNA, 25 bp, Artificial Sequence
  Description of Artificial Sequence Synthetic primer
ggctttatcgacgtagagcaaattg

SEQ ID NO: 694 - DNA, 26 bp, Artificial Sequence
  Description of Artificial Sequence Synthetic primer
agaaaaaacaagaacgggcaaaagaa

SEQ ID NO: 695 - DNA, 26 bp, Artificial Sequence
  Description of Artificial Sequence Synthetic primer
atacgttacttgttgtggatgaaggt

SEQ ID NO: 696 - DNA, 25 bp, Artificial Sequence
  Description of Artificial Sequence Synthetic primer
ttgtgggaaaacgacaacaatgaag

SEQ ID NO: 697 - DNA, 29 bp, Artificial Sequence
  Description of Artificial Sequence Synthetic primer
tcgaacacgtaacaaagacttataaaggg

SEQ ID NO: 698 - DNA, 29 bp, Artificial Sequence
  Description of Artificial Sequence Synthetic primer
gtgttaaaattcgaacacgtaacaaagac

SEQ ID NO: 699 - DNA, 25 bp, Artificial Sequence
  Description of Artificial Sequence Synthetic primer
ccacatatgacgattcgcgaaaata

SEQ ID NO: 700 - DNA, 28 bp, Artificial Sequence
  Description of Artificial Sequence Synthetic primer
tggttatgttattcagcaaattggcttg

SEQ ID NO: 701 - DNA, 26 bp, Artificial Sequence
  Description of Artificial Sequence Synthetic primer
ccaattactcgtgattccctacaaga

SEQ ID NO: 702 - DNA, 25 bp, Artificial Sequence
  Description of Artificial Sequence Synthetic primer
cgtgccagcttagttgatatcgtat

SEQ ID NO: 703 - DNA, 26 bp, Artificial Sequence
  Description of Artificial Sequence Synthetic primer
ggatttatcgacgtagagcaaatcga

SEQ ID NO: 704 - DNA, 25 bp, Artificial Sequence
  Description of Artificial Sequence Synthetic primer
cttgtagtcgacgaagggaatgtat

SEQ ID NO: 705 - DNA, 27 bp, Artificial Sequence
  Description of Artificial Sequence Synthetic primer
gatgaagcaataaaacttgcagatcgt

SEQ ID NO: 706 - DNA, 27 bp, Artificial Sequence
  Description of Artificial Sequence Synthetic primer
gggcaaaaaagctgttaatgatctaac

SEQ ID NO: 707 - DNA, 21 bp, Artificial Sequence
  Description of Artificial Sequence Synthetic primer
caagtggttgtgggaaaacga

SEQ ID NO: 708 - DNA, 25 bp, Artificial Sequence
  Description of Artificial Sequence Synthetic primer
acggattctaaaacgcggctataaa

SEQ ID NO: 709 - DNA, 25 bp, Artificial Sequence
  Description of Artificial Sequence Synthetic primer
tgggaaaacgacaacaatgaagatg

SEQ ID NO: 710 - DNA, 23 bp, Artificial Sequence
  Description of Artificial Sequence Synthetic primer
cgacacgaacaaactcacaaaga

SEQ ID NO: 711 - DNA, 24 bp, Artificial Sequence
  Description of Artificial Sequence Synthetic primer
cagcgaagaacaactagctactct

SEQ ID NO: 712 - DNA, 29 bp, Artificial Sequence
  Description of Artificial Sequence Synthetic primer
gagttgtagaaattcactatgttgacgaa

SEQ ID NO: 713 - DNA, 24 bp, Artificial Sequence
  Description of Artificial Sequence Synthetic primer
acacaagtagccaaacagtgacat

SEQ ID NO: 714 - DNA, 23 bp, Artificial Sequence
  Description of Artificial Sequence Synthetic primer
ccaatgcaactggacaattcaca

SEQ ID NO: 715 - DNA, 23 bp, Artificial Sequence
  Description of Artificial Sequence Synthetic primer
cgacacgaacaaactcacaaagt

SEQ ID NO: 716 - DNA, 18 bp, Artificial Sequence
  Description of Artificial Sequence Synthetic primer
ccgccgaaccaaccaatg

SEQ ID NO: 717 - DNA, 25 bp, Artificial Sequence
  Description of Artificial Sequence Synthetic primer
cagcagagcctgtaacagtgaatta

SEQ ID NO: 718 - DNA, 23 bp, Artificial Sequence
  Description of Artificial Sequence Synthetic primer
cgacacgaacaaactcacaaagt

SEQ ID NO: 719 - DNA, 25 bp, Artificial Sequence
  Description of Artificial Sequence Synthetic primer
accgttaattacgtggacgatactg

SEQ ID NO: 720 - DNA, 26 bp, Artificial Sequence
  Description of Artificial Sequence Synthetic primer
accccgcttacacagttaacatattt

SEQ ID NO: 721 - DNA, 17 bp, Artificial Sequence
  Description of Artificial Sequence Synthetic primer
cgcgcaaaccgtcaact

SEQ ID NO: 722 - DNA, 22 bp, Artificial Sequence
  Description of Artificial Sequence Synthetic primer
aggagaaaaattggcggctgat

SEQ ID NO: 723 - DNA, 26 bp, Artificial Sequence
  Description of Artificial Sequence Synthetic primer
agtaggaaccgtaacaactccatttg

SEQ ID NO: 724 - DNA, 21 bp, Artificial Sequence
  Description of Artificial Sequence Synthetic primer
gtagcagcagagcctgtaaca

SEQ ID NO: 725 - DNA, 28 bp, Artificial Sequence
  Description of Artificial Sequence Synthetic primer
agctaactttcctaaattgctccagtaa

SEQ ID NO: 726 - DNA, 20 bp, Artificial Sequence
  Description of Artificial Sequence Synthetic primer
aagcagcagagcctgtaaca

SEQ ID NO: 727 - DNA, 24 bp, Artificial Sequence
  Description of Artificial Sequence Synthetic primer
acacaagtagccaaacagtgacat

SEQ ID NO: 728 - DNA, 24 bp, Artificial Sequence
  Description of Artificial Sequence Synthetic primer
caatgcgactggacaattcacaag

SEQ ID NO: 729 - DNA, 28 bp, Artificial Sequence
  Description of Artificial Sequence Synthetic primer
gatctttcacaaaaccccaaattagtct

SEQ ID NO: 730 - DNA, 20 bp, Artificial Sequence
  Description of Artificial Sequence Synthetic primer
aacaggcgattccacaccat

SEQ ID NO: 731 - DNA, 22 bp, Artificial Sequence
  Description of Artificial Sequence Synthetic primer
gcgcttggatggatgaatatcg

SEQ ID NO: 732 - DNA, 23 bp, Artificial Sequence
  Description of Artificial Sequence Synthetic primer
gcgcttggatggatgaatattgg

SEQ ID NO: 733 - DNA, 26 bp, Artificial Sequence
  Description of Artificial Sequence Synthetic primer
gcagcatttactgcgtttaacttagt

SEQ ID NO: 734 - DNA, 28 bp, Artificial Sequence
  Description of Artificial Sequence Synthetic primer
caatggttattaacatgatgtccgctaa

SEQ ID NO: 735 - DNA, 24 bp, Artificial Sequence
  Description of Artificial Sequence Synthetic primer
aacgtagctggtggatttatcgtt

SEQ ID NO: 736 - DNA, 25 bp, Artificial Sequence
  Description of Artificial Sequence Synthetic primer
gttggtacacaagcagtcgaaaatt

SEQ ID NO: 737 - DNA, 22 bp, Artificial Sequence
  Description of Artificial Sequence Synthetic primer
cttgaaatgatcgcgcttggat

SEQ ID NO: 738 - DNA, 21 bp, Artificial Sequence
  Description of Artificial Sequence Synthetic primer
agttggtactagcgccgtaga

SEQ ID NO: 739 - DNA, 25 bp, Artificial Sequence
  Description of Artificial Sequence Synthetic primer
ggtggatttatcgttgttgttggtt

SEQ ID NO: 740 - DNA, 25 bp, Artificial Sequence
  Description of Artificial Sequence Synthetic primer
gactggattcatgtttcagcactac

SEQ ID NO: 741 - DNA, 25 bp, Artificial Sequence
  Description of Artificial Sequence Synthetic primer
agtctcgactggattcatgtttcag

SEQ ID NO: 742 - DNA, 26 bp, Artificial Sequence
  Description of Artificial Sequence Synthetic primer
cctttcttcttcttaggctttgttgt

SEQ ID NO: 743 - DNA, 28 bp, Artificial Sequence
  Description of Artificial Sequence Synthetic primer
agcctgtacattaattggtcttgttctt

SEQ ID NO: 744 - DNA, 25 bp, Artificial Sequence
  Description of Artificial Sequence Synthetic primer
gggaaagaaggaaatttgcgaagtc

SEQ ID NO: 745 - DNA, 25 bp, Artificial Sequence
  Description of Artificial Sequence Synthetic primer
gctgctcttggtcaagtacttacaa

SEQ ID NO: 746 - DNA, 22 bp, Artificial Sequence
  Description of Artificial Sequence Synthetic primer
gcgcttggatggatgaatatcg

SEQ ID NO: 747 - DNA, 24 bp, Artificial Sequence
  Description of Artificial Sequence Synthetic primer
ggtaaggaaggtaatctgcgaagt

SEQ ID NO: 748 - DNA, 26 bp, Artificial Sequence
  Description of Artificial Sequence Synthetic primer
gcagcattcactcaattcaacttagt

SEQ ID NO: 749 - DNA, 25 bp, Artificial Sequence
  Description of Artificial Sequence Synthetic primer
gctgctcttggtcaggtacttacta

SEQ ID NO: 750 - DNA, 26 bp, Artificial Sequence
  Description of Artificial Sequence Synthetic primer
gtagtattttggatgagtggcaaacg

SEQ ID NO: 751 - DNA, 19 bp, Artificial Sequence
  Description of Artificial Sequence Synthetic primer
gaaatgatcgcgcttggct

SEQ ID NO: 752 - DNA, 23 bp, Artificial Sequence
  Description of Artificial Sequence Synthetic primer
cgcaaacgatgtcattgtaccaa

SEQ ID NO: 753 - DNA, 26 bp, Artificial Sequence
  Description of Artificial Sequence Synthetic primer
gaaccacacaaaaaagccattagtca

SEQ ID NO: 754 - DNA, 23 bp, Artificial Sequence
  Description of Artificial Sequence Synthetic primer
gaagcaggaatacggtacatcga

SEQ ID NO: 755 - DNA, 25 bp, Artificial Sequence
  Description of Artificial Sequence Synthetic primer
gcgggaaagcatattcgcttaataa

SEQ ID NO: 756 - DNA, 25 bp, Artificial Sequence
  Description of Artificial Sequence Synthetic primer
ccagaactgacacgagcaataaaat

SEQ ID NO: 757 - DNA, 25 bp, Artificial Sequence
  Description of Artificial Sequence Synthetic primer
agacgagcaaaatagcaacgatagt

SEQ ID NO: 758 - DNA, 25 bp, Artificial Sequence
  Description of Artificial Sequence Synthetic primer
accaacaactagaagcaggaatacg

SEQ ID NO: 759 - DNA, 25 bp, Artificial Sequence
  Description of Artificial Sequence Synthetic primer
caatggtccgagtgtgaaaacaaaa

SEQ ID NO: 760 - DNA, 23 bp, Artificial Sequence
  Description of Artificial Sequence Synthetic primer
cgagcaaaacagcaacgatagtt

SEQ ID NO: 761 - DNA, 29 bp, Artificial Sequence
  Description of Artificial Sequence Synthetic primer
cggatccaaccattaatcaacatttacaa

SEQ ID NO: 762 - DNA, 27 bp, Artificial Sequence
  Description of Artificial Sequence Synthetic primer
gcatcactttcaggtgtattagaaacg

SEQ ID NO: 763 - DNA, 25 bp, Artificial Sequence
  Description of Artificial Sequence Synthetic primer
ccagaactgacacgagcaataaaat

SEQ ID NO: 764 - DNA, 24 bp, Artificial Sequence
  Description of Artificial Sequence Synthetic primer
cagcactctctataccaggtacac

SEQ ID NO: 765 - DNA, 26 bp, Artificial Sequence
  Description of Artificial Sequence Synthetic primer
tgatacgatgagctataacggagaca

SEQ ID NO: 766 - DNA, 27 bp, Artificial Sequence
  Description of Artificial Sequence Synthetic primer
caatctcaaaatttaccatgggccaat

SEQ ID NO: 767 - DNA, 29 bp, Artificial Sequence
  Description of Artificial Sequence Synthetic primer
ttgtataagaattatttgcaacgcacatt

SEQ ID NO: 768 - DNA, 25 bp, Artificial Sequence
  Description of Artificial Sequence Synthetic primer
gatacgatgagttacaacggagaca

SEQ ID NO: 769 - DNA, 24 bp, Artificial Sequence
  Description of Artificial Sequence Synthetic primer
aaaccactagctcaaacacaaacg

SEQ ID NO: 770 - DNA, 23 bp, Artificial Sequence
  Description of Artificial Sequence Synthetic primer
gaagcaggaatacggtacatcga

SEQ ID NO: 771 - DNA, 26 bp, Artificial Sequence
  Description of Artificial Sequence Synthetic primer
attcaaagagattgtccagactgctt

SEQ ID NO: 772 - DNA, 25 bp, Artificial Sequence
  Description of Artificial Sequence Synthetic primer
acgcaaacgatgtcattgtatcaac

SEQ ID NO: 773 - DNA, 25 bp, Artificial Sequence
  Description of Artificial Sequence Synthetic primer
cggtatcctagctgaaacatatcca

SEQ ID NO: 774 - DNA, 26 bp, Artificial Sequence
  Description of Artificial Sequence Synthetic primer
aaaaccattgcaagcattaagacgaa

SEQ ID NO: 775 - DNA, 24 bp, Artificial Sequence
  Description of Artificial Sequence Synthetic primer
ccgttttcattgtggagcttgatc

SEQ ID NO: 776 - DNA, 24 bp, Artificial Sequence
  Description of Artificial Sequence Synthetic primer
ccgttttcattgtggagcttgatc

SEQ ID NO: 777 - DNA, 23 bp, Artificial Sequence
  Description of Artificial Sequence Synthetic primer
gcaaacggtggtcgtaagaaaac

SEQ ID NO: 778 - DNA, 26 bp, Artificial Sequence
  Description of Artificial Sequence Synthetic primer
aaaaccattgcaagcattaagacgaa

SEQ ID NO: 779 - DNA, 26 bp, Artificial Sequence
  Description of Artificial Sequence Synthetic primer
aaaaccattgcaagcattaagacgaa

SEQ ID NO: 780 - DNA, 17 bp, Artificial Sequence
  Description of Artificial Sequence Synthetic primer
agcaaacggcggtcgta

SEQ ID NO: 781 - DNA, 22 bp, Artificial Sequence
  Description of Artificial Sequence Synthetic primer
caaacggcggtcgtaagaaaac

SEQ ID NO: 782 - DNA, 24 bp, Artificial Sequence
  Description of Artificial Sequence Synthetic primer
ccgttttcattgtggagcttgatc

SEQ ID NO: 783 - DNA, 26 bp, Artificial Sequence
  Description of Artificial Sequence Synthetic primer
aaaaccattgcaagcattaagacgaa

SEQ ID NO: 784 - DNA, 24 bp, Artificial Sequence
  Description of Artificial Sequence Synthetic primer
ccgttttcattgtggagcttgatc

SEQ ID NO: 785 - DNA, 25 bp, Artificial Sequence
  Description of Artificial Sequence Synthetic primer
cggtatcctagctgaaacatatcca

SEQ ID NO: 786 - DNA, 23 bp, Artificial Sequence
  Description of Artificial Sequence Synthetic primer
cgattgaacgttgcggtatccta

SEQ ID NO: 787 - DNA, 21 bp, Artificial Sequence
  Description of Artificial Sequence Synthetic primer
tgaaagcaaacggtggtcgta

SEQ ID NO: 788 - DNA, 26 bp, Artificial Sequence
  Description of Artificial Sequence Synthetic primer
aaaaccattgcaagcattaagacgaa

SEQ ID NO: 789 - DNA, 23 bp, Artificial Sequence
  Description of Artificial Sequence Synthetic primer
cgattgaacgttgcggtatccta

SEQ ID NO: 790 - DNA, 26 bp, Artificial Sequence
  Description of Artificial Sequence Synthetic primer
aaaaccattgcaagcattaagacgaa

SEQ ID NO: 791 - DNA, 23 bp, Artificial Sequence
  Description of Artificial Sequence Synthetic primer
gcgatactttaccaggagtagct

SEQ ID NO: 792 - DNA, 25 bp, Artificial Sequence
  Description of Artificial Sequence Synthetic primer
cgaaacagattttggtccacttgaa

SEQ ID NO: 793 - DNA, 25 bp, Artificial Sequence
  Description of Artificial Sequence Synthetic primer
gcggaccgcaattttgaattatcta

SEQ ID NO: 794 - DNA, 27 bp, Artificial Sequence
  Description of Artificial Sequence Synthetic primer
caaacagaccaagtaaatccaattgct

SEQ ID NO: 795 - DNA, 24 bp, Artificial Sequence
  Description of Artificial Sequence Synthetic primer
caacagattacggtagcagctttt

SEQ ID NO: 796 - DNA, 25 bp, Artificial Sequence
  Description of Artificial Sequence Synthetic primer
cgaaacagattttggtccacttgaa

SEQ ID NO: 797 - DNA, 25 bp, Artificial Sequence
  Description of Artificial Sequence Synthetic primer
cggtgtctagaaatgccactgttaa

SEQ ID NO: 798 - DNA, 25 bp, Artificial Sequence
  Description of Artificial Sequence Synthetic primer
ccattgatcaaacaacaggaacgat

SEQ ID NO: 799 - DNA, 26 bp, Artificial Sequence
  Description of Artificial Sequence Synthetic primer
gttgacacctattgataatgccgttt

SEQ ID NO: 800 - DNA, 30 bp, Artificial Sequence
  Description of Artificial Sequence Synthetic primer
gacgaaacaaaaaagctatacgaataaagc

SEQ ID NO: 801 - DNA, 23 bp, Artificial Sequence
  Description of Artificial Sequence Synthetic primer
caacagattacggcagcagtttt

SEQ ID NO: 802 - DNA, 25 bp, Artificial Sequence
  Description of Artificial Sequence Synthetic primer
cataacctccttggaaatgctacca

SEQ ID NO: 803 - DNA, 29 bp, Artificial Sequence
  Description of Artificial Sequence Synthetic primer
atgaaaaaaagatggttggtatttgcaat

SEQ ID NO: 804 - DNA, 30 bp, Artificial Sequence
  Description of Artificial Sequence Synthetic primer
acttacgatactaaaatcaccacgaaacaa

SEQ ID NO: 805 - DNA, 25 bp, Artificial Sequence
  Description of Artificial Sequence Synthetic primer
gccggtcaaaaattacaactaacga

SEQ ID NO: 806 - DNA, 26 bp, Artificial Sequence
  Description of Artificial Sequence Synthetic primer
cagactttggaccaattgaagttgtc

SEQ ID NO: 807 - DNA, 25 bp, Artificial Sequence
  Description of Artificial Sequence Synthetic primer
cggatactttacaagccggtcaaaa

SEQ ID NO: 808 - DNA, 25 bp, Artificial Sequence
  Description of Artificial Sequence Synthetic primer
ctgtgaaaaatacgacccaaacgat

SEQ ID NO: 809 - DNA, 25 bp, Artificial Sequence
  Description of Artificial Sequence Synthetic primer
ccaccaatatccaaccaattacgct

SEQ ID NO: 810 - DNA, 25 bp, Artificial Sequence
  Description of Artificial Sequence Synthetic primer
ggcgaatggatgataaagacttgag

SEQ ID NO: 811 - DNA, 25 bp, Artificial Sequence
  Description of Artificial Sequence Synthetic primer
agttgcaagtggagaaacaatgact

SEQ ID NO: 812 - DNA, 24 bp, Artificial Sequence
  Description of Artificial Sequence Synthetic primer
gatggtattacagtcagcggtgta

SEQ ID NO: 813 - DNA, 27 bp, Artificial Sequence
  Description of Artificial Sequence Synthetic primer
gccagctttaaatacgcttttcttaca

SEQ ID NO: 814 - DNA, 25 bp, Artificial Sequence
  Description of Artificial Sequence Synthetic primer
ggaagctggaaatctggtaacacta

SEQ ID NO: 815 - DNA, 25 bp, Artificial Sequence
  Description of Artificial Sequence Synthetic primer
cgcgacacttacaagcgattttaat

SEQ ID NO: 816 - DNA, 24 bp, Artificial Sequence
  Description of Artificial Sequence Synthetic primer
ttaagcttgatgtaccaggcgaat

SEQ ID NO: 817 - DNA, 25 bp, Artificial Sequence
  Description of Artificial Sequence Synthetic primer
acacgttagatttaacagcggatga

SEQ ID NO: 818 - DNA, 25 bp, Artificial Sequence
  Description of Artificial Sequence Synthetic primer
gggcggagacaatgtaaaagatagt

SEQ ID NO: 819 - DNA, 27 bp, Artificial Sequence
  Description of Artificial Sequence Synthetic primer
caggtgcgaattatagcgatttaacag

SEQ ID NO: 820 - DNA, 25 bp, Artificial Sequence
  Description of Artificial Sequence Synthetic primer
gctggtctaaaagcggatccaataa

SEQ ID NO: 821 - DNA, 27 bp, Artificial Sequence
  Description of Artificial Sequence Synthetic primer
ccaaacacttgaaccgattaaaaacca

SEQ ID NO: 822 - DNA, 23 bp, Artificial Sequence
  Description of Artificial Sequence Synthetic primer
ttgccagcattaacaacgctttt

SEQ ID NO: 823 - DNA, 25 bp, Artificial Sequence
  Description of Artificial Sequence Synthetic primer
ggttggtggagacaatgtaacagat

SEQ ID NO: 824 - DNA, 27 bp, Artificial Sequence
  Description of Artificial Sequence Synthetic primer
ccagccttaacaacacttttcttacaa

SEQ ID NO: 825 - DNA, 24 bp, Artificial Sequence
  Description of Artificial Sequence Synthetic primer
gaagaaaccgacccaacaatcatc

SEQ ID NO: 826 - DNA, 22 bp, Artificial Sequence
  Description of Artificial Sequence Synthetic primer
agcctctttgccagcattaaca

SEQ ID NO: 827 - DNA, 25 bp, Artificial Sequence
  Description of Artificial Sequence Synthetic primer
cccaggcgaatataccgtaacttta

SEQ ID NO: 828 - DNA, 29 bp, Artificial Sequence
  Description of Artificial Sequence Synthetic primer
cgacttccgcaagtaatacttatttagga

SEQ ID NO: 829 - DNA, 25 bp, Artificial Sequence
  Description of Artificial Sequence Synthetic primer
cacggtcacattaaacgcagaaaat

SEQ ID NO: 830 - DNA, 19 bp, Artificial Sequence
  Description of Artificial Sequence Synthetic primer
tggcacggttgctcctttt

SEQ ID NO: 831 - DNA, 25 bp, Artificial Sequence
  Description of Artificial Sequence Synthetic primer
ctggaaatgccgttttaacaagtga

SEQ ID NO: 832 - DNA, 23 bp, Artificial Sequence
  Description of Artificial Sequence Synthetic primer
agaccctgtaaccgtagttgtga

SEQ ID NO: 833 - DNA, 25 bp, Artificial Sequence
  Description of Artificial Sequence Synthetic primer
cgactgttgtagatctttccaaacc

SEQ ID NO: 834 - DNA, 25 bp, Artificial Sequence
  Description of Artificial Sequence Synthetic primer
aggactaaaagctagccaagatgtc

SEQ ID NO: 835 - DNA, 25 bp, Artificial Sequence
  Description of Artificial Sequence Synthetic primer
cgggcttagtaatcttgaacgctta

SEQ ID NO: 836 - DNA, 25 bp, Artificial Sequence
  Description of Artificial Sequence Synthetic primer
ggaaccgcgattacaagtgattttg

SEQ ID NO: 837 - DNA, 25 bp, Artificial Sequence
  Description of Artificial Sequence Synthetic primer
caaccagtgacagaagcagaatttt

SEQ ID NO: 838 - DNA, 30 bp, Artificial Sequence
  Description of Artificial Sequence Synthetic primer
tgctttacctaaaacaggagattctttacc

SEQ ID NO: 839 - DNA, 25 bp, Artificial Sequence
  Description of Artificial Sequence Synthetic primer
cagtgcaagtgatggttattgttga

SEQ ID NO: 840 - DNA, 25 bp, Artificial Sequence
  Description of Artificial Sequence Synthetic primer
tgtgcatgattatcgagggattgaa

SEQ ID NO: 841 - DNA, 25 bp, Artificial Sequence
  Description of Artificial Sequence Synthetic primer
tgggacgtacacagttactctacaa

SEQ ID NO: 842 - DNA, 28 bp, Artificial Sequence
  Description of Artificial Sequence Synthetic primer
gaatcaactgtatgcattttcccaaact

SEQ ID NO: 843 - DNA, 24 bp, Artificial Sequence
  Description of Artificial Sequence Synthetic primer
aacatctcagctgacagtggaaaa

SEQ ID NO: 844 - DNA, 25 bp, Artificial Sequence
  Description of Artificial Sequence Synthetic primer
gcacagtgcacatgatgattctatt

SEQ ID NO: 845 - DNA, 22 bp, Artificial Sequence
  Description of Artificial Sequence Synthetic primer
cgctgccagaacttaagagctt

SEQ ID NO: 846 - DNA, 25 bp, Artificial Sequence
  Description of Artificial Sequence Synthetic primer
cgcattatgggagctgatgttacat

SEQ ID NO: 847 - DNA, 24 bp, Artificial Sequence
  Description of Artificial Sequence Synthetic primer
actcctggaacgtacacagttact

SEQ ID NO: 848 - DNA, 25 bp, Artificial Sequence
  Description of Artificial Sequence Synthetic primer
ttcacacagtgctcatgatgattct

SEQ ID NO: 849 - DNA, 23 bp, Artificial Sequence
  Description of Artificial Sequence Synthetic primer
ccagaagtgccaagtcacaaaat

SEQ ID NO: 850 - DNA, 25 bp, Artificial Sequence
  Description of Artificial Sequence Synthetic primer
gtgccaagtcacaaaattccatctt

SEQ ID NO: 851 - DNA, 22 bp, Artificial Sequence
  Description of Artificial Sequence Synthetic primer
tggagtacaacgcgaaaatcga

SEQ ID NO: 852 - DNA, 28 bp, Artificial Sequence
  Description of Artificial Sequence Synthetic primer
aatcttacaatattccggaacagttcca

SEQ ID NO: 853 - DNA, 29 bp, Artificial Sequence
  Description of Artificial Sequence Synthetic primer
gtaacattaaatgcggaaaatgatttgca

SEQ ID NO: 854 - DNA, 20 bp, Artificial Sequence
  Description of Artificial Sequence Synthetic primer
ccagatttttcggcaaagct

SEQ ID NO: 855 - DNA, 25 bp, Artificial Sequence
  Description of Artificial Sequence Synthetic primer
gttccttttccaatggtgacagatt

SEQ ID NO: 856 - DNA, 29 bp, Artificial Sequence
  Description of Artificial Sequence Synthetic primer
tgttacttatttggttagctcccagtttt

SEQ ID NO: 857 - DNA, 24 bp, Artificial Sequence
  Description of Artificial Sequence Synthetic primer
gaagcgtcgtaacttggtctagat

SEQ ID NO: 858 - DNA, 25 bp, Artificial Sequence
  Description of Artificial Sequence Synthetic primer
tgcttttttagtaagagccactgca

SEQ ID NO: 859 - DNA, 23 bp, Artificial Sequence
  Description of Artificial Sequence Synthetic primer
gctggcattttatctgtcgcaaa

SEQ ID NO: 860 - DNA, 26 bp, Artificial Sequence
  Description of Artificial Sequence Synthetic primer
gtgttactggatagttctagccgatt

SEQ ID NO: 861 - DNA, 25 bp, Artificial Sequence
  Description of Artificial Sequence Synthetic primer
actcgccaatgtgcctatatctttt

SEQ ID NO: 862 - DNA, 26 bp, Artificial Sequence
  Description of Artificial Sequence Synthetic primer
tcgtttttcctaagaccgtcttcatt

SEQ ID NO: 863 - DNA, 25 bp, Artificial Sequence
  Description of Artificial Sequence Synthetic primer
cgagcttacgtcacttaccttgtta

SEQ ID NO: 864 - DNA, 25 bp, Artificial Sequence
  Description of Artificial Sequence Synthetic primer
ccgacagtggtgctagattactaat

SEQ ID NO: 865 - DNA, 27 bp, Artificial Sequence
  Description of Artificial Sequence Synthetic primer
gggactgatgttacttatttggttagc

SEQ ID NO: 866 - DNA, 25 bp, Artificial Sequence
  Description of Artificial Sequence Synthetic primer
gccgtccacatgaaacttagcatta

SEQ ID NO: 867 - DNA, 31 bp, Artificial Sequence
  Description of Artificial Sequence Synthetic primer
gttaaattgttcaagtattccaatccatcga

SEQ ID NO: 868 - DNA, 24 bp, Artificial Sequence
  Description of Artificial Sequence Synthetic primer
agttactggatttgcaggcatctt

SEQ ID NO: 869 - DNA, 25 bp, Artificial Sequence
  Description of Artificial Sequence Synthetic primer
ccctaacaaaaggtagagcgcatta

SEQ ID NO: 870 - DNA, 24 bp, Artificial Sequence
  Description of Artificial Sequence Synthetic primer
gcgttcattgtacttgttgtgcta

SEQ ID NO: 871 - DNA, 29 bp, Artificial Sequence
  Description of Artificial Sequence Synthetic primer
tctttaagtggcgttatatccgtaagttg

SEQ ID NO: 872 - DNA, 18 bp, Artificial Sequence
  Description of Artificial Sequence Synthetic primer
gctgccggttctgtcaga

SEQ ID NO: 873 - DNA, 31 bp, Artificial Sequence
  Description of Artificial Sequence Synthetic primer
gtgagatttttcaagttagaaattgggctaa

SEQ ID NO: 874 - DNA, 18 bp, Artificial Sequence
  Description of Artificial Sequence Synthetic primer
agtggctgcgtcacagtt

SEQ ID NO: 875 - DNA, 25 bp, Artificial Sequence
  Description of Artificial Sequence Synthetic primer
gttttttcgtcataccagcctttga

SEQ ID NO: 876 - DNA, 23 bp, Artificial Sequence
  Description of Artificial Sequence Synthetic primer
gttttctttcccttgcgtacgaa

SEQ ID NO: 877 - DNA, 25 bp, Artificial Sequence
  Description of Artificial Sequence Synthetic primer
atcgcagaagcataaggtgtaagtt

SEQ ID NO: 878 - DNA, 23 bp, Artificial Sequence
  Description of Artificial Sequence Synthetic primer
gcaaaatcgcagaagcataaggt

SEQ ID NO: 879 - DNA, 25 bp, Artificial Sequence
  Description of Artificial Sequence Synthetic primer
cagcatctccgccattttctttaat

SEQ ID NO: 880 - DNA, 25 bp, Artificial Sequence
  Description of Artificial Sequence Synthetic primer
ggagtagttgcgtcatatcctgttt

SEQ ID NO: 881 - DNA, 30 bp, Artificial Sequence
  Description of Artificial Sequence Synthetic primer
cgaggtttagagaattctttaatggaagct

SEQ ID NO: 882 - DNA, 24 bp, Artificial Sequence
  Description of Artificial Sequence Synthetic primer
agttaacagtccgctaccttcatg

SEQ ID NO: 883 - DNA, 23 bp, Artificial Sequence
  Description of Artificial Sequence Synthetic primer
gcatctccgccgttttctttaat

SEQ ID NO: 884 - DNA, 25 bp, Artificial Sequence
  Description of Artificial Sequence Synthetic primer
cttccgcaaaaccttccacatattt

SEQ ID NO: 885 - DNA, 22 bp, Artificial Sequence
  Description of Artificial Sequence Synthetic primer
tcgataccatcagcccaagttg

SEQ ID NO: 886 - DNA, 21 bp, Artificial Sequence
  Description of Artificial Sequence Synthetic primer
aatggcaagtgctgcaatgac

SEQ ID NO: 887 - DNA, 23 bp, Artificial Sequence
  Description of Artificial Sequence Synthetic primer
agctttgattgctggagtaccat

SEQ ID NO: 888 - DNA, 25 bp, Artificial Sequence
  Description of Artificial Sequence Synthetic primer
ctcgtcttgcgtataagctacttca

SEQ ID NO: 889 - DNA, 24 bp, Artificial Sequence
  Description of Artificial Sequence Synthetic primer
aagttaaaagtccgctaccttcgt

SEQ ID NO: 890 - DNA, 22 bp, Artificial Sequence
  Description of Artificial Sequence Synthetic primer
gttgcacgaccacaaagtacac

SEQ ID NO: 891 - DNA, 22 bp, Artificial Sequence
  Description of Artificial Sequence Synthetic primer
gggcttaagtcagagcactctt

SEQ ID NO: 892 - DNA, 28 bp, Artificial Sequence
  Description of Artificial Sequence Synthetic primer
tgcatctccaccattttctttaatacga

SEQ ID NO: 893 - DNA, 20 bp, Artificial Sequence
  Description of Artificial Sequence Synthetic primer
ccttgcgtacgaagccattc

SEQ ID NO: 894 - DNA, 25 bp, Artificial Sequence
  Description of Artificial Sequence Synthetic primer
accttcagcaaaaccttccacatat

SEQ ID NO: 895 - DNA, 25 bp, Artificial Sequence
  Description of Artificial Sequence Synthetic primer
accttcagcaaaaccttccacatat

SEQ ID NO: 896 - DNA, 24 bp, Artificial Sequence
  Description of Artificial Sequence Synthetic primer
cgacattcagcaccaattctttct

SEQ ID NO: 897 - DNA, 25 bp, Artificial Sequence
  Description of Artificial Sequence Synthetic primer
cttcgcctttagctttttgagcttt

SEQ ID NO: 898 - DNA, 27 bp, Artificial Sequence
  Description of Artificial Sequence Synthetic primer
tttgcctttatcatctgttgctttacc

SEQ ID NO: 899 - DNA, 25 bp, Artificial Sequence
  Description of Artificial Sequence Synthetic primer
tttagcgtcaccggttttatcttct

SEQ ID NO: 900 - DNA, 20 bp, Artificial Sequence
  Description of Artificial Sequence Synthetic primer
gcgtcgccagttttgtcttc

SEQ ID NO: 901 - DNA, 20 bp, Artificial Sequence
  Description of Artificial Sequence Synthetic primer
agcgtcgcccgttttatctt

SEQ ID NO: 902 - DNA, 24 bp, Artificial Sequence
  Description of Artificial Sequence Synthetic primer
ctgttgctttcccgaatttgtctt

SEQ ID NO: 903 - DNA, 24 bp, Artificial Sequence
  Description of Artificial Sequence Synthetic primer
gtcgccagttttgtcttctacttc

SEQ ID NO: 904 - DNA, 24 bp, Artificial Sequence
  Description of Artificial Sequence Synthetic primer
ctgttgctttcccgaatttgtctt

SEQ ID NO: 905 - DNA, 23 bp, Artificial Sequence
  Description of Artificial Sequence Synthetic primer
gctttgtcgtctgttgctttacc

SEQ ID NO: 906 - DNA, 24 bp, Artificial Sequence
  Description of Artificial Sequence Synthetic primer
catctgttgctttaccgaacttgt

SEQ ID NO: 907 - DNA, 25 bp, Artificial Sequence
  Description of Artificial Sequence Synthetic primer
ttttgtcttctacttcgcctttagc

SEQ ID NO: 908 - DNA, 26 bp, Artificial Sequence
  Description of Artificial Sequence Synthetic primer
tctttttagcgtcaccggttttatct

SEQ ID NO: 909 - DNA, 20 bp, Artificial Sequence
  Description of Artificial Sequence Synthetic primer
gcgtcgcccgttttatcttc

SEQ ID NO: 910 - DNA, 24 bp, Artificial Sequence
  Description of Artificial Sequence Synthetic primer
tcgtctgttgctttaccgaatttg

SEQ ID NO: 911 - DNA, 26 bp, Artificial Sequence
  Description of Artificial Sequence Synthetic primer
gctttaccttcaacttgtttgccttt

SEQ ID NO: 912 - DNA, 23 bp, Artificial Sequence
  Description of Artificial Sequence Synthetic primer
gcctttgtcatctgttgctttcc

SEQ ID NO: 913 - DNA, 24 bp, Artificial Sequence
  Description of Artificial Sequence Synthetic primer
catctgttgctttaccgaacttgt

SEQ ID NO: 914 - DNA, 24 bp, Artificial Sequence
  Description of Artificial Sequence Synthetic primer
tttgcctttgtcatctgttgcttt

SEQ ID NO: 915 - DNA, 25 bp, Artificial Sequence
  Description of Artificial Sequence Synthetic primer
tcatctgttgctttaccgaacttgt

SEQ ID NO: 916 - DNA, 24 bp, Artificial Sequence
  Description of Artificial Sequence Synthetic primer
ggcgtaacaaccacaacttctttg

SEQ ID NO: 917 - DNA, 25 bp, Artificial Sequence
  Description of Artificial Sequence Synthetic primer
cactcgaaagaacgcgataattgtt

SEQ ID NO: 918 - DNA, 26 bp, Artificial Sequence
  Description of Artificial Sequence Synthetic primer
tccatctttcacacattccactacaa

SEQ ID NO: 919 - DNA, 26 bp, Artificial Sequence
  Description of Artificial Sequence Synthetic primer
tccatctttcacacattccactacaa

SEQ ID NO: 920 - DNA, 27 bp, Artificial Sequence
  Description of Artificial Sequence Synthetic primer
tgctttcacaaaacgagacatagatga

SEQ ID NO: 921 - DNA, 23 bp, Artificial Sequence
  Description of Artificial Sequence Synthetic primer
caccaacatcacacagacctttt

SEQ ID NO: 922 - DNA, 24 bp, Artificial Sequence
  Description of Artificial Sequence Synthetic primer
ccaccaacatcacaaagacctttt

SEQ ID NO: 923 - DNA, 23 bp, Artificial Sequence
  Description of Artificial Sequence Synthetic primer
cgttttgatcagccatcaaccaa

SEQ ID NO: 924 - DNA, 25 bp, Artificial Sequence
  Description of Artificial Sequence Synthetic primer
cactcgaaagaacgcgataattgtt

SEQ ID NO: 925 - DNA, 25 bp, Artificial Sequence
  Description of Artificial Sequence Synthetic primer
tggagatagcggcaaattttcacta

SEQ ID NO: 926 - DNA, 23 bp, Artificial Sequence
  Description of Artificial Sequence Synthetic primer
ttggccaatgctttctgattcag

SEQ ID NO: 927 - DNA, 23 bp, Artificial Sequence
  Description of Artificial Sequence Synthetic primer
tctgtttgatcagccatcaacca

SEQ ID NO: 928 - DNA, 27 bp, Artificial Sequence
  Description of Artificial Sequence Synthetic primer
tgctttctgattcagaatcacctgaaa

SEQ ID NO: 929 - DNA, 27 bp, Artificial Sequence
  Description of Artificial Sequence Synthetic primer
aaaagttgctttcacaaaacgagacat

SEQ ID NO: 930 - DNA, 19 bp, Artificial Sequence
  Description of Artificial Sequence Synthetic primer
tcgccaggtaagccgattc

SEQ ID NO: 931 - DNA, 19 bp, Artificial Sequence
  Description of Artificial Sequence Synthetic primer
gtaagccgattccgcccat

SEQ ID NO: 932 - DNA, 25 bp, Artificial Sequence
  Description of Artificial Sequence Synthetic primer
ccacaccagtaatttgactgtttcc

SEQ ID NO: 933 - DNA, 22 bp, Artificial Sequence
  Description of Artificial Sequence Synthetic primer
tccccaccaacatcacaaagac

SEQ ID NO: 934 - DNA, 25 bp, Artificial Sequence
  Description of Artificial Sequence Synthetic primer
tcgctcgaaagaacacgataattgt

SEQ ID NO: 935 - DNA, 27 bp, Artificial Sequence
  Description of Artificial Sequence Synthetic primer
gctttcacaaaacgagacatagaagac

SEQ ID NO: 936 - DNA, 25 bp, Artificial Sequence
  Description of Artificial Sequence Synthetic primer
tccgctttagtccagccaatatttt

SEQ ID NO: 937 - DNA, 29 bp, Artificial Sequence
  Description of Artificial Sequence Synthetic primer
acttataccattatgctccaaagaaagtg

SEQ ID NO: 938 - DNA, 20 bp, Artificial Sequence
  Description of Artificial Sequence Synthetic primer
cggtgatagtctccgcttgt

SEQ ID NO: 939 - DNA, 25 bp, Artificial Sequence
  Description of Artificial Sequence Synthetic primer
gcttcgtttactttgttcgcaatca

SEQ ID NO: 940 - DNA, 18 bp, Artificial Sequence
  Description of Artificial Sequence Synthetic primer
gcttgattggcgttggca

SEQ ID NO: 941 - DNA, 22 bp, Artificial Sequence
  Description of Artificial Sequence Synthetic primer
gcgtgttaagttcaccgctttt

SEQ ID NO: 942 - DNA, 26 bp, Artificial Sequence
  Description of Artificial Sequence Synthetic primer
caaatacaaactttccagctgtggta

SEQ ID NO: 943 - DNA, 25 bp, Artificial Sequence
  Description of Artificial Sequence Synthetic primer
gttgcttgtttatccacggttagtt

SEQ ID NO: 944 - DNA, 19 bp, Artificial Sequence
  Description of Artificial Sequence Synthetic primer
accttcaacggtggctgtt

SEQ ID NO: 945 - DNA, 25 bp, Artificial Sequence
  Description of Artificial Sequence Synthetic primer
cggtttttcataatcgccatcatca

SEQ ID NO: 946 - DNA, 25 bp, Artificial Sequence
  Description of Artificial Sequence Synthetic primer
gcttttccaatagtgactggctgat

SEQ ID NO: 947 - DNA, 21 bp, Artificial Sequence
  Description of Artificial Sequence Synthetic primer
agcgggttaagttgactgctt

SEQ ID NO: 948 - DNA, 23 bp, Artificial Sequence
  Description of Artificial Sequence Synthetic primer
catagccttgtttagtcggaggt

SEQ ID NO: 949 - DNA, 21 bp, Artificial Sequence
  Description of Artificial Sequence Synthetic primer
tgttaagtgcacgcgtatcga

SEQ ID NO: 950 - DNA, 28 bp, Artificial Sequence
  Description of Artificial Sequence Synthetic primer
cgtacaaagtaaaatcatttccggacat

SEQ ID NO: 951 - DNA, 25 bp, Artificial Sequence
  Description of Artificial Sequence Synthetic primer
cttgagcgaacttaggtccttaact

SEQ ID NO: 952 - DNA, 25 bp, Artificial Sequence
  Description of Artificial Sequence Synthetic primer
attttgtgtcactagatccgtcaca

SEQ ID NO: 953 - DNA, 22 bp, Artificial Sequence
  Description of Artificial Sequence Synthetic primer
gcgggttaagttcactgctttt

SEQ ID NO: 954 - DNA, 28 bp, Artificial Sequence
  Description of Artificial Sequence Synthetic primer
tcactgtattcggaacaaccaaattaga

SEQ ID NO: 955 - DNA, 22 bp, Artificial Sequence
  Description of Artificial Sequence Synthetic primer
agtttgtagatccccgcattcc

SEQ ID NO: 956 - DNA, 25 bp, Artificial Sequence
  Description of Artificial Sequence Synthetic primer
cgtttccggacatataatccgtact

SEQ ID NO: 957 - DNA, 23 bp, Artificial Sequence
  Description of Artificial Sequence Synthetic primer
cgccaattgtacgcataatacca

SEQ ID NO: 958 - DNA, 24 bp, Artificial Sequence
  Description of Artificial Sequence Synthetic primer
cgataccaattgcggaaacaactg

SEQ ID NO: 959 - DNA, 27 bp, Artificial Sequence
  Description of Artificial Sequence Synthetic primer
ccacataccgaaaagtaatacgagtgt

SEQ ID NO: 960 - DNA, 25 bp, Artificial Sequence
  Description of Artificial Sequence Synthetic primer
gatggataaaatcccgtcagctagt

SEQ ID NO: 961 - DNA, 17 bp, Artificial Sequence
  Description of Artificial Sequence Synthetic primer
gttgcgattgcgccgat

SEQ ID NO: 962 - DNA, 26 bp, Artificial Sequence
  Description of Artificial Sequence Synthetic primer
caagtgttaacgttccctcaaattca

SEQ ID NO: 963 - DNA, 29 bp, Artificial Sequence
  Description of Artificial Sequence Synthetic primer
ggaggttaataatgaaattcctggatgga

SEQ ID NO: 964 - DNA, 25 bp, Artificial Sequence
  Description of Artificial Sequence Synthetic primer
gcggagacaactggattgaatagag

SEQ ID NO: 965 - DNA, 25 bp, Artificial Sequence
  Description of Artificial Sequence Synthetic primer
gcggaaacaactggattgaatagag

SEQ ID NO: 966 - DNA, 23 bp, Artificial Sequence
  Description of Artificial Sequence Synthetic primer
cccagccagaaacattttgtgat

SEQ ID NO: 967 - DNA, 24 bp, Artificial Sequence
  Description of Artificial Sequence Synthetic primer
gttgttttgtttggcagtgaatgc

SEQ ID NO: 968 - DNA, 21 bp, Artificial Sequence
  Description of Artificial Sequence Synthetic primer
cagtgaatgcgccgattgtac

SEQ ID NO: 969 - DNA, 29 bp, Artificial Sequence
  Description of Artificial Sequence Synthetic primer
gtaagatggataaaattccgtcagctagt

SEQ ID NO: 970 - DNA, 24 bp, Artificial Sequence
  Description of Artificial Sequence Synthetic primer
acccacccagaaacattttgtgat

SEQ ID NO: 971 - DNA, 27 bp, Artificial Sequence
  Description of Artificial Sequence Synthetic primer
gagacaactggattgaatagagcgata

SEQ ID NO: 972 - DNA, 21 bp, Artificial Sequence
  Description of Artificial Sequence Synthetic primer
tcgcaacgacaaggacgatac

SEQ ID NO: 973 - DNA, 19 bp, Artificial Sequence
  Description of Artificial Sequence Synthetic primer
gttgcgattgcgccgatac

SEQ ID NO: 974 - DNA, 23 bp, Artificial Sequence
  Description of Artificial Sequence Synthetic primer
gcaattgctccgataccttgaac

SEQ ID NO: 975 - DNA, 27 bp, Artificial Sequence
  Description of Artificial Sequence Synthetic primer
ccacataccgaaaagtaatacgagtgt

SEQ ID NO: 976 - DNA, 30 bp, Artificial Sequence
  Description of Artificial Sequence Synthetic primer
accagaaatcagtaataagaaaccgaacat

SEQ ID NO: 977 - DNA, 28 bp, Artificial Sequence
  Description of Artificial Sequence Synthetic primer
tgcaacgataagtactattccaattgct

SEQ ID NO: 978 - DNA, 27 bp, Artificial Sequence
  Description of Artificial Sequence Synthetic primer
ttaacgcttcagcaataaatggagttg

SEQ ID NO: 979 - DNA, 25 bp, Artificial Sequence
  Description of Artificial Sequence Synthetic primer
gtgcagtttcatgtgcagaatcata

SEQ ID NO: 980 - DNA, 26 bp, Artificial Sequence
  Description of Artificial Sequence Synthetic primer
ttgttctggatcatcaatgtcccaat

SEQ ID NO: 981 - DNA, 26 bp, Artificial Sequence
  Description of Artificial Sequence Synthetic primer
aagcccatttttttggtaacgctatt

SEQ ID NO: 982 - DNA, 25 bp, Artificial Sequence
  Description of Artificial Sequence Synthetic primer
gtgcagtttcatgtgcagaatcata

SEQ ID NO: 983 - DNA, 25 bp, Artificial Sequence
  Description of Artificial Sequence Synthetic primer
ccaagaaccgctaatgaatttgaca

SEQ ID NO: 984 - DNA, 20 bp, Artificial Sequence
  Description of Artificial Sequence Synthetic primer
gctttcaggtggctcaatcg

SEQ ID NO: 985 - DNA, 23 bp, Artificial Sequence
  Description of Artificial Sequence Synthetic primer
catctgccagtagttctgagcta

SEQ ID NO: 986 - DNA, 27 bp, Artificial Sequence
  Description of Artificial Sequence Synthetic primer
ctaagcctctatcaatacgtgctctaa

SEQ ID NO: 987 - DNA, 26 bp, Artificial Sequence
  Description of Artificial Sequence Synthetic primer
acatacttcttgataactcgcccatt

SEQ ID NO: 988 - DNA, 25 bp, Artificial Sequence
  Description of Artificial Sequence Synthetic primer
gttccggatcatcaatgtcccaata

SEQ ID NO: 989 - DNA, 27 bp, Artificial Sequence
  Description of Artificial Sequence Synthetic primer
ctaagcctctatcaatacgtgctctaa

SEQ ID NO: 990 - DNA, 25 bp, Artificial Sequence
  Description of Artificial Sequence Synthetic primer
gtgcagtttcatgtgcagaatcata

SEQ ID NO: 991 - DNA, 25 bp, Artificial Sequence
  Description of Artificial Sequence Synthetic primer
gttccggatcatcaatgtcccaata

SEQ ID NO: 992 - DNA, 21 bp, Artificial Sequence
  Description of Artificial Sequence Synthetic primer
caagcttcagcaatggcactt

SEQ ID NO: 993 - DNA, 26 bp, Artificial Sequence
  Description of Artificial Sequence Synthetic primer
ttgttctggatcatcaatgtcccaat

SEQ ID NO: 994 - DNA, 27 bp, Artificial Sequence
  Description of Artificial Sequence Synthetic primer
ctaagcctctatcaatacgtgctctaa

SEQ ID NO: 995 - DNA, 25 bp, Artificial Sequence
  Description of Artificial Sequence Synthetic primer
agacttcttgataactcgcccattt

SEQ ID NO: 996 - DNA, 25 bp, Artificial Sequence
  Description of Artificial Sequence Synthetic primer
ctgcttcgtgtgctgaatcataaat

SEQ ID NO: 997 - DNA, 22 bp, Artificial Sequence
  Description of Artificial Sequence Synthetic primer
gcaggaaattgtggtgcagttt

SEQ ID NO: 998 - DNA, 23 bp, Artificial Sequence
  Description of Artificial Sequence Synthetic primer
atcagcatctgccagtagttcag

SEQ ID NO: 999 - DNA, 25 bp, Artificial Sequence
  Description of Artificial Sequence Synthetic primer
tgcaccagcattacttgtcatgata

SEQ ID NO: 1000 - DNA, 28 bp, Artificial Sequence
  Description of Artificial Sequence Synthetic primer
ggaaccgattaatttagagatgctgtgt

SEQ ID NO: 1001 - DNA, 18 bp, Artificial Sequence
  Description of Artificial Sequence Synthetic primer
cgcttgctcctgggaact

SEQ ID NO: 1002 - DNA, 25 bp, Artificial Sequence
  Description of Artificial Sequence Synthetic primer
acgtctttctagtgcagcatctttt

SEQ ID NO: 1003 - DNA, 28 bp, Artificial Sequence
  Description of Artificial Sequence Synthetic primer
gtcaacgagcattaagtcgataatttgt

SEQ ID NO: 1004 - DNA, 25 bp, Artificial Sequence
  Description of Artificial Sequence Synthetic primer
gctgctgttaaagcttcaggagaat

SEQ ID NO: 1005 - DNA, 26 bp, Artificial Sequence
  Description of Artificial Sequence Synthetic primer
ctttagctacttttttcacggcatct

SEQ ID NO: 1006 - DNA, 23 bp, Artificial Sequence
  Description of Artificial Sequence Synthetic primer
cgctacttttttcacggcatctt

SEQ ID NO: 1007 - DNA, 24 bp, Artificial Sequence
  Description of Artificial Sequence Synthetic primer
ttctttgttcacggtttgcttgac

SEQ ID NO: 1008 - DNA, 30 bp, Artificial Sequence
  Description of Artificial Sequence Synthetic primer
gaatcgtgtttttacgttcttgtagttctt

SEQ ID NO: 1009 - DNA, 21 bp, Artificial Sequence
  Description of Artificial Sequence Synthetic primer
cttggatagtccggcgtagtg

SEQ ID NO: 1010 - DNA, 25 bp, Artificial Sequence
  Description of Artificial Sequence Synthetic primer
cccaacgatagtatgcacttcatca

SEQ ID NO: 1011 - DNA, 21 bp, Artificial Sequence
  Description of Artificial Sequence Synthetic primer
cctgcgacaattgcatttgca

SEQ ID NO: 1012 - DNA, 25 bp, Artificial Sequence
  Description of Artificial Sequence Synthetic primer
gcgagctaaggctggttttaaaata

SEQ ID NO: 1013 - DNA, 24 bp, Artificial Sequence
  Description of Artificial Sequence Synthetic primer
ggtccaacaaataggaaggaacca

SEQ ID NO: 1014 - DNA, 25 bp, Artificial Sequence
  Description of Artificial Sequence Synthetic primer
gctgccttttcgtaatcttccattt

SEQ ID NO: 1015 - DNA, 18 bp, Artificial Sequence
  Description of Artificial Sequence Synthetic primer
cggcgaagtggtctagca

SEQ ID NO: 1016 - DNA, 24 bp, Artificial Sequence
  Description of Artificial Sequence Synthetic primer
ccaacaatcgtatgcacttcatca

SEQ ID NO: 1017 - DNA, 25 bp, Artificial Sequence
  Description of Artificial Sequence Synthetic primer
gcttggatatcagatgcttggatga

SEQ ID NO: 1018 - DNA, 29 bp, Artificial Sequence
  Description of Artificial Sequence Synthetic primer
cagaaagattgtattttgagccaacttca

SEQ ID NO: 1019 - DNA, 20 bp, Artificial Sequence
  Description of Artificial Sequence Synthetic primer
gaggcgctccaactaaacga

SEQ ID NO: 1020 - DNA, 29 bp, Artificial Sequence
  Description of Artificial Sequence Synthetic primer
gatagttccatttgtgatacaaggtctgt

SEQ ID NO: 1021 - DNA, 25 bp, Artificial Sequence
  Description of Artificial Sequence Synthetic primer
gagtcagtatctctgagttcgttgt

SEQ ID NO: 1022 - DNA, 23 bp, Artificial Sequence
  Description of Artificial Sequence Synthetic primer
ccgtctacataattcccgccatt

SEQ ID NO: 1023 - DNA, 25 bp, Artificial Sequence
  Description of Artificial Sequence Synthetic primer
ccgcaagagattgaatgttgctatt

SEQ ID NO: 1024 - DNA, 26 bp, Artificial Sequence
  Description of Artificial Sequence Synthetic primer
gttcattcacacatttctgaccagtt

SEQ ID NO: 1025 - DNA, 20 bp, Artificial Sequence
  Description of Artificial Sequence Synthetic primer
ctaggccgggatctggaaaa

SEQ ID NO: 1026 - DNA, 29 bp, Artificial Sequence
  Description of Artificial Sequence Synthetic primer
aggtcacttatttgattgtgggatagatg

SEQ ID NO: 1027 - DNA, 22 bp, Artificial Sequence
  Description of Artificial Sequence Synthetic primer
tgcattccggcaagagattgaa

SEQ ID NO: 1028 - DNA, 25 bp, Artificial Sequence
  Description of Artificial Sequence Synthetic primer
gcgtgataaacaagcacttggaatt

SEQ ID NO: 1029 - DNA, 29 bp, Artificial Sequence
  Description of Artificial Sequence Synthetic primer
agcatttgctatatacaattctggttggt

SEQ ID NO: 1030 - DNA, 25 bp, Artificial Sequence
  Description of Artificial Sequence Synthetic primer
ggttaataggcgttggttgttgaat

SEQ ID NO: 1031 - DNA, 30 bp, Artificial Sequence
  Description of Artificial Sequence Synthetic primer
ggactaaggtcacttatttgattatgggat

SEQ ID NO: 1032 - DNA, 36 bp, Artificial Sequence
  Description of Artificial Sequence Synthetic primer
agttagatcatttaaaggactaaggtcacttatttg

SEQ ID NO: 1033 - DNA, 20 bp, Artificial Sequence
  Description of Artificial Sequence Synthetic primer
ctaggccgggatctggaaaa

SEQ ID NO: 1034 - DNA, 25 bp, Artificial Sequence
  Description of Artificial Sequence Synthetic primer
ccacaggacacaaccatctacataa

SEQ ID NO: 1035 - DNA, 31 bp, Artificial Sequence
  Description of Artificial Sequence Synthetic primer
acagtgtttgttatatacaattctggttggt

SEQ ID NO: 1036 - DNA, 20 bp, Artificial Sequence
  Description of Artificial Sequence Synthetic primer
ctaggccgggatctggaaaa

SEQ ID NO: 1037 - DNA, 25 bp, Artificial Sequence
  Description of Artificial Sequence Synthetic primer
tccggcaagagatttaatgttgcta

SEQ ID NO: 1038 - DNA, 24 bp, Artificial Sequence
  Description of Artificial Sequence Synthetic primer
cctcagtctccccaacgtttatat

SEQ ID NO: 1039 - DNA, 25 bp, Artificial Sequence
  Description of Artificial Sequence Synthetic primer
caaccgtctacataattcccaccat

SEQ ID NO: 1040 - DNA, 24 bp, Artificial Sequence
  Description of Artificial Sequence Synthetic primer
gttaataggcgttggtcgttgaat

SEQ ID NO: 1041 - DNA, 26 bp, Artificial Sequence
  Description of Artificial Sequence Synthetic primer
ccggatatccatctttgagtaaagca

SEQ ID NO: 1042 - DNA, 21 bp, Artificial Sequence
  Description of Artificial Sequence Synthetic primer
cgctttcgaagtggcaattgg

SEQ ID NO: 1043 - DNA, 20 bp, Artificial Sequence
  Description of Artificial Sequence Synthetic primer
tctgctttcgaggtggcaat

SEQ ID NO: 1044 - DNA, 26 bp, Artificial Sequence
  Description of Artificial Sequence Synthetic primer
catcttttaatagggcattgccgatt

SEQ ID NO: 1045 - DNA, 26 bp, Artificial Sequence
  Description of Artificial Sequence Synthetic primer
ccattcttctgcttttgaagtagcaa

SEQ ID NO: 1046 - DNA, 19 bp, Artificial Sequence
  Description of Artificial Sequence Synthetic primer
agcgcggctttctgagtag

SEQ ID NO: 1047 - DNA, 19 bp, Artificial Sequence
  Description of Artificial Sequence Synthetic primer
gcgcggctttctgaatagc

SEQ ID NO: 1048 - DNA, 23 bp, Artificial Sequence
  Description of Artificial Sequence Synthetic primer
tctgcttttgaagtggcaattgg

SEQ ID NO: 1049 - DNA, 18 bp, Artificial Sequence
  Description of Artificial Sequence Synthetic primer
gcgcggctttctgagtag

SEQ ID NO: 1050 - DNA, 23 bp, Artificial Sequence
  Description of Artificial Sequence Synthetic primer
cgttgccgatttcaattgccttt

SEQ ID NO: 1051 - DNA, 30 bp, Artificial Sequence
  Description of Artificial Sequence Synthetic primer
tgagtaaagcattaccaatctcaatagctt

SEQ ID NO: 1052 - DNA, 18 bp, Artificial Sequence
  Description of Artificial Sequence Synthetic primer
gcgcggctttctgagtag

SEQ ID NO: 1053 - DNA, 25 bp, Artificial Sequence
  Description of Artificial Sequence Synthetic primer
gtggcaataggtatagctcgatctt

SEQ ID NO: 1054 - DNA, 18 bp, Artificial Sequence
  Description of Artificial Sequence Synthetic primer
gcgcggctttctgagtag

SEQ ID NO: 1055 - DNA, 33 bp, Artificial Sequence
  Description of Artificial Sequence Synthetic primer
cgattccttatgatttttataccattcttctgc

SEQ ID NO: 1056 - DNA, 18 bp, Artificial Sequence
  Description of Artificial Sequence Synthetic primer
gcgcggctttctgagtag

SEQ ID NO: 1057 - DNA, 25 bp, Artificial Sequence
  Description of Artificial Sequence Synthetic primer
ttttaatagcgcattgccgatttca

SEQ ID NO: 1058 - DNA, 26 bp, Artificial Sequence
  Description of Artificial Sequence Synthetic primer
ccattcttctgcttttgaagtagcaa

SEQ ID NO: 1059 - DNA, 19 bp, Artificial Sequence
  Description of Artificial Sequence Synthetic primer
agcgcggctttctgagtag

SEQ ID NO: 1060 - DNA, 19 bp, Artificial Sequence
  Description of Artificial Sequence Synthetic primer
gcgcggctttctgaatagc

SEQ ID NO: 1061 - DNA, 22 bp, Artificial Sequence
  Description of Artificial Sequence Synthetic primer
tgctgtgctggttgttctgtag

SEQ ID NO: 1062 - DNA, 25 bp, Artificial Sequence
  Description of Artificial Sequence Synthetic primer
cccattcctggttcttctttactgt

SEQ ID NO: 1063 - DNA, 25 bp, Artificial Sequence
  Description of Artificial Sequence Synthetic primer
acttgcaatgtttggttcggaaaaa

SEQ ID NO: 1064 - DNA, 30 bp, Artificial Sequence
  Description of Artificial Sequence Synthetic primer
gtttggtttggaaaaattaagtttgagctt

SEQ ID NO: 1065 - DNA, 26 bp, Artificial Sequence
  Description of Artificial Sequence Synthetic primer
atttgttgttttcagaaggtgtgctt

SEQ ID NO: 1066 - DNA, 23 bp, Artificial Sequence
  Description of Artificial Sequence Synthetic primer
tctacagcaataacgcgtggatt

SEQ ID NO: 1067 - DNA, 27 bp, Artificial Sequence
  Description of Artificial Sequence Synthetic primer
catttaaatcaattccagtcgctgtca

SEQ ID NO: 1068 - DNA, 25 bp, Artificial Sequence
  Description of Artificial Sequence Synthetic primer
ctgttgcagttaccgttaactcttt

SEQ ID NO: 1069 - DNA, 25 bp, Artificial Sequence
  Description of Artificial Sequence Synthetic primer
cttgtgctgatgaagaactgtttga

SEQ ID NO: 1070 - DNA, 25 bp, Artificial Sequence
  Description of Artificial Sequence Synthetic primer
gtgtgtcaccagctacaactgtata

SEQ ID NO: 1071 - DNA, 19 bp, Artificial Sequence
  Description of Artificial Sequence Synthetic primer
tccctgtcgctgtcatgtg

SEQ ID NO: 1072 - DNA, 24 bp, Artificial Sequence
  Description of Artificial Sequence Synthetic primer
tgttacaccattatcaacggcgat

SEQ ID NO: 1073 - DNA, 25 bp, Artificial Sequence
  Description of Artificial Sequence Synthetic primer
tggatcaactgcaattactcgtgaa

SEQ ID NO: 1074 - DNA, 22 bp, Artificial Sequence
  Description of Artificial Sequence Synthetic primer
gtagccgggctctcattagatt

SEQ ID NO: 1075 - DNA, 25 bp, Artificial Sequence
  Description of Artificial Sequence Synthetic primer
acgatttgtttgaagatgctgcttt

SEQ ID NO: 1076 - DNA, 24 bp, Artificial Sequence
  Description of Artificial Sequence Synthetic primer
ccccagttgttagcttcttgtact

SEQ ID NO: 1077 - DNA, 25 bp, Artificial Sequence
  Description of Artificial Sequence Synthetic primer
gctgaagaactgtttgaagatgctt

SEQ ID NO: 1078 - DNA, 27 bp, Artificial Sequence
  Description of Artificial Sequence Synthetic primer
tctttactgtatgcagttgctgttaca

SEQ ID NO: 1079 - DNA, 27 bp, Artificial Sequence
  Description of Artificial Sequence Synthetic primer
tgttccgcattatttgtagatggctta

SEQ ID NO: 1080 - DNA, 23 bp, Artificial Sequence
  Description of Artificial Sequence Synthetic primer
ggtgtgctttcactagctttagc

SEQ ID NO: 1081 - DNA, 24 bp, Artificial Sequence
  Description of Artificial Sequence Synthetic primer
ccccagttgttagcttcttgtact

SEQ ID NO: 1082 - DNA, 21 bp, Artificial Sequence
  Description of Artificial Sequence Synthetic primer
acgtcgaaagtcactgcttga

SEQ ID NO: 1083 - DNA, 25 bp, Artificial Sequence
  Description of Artificial Sequence Synthetic primer
ctttttctacattagctgcctgagc

SEQ ID NO: 1084 - DNA, 25 bp, Artificial Sequence
  Description of Artificial Sequence Synthetic primer
tcttcaacgtcgaaagttactgctt

SEQ ID NO: 1085 - DNA, 25 bp, Artificial Sequence
  Description of Artificial Sequence Synthetic primer
cgctttgaccttcgtctaaagtttt

SEQ ID NO: 1086 - DNA, 25 bp, Artificial Sequence
  Description of Artificial Sequence Synthetic primer
gcgattaccttcgacaacttcaaat

SEQ ID NO: 1087 - DNA, 25 bp, Artificial Sequence
  Description of Artificial Sequence Synthetic primer
cgatagcgctgaagtgtacgaatat

SEQ ID NO: 1088 - DNA, 15 bp, Artificial Sequence
  Description of Artificial Sequence Synthetic primer
tgagcgccgcggtta

SEQ ID NO: 1089 - DNA, 18 bp, Artificial Sequence
  Description of Artificial Sequence Synthetic primer
cttgtgggccacgttgac

SEQ ID NO: 1090 - DNA, 20 bp, Artificial Sequence
  Description of Artificial Sequence Synthetic primer
tttgaatccgtcgccttgga

SEQ ID NO: 1091 - DNA, 18 bp, Artificial Sequence
  Description of Artificial Sequence Synthetic primer
cttgtgggccacgttgac

SEQ ID NO: 1092 - DNA, 25 bp, Artificial Sequence
  Description of Artificial Sequence Synthetic primer
cgatagcgctgaagtgtacgaatac

SEQ ID NO: 1093 - DNA, 22 bp, Artificial Sequence
  Description of Artificial Sequence Synthetic primer
catcgtctccgccttctacttc

SEQ ID NO: 1094 - DNA, 23 bp, Artificial Sequence
  Description of Artificial Sequence Synthetic primer
tttttccacattggctgcttgag

SEQ ID NO: 1095 - DNA, 24 bp, Artificial Sequence
  Description of Artificial Sequence Synthetic primer
cgtcgaaagttactgcttgacctt

SEQ ID NO: 1096 - DNA, 25 bp, Artificial Sequence
  Description of Artificial Sequence Synthetic primer
gttggccttcaacgatttcaaattc

SEQ ID NO: 1097 - DNA, 25 bp, Artificial Sequence
  Description of Artificial Sequence Synthetic primer
cgctttgaccttcgtctaaagtttt

SEQ ID NO: 1098 - DNA, 25 bp, Artificial Sequence
  Description of Artificial Sequence Synthetic primer
ccgcgattaccttcaactacttcaa

SEQ ID NO: 1099 - DNA, 22 bp, Artificial Sequence
  Description of Artificial Sequence Synthetic primer
accgcttggccttcttctaaag

SEQ ID NO: 1100 - DNA, 22 bp, Artificial Sequence
  Description of Artificial Sequence Synthetic primer
accgcttggccttcttctaaag

SEQ ID NO: 1101 - DNA, 17 bp, Artificial Sequence
  Description of Artificial Sequence Synthetic primer
ttcgccgccgtctgatt

SEQ ID NO: 1102 - DNA, 27 bp, Artificial Sequence
  Description of Artificial Sequence Synthetic primer
cttcaacgatttcaaattcaacgcttt

SEQ ID NO: 1103 - DNA, 29 bp, Artificial Sequence
  Description of Artificial Sequence Synthetic primer
ctgattggaagatacacccaacatattca

SEQ ID NO: 1104 - DNA, 25 bp, Artificial Sequence
  Description of Artificial Sequence Synthetic primer
cgttagattagcaagaggtgtcaca

SEQ ID NO: 1105 - DNA, 25 bp, Artificial Sequence
  Description of Artificial Sequence Synthetic primer
tggtcaatcttgaaagcggagttaa

SEQ ID NO: 1106 - DNA, 25 bp, Artificial Sequence
  Description of Artificial Sequence Synthetic primer
tgtcatattcgtagccggcatttta

SEQ ID NO: 1107 - DNA, 24 bp, Artificial Sequence
  Description of Artificial Sequence Synthetic primer
aatcccattcattcccacctgttt

SEQ ID NO: 1108 - DNA, 25 bp, Artificial Sequence
  Description of Artificial Sequence Synthetic primer
gcgctgtcgttagctttaattgtta

SEQ ID NO: 1109 - DNA, 22 bp, Artificial Sequence
  Description of Artificial Sequence Synthetic primer
gcgcggctgttatttgttgttt

SEQ ID NO: 1110 - DNA, 25 bp, Artificial Sequence
  Description of Artificial Sequence Synthetic primer
gcttctactatcggttgaacaacag

SEQ ID NO: 1111 - DNA, 27 bp, Artificial Sequence
  Description of Artificial Sequence Synthetic primer
ctcactcgtgaaactatctaagttcca

SEQ ID NO: 1112 - DNA, 23 bp, Artificial Sequence
  Description of Artificial Sequence Synthetic primer
tccgcatcataccaaccagaaaa

SEQ ID NO: 1113 - DNA, 29 bp, Artificial Sequence
  Description of Artificial Sequence Synthetic primer
ttttggaagtttagtactcgtcgtatctg

SEQ ID NO: 1114 - DNA, 30 bp, Artificial Sequence
  Description of Artificial Sequence Synthetic primer
gacttgcatcacttatttgattttctcgta

SEQ ID NO: 1115 - DNA, 22 bp, Artificial Sequence
  Description of Artificial Sequence Synthetic primer
agcggccgttatttgttgtttt

SEQ ID NO: 1116 - DNA, 23 bp, Artificial Sequence
  Description of Artificial Sequence Synthetic primer
gtttctccgtccacatcaaatgc

SEQ ID NO: 1117 - DNA, 25 bp, Artificial Sequence
  Description of Artificial Sequence Synthetic primer
aatactctctgcctgagctttcatc

SEQ ID NO: 1118 - DNA, 23 bp, Artificial Sequence
  Description of Artificial Sequence Synthetic primer
gctaatgctggatccgtgaaaat

SEQ ID NO: 1119 - DNA, 23 bp, Artificial Sequence
  Description of Artificial Sequence Synthetic primer
gccggcattttatctactgcaaa

SEQ ID NO: 1120 - DNA, 25 bp, Artificial Sequence
  Description of Artificial Sequence Synthetic primer
accagtttttgcatcataccatcca

SEQ ID NO: 1121 - DNA, 24 bp, Artificial Sequence
  Description of Artificial Sequence Synthetic primer
cggttcagcaggttcggttattaa

SEQ ID NO: 1122 - DNA, 30 bp, Artificial Sequence
  Description of Artificial Sequence Synthetic primer
catcacttgtttttggaagtttagtactcg

SEQ ID NO: 1123 - DNA, 24 bp, Artificial Sequence
  Description of Artificial Sequence Synthetic primer
cacgtagaagtgctgggttagttt

SEQ ID NO: 1124 - DNA, 24 bp, Artificial Sequence
  Description of Artificial Sequence Synthetic primer
cgtctttaagctttcgaggtgttc

SEQ ID NO: 1125 - DNA, 24 bp, Artificial Sequence
  Description of Artificial Sequence Synthetic primer
tggtgagattggatgcactgattt

SEQ ID NO: 1126 - DNA, 25 bp, Artificial Sequence
  Description of Artificial Sequence Synthetic primer
cggctttttggttaccactttttcc

SEQ ID NO: 1127 - DNA, 25 bp, Artificial Sequence
  Description of Artificial Sequence Synthetic primer
atgcgcattgtactaatccaaaact

SEQ ID NO: 1128 - DNA, 25 bp, Artificial Sequence
  Description of Artificial Sequence Synthetic primer
gtagaagtgctgggttagtttcgat

SEQ ID NO: 1129 - DNA, 23 bp, Artificial Sequence
  Description of Artificial Sequence Synthetic primer
cctttgagtgtatcgcgtatgtg

SEQ ID NO: 1130 - DNA, 25 bp, Artificial Sequence
  Description of Artificial Sequence Synthetic primer
gtctttgagtgcttcgcagaatttt

SEQ ID NO: 1131 - DNA, 21 bp, Artificial Sequence
  Description of Artificial Sequence Synthetic primer
gctctgttcgccctgtatgac

SEQ ID NO: 1132 - DNA, 20 bp, Artificial Sequence
  Description of Artificial Sequence Synthetic primer
cagaagcacgtcgcctactt

SEQ ID NO: 1133 - DNA, 17 bp, Artificial Sequence
  Description of Artificial Sequence Synthetic primer
cacgaatgcgcgcttct

SEQ ID NO: 1134 - DNA, 24 bp, Artificial Sequence
  Description of Artificial Sequence Synthetic primer
ctgggaaacaaactccatgctttt

SEQ ID NO: 1135 - DNA, 22 bp, Artificial Sequence
  Description of Artificial Sequence Synthetic primer
gtgttccgcaccaaatttctct

SEQ ID NO: 1136 - DNA, 27 bp, Artificial Sequence
  Description of Artificial Sequence Synthetic primer
cacgtatatgagtttctgtcagaagca

SEQ ID NO: 1137 - DNA, 25 bp, Artificial Sequence
  Description of Artificial Sequence Synthetic primer
cgcatcaaaattccgtcgtttatca

SEQ ID NO: 1138 - DNA, 26 bp, Artificial Sequence
  Description of Artificial Sequence Synthetic primer
tggatttccacctgataaacattcgt

SEQ ID NO: 1139 - DNA, 24 bp, Artificial Sequence
  Description of Artificial Sequence Synthetic primer
tttctcggtgacatagcctttgag

SEQ ID NO: 1140 - DNA, 25 bp, Artificial Sequence
  Description of Artificial Sequence Synthetic primer
gtctttgagtgcttcgcagaatttt

SEQ ID NO: 1141 - DNA, 25 bp, Artificial Sequence
  Description of Artificial Sequence Synthetic primer
cgcatcaaaattccgtcgtttatca

SEQ ID NO: 1142 - DNA, 23 bp, Artificial Sequence
  Description of Artificial Sequence Synthetic primer
cgcaaaagacacttgatctgcaa

SEQ ID NO: 1143 - DNA, 25 bp, Artificial Sequence
  Description of Artificial Sequence Synthetic primer
catttttcggtgacatagcctttga

SEQ ID NO: 1144 - DNA, 25 bp, Artificial Sequence
  Description of Artificial Sequence Synthetic primer
cttgaagtgatttgtcggctgtaat

SEQ ID NO: 1145 - DNA, 24 bp, Artificial Sequence
  Description of Artificial Sequence Synthetic primer
gtagttttcccacaaccacttgga

SEQ ID NO: 1146 - DNA, 29 bp, Artificial Sequence
  Description of Artificial Sequence Synthetic primer
gttgaatgacatatccaatagagcgtctt

SEQ ID NO: 1147 - DNA, 28 bp, Artificial Sequence
  Description of Artificial Sequence Synthetic primer
accttcatccactacaagtaacgtatct

SEQ ID NO: 1148 - DNA, 24 bp, Artificial Sequence
  Description of Artificial Sequence Synthetic primer
cagtgtcacgaagcaaggtatctt

SEQ ID NO: 1149 - DNA, 24 bp, Artificial Sequence
  Description of Artificial Sequence Synthetic primer
tgctgtccaccacttaattcgtaa

SEQ ID NO: 1150 - DNA, 25 bp, Artificial Sequence
  Description of Artificial Sequence Synthetic primer
acagaagttgctgtacgacgattta

SEQ ID NO: 1151 - DNA, 25 bp, Artificial Sequence
  Description of Artificial Sequence Synthetic primer
ggatcttccgccatgatgtctttat

SEQ ID NO: 1152 - DNA, 25 bp, Artificial Sequence
  Description of Artificial Sequence Synthetic primer
ccacttggaccgataaaacaaacaa

SEQ ID NO: 1153 - DNA, 25 bp, Artificial Sequence
  Description of Artificial Sequence Synthetic primer
ccacttggaccgataaaacaaacaa

SEQ ID NO: 1154 - DNA, 28 bp, Artificial Sequence
  Description of Artificial Sequence Synthetic primer
ctttcttgttttttctcttcggaccatt

SEQ ID NO: 1155 - DNA, 29 bp, Artificial Sequence
  Description of Artificial Sequence Synthetic primer
tctcttcggaccatttaagtaattttggt

SEQ ID NO: 1156 - DNA, 23 bp, Artificial Sequence
  Description of Artificial Sequence Synthetic primer
gccagtttaatcgcttcatccat

SEQ ID NO: 1157 - DNA, 21 bp, Artificial Sequence
  Description of Artificial Sequence Synthetic primer
cgttttggaatccgcctgttc

SEQ ID NO: 1158 - DNA, 27 bp, Artificial Sequence
  Description of Artificial Sequence Synthetic primer
acggagtaaggtatcttcatacacgta

SEQ ID NO: 1159 - DNA, 24 bp, Artificial Sequence
  Description of Artificial Sequence Synthetic primer
acagaagttgcagtacgacgattt

SEQ ID NO: 1160 - DNA, 25 bp, Artificial Sequence
  Description of Artificial Sequence Synthetic primer
cgctggattacgcaaaatttcatct

SEQ ID NO: 1161 - DNA, 20 bp, Artificial Sequence
  Description of Artificial Sequence Synthetic primer
tcgtcgttttcccacaacca

SEQ ID NO: 1162 - DNA, 25 bp, Artificial Sequence
  Description of Artificial Sequence Synthetic primer
ggatcttccgccatgatgtctttat

SEQ ID NO: 1163 - DNA, 24 bp, Artificial Sequence
  Description of Artificial Sequence Synthetic primer
actaagctggcacgagtaacaatt

SEQ ID NO: 1164 - DNA, 25 bp, Artificial Sequence
  Description of Artificial Sequence Synthetic primer
ggatcttccgccatgatgtctttat

SEQ ID NO: 1165 - DNA, 25 bp, Artificial Sequence
  Description of Artificial Sequence Synthetic primer
ttgcgtgcgcagtttaaataagtta

SEQ ID NO: 1166 - DNA, 25 bp, Artificial Sequence
  Description of Artificial Sequence Synthetic primer
ggtggtaatgttgttacttgtgcaa

SEQ ID NO: 1167 - DNA, 29 bp, Artificial Sequence
  Description of Artificial Sequence Synthetic primer
cagttgtgtaattatctcctactgttcca

SEQ ID NO: 1168 - DNA, 24 bp, Artificial Sequence
  Description of Artificial Sequence Synthetic primer
gttttgccattagcatccacgtaa

SEQ ID NO: 1169 - DNA, 25 bp, Artificial Sequence
  Description of Artificial Sequence Synthetic primer
gtaaaggttggtcgatgtttgtgtt

SEQ ID NO: 1170 - DNA, 25 bp, Artificial Sequence
  Description of Artificial Sequence Synthetic primer
ggctgacatctatttcggttaaggt

SEQ ID NO: 1171 - DNA, 16 bp, Artificial Sequence
  Description of Artificial Sequence Synthetic primer
ggtttgcgcgctgctt

SEQ ID NO: 1172 - DNA, 25 bp, Artificial Sequence
  Description of Artificial Sequence Synthetic primer
gccaatagttccgtttaggatttcg

SEQ ID NO: 1173 - DNA, 25 bp, Artificial Sequence
  Description of Artificial Sequence Synthetic primer
ggttaattgtgtattgtggctgaca

SEQ ID NO: 1174 - DNA, 22 bp, Artificial Sequence
  Description of Artificial Sequence Synthetic primer
gtgtcgccaacatttccgttta

SEQ ID NO: 1175 - DNA, 28 bp, Artificial Sequence
  Description of Artificial Sequence Synthetic primer
ttgtgtgttgtgtgttaggtctatttct

SEQ ID NO: 1176 - DNA, 30 bp, Artificial Sequence
  Description of Artificial Sequence Synthetic primer
cgtcaacatagtgaatttctacaactcctt

SEQ ID NO: 1177 - DNA, 23 bp, Artificial Sequence
  Description of Artificial Sequence Synthetic primer
cgcgctagaagtataaggatcgt

SEQ ID NO: 1178 - DNA, 25 bp, Artificial Sequence
  Description of Artificial Sequence Synthetic primer
cgccaatttttctcctttgtcatca

SEQ ID NO: 1179 - DNA, 22 bp, Artificial Sequence
  Description of Artificial Sequence Synthetic primer
ggagcgagcattttgccattag

SEQ ID NO: 1180 - DNA, 32 bp, Artificial Sequence
  Description of Artificial Sequence Synthetic primer
cgctacaatcaaaatatgttaactgtgtaagc

SEQ ID NO: 1181 - DNA, 20 bp, Artificial Sequence
  Description of Artificial Sequence Synthetic primer
agcgagtgttttgccggtat

SEQ ID NO: 1182 - DNA, 24 bp, Artificial Sequence
  Description of Artificial Sequence Synthetic primer
gttttgccattagcatccacgtaa

SEQ ID NO: 1183 - DNA, 26 bp, Artificial Sequence
  Description of Artificial Sequence Synthetic primer
tggtaaaggttgatctgtgtttgtgt

SEQ ID NO: 1184 - DNA, 24 bp, Artificial Sequence
  Description of Artificial Sequence Synthetic primer
cgcaagacaaacttttcagctttg

SEQ ID NO: 1185 - DNA, 31 bp, Artificial Sequence
  Description of Artificial Sequence Synthetic primer
ctattttttctttttccagataactagagct

SEQ ID NO: 1186 - DNA, 24 bp, Artificial Sequence
  Description of Artificial Sequence Synthetic primer
gctgatatcttgtcccccagtaat

SEQ ID NO: 1187 - DNA, 24 bp, Artificial Sequence
  Description of Artificial Sequence Synthetic primer
gctgatatcttgtccccctgtaat

SEQ ID NO: 1188 - DNA, 25 bp, Artificial Sequence
  Description of Artificial Sequence Synthetic primer
gccgtattgttggatagcttctttc

SEQ ID NO: 1189 - DNA, 25 bp, Artificial Sequence
  Description of Artificial Sequence Synthetic primer
gctgcaactagtcctaaaacaccta

SEQ ID NO: 1190 - DNA, 25 bp, Artificial Sequence
  Description of Artificial Sequence Synthetic primer
ctgcgacaacaaaacctaagaagaa

SEQ ID NO: 1191 - DNA, 24 bp, Artificial Sequence
  Description of Artificial Sequence Synthetic primer
acgataaatccaccagctacgttt

SEQ ID NO: 1192 - DNA, 18 bp, Artificial Sequence
  Description of Artificial Sequence Synthetic primer
gggcagcatctggagcaa

SEQ ID NO: 1193 - DNA, 25 bp, Artificial Sequence
  Description of Artificial Sequence Synthetic primer
ccaccagctacgtttagaccattaa

SEQ ID NO: 1194 - DNA, 25 bp, Artificial Sequence
  Description of Artificial Sequence Synthetic primer
ctgcgacaacaaaacctaagaagaa

SEQ ID NO: 1195 - DNA, 22 bp, Artificial Sequence
  Description of Artificial Sequence Synthetic primer
ggtgactgccacgataattgct

SEQ ID NO: 1196 - DNA, 23 bp, Artificial Sequence
  Description of Artificial Sequence Synthetic primer
actgccacgataattgctggaat

SEQ ID NO: 1197 - DNA, 25 bp, Artificial Sequence
  Description of Artificial Sequence Synthetic primer
gcaacgagtcctataacacctagtg

SEQ ID NO: 1198 - DNA, 22 bp, Artificial Sequence
  Description of Artificial Sequence Synthetic primer
cgcagctccgatattcatccat

SEQ ID NO: 1199 - DNA, 25 bp, Artificial Sequence
  Description of Artificial Sequence Synthetic primer
cgatacgcattgcttgaagtagtag

SEQ ID NO: 1200 - DNA, 24 bp, Artificial Sequence
  Description of Artificial Sequence Synthetic primer
cttaccagctttatcagccatgtg

SEQ ID NO: 1201 - DNA, 26 bp, Artificial Sequence
  Description of Artificial Sequence Synthetic primer
tgatatcttgtccccccgtaataact

SEQ ID NO: 1202 - DNA, 25 bp, Artificial Sequence
  Description of Artificial Sequence Synthetic primer
gcgatacgcatagcttgaagtagta

SEQ ID NO: 1203 - DNA, 25 bp, Artificial Sequence
  Description of Artificial Sequence Synthetic primer
ccgtattgctcgatagcttctttca

SEQ ID NO: 1204 - DNA, 22 bp, Artificial Sequence
  Description of Artificial Sequence Synthetic primer
tcccagctttatcagccatgtg

SEQ ID NO: 1205 - DNA, 25 bp, Artificial Sequence
  Description of Artificial Sequence Synthetic primer
cccccaataatgataccggttgtaa

SEQ ID NO: 1206 - DNA, 19 bp, Artificial Sequence
  Description of Artificial Sequence Synthetic primer
gcagcatcaggtgcaacag

SEQ ID NO: 1207 - DNA, 25 bp, Artificial Sequence
  Description of Artificial Sequence Synthetic primer
ggcccatggtaaattttgagattgt

SEQ ID NO: 1208 - DNA, 25 bp, Artificial Sequence
  Description of Artificial Sequence Synthetic primer
ctcggaccattgtagtcatcttgaa

SEQ ID NO: 1209 - DNA, 28 bp, Artificial Sequence
  Description of Artificial Sequence Synthetic primer
cgtttctaatacacctgaaagtgatgca

SEQ ID NO: 1210 - DNA, 20 bp, Artificial Sequence
  Description of Artificial Sequence Synthetic primer
gcgctgctaggtttgttgtg

SEQ ID NO: 1211 - DNA, 25 bp, Artificial Sequence
  Description of Artificial Sequence Synthetic primer
ggcttttttgtgtggttctctgaaa

SEQ ID NO: 1212 - DNA, 27 bp, Artificial Sequence
  Description of Artificial Sequence Synthetic primer
agggattttattgctcgtgtcatttct

SEQ ID NO: 1213 - DNA, 24 bp, Artificial Sequence
  Description of Artificial Sequence Synthetic primer
tggcccatggtaaatgttgagatt

SEQ ID NO: 1214 - DNA, 25 bp, Artificial Sequence
  Description of Artificial Sequence Synthetic primer
tgttgtccgctttagaagcttgata

SEQ ID NO: 1215 - DNA, 24 bp, Artificial Sequence
  Description of Artificial Sequence Synthetic primer
cgtgtcagttctgggagtagtgta

SEQ ID NO: 1216 - DNA, 25 bp, Artificial Sequence
  Description of Artificial Sequence Synthetic primer
cgcggacatcttttaatgtagggat

SEQ ID NO: 1217 - DNA, 24 bp, Artificial Sequence
  Description of Artificial Sequence Synthetic primer
tggttgggtccgataatcaaaact

SEQ ID NO: 1218 - DNA, 25 bp, Artificial Sequence
  Description of Artificial Sequence Synthetic primer
ggcttttttgtgtggttctctgaaa

SEQ ID NO: 1219 - DNA, 25 bp, Artificial Sequence
  Description of Artificial Sequence Synthetic primer
tgtgtttgagctagtggtttggtta

SEQ ID NO: 1220 - DNA, 22 bp, Artificial Sequence
  Description of Artificial Sequence Synthetic primer
gtttgtgtttgagccagtggtt

SEQ ID NO: 1221 - DNA, 25 bp, Artificial Sequence
  Description of Artificial Sequence Synthetic primer
gttctgctcgtcttttaaacgcata

SEQ ID NO: 1222 - DNA, 23 bp, Artificial Sequence
  Description of Artificial Sequence Synthetic primer
gcgaatatgcttttccgcctaat

SEQ ID NO: 1223 - DNA, 25 bp, Artificial Sequence
  Description of Artificial Sequence Synthetic primer
cgtattcctgcttctagttgttggt

SEQ ID NO: 1224 - DNA, 28 bp, Artificial Sequence
  Description of Artificial Sequence Synthetic primer
tgctctaatatcgatgtaccgtattcct

SEQ ID NO: 1225 - DNA, 28 bp, Artificial Sequence
  Description of Artificial Sequence Synthetic primer
atcgttcctaatacacctgaaagtgatg

SEQ ID NO: 1226 - DNA, 25 bp, Artificial Sequence
  Description of Artificial Sequence Synthetic primer
gtgaaagttaatgaagtggcgctaa

SEQ ID NO: 1227 - DNA, 25 bp, Artificial Sequence
  Description of Artificial Sequence Synthetic primer
ggcccatggtaaattttgagattgt

SEQ ID NO: 1228 - DNA, 28 bp, Artificial Sequence
  Description of Artificial Sequence Synthetic primer
tctactgcgtctgttaagatatccgtat

SEQ ID NO: 1229 - DNA, 20 bp, Artificial Sequence
  Description of Artificial Sequence Synthetic primer
acgaccaccgtttgctttca

SEQ ID NO: 1230 - DNA, 28 bp, Artificial Sequence
  Description of Artificial Sequence Synthetic primer
tctactgcgtctgttaagatatccgtat

SEQ ID NO: 1231 - DNA, 28 bp, Artificial Sequence
  Description of Artificial Sequence Synthetic primer
tgtaaatacaagttctactgcgtctgtt

SEQ ID NO: 1232 - DNA, 25 bp, Artificial Sequence
  Description of Artificial Sequence Synthetic primer
ttgatcaagctccacaatgaaaacg

SEQ ID NO: 1233 - DNA, 24 bp, Artificial Sequence
  Description of Artificial Sequence Synthetic primer
gcaacgttcaatcgttttcttacg

SEQ ID NO: 1234 - DNA, 25 bp, Artificial Sequence
  Description of Artificial Sequence Synthetic primer
gcaacgttcaatcgttttcttacga

SEQ ID NO: 1235 - DNA, 25 bp, Artificial Sequence
  Description of Artificial Sequence Synthetic primer
cggatggatatgtttcagctaggat

SEQ ID NO: 1236 - DNA, 25 bp, Artificial Sequence
  Description of Artificial Sequence Synthetic primer
ttgatcaagctccacaatgaaaacg

SEQ ID NO: 1237 - DNA, 28 bp, Artificial Sequence
  Description of Artificial Sequence Synthetic primer
tgtaaatacaagttctactgcgtctgtt

SEQ ID NO: 1238 - DNA, 25 bp, Artificial Sequence
  Description of Artificial Sequence Synthetic primer
gcaacgttcaatcgttttcttacga

SEQ ID NO: 1239 - DNA, 25 bp, Artificial Sequence
  Description of Artificial Sequence Synthetic primer
ctgcgtctgttaagatatccgtgta

SEQ ID NO: 1240 - DNA, 25 bp, Artificial Sequence
  Description of Artificial Sequence Synthetic primer
ctgcgtctgttaagatatccgtgta

SEQ ID NO: 1241 - DNA, 32 bp, Artificial Sequence
  Description of Artificial Sequence Synthetic primer
aggatactctttcaaagttgttttcatcttga

SEQ ID NO: 1242 - DNA, 25 bp, Artificial Sequence
  Description of Artificial Sequence Synthetic primer
cggatggatatgtttcagctaggat

SEQ ID NO: 1243 - DNA, 18 bp, Artificial Sequence
  Description of Artificial Sequence Synthetic primer
gaccgccgtttgctttca

SEQ ID NO: 1244 - DNA, 25 bp, Artificial Sequence
  Description of Artificial Sequence Synthetic primer
ctgcgtctgttaagatatccgtgta

SEQ ID NO: 1245 - DNA, 22 bp, Artificial Sequence
  Description of Artificial Sequence Synthetic primer
ccaccgtttgctttcaatgtca

SEQ ID NO: 1246 - DNA, 25 bp, Artificial Sequence
  Description of Artificial Sequence Synthetic primer
gtctgatgtaaggttgttccagtct

SEQ ID NO: 1247 - DNA, 25 bp, Artificial Sequence
  Description of Artificial Sequence Synthetic primer
ctccaggtaatgtatcgccatcttt

SEQ ID NO: 1248 - DNA, 28 bp, Artificial Sequence
  Description of Artificial Sequence Synthetic primer
agcttttttgtttcgtcgtaattttcgt

SEQ ID NO: 1249 - DNA, 25 bp, Artificial Sequence
  Description of Artificial Sequence Synthetic primer
ccgccaccagtattaactttgctaa

SEQ ID NO: 1250 - DNA, 25 bp, Artificial Sequence
  Description of Artificial Sequence Synthetic primer
ctacccgcactttgctattttcttt

SEQ ID NO: 1251 - DNA, 25 bp, Artificial Sequence
  Description of Artificial Sequence Synthetic primer
caggtaatgtatcgccatctttcac

SEQ ID NO: 1252 - DNA, 21 bp, Artificial Sequence
  Description of Artificial Sequence Synthetic primer
gtggtgtggttcctgatcctt

SEQ ID NO: 1253 - DNA, 25 bp, Artificial Sequence
  Description of Artificial Sequence Synthetic primer
ggctgccacttaaatttgtatgtgt

SEQ ID NO: 1254 - DNA, 25 bp, Artificial Sequence
  Description of Artificial Sequence Synthetic primer
cggtatttgattgttgcacttccaa

SEQ ID NO: 1255 - DNA, 25 bp, Artificial Sequence
  Description of Artificial Sequence Synthetic primer
cttgtccaccactaccgtaatcatt

SEQ ID NO: 1256 - DNA, 19 bp, Artificial Sequence
  Description of Artificial Sequence Synthetic primer
cgttgtttgctccccgttt

SEQ ID NO: 1257 - DNA, 21 bp, Artificial Sequence
  Description of Artificial Sequence Synthetic primer
tgcggttactatctagcgcaa

SEQ ID NO: 1258 - DNA, 22 bp, Artificial Sequence
  Description of Artificial Sequence Synthetic primer
cccgtaatctgttgcagcttct

SEQ ID NO: 1259 - DNA, 24 bp, Artificial Sequence
  Description of Artificial Sequence Synthetic primer
cggttgcatttcttgaaactgcat

SEQ ID NO: 1260 - DNA, 23 bp, Artificial Sequence
  Description of Artificial Sequence Synthetic primer
cgcttgttgtgctagtcactttt

SEQ ID NO: 1261 - DNA, 21 bp, Artificial Sequence
  Description of Artificial Sequence Synthetic primer
gcaacgcctggtaaagtatcg

SEQ ID NO: 1262 - DNA, 19 bp, Artificial Sequence
  Description of Artificial Sequence Synthetic primer
gcacaggtggcacggtaat

SEQ ID NO: 1263 - DNA, 19 bp, Artificial Sequence
  Description of Artificial Sequence Synthetic primer
cctgtaccgccaccagtat

SEQ ID NO: 1264 - DNA, 29 bp, Artificial Sequence
  Description of Artificial Sequence Synthetic primer
tgtttcgtcgtaattttcgtatcatacgt

SEQ ID NO: 1265 - DNA, 25 bp, Artificial Sequence
  Description of Artificial Sequence Synthetic primer
cattatcaataggtgtcagcgcatt

SEQ ID NO: 1266 - DNA, 25 bp, Artificial Sequence
  Description of Artificial Sequence Synthetic primer
gtcattcactggaaaaccaccaaaa

SEQ ID NO: 1267 - DNA, 25 bp, Artificial Sequence
  Description of Artificial Sequence Synthetic primer
agtcggaaaatcatagcgtgcatta

SEQ ID NO: 1268 - DNA, 25 bp, Artificial Sequence
  Description of Artificial Sequence Synthetic primer
cgaatgcagcaagtgcttttaaatt

SEQ ID NO: 1269 - DNA, 24 bp, Artificial Sequence
  Description of Artificial Sequence Synthetic primer
ccgcccactgttaagtaagtcaaa

SEQ ID NO: 1270 - DNA, 24 bp, Artificial Sequence
  Description of Artificial Sequence Synthetic primer
ccgcgctcattttcagcatttaat

SEQ ID NO: 1271 - DNA, 23 bp, Artificial Sequence
  Description of Artificial Sequence Synthetic primer
ctttgagtcctgcagcattttct

SEQ ID NO: 1272 - DNA, 23 bp, Artificial Sequence
  Description of Artificial Sequence Synthetic primer
ccatcgtccgtttttgcttgaac

SEQ ID NO: 1273 - DNA, 24 bp, Artificial Sequence
  Description of Artificial Sequence Synthetic primer
tcatggagttttgcgcatagagat

SEQ ID NO: 1274 - DNA, 25 bp, Artificial Sequence
  Description of Artificial Sequence Synthetic primer
gctagtgtttggatgctcgtgttat

SEQ ID NO: 1275 - DNA, 23 bp, Artificial Sequence
  Description of Artificial Sequence Synthetic primer
tgtcggatctgctgtgataactg

SEQ ID NO: 1276 - DNA, 34 bp, Artificial Sequence
  Description of Artificial Sequence Synthetic primer
acaagtccatttaaatctacgaaaagactatctt

SEQ ID NO: 1277 - DNA, 26 bp, Artificial Sequence
  Description of Artificial Sequence Synthetic primer
tttgtccaaatgctgctaaagctttt

SEQ ID NO: 1278 - DNA, 40 bp, Artificial Sequence
  Description of Artificial Sequence Synthetic primer
gactaacatttttactgctaatacttatacttttcaagtt

SEQ ID NO: 1279 - DNA, 23 bp, Artificial Sequence
  Description of Artificial Sequence Synthetic primer
gagggctttcaagttgccatttt

SEQ ID NO: 1280 - DNA, 30 bp, Artificial Sequence
  Description of Artificial Sequence Synthetic primer
ttgcattattttctttcacttggtcttctg

SEQ ID NO: 1281 - DNA, 26 bp, Artificial Sequence
  Description of Artificial Sequence Synthetic primer
tttgtccaaatgctgctaaagctttt

SEQ ID NO: 1282 - DNA, 23 bp, Artificial Sequence
  Description of Artificial Sequence Synthetic primer
gtcgcttgtagtcactgttacga

SEQ ID NO: 1283 - DNA, 24 bp, Artificial Sequence
  Description of Artificial Sequence Synthetic primer
caccgctgactgtaataccatcat

SEQ ID NO: 1284 - DNA, 25 bp, Artificial Sequence
  Description of Artificial Sequence Synthetic primer
gtaggatctgctgtgataacaggtt

SEQ ID NO: 1285 - DNA, 23 bp, Artificial Sequence
  Description of Artificial Sequence Synthetic primer
cggtaatggaaagacgtgagctt

SEQ ID NO: 1286 - DNA, 24 bp, Artificial Sequence
  Description of Artificial Sequence Synthetic primer
gccgcggtcatttattgcatttaa

SEQ ID NO: 1287 - DNA, 23 bp, Artificial Sequence
  Description of Artificial Sequence Synthetic primer
gttggtggaaccggattaacatg

SEQ ID NO: 1288 - DNA, 21 bp, Artificial Sequence
  Description of Artificial Sequence Synthetic primer
acttgcactggcaaagctttt

SEQ ID NO: 1289 - DNA, 25 bp, Artificial Sequence
  Description of Artificial Sequence Synthetic primer
gcatcagcagatttgccagttaaaa

SEQ ID NO: 1290 - DNA, 29 bp, Artificial Sequence
  Description of Artificial Sequence Synthetic primer
ccagtagagttaaattagtcaacccactt

SEQ ID NO: 1291 - DNA, 26 bp, Artificial Sequence
  Description of Artificial Sequence Synthetic primer
gctttttgcaaatcattttccgcatt

SEQ ID NO: 1292 - DNA, 25 bp, Artificial Sequence
  Description of Artificial Sequence Synthetic primer
cgttccaggagtcttgaaatctact

SEQ ID NO: 1293 - DNA, 29 bp, Artificial Sequence
  Description of Artificial Sequence Synthetic primer
tctccttgaaacaataaagcttaatccga

SEQ ID NO: 1294 - DNA, 25 bp, Artificial Sequence
  Description of Artificial Sequence Synthetic primer
ggattaggtgttggacttggatctg

SEQ ID NO: 1295 - DNA, 24 bp, Artificial Sequence
  Description of Artificial Sequence Synthetic primer
ttttaccgcctatggtttggctat

SEQ ID NO: 1296 - DNA, 25 bp, Artificial Sequence
  Description of Artificial Sequence Synthetic primer
gcagtaatcgttgtcttcgctttaa

SEQ ID NO: 1297 - DNA, 23 bp, Artificial Sequence
  Description of Artificial Sequence Synthetic primer
gtcatcaagctgaatgggacgta

SEQ ID NO: 1298 - DNA, 25 bp, Artificial Sequence
  Description of Artificial Sequence Synthetic primer
gttgatccgtcatctgtttttgcat

SEQ ID NO: 1299 - DNA, 28 bp, Artificial Sequence
  Description of Artificial Sequence Synthetic primer
ttttaagtggcatgatatccgttattgc

SEQ ID NO: 1300 - DNA, 25 bp, Artificial Sequence
  Description of Artificial Sequence Synthetic primer
tggccatatgcatacagttgattca

SEQ ID NO: 1301 - DNA, 23 bp, Artificial Sequence
  Description of Artificial Sequence Synthetic primer
atgagcactgtgtgaaagatcca

SEQ ID NO: 1302 - DNA, 25 bp, Artificial Sequence
  Description of Artificial Sequence Synthetic primer
gccgtaatcgctgtcttctctttaa

SEQ ID NO: 1303 - DNA, 28 bp, Artificial Sequence
  Description of Artificial Sequence Synthetic primer
ggcataatatctgttattgcaccattgt

SEQ ID NO: 1304 - DNA, 25 bp, Artificial Sequence
  Description of Artificial Sequence Synthetic primer
ctctttgcttgtttctgctttagct

SEQ ID NO: 1305 - DNA, 21 bp, Artificial Sequence
  Description of Artificial Sequence Synthetic primer
aggcaaggaatcccctgtttt

SEQ ID NO: 1306 - DNA, 25 bp, Artificial Sequence
  Description of Artificial Sequence Synthetic primer
cagatatattgtacgccccaccatt

SEQ ID NO: 1307 - DNA, 26 bp, Artificial Sequence
  Description of Artificial Sequence Synthetic primer
gatgttcaacgaatggtctacagtga

SEQ ID NO: 1308 - DNA, 25 bp, Artificial Sequence
  Description of Artificial Sequence Synthetic primer
tcggatctggtattggtgtttcttt

SEQ ID NO: 1309 - DNA, 21 bp, Artificial Sequence
  Description of Artificial Sequence Synthetic primer
tgcaggaggattttctgcatt

SEQ ID NO: 1310 - DNA, 21 bp, Artificial Sequence
  Description of Artificial Sequence Synthetic probe
cctgatataacatggaattta

SEQ ID NO: 1311 - DNA, 20 bp, Artificial Sequence
  Description of Artificial Sequence Synthetic probe
tcgggtctaacaaaactaac

SEQ ID NO: 1312 - DNA, 17 bp, Artificial Sequence
  Description of Artificial Sequence Synthetic probe
cacggtctcacaaacag

SEQ ID NO: 1313 - DNA, 18 bp, Artificial Sequence
  Description of Artificial Sequence Synthetic probe
tcctactgctaataaccc

SEQ ID NO: 1314 - DNA, 20 bp, Artificial Sequence
  Description of Artificial Sequence Synthetic probe
tttcgtcataccaacctttg

SEQ ID NO: 1315 - DNA, 17 bp, Artificial Sequence
  Description of Artificial Sequence Synthetic probe
aagcgggtctatatccg

SEQ ID NO: 1316 - DNA, 23 bp, Artificial Sequence
  Description of Artificial Sequence Synthetic probe
ttggacgaattatccttaaatgg

SEQ ID NO: 1317 - DNA, 18 bp, Artificial Sequence
  Description of Artificial Sequence Synthetic probe
ccgctagagctgtatctg

SEQ ID NO: 1318 - DNA, 20 bp, Artificial Sequence
  Description of Artificial Sequence Synthetic probe
aagcccagtttctagtttaa

SEQ ID NO: 1319 - DNA, 17 bp, Artificial Sequence
  Description of Artificial Sequence Synthetic probe
acattggcgagtttaac

SEQ ID NO: 1320 - DNA, 24 bp, Artificial Sequence
  Description of Artificial Sequence Synthetic probe
aactcagttagttttgttagaccc

SEQ ID NO: 1321 - DNA, 17 bp, Artificial Sequence
  Description of Artificial Sequence Synthetic probe
cacggttccactaaatg

SEQ ID NO: 1322 - DNA, 20 bp, Artificial Sequence
  Description of Artificial Sequence Synthetic probe
accaagttacaacgcttcag

SEQ ID NO: 1323 - DNA, 15 bp, Artificial Sequence
  Description of Artificial Sequence Synthetic probe
tcttgagcaaaatcc

SEQ ID NO: 1324 - DNA, 18 bp, Artificial Sequence
  Description of Artificial Sequence Synthetic probe
cctacaactggcgatagc

SEQ ID NO: 1325 - DNA, 18 bp, Artificial Sequence
  Description of Artificial Sequence Synthetic probe
atcaactgggaattcagc

SEQ ID NO: 1326 - DNA, 23 bp, Artificial Sequence
  Description of Artificial Sequence Synthetic probe
cacaaataaatttcagcaataat

SEQ ID NO: 1327 - DNA, 20 bp, Artificial Sequence
  Description of Artificial Sequence Synthetic probe
tcgttgttgttccttctttg

SEQ ID NO: 1328 - DNA, 20 bp, Artificial Sequence
  Description of Artificial Sequence Synthetic probe
cttagagctaaatgaaaatc

SEQ ID NO: 1329 - DNA, 19 bp, Artificial Sequence
  Description of Artificial Sequence Synthetic probe
aaggaacgacaacatttag

SEQ ID NO: 1330 - DNA, 17 bp, Artificial Sequence
  Description of Artificial Sequence Synthetic probe
acgaaagccggatatac

SEQ ID NO: 1331 - DNA, 16 bp, Artificial Sequence
  Description of Artificial Sequence Synthetic probe
ccattcgcgtaaagcg

SEQ ID NO: 1332 - DNA, 19 bp, Artificial Sequence
  Description of Artificial Sequence Synthetic probe
aaaagatgtggaagacttc

SEQ ID NO: 1333 - DNA, 20 bp, Artificial Sequence
  Description of Artificial Sequence Synthetic probe
cttgtttctgaagaacttac

SEQ ID NO: 1334 - DNA, 17 bp, Artificial Sequence
  Description of Artificial Sequence Synthetic probe
aagtgccgataaatctt

SEQ ID NO: 1335 - DNA, 18 bp, Artificial Sequence
  Description of Artificial Sequence Synthetic probe
acgaaggtagcggacttt

SEQ ID NO: 1336 - DNA, 18 bp, Artificial Sequence
  Description of Artificial Sequence Synthetic probe
tttgctggttttaatttc

SEQ ID NO: 1337 - DNA, 16 bp, Artificial Sequence
  Description of Artificial Sequence Synthetic probe
ccagcaatcaaagctc

SEQ ID NO: 1338 - DNA, 20 bp, Artificial Sequence
  Description of Artificial Sequence Synthetic probe
acgaagtgcagataagtctt

SEQ ID NO: 1339 - DNA, 21 bp, Artificial Sequence
  Description of Artificial Sequence Synthetic probe
atggtgtagatgtacttaaac

SEQ ID NO: 1340 - DNA, 16 bp, Artificial Sequence
  Description of Artificial Sequence Synthetic probe
cacgaccgcaaagtac

SEQ ID NO: 1341 - DNA, 19 bp, Artificial Sequence
  Description of Artificial Sequence Synthetic probe
tatcgttggagagtctttg

SEQ ID NO: 1342 - DNA, 19 bp, Artificial Sequence
  Description of Artificial Sequence Synthetic probe
tctaaatcaagcaaaatcg

SEQ ID NO: 1343 - DNA, 17 bp, Artificial Sequence
  Description of Artificial Sequence Synthetic probe
ccttcagcaaaaccttc

SEQ ID NO: 1344 - DNA, 16 bp, Artificial Sequence
  Description of Artificial Sequence Synthetic probe
ccagcaatcaaagctc

SEQ ID NO: 1345 - DNA, 23 bp, Artificial Sequence
  Description of Artificial Sequence Synthetic probe
aatcaacataacgtacaatatag

SEQ ID NO: 1346 - DNA, 18 bp, Artificial Sequence
  Description of Artificial Sequence Synthetic probe
aagcagctcgtcattttg

SEQ ID NO: 1347 - DNA, 19 bp, Artificial Sequence
  Description of Artificial Sequence Synthetic probe
caggatatgacgcaactac

SEQ ID NO: 1348 - DNA, 16 bp, Artificial Sequence
  Description of Artificial Sequence Synthetic probe
catcgccttgtttgcc

SEQ ID NO: 1349 - DNA, 18 bp, Artificial Sequence
  Description of Artificial Sequence Synthetic probe
caccgtaacgaggtttag

SEQ ID NO: 1350 - DNA, 19 bp, Artificial Sequence
  Description of Artificial Sequence Synthetic probe
acaccataacgaggtttag

SEQ ID NO: 1351 - DNA, 18 bp, Artificial Sequence
  Description of Artificial Sequence Synthetic probe
atgagcctgctgaaatta

SEQ ID NO: 1352 - DNA, 20 bp, Artificial Sequence
  Description of Artificial Sequence Synthetic probe
tcggtaaagcaacagatgat

SEQ ID NO: 1353 - DNA, 17 bp, Artificial Sequence
  Description of Artificial Sequence Synthetic probe
acgcaaaagacaagttc

SEQ ID NO: 1354 - DNA, 18 bp, Artificial Sequence
  Description of Artificial Sequence Synthetic probe
acttcgcctttagctttt

SEQ ID NO: 1355 - DNA, 18 bp, Artificial Sequence
  Description of Artificial Sequence Synthetic probe
acttcgcctttagctttt

SEQ ID NO: 1356 - DNA, 20 bp, Artificial Sequence
  Description of Artificial Sequence Synthetic probe
acaagtagaaggtaaagctc

SEQ ID NO: 1357 - DNA, 22 bp, Artificial Sequence
  Description of Artificial Sequence Synthetic probe
ttgcgtcacctactactttatc

SEQ ID NO: 1358 - DNA, 18 bp, Artificial Sequence
  Description of Artificial Sequence Synthetic probe
accttctgcttgtttcgc

SEQ ID NO: 1359 - DNA, 22 bp, Artificial Sequence
  Description of Artificial Sequence Synthetic probe
ttgcgtcacctactactttatc

SEQ ID NO: 1360 - DNA, 17 bp, Artificial Sequence
  Description of Artificial Sequence Synthetic probe
acgcaaaagacaaattc

SEQ ID NO: 1361 - DNA, 22 bp, Artificial Sequence
  Description of Artificial Sequence Synthetic probe
ttgcgtcacctactactttgtc

SEQ ID NO: 1362 - DNA, 17 bp, Artificial Sequence
  Description of Artificial Sequence Synthetic probe
caacagacgacaaagcg

SEQ ID NO: 1363 - DNA, 19 bp, Artificial Sequence
  Description of Artificial Sequence Synthetic probe
acaagttgaaggtaaagct

SEQ ID NO: 1364 - DNA, 18 bp, Artificial Sequence
  Description of Artificial Sequence Synthetic probe
acttcgcctttagctttt

SEQ ID NO: 1365 - DNA, 22 bp, Artificial Sequence
  Description of Artificial Sequence Synthetic probe
ttgcgtcacctactactttgtc

SEQ ID NO: 1366 - DNA, 17 bp, Artificial Sequence
  Description of Artificial Sequence Synthetic probe
tcatctgttgctttccc

SEQ ID NO: 1367 - DNA, 17 bp, Artificial Sequence
  Description of Artificial Sequence Synthetic probe
acgcaaaagacaaattc

SEQ ID NO: 1368 - DNA, 21 bp, Artificial Sequence
  Description of Artificial Sequence Synthetic probe
ttgcgtcacctactactttgt

SEQ ID NO: 1369 - DNA, 17 bp, Artificial Sequence
  Description of Artificial Sequence Synthetic probe
acgcaaaagacaaattc

SEQ ID NO: 1370 - DNA, 23 bp, Artificial Sequence
  Description of Artificial Sequence Synthetic probe
aagcaaaaggacttaaagacaaa

SEQ ID NO: 1371 - DNA, 16 bp, Artificial Sequence
  Description of Artificial Sequence Synthetic probe
tttggaaggaatttcg

SEQ ID NO: 1372 - DNA, 25 bp, Artificial Sequence
  Description of Artificial Sequence Synthetic probe
caacatttgattaccaactatttaa

SEQ ID NO: 1373 - DNA, 22 bp, Artificial Sequence
  Description of Artificial Sequence Synthetic probe
atggctgatcaaaacgaatcta

SEQ ID NO: 1374 - DNA, 20 bp, Artificial Sequence
  Description of Artificial Sequence Synthetic probe
atggctgatcaaactgaatc

SEQ ID NO: 1375 - DNA, 18 bp, Artificial Sequence
  Description of Artificial Sequence Synthetic probe
caggtaagccaatccccc

SEQ ID NO: 1376 - DNA, 16 bp, Artificial Sequence
  Description of Artificial Sequence Synthetic probe
ccgagcctaaaatatg

SEQ ID NO: 1377 - DNA, 16 bp, Artificial Sequence
  Description of Artificial Sequence Synthetic probe
ccgagcctaaaatatg

SEQ ID NO: 1378 - DNA, 20 bp, Artificial Sequence
  Description of Artificial Sequence Synthetic probe
ttgccgctatctcctttaca

SEQ ID NO: 1379 - DNA, 16 bp, Artificial Sequence
  Description of Artificial Sequence Synthetic probe
cacgccaacaggatta

SEQ ID NO: 1380 - DNA, 23 bp, Artificial Sequence
  Description of Artificial Sequence Synthetic probe
cagagaatcaatctcgtaaatat

SEQ ID NO: 1381 - DNA, 17 bp, Artificial Sequence
  Description of Artificial Sequence Synthetic probe
acctgaaacagaattca

SEQ ID NO: 1382 - DNA, 18 bp, Artificial Sequence
  Description of Artificial Sequence Synthetic probe
ccgctgtctccattacat

SEQ ID NO: 1383 - DNA, 21 bp, Artificial Sequence
  Description of Artificial Sequence Synthetic probe
cttttaccaaattgaattctg

SEQ ID NO: 1384 - DNA, 21 bp, Artificial Sequence
  Description of Artificial Sequence Synthetic probe
cttacctggtgatttatcttc

SEQ ID NO: 1385 - DNA, 19 bp, Artificial Sequence
  Description of Artificial Sequence Synthetic probe
cccacgactataagcatcc

SEQ ID NO: 1386 - DNA, 19 bp, Artificial Sequence
  Description of Artificial Sequence Synthetic probe
cccacgactataagcatcc

SEQ ID NO: 1387 - DNA, 16 bp, Artificial Sequence
  Description of Artificial Sequence Synthetic probe
ctcgtgttgcaatatc

SEQ ID NO: 1388 - DNA, 23 bp, Artificial Sequence
  Description of Artificial Sequence Synthetic probe
ctttttgttgttccaccgagcct

SEQ ID NO: 1389 - DNA, 24 bp, Artificial Sequence
  Description of Artificial Sequence Synthetic probe
caaataatccaacatttgattacc

SEQ ID NO: 1390 - DNA, 18 bp, Artificial Sequence
  Description of Artificial Sequence Synthetic probe
aagccgattccgcccatc

SEQ ID NO: 1391 - DNA, 17 bp, Artificial Sequence
  Description of Artificial Sequence Synthetic probe
cctgtaccataatttcc

SEQ ID NO: 1392 - DNA, 16 bp, Artificial Sequence
  Description of Artificial Sequence Synthetic probe
ccttgagcgaacttag

SEQ ID NO: 1393 - DNA, 16 bp, Artificial Sequence
  Description of Artificial Sequence Synthetic probe
ctttcgccccgtttcc

SEQ ID NO: 1394 - DNA, 22 bp, Artificial Sequence
  Description of Artificial Sequence Synthetic probe
aagcatggagaaagatactaat

SEQ ID NO: 1395 - DNA, 17 bp, Artificial Sequence
  Description of Artificial Sequence Synthetic probe
caagcggagactatcac

SEQ ID NO: 1396 - DNA, 20 bp, Artificial Sequence
  Description of Artificial Sequence Synthetic probe
cagtcgtttccactttaaac

SEQ ID NO: 1397 - DNA, 23 bp, Artificial Sequence
  Description of Artificial Sequence Synthetic probe
atgaacaagtccgtttatatcac

SEQ ID NO: 1398 - DNA, 23 bp, Artificial Sequence
  Description of Artificial Sequence Synthetic probe
tttataagcagctaaagtgcccc

SEQ ID NO: 1399 - DNA, 25 bp, Artificial Sequence
  Description of Artificial Sequence Synthetic probe
cgatcgacaattaatgtttgatttt

SEQ ID NO: 1400 - DNA, 19 bp, Artificial Sequence
  Description of Artificial Sequence Synthetic probe
aacgacccatcagtgtttt

SEQ ID NO: 1401 - DNA, 20 bp, Artificial Sequence
  Description of Artificial Sequence Synthetic probe
atggcatttaccagaattta

SEQ ID NO: 1402 - DNA, 20 bp, Artificial Sequence
  Description of Artificial Sequence Synthetic probe
tttcggtcgtttccgcttta

SEQ ID NO: 1403 - DNA, 20 bp, Artificial Sequence
  Description of Artificial Sequence Synthetic probe
acgcggataactgcacctaa

SEQ ID NO: 1404 - DNA, 23 bp, Artificial Sequence
  Description of Artificial Sequence Synthetic probe
ttagcattgatggtaaagtaatt

SEQ ID NO: 1405 - DNA, 17 bp, Artificial Sequence
  Description of Artificial Sequence Synthetic probe
ctcatgcccaccatttt

SEQ ID NO: 1406 - DNA, 22 bp, Artificial Sequence
  Description of Artificial Sequence Synthetic probe
atggctttttttagacgaaaat

SEQ ID NO: 1407 - DNA, 22 bp, Artificial Sequence
  Description of Artificial Sequence Synthetic probe
cagaaactatcaaagacaattt

SEQ ID NO: 1408 - DNA, 19 bp, Artificial Sequence
  Description of Artificial Sequence Synthetic probe
tcagtcgtttccgctttaa

SEQ ID NO: 1409 - DNA, 21 bp, Artificial Sequence
  Description of Artificial Sequence Synthetic probe
aagacattcttggctaaataa

SEQ ID NO: 1410 - DNA, 18 bp, Artificial Sequence
  Description of Artificial Sequence Synthetic probe
atagcgtgttaaattcac

SEQ ID NO: 1411 - DNA, 16 bp, Artificial Sequence
  Description of Artificial Sequence Synthetic probe
atgcccaccatttttt

SEQ ID NO: 1412 - DNA, 17 bp, Artificial Sequence
  Description of Artificial Sequence Synthetic probe
cccacataccgaaaagt

SEQ ID NO: 1413 - DNA, 16 bp, Artificial Sequence
  Description of Artificial Sequence Synthetic probe
atagcgcgataaaacc

SEQ ID NO: 1414 - DNA, 16 bp, Artificial Sequence
  Description of Artificial Sequence Synthetic probe
tagctgacggaatttt

SEQ ID NO: 1415 - DNA, 15 bp, Artificial Sequence
  Description of Artificial Sequence Synthetic probe
acccagccagaaaca

SEQ ID NO: 1416 - DNA, 19 bp, Artificial Sequence
  Description of Artificial Sequence Synthetic probe
accttgaacgatgaagaaa

SEQ ID NO: 1417 - DNA, 16 bp, Artificial Sequence
  Description of Artificial Sequence Synthetic probe
tagctgacggaatttt

SEQ ID NO: 1418 - DNA, 21 bp, Artificial Sequence
  Description of Artificial Sequence Synthetic probe
ccaaacccctagtataatcat

SEQ ID NO: 1419 - DNA, 21 bp, Artificial Sequence
  Description of Artificial Sequence Synthetic probe
accaacaatgataccaataat

SEQ ID NO: 1420 - DNA, 19 bp, Artificial Sequence
  Description of Artificial Sequence Synthetic probe
caataatgccgataatacc

SEQ ID NO: 1421 - DNA, 19 bp, Artificial Sequence
  Description of Artificial Sequence Synthetic probe
actgatttctggtattttc

SEQ ID NO: 1422 - DNA, 21 bp, Artificial Sequence
  Description of Artificial Sequence Synthetic probe
ctggtattatgcgtacaattg

SEQ ID NO: 1423 - DNA, 19 bp, Artificial Sequence
  Description of Artificial Sequence Synthetic probe
ttgtttgctggtattatgc

SEQ ID NO: 1424 - DNA, 17 bp, Artificial Sequence
  Description of Artificial Sequence Synthetic probe
cccaaccagaaacattt

SEQ ID NO: 1425 - DNA, 18 bp, Artificial Sequence
  Description of Artificial Sequence Synthetic probe
attttccacacaatttct

SEQ ID NO: 1426 - DNA, 19 bp, Artificial Sequence
  Description of Artificial Sequence Synthetic probe
caataatgccgataatacc

SEQ ID NO: 1427 - DNA, 17 bp, Artificial Sequence
  Description of Artificial Sequence Synthetic probe
caattgcggaaacaact

SEQ ID NO: 1428 - DNA, 19 bp, Artificial Sequence
  Description of Artificial Sequence Synthetic probe
caacgacaaggacgatacc

SEQ ID NO: 1429 - DNA, 21 bp, Artificial Sequence
  Description of Artificial Sequence Synthetic probe
ttgcaacgataagtactattc

SEQ ID NO: 1430 - DNA, 21 bp, Artificial Sequence
  Description of Artificial Sequence Synthetic probe
ccgtcaaattcattaaataat

SEQ ID NO: 1431 - DNA, 19 bp, Artificial Sequence
  Description of Artificial Sequence Synthetic probe
cctggatggaataagaacc

SEQ ID NO: 1432 - DNA, 17 bp, Artificial Sequence
  Description of Artificial Sequence Synthetic probe
aagagagcgataaatcc

SEQ ID NO: 1433 - DNA, 19 bp, Artificial Sequence
  Description of Artificial Sequence Synthetic probe
atgccaagatccgctaatg

SEQ ID NO: 1434 - DNA, 18 bp, Artificial Sequence
  Description of Artificial Sequence Synthetic probe
ccctagctcagaactact

SEQ ID NO: 1435 - DNA, 16 bp, Artificial Sequence
  Description of Artificial Sequence Synthetic probe
ttagctggaaattttg

SEQ ID NO: 1436 - DNA, 18 bp, Artificial Sequence
  Description of Artificial Sequence Synthetic probe
ctggatcatcaatgtccc

SEQ ID NO: 1437 - DNA, 22 bp, Artificial Sequence
  Description of Artificial Sequence Synthetic probe
cagatgctgatctcattgtaac

SEQ ID NO: 1438 - DNA, 17 bp, Artificial Sequence
  Description of Artificial Sequence Synthetic probe
ttgggcgaaaagacttt

SEQ ID NO: 1439 - DNA, 19 bp, Artificial Sequence
  Description of Artificial Sequence Synthetic probe
actcattcaacgcttctgc

SEQ ID NO: 1440 - DNA, 21 bp, Artificial Sequence
  Description of Artificial Sequence Synthetic probe
ctgaaagcttgtcttattctc

SEQ ID NO: 1441 - DNA, 22 bp, Artificial Sequence
  Description of Artificial Sequence Synthetic probe
caagaagtatgtgataacattg

SEQ ID NO: 1442 - DNA, 18 bp, Artificial Sequence
  Description of Artificial Sequence Synthetic probe
ctggatcatcaatgtccc

SEQ ID NO: 1443 - DNA, 15 bp, Artificial Sequence
  Description of Artificial Sequence Synthetic probe
ctgcaccaaaatttc

SEQ ID NO: 1444 - DNA, 21 bp, Artificial Sequence
  Description of Artificial Sequence Synthetic probe
caatgttatcacagacttctt

SEQ ID NO: 1445 - DNA, 22 bp, Artificial Sequence
  Description of Artificial Sequence Synthetic probe
cagatgctgatctcattgtaac

SEQ ID NO: 1446 - DNA, 20 bp, Artificial Sequence
  Description of Artificial Sequence Synthetic probe
ccagctaacatacaagaaaa

SEQ ID NO: 1447 - DNA, 22 bp, Artificial Sequence
  Description of Artificial Sequence Synthetic probe
ttttatcacaaacgcatattcg

SEQ ID NO: 1448 - DNA, 21 bp, Artificial Sequence
  Description of Artificial Sequence Synthetic probe
ctgctaacatacaagaaaaaa

SEQ ID NO: 1449 - DNA, 17 bp, Artificial Sequence
  Description of Artificial Sequence Synthetic probe
aaatgggcgagttatca

SEQ ID NO: 1450 - DNA, 18 bp, Artificial Sequence
  Description of Artificial Sequence Synthetic probe
tccagaacaagaaatagc

SEQ ID NO: 1451 - DNA, 21 bp, Artificial Sequence
  Description of Artificial Sequence Synthetic probe
cagaggctgatctcattgtta

SEQ ID NO: 1452 - DNA, 22 bp, Artificial Sequence
  Description of Artificial Sequence Synthetic probe
cagatgctgatctcattgtaac

SEQ ID NO: 1453 - DNA, 19 bp, Artificial Sequence
  Description of Artificial Sequence Synthetic probe
aaagcttgtcttattcccc

SEQ ID NO: 1454 - DNA, 16 bp, Artificial Sequence
  Description of Artificial Sequence Synthetic probe
acgcccttgtgaatcg

SEQ ID NO: 1455 - DNA, 20 bp, Artificial Sequence
  Description of Artificial Sequence Synthetic probe
ctcatgtctaaacgaatcat

SEQ ID NO: 1456 - DNA, 16 bp, Artificial Sequence
  Description of Artificial Sequence Synthetic probe
cattcgccccaaagtt

SEQ ID NO: 1457 - DNA, 18 bp, Artificial Sequence
  Description of Artificial Sequence Synthetic probe
tcggtgcaactacattgg

SEQ ID NO: 1458 - DNA, 20 bp, Artificial Sequence
  Description of Artificial Sequence Synthetic probe
ccgtctagatagcgttattg

SEQ ID NO: 1459 - DNA, 21 bp, Artificial Sequence
  Description of Artificial Sequence Synthetic probe
atggtctgaaaacaaagtatg

SEQ ID NO: 1460 - DNA, 20 bp, Artificial Sequence
  Description of Artificial Sequence Synthetic probe
tcttgaccaattacttttcc

SEQ ID NO: 1461 - DNA, 24 bp, Artificial Sequence
  Description of Artificial Sequence Synthetic probe
acttaacaggtaaagttattggcc

SEQ ID NO: 1462 - DNA, 18 bp, Artificial Sequence
  Description of Artificial Sequence Synthetic probe
tagcagcaccactcaatt

SEQ ID NO: 1463 - DNA, 14 bp, Artificial Sequence
  Description of Artificial Sequence Synthetic probe
ccgcggtcaatttg

SEQ ID NO: 1464 - DNA, 21 bp, Artificial Sequence
  Description of Artificial Sequence Synthetic probe
ctccgaatttagggtcataac

SEQ ID NO: 1465 - DNA, 19 bp, Artificial Sequence
  Description of Artificial Sequence Synthetic probe
acttcaagaacgtaaaaac

SEQ ID NO: 1466 - DNA, 17 bp, Artificial Sequence
  Description of Artificial Sequence Synthetic probe
caggtgttggtaaaact

SEQ ID NO: 1467 - DNA, 16 bp, Artificial Sequence
  Description of Artificial Sequence Synthetic probe
ctgcgtccattgaacc

SEQ ID NO: 1468 - DNA, 21 bp, Artificial Sequence
  Description of Artificial Sequence Synthetic probe
attgggcgattttttgatttg

SEQ ID NO: 1469 - DNA, 18 bp, Artificial Sequence
  Description of Artificial Sequence Synthetic probe
aagcgaacgtgttgcccg

SEQ ID NO: 1470 - DNA, 24 bp, Artificial Sequence
  Description of Artificial Sequence Synthetic probe
ctcgattaaatgttctttaacttc

SEQ ID NO: 1471 - DNA, 18 bp, Artificial Sequence
  Description of Artificial Sequence Synthetic probe
aagaacgcatgaaacaac

SEQ ID NO: 1472 - DNA, 19 bp, Artificial Sequence
  Description of Artificial Sequence Synthetic probe
caattccttctcagaacgc

SEQ ID NO: 1473 - DNA, 18 bp, Artificial Sequence
  Description of Artificial Sequence Synthetic probe
catttgccagataaagca

SEQ ID NO: 1474 - DNA, 20 bp, Artificial Sequence
  Description of Artificial Sequence Synthetic probe
catatcaatccgaatcatcg

SEQ ID NO: 1475 - DNA, 18 bp, Artificial Sequence
  Description of Artificial Sequence Synthetic probe
ttgcttccctaaattttg

SEQ ID NO: 1476 - DNA, 17 bp, Artificial Sequence
  Description of Artificial Sequence Synthetic probe
aaacaagcgagataaac

SEQ ID NO: 1477 - DNA, 24 bp, Artificial Sequence
  Description of Artificial Sequence Synthetic probe
atggatttccccttattatatcag

SEQ ID NO: 1478 - DNA, 20 bp, Artificial Sequence
  Description of Artificial Sequence Synthetic probe
cagaccttgtatcacaaaag

SEQ ID NO: 1479 - DNA, 21 bp, Artificial Sequence
  Description of Artificial Sequence Synthetic probe
caggtggactaactagattga

SEQ ID NO: 1480 - DNA, 20 bp, Artificial Sequence
  Description of Artificial Sequence Synthetic probe
caacaaccaacgcctattaa

SEQ ID NO: 1481 - DNA, 15 bp, Artificial Sequence
  Description of Artificial Sequence Synthetic probe
ccggaatgcaatttt

SEQ ID NO: 1482 - DNA, 22 bp, Artificial Sequence
  Description of Artificial Sequence Synthetic probe
actccagatagttccttttgtg

SEQ ID NO: 1483 - DNA, 22 bp, Artificial Sequence
  Description of Artificial Sequence Synthetic probe
acagatagctcttctaacttag

SEQ ID NO: 1484 - DNA, 20 bp, Artificial Sequence
  Description of Artificial Sequence Synthetic probe
cagaaatgtgtgaatgaacc

SEQ ID NO: 1485 - DNA, 17 bp, Artificial Sequence
  Description of Artificial Sequence Synthetic probe
tcgcagcttgtactttt

SEQ ID NO: 1486 - DNA, 16 bp, Artificial Sequence
  Description of Artificial Sequence Synthetic probe
ttcccgcaagagattg

SEQ ID NO: 1487 - DNA, 20 bp, Artificial Sequence
  Description of Artificial Sequence Synthetic probe
ttgtgggatagatgaagttc

SEQ ID NO: 1488 - DNA, 19 bp, Artificial Sequence
  Description of Artificial Sequence Synthetic probe
ctctcagcttgtacttttg

SEQ ID NO: 1489 - DNA, 23 bp, Artificial Sequence
  Description of Artificial Sequence Synthetic probe
actgatataatatggagatatcc

SEQ ID NO: 1490 - DNA, 20 bp, Artificial Sequence
  Description of Artificial Sequence Synthetic probe
ctggttcattcacacatttc

SEQ ID NO: 1491 - DNA, 15 bp, Artificial Sequence
  Description of Artificial Sequence Synthetic probe
tcaacgaccaacccc

SEQ ID NO: 1492 - DNA, 21 bp, Artificial Sequence
  Description of Artificial Sequence Synthetic probe
ccccagatagttccttttgtg

SEQ ID NO: 1493 - DNA, 20 bp, Artificial Sequence
  Description of Artificial Sequence Synthetic probe
ctgtataaactggcaattcc

SEQ ID NO: 1494 - DNA, 23 bp, Artificial Sequence
  Description of Artificial Sequence Synthetic probe
actgatataatatggagatatcc

SEQ ID NO: 1495 - DNA, 19 bp, Artificial Sequence
  Description of Artificial Sequence Synthetic probe
ctctcagcttgtacttttg

SEQ ID NO: 1496 - DNA, 20 bp, Artificial Sequence
  Description of Artificial Sequence Synthetic probe
ccaatctcaatagctttttc

SEQ ID NO: 1497 - DNA, 18 bp, Artificial Sequence
  Description of Artificial Sequence Synthetic probe
tcagaaagccgcgccatc

SEQ ID NO: 1498 - DNA, 19 bp, Artificial Sequence
  Description of Artificial Sequence Synthetic probe
ccggaagatcgagctatac

SEQ ID NO: 1499 - DNA, 19 bp, Artificial Sequence
  Description of Artificial Sequence Synthetic probe
tcggtctttcttagatttt

SEQ ID NO: 1500 - DNA, 17 bp, Artificial Sequence
  Description of Artificial Sequence Synthetic probe
cagaaagccgcgccatc

SEQ ID NO: 1501 - DNA, 20 bp, Artificial Sequence
  Description of Artificial Sequence Synthetic probe
ccgatttcaatagctttttc

SEQ ID NO: 1502 - DNA, 20 bp, Artificial Sequence
  Description of Artificial Sequence Synthetic probe
ttgccgatttcaatagcttt

SEQ ID NO: 1503 - DNA, 18 bp, Artificial Sequence
  Description of Artificial Sequence Synthetic probe
tcagaaagccgcgccatc

SEQ ID NO: 1504 - DNA, 17 bp, Artificial Sequence
  Description of Artificial Sequence Synthetic probe
caacgcgctattaaaag

SEQ ID NO: 1505 - DNA, 17 bp, Artificial Sequence
  Description of Artificial Sequence Synthetic probe
tcgcgttcggtctttct

SEQ ID NO: 1506 - DNA, 16 bp, Artificial Sequence
  Description of Artificial Sequence Synthetic probe
tcgcgttctgattttt

SEQ ID NO: 1507 - DNA, 18 bp, Artificial Sequence
  Description of Artificial Sequence Synthetic probe
acgcgttactaaaagaag

SEQ ID NO: 1508 - DNA, 22 bp, Artificial Sequence
  Description of Artificial Sequence Synthetic probe
atccatctttgagtaaagcatt

SEQ ID NO: 1509 - DNA, 17 bp, Artificial Sequence
  Description of Artificial Sequence Synthetic probe
ttgccgatttcaattgc

SEQ ID NO: 1510 - DNA, 18 bp, Artificial Sequence
  Description of Artificial Sequence Synthetic probe
tcccaattgccacttcaa

SEQ ID NO: 1511 - DNA, 20 bp, Artificial Sequence
  Description of Artificial Sequence Synthetic probe
ccgatttcaattgccttttc

SEQ ID NO: 1512 - DNA, 21 bp, Artificial Sequence
  Description of Artificial Sequence Synthetic probe
ctgttttctttagatttttcc

SEQ ID NO: 1513 - DNA, 14 bp, Artificial Sequence
  Description of Artificial Sequence Synthetic probe
ccgcgccatcccga

SEQ ID NO: 1514 - DNA, 19 bp, Artificial Sequence
  Description of Artificial Sequence Synthetic probe
atcggcaatgccctattaa

SEQ ID NO: 1515 - DNA, 19 bp, Artificial Sequence
  Description of Artificial Sequence Synthetic probe
caatgcgctattaaaagat

SEQ ID NO: 1516 - DNA, 17 bp, Artificial Sequence
  Description of Artificial Sequence Synthetic probe
caccttctgaaaacaac

SEQ ID NO: 1517 - DNA, 21 bp, Artificial Sequence
  Description of Artificial Sequence Synthetic probe
ctgcatctcaaggtaatgtat

SEQ ID NO: 1518 - DNA, 24 bp, Artificial Sequence
  Description of Artificial Sequence Synthetic probe
caattgaaagaagataacaatttg

SEQ ID NO: 1519 - DNA, 25 bp, Artificial Sequence
  Description of Artificial Sequence Synthetic probe
aagttgttatcttctttcaattgtt

SEQ ID NO: 1520 - DNA, 20 bp, Artificial Sequence
  Description of Artificial Sequence Synthetic probe
tcactagctttagcattttc

SEQ ID NO: 1521 - DNA, 21 bp, Artificial Sequence
  Description of Artificial Sequence Synthetic probe
ctgcatacagtaaagaagaac

SEQ ID NO: 1522 - DNA, 18 bp, Artificial Sequence
  Description of Artificial Sequence Synthetic probe
acagcatacagtaaatct

SEQ ID NO: 1523 - DNA, 17 bp, Artificial Sequence
  Description of Artificial Sequence Synthetic probe
ccttgtgctgaagaact

SEQ ID NO: 1524 - DNA, 16 bp, Artificial Sequence
  Description of Artificial Sequence Synthetic probe
cagctccagcaactca

SEQ ID NO: 1525 - DNA, 21 bp, Artificial Sequence
  Description of Artificial Sequence Synthetic probe
ttactagtagcagcttttttc

SEQ ID NO: 1526 - DNA, 17 bp, Artificial Sequence
  Description of Artificial Sequence Synthetic probe
atgccaggttcagcttt

SEQ ID NO: 1527 - DNA, 18 bp, Artificial Sequence
  Description of Artificial Sequence Synthetic probe
atgtccaagggtatctcc

SEQ ID NO: 1528 - DNA, 18 bp, Artificial Sequence
  Description of Artificial Sequence Synthetic probe
tcgctgtcatgtggccca

SEQ ID NO: 1529 - DNA, 19 bp, Artificial Sequence
  Description of Artificial Sequence Synthetic probe
tctatcggaagtaaagctg

SEQ ID NO: 1530 - DNA, 18 bp, Artificial Sequence
  Description of Artificial Sequence Synthetic probe
ttggagcagctttttctt

SEQ ID NO: 1531 - DNA, 18 bp, Artificial Sequence
  Description of Artificial Sequence Synthetic probe
ttgcaccaccagtatcag

SEQ ID NO: 1532 - DNA, 18 bp, Artificial Sequence
  Description of Artificial Sequence Synthetic probe
ccgccttttcttctttcg

SEQ ID NO: 1533 - DNA, 20 bp, Artificial Sequence
  Description of Artificial Sequence Synthetic probe
aacaaatcgtctgcatctca

SEQ ID NO: 1534 - DNA, 17 bp, Artificial Sequence
  Description of Artificial Sequence Synthetic probe
ccgggctctcattagat

SEQ ID NO: 1535 - DNA, 17 bp, Artificial Sequence
  Description of Artificial Sequence Synthetic probe
cacctacagacaatttt

SEQ ID NO: 1536 - DNA, 17 bp, Artificial Sequence
  Description of Artificial Sequence Synthetic probe
ttgcaccgccagtatca

SEQ ID NO: 1537 - DNA, 16 bp, Artificial Sequence
  Description of Artificial Sequence Synthetic probe
ccaaggtgacggattc

SEQ ID NO: 1538 - DNA, 19 bp, Artificial Sequence
  Description of Artificial Sequence Synthetic probe
attaccttcgacaacttca

SEQ ID NO: 1539 - DNA, 19 bp, Artificial Sequence
  Description of Artificial Sequence Synthetic probe
accttcgtctaaagatttg

SEQ ID NO: 1540 - DNA, 17 bp, Artificial Sequence
  Description of Artificial Sequence Synthetic probe
acacttcagcgctatcg

SEQ ID NO: 1541 - DNA, 15 bp, Artificial Sequence
  Description of Artificial Sequence Synthetic probe
ccgcttggccttctt

SEQ ID NO: 1542 - DNA, 18 bp, Artificial Sequence
  Description of Artificial Sequence Synthetic probe
aaggcttcggtttcatcg

SEQ ID NO: 1543 - DNA, 21 bp, Artificial Sequence
  Description of Artificial Sequence Synthetic probe
ccttcaactacttcaaatgtt

SEQ ID NO: 1544 - DNA, 17 bp, Artificial Sequence
  Description of Artificial Sequence Synthetic probe
caacgctttgaccttcg

SEQ ID NO: 1545 - DNA, 20 bp, Artificial Sequence
  Description of Artificial Sequence Synthetic probe
acgatgtattcgtacatttc

SEQ ID NO: 1546 - DNA, 20 bp, Artificial Sequence
  Description of Artificial Sequence Synthetic probe
aaagcgttgaatttgaaatc

SEQ ID NO: 1547 - DNA, 17 bp, Artificial Sequence
  Description of Artificial Sequence Synthetic probe
ctccgccttctacttcg

SEQ ID NO: 1548 - DNA, 18 bp, Artificial Sequence
  Description of Artificial Sequence Synthetic probe
aaaggcttcggtttcatc

SEQ ID NO: 1549 - DNA, 21 bp, Artificial Sequence
  Description of Artificial Sequence Synthetic probe
accttcaactacttcaaatgt

SEQ ID NO: 1550 - DNA, 16 bp, Artificial Sequence
  Description of Artificial Sequence Synthetic probe
tcgccttggatagcgc

SEQ ID NO: 1551 - DNA, 18 bp, Artificial Sequence
  Description of Artificial Sequence Synthetic probe
tcgaaggtgaaggattca

SEQ ID NO: 1552 - DNA, 17 bp, Artificial Sequence
  Description of Artificial Sequence Synthetic probe
acacttcagcgctatcg

SEQ ID NO: 1553 - DNA, 15 bp, Artificial Sequence
  Description of Artificial Sequence Synthetic probe
ccgcttggccttctt

SEQ ID NO: 1554 - DNA, 18 bp, Artificial Sequence
  Description of Artificial Sequence Synthetic probe
cacttcacagcgatccaa

SEQ ID NO: 1555 - DNA, 17 bp, Artificial Sequence
  Description of Artificial Sequence Synthetic probe
tcgctgtgaagtgaacg

SEQ ID NO: 1556 - DNA, 19 bp, Artificial Sequence
  Description of Artificial Sequence Synthetic probe
aagggttacggttttatcg

SEQ ID NO: 1557 - DNA, 18 bp, Artificial Sequence
  Description of Artificial Sequence Synthetic probe
tcgaaggtgaaggattca

SEQ ID NO: 1558 - DNA, 19 bp, Artificial Sequence
  Description of Artificial Sequence Synthetic probe
acgacaaaggaataaattc

SEQ ID NO: 1559 - DNA, 26 bp, Artificial Sequence
  Description of Artificial Sequence Synthetic probe
cattcgtacaattatctatcaatcaa

SEQ ID NO: 1560 - DNA, 23 bp, Artificial Sequence
  Description of Artificial Sequence Synthetic probe
ctctttatatttagggaaaaatc

SEQ ID NO: 1561 - DNA, 15 bp, Artificial Sequence
  Description of Artificial Sequence Synthetic probe
ctactgcaaaatccc

SEQ ID NO: 1562 - DNA, 19 bp, Artificial Sequence
  Description of Artificial Sequence Synthetic probe
ttgcatcgtaccatcctac

SEQ ID NO: 1563 - DNA, 19 bp, Artificial Sequence
  Description of Artificial Sequence Synthetic probe
caattctcaaacagctccc

SEQ ID NO: 1564 - DNA, 21 bp, Artificial Sequence
  Description of Artificial Sequence Synthetic probe
tcaaataagtgatgcaagtcc

SEQ ID NO: 1565 - DNA, 18 bp, Artificial Sequence
  Description of Artificial Sequence Synthetic probe
atgggaaggtaacttttg

SEQ ID NO: 1566 - DNA, 18 bp, Artificial Sequence
  Description of Artificial Sequence Synthetic probe
ttggactagcaaaggttc

SEQ ID NO: 1567 - DNA, 21 bp, Artificial Sequence
  Description of Artificial Sequence Synthetic probe
accgacaaaagaaggttatac

SEQ ID NO: 1568 - DNA, 20 bp, Artificial Sequence
  Description of Artificial Sequence Synthetic probe
ctgtcgttagctttaattgt

SEQ ID NO: 1569 - DNA, 21 bp, Artificial Sequence
  Description of Artificial Sequence Synthetic probe
atgtgacacctcttgctaatc

SEQ ID NO: 1570 - DNA, 20 bp, Artificial Sequence
  Description of Artificial Sequence Synthetic probe
aaggacttgcatcacttatt

SEQ ID NO: 1571 - DNA, 18 bp, Artificial Sequence
  Description of Artificial Sequence Synthetic probe
ccgtgtagctatttattg

SEQ ID NO: 1572 - DNA, 20 bp, Artificial Sequence
  Description of Artificial Sequence Synthetic probe
cttgcatttacccataaact

SEQ ID NO: 1573 - DNA, 17 bp, Artificial Sequence
  Description of Artificial Sequence Synthetic probe
cagcgccaattaacgaa

SEQ ID NO: 1574 - DNA, 14 bp, Artificial Sequence
  Description of Artificial Sequence Synthetic probe
cccaccagtttccg

SEQ ID NO: 1575 - DNA, 17 bp, Artificial Sequence
  Description of Artificial Sequence Synthetic probe
ctgagccaacaaaagaa

SEQ ID NO: 1576 - DNA, 19 bp, Artificial Sequence
  Description of Artificial Sequence Synthetic probe
tcgtcgtcgttccatccac

SEQ ID NO: 1577 - DNA, 16 bp, Artificial Sequence
  Description of Artificial Sequence Synthetic probe
tcgcatctgcgctgtc

SEQ ID NO: 1578 - DNA, 19 bp, Artificial Sequence
  Description of Artificial Sequence Synthetic probe
ccagaaatcgttaatatcg

SEQ ID NO: 1579 - DNA, 19 bp, Artificial Sequence
  Description of Artificial Sequence Synthetic probe
cataaaccagagaaatttg

SEQ ID NO: 1580 - DNA, 20 bp, Artificial Sequence
  Description of Artificial Sequence Synthetic probe
aagcactcaaagacagtttt

SEQ ID NO: 1581 - DNA, 22 bp, Artificial Sequence
  Description of Artificial Sequence Synthetic probe
ctcatatacgtgacacactcaa

SEQ ID NO: 1582 - DNA, 19 bp, Artificial Sequence
  Description of Artificial Sequence Synthetic probe
cacatcaccgaacgacatg

SEQ ID NO: 1583 - DNA, 20 bp, Artificial Sequence
  Description of Artificial Sequence Synthetic probe
atgcaccgatttaaaactgt

SEQ ID NO: 1584 - DNA, 20 bp, Artificial Sequence
  Description of Artificial Sequence Synthetic probe
acgtccttctaacagaaaca

SEQ ID NO: 1585 - DNA, 17 bp, Artificial Sequence
  Description of Artificial Sequence Synthetic probe
cagctcttcaaaaacag

SEQ ID NO: 1586 - DNA, 20 bp, Artificial Sequence
  Description of Artificial Sequence Synthetic probe
aacggctttttggttaccac

SEQ ID NO: 1587 - DNA, 21 bp, Artificial Sequence
  Description of Artificial Sequence Synthetic probe
caagcttattcagttctttcc

SEQ ID NO: 1588 - DNA, 20 bp, Artificial Sequence
  Description of Artificial Sequence Synthetic probe
ttttcgacttgatgcaattc

SEQ ID NO: 1589 - DNA, 20 bp, Artificial Sequence
  Description of Artificial Sequence Synthetic probe
atgcaatcgtctttaagctt

SEQ ID NO: 1590 - DNA, 18 bp, Artificial Sequence
  Description of Artificial Sequence Synthetic probe
catagggatgaaagcgcc

SEQ ID NO: 1591 - DNA, 20 bp, Artificial Sequence
  Description of Artificial Sequence Synthetic probe
ccaacttctagcatattaag

SEQ ID NO: 1592 - DNA, 22 bp, Artificial Sequence
  Description of Artificial Sequence Synthetic probe
tttctcagtagaagagattatg

SEQ ID NO: 1593 - DNA, 18 bp, Artificial Sequence
  Description of Artificial Sequence Synthetic probe
ccggattaccgagatcaa

SEQ ID NO: 1594 - DNA, 18 bp, Artificial Sequence
  Description of Artificial Sequence Synthetic probe
tcgcgtatatgggtttct

SEQ ID NO: 1595 - DNA, 19 bp, Artificial Sequence
  Description of Artificial Sequence Synthetic probe
atttcaacgtcagctcttc

SEQ ID NO: 1596 - DNA, 22 bp, Artificial Sequence
  Description of Artificial Sequence Synthetic probe
acgacattctcaagtttattca

SEQ ID NO: 1597 - DNA, 20 bp, Artificial Sequence
  Description of Artificial Sequence Synthetic probe
ttcggtatgaattggaatcc

SEQ ID NO: 1598 - DNA, 18 bp, Artificial Sequence
  Description of Artificial Sequence Synthetic probe
cacacatacgcgatacac

SEQ ID NO: 1599 - DNA, 16 bp, Artificial Sequence
  Description of Artificial Sequence Synthetic probe
ccgggttggtattcat

SEQ ID NO: 1600 - DNA, 20 bp, Artificial Sequence
  Description of Artificial Sequence Synthetic probe
ccgataaaacaaacaaattc

SEQ ID NO: 1601 - DNA, 20 bp, Artificial Sequence
  Description of Artificial Sequence Synthetic probe
ccgccatgatgtctttatca

SEQ ID NO: 1602 - DNA, 19 bp, Artificial Sequence
  Description of Artificial Sequence Synthetic probe
cagtcatgaaagaaaaacg

SEQ ID NO: 1603 - DNA, 17 bp, Artificial Sequence
  Description of Artificial Sequence Synthetic probe
tcgtacagcaacttctg

SEQ ID NO: 1604 - DNA, 20 bp, Artificial Sequence
  Description of Artificial Sequence Synthetic probe
aaaactcttctggtaaatct

SEQ ID NO: 1605 - DNA, 22 bp, Artificial Sequence
  Description of Artificial Sequence Synthetic probe
tcgataaagccttttaaaacat

SEQ ID NO: 1606 - DNA, 19 bp, Artificial Sequence
  Description of Artificial Sequence Synthetic probe
caataagccggttaatcat

SEQ ID NO: 1607 - DNA, 22 bp, Artificial Sequence
  Description of Artificial Sequence Synthetic probe
aatgatctaacactaaacatcg

SEQ ID NO: 1608 - DNA, 20 bp, Artificial Sequence
  Description of Artificial Sequence Synthetic probe
actgcttttttgcccccttt

SEQ ID NO: 1609 - DNA, 17 bp, Artificial Sequence
  Description of Artificial Sequence Synthetic probe
ccttgtaccaaaattac

SEQ ID NO: 1610 - DNA, 19 bp, Artificial Sequence
  Description of Artificial Sequence Synthetic probe
atgccacatatgacgattc

SEQ ID NO: 1611 - DNA, 20 bp, Artificial Sequence
  Description of Artificial Sequence Synthetic probe
cttgccaagttctttttgca

SEQ ID NO: 1612 - DNA, 17 bp, Artificial Sequence
  Description of Artificial Sequence Synthetic probe
atgcgacagaaaaccag

SEQ ID NO: 1613 - DNA, 20 bp, Artificial Sequence
  Description of Artificial Sequence Synthetic probe
tttaaatcgccgcacagcaa

SEQ ID NO: 1614 - DNA, 20 bp, Artificial Sequence
  Description of Artificial Sequence Synthetic probe
tttgttctacgtcaataaag

SEQ ID NO: 1615 - DNA, 18 bp, Artificial Sequence
  Description of Artificial Sequence Synthetic probe
atgaaagctggtgaaatc

SEQ ID NO: 1616 - DNA, 20 bp, Artificial Sequence
  Description of Artificial Sequence Synthetic probe
ccgataaaacaaacaaattc

SEQ ID NO: 1617 - DNA, 19 bp, Artificial Sequence
  Description of Artificial Sequence Synthetic probe
aagccggttaatcatcttc

SEQ ID NO: 1618 - DNA, 17 bp, Artificial Sequence
  Description of Artificial Sequence Synthetic probe
ccgacaagtcgtttatc

SEQ ID NO: 1619 - DNA, 20 bp, Artificial Sequence
  Description of Artificial Sequence Synthetic probe
accggcttattgaacctaca

SEQ ID NO: 1620 - DNA, 24 bp, Artificial Sequence
  Description of Artificial Sequence Synthetic probe
acaatggattttgacttacatcta

SEQ ID NO: 1621 - DNA, 19 bp, Artificial Sequence
  Description of Artificial Sequence Synthetic probe
tcggttatggatgaattat

SEQ ID NO: 1622 - DNA, 19 bp, Artificial Sequence
  Description of Artificial Sequence Synthetic probe
acaactgagctccgccact

SEQ ID NO: 1623 - DNA, 19 bp, Artificial Sequence
  Description of Artificial Sequence Synthetic probe
tacaggctctgctgctacg

SEQ ID NO: 1624 - DNA, 19 bp, Artificial Sequence
  Description of Artificial Sequence Synthetic probe
aaccgtcaactacatttac

SEQ ID NO: 1625 - DNA, 24 bp, Artificial Sequence
  Description of Artificial Sequence Synthetic probe
ccactgttaacttatttaaactgc

SEQ ID NO: 1626 - DNA, 16 bp, Artificial Sequence
  Description of Artificial Sequence Synthetic probe
caactggacaattcac

SEQ ID NO: 1627 - DNA, 16 bp, Artificial Sequence
  Description of Artificial Sequence Synthetic probe
atggagcgagcatttt

SEQ ID NO: 1628 - DNA, 20 bp, Artificial Sequence
  Description of Artificial Sequence Synthetic probe
caacaccttaaccgaaatag

SEQ ID NO: 1629 - DNA, 17 bp, Artificial Sequence
  Description of Artificial Sequence Synthetic probe
ctctccatccgaaatat

SEQ ID NO: 1630 - DNA, 19 bp, Artificial Sequence
  Description of Artificial Sequence Synthetic probe
ctacgctttcaaaattaac

SEQ ID NO: 1631 - DNA, 21 bp, Artificial Sequence
  Description of Artificial Sequence Synthetic probe
ctggattttttgtgtaaatat

SEQ ID NO: 1632 - DNA, 19 bp, Artificial Sequence
  Description of Artificial Sequence Synthetic probe
aattaccgcttaaaacttc

SEQ ID NO: 1633 - DNA, 17 bp, Artificial Sequence
  Description of Artificial Sequence Synthetic probe
aagcccctcaacccatc

SEQ ID NO: 1634 - DNA, 16 bp, Artificial Sequence
  Description of Artificial Sequence Synthetic probe
catccacgtaattcac

SEQ ID NO: 1635 - DNA, 20 bp, Artificial Sequence
  Description of Artificial Sequence Synthetic probe
accgaaatagatgtaacccc

SEQ ID NO: 1636 - DNA, 16 bp, Artificial Sequence
  Description of Artificial Sequence Synthetic probe
catccacgtaattcac

SEQ ID NO: 1637 - DNA, 15 bp, Artificial Sequence
  Description of Artificial Sequence Synthetic probe
aagcagcagagcctg

SEQ ID NO: 1638 - DNA, 16 bp, Artificial Sequence
  Description of Artificial Sequence Synthetic probe
tagcgcacaaactgtc

SEQ ID NO: 1639 - DNA, 19 bp, Artificial Sequence
  Description of Artificial Sequence Synthetic probe
atgagaaacgtctaatttc

SEQ ID NO: 1640 - DNA, 19 bp, Artificial Sequence
  Description of Artificial Sequence Synthetic probe
aagaccccaagtagagctg

SEQ ID NO: 1641 - DNA, 17 bp, Artificial Sequence
  Description of Artificial Sequence Synthetic probe
cagcatctggagcaacc

SEQ ID NO: 1642 - DNA, 17 bp, Artificial Sequence
  Description of Artificial Sequence Synthetic probe
cagtggcacccgatgct

SEQ ID NO: 1643 - DNA, 20 bp, Artificial Sequence
  Description of Artificial Sequence Synthetic probe
ctgcaactagtcctaaaaca

SEQ ID NO: 1644 - DNA, 21 bp, Artificial Sequence
  Description of Artificial Sequence Synthetic probe
ctgcgacaacaaaacctaaga

SEQ ID NO: 1645 - DNA, 18 bp, Artificial Sequence
  Description of Artificial Sequence Synthetic probe
cattgcgtaaccaacaac

SEQ ID NO: 1646 - DNA, 19 bp, Artificial Sequence
  Description of Artificial Sequence Synthetic probe
ccggacgttattgttaatg

SEQ ID NO: 1647 - DNA, 18 bp, Artificial Sequence
  Description of Artificial Sequence Synthetic probe
cagctccgatattcatcc

SEQ ID NO: 1648 - DNA, 18 bp, Artificial Sequence
  Description of Artificial Sequence Synthetic probe
atggcattgagtaaattt

SEQ ID NO: 1649 - DNA, 22 bp, Artificial Sequence
  Description of Artificial Sequence Synthetic probe
ttagcggacatcatgttaataa

SEQ ID NO: 1650 - DNA, 17 bp, Artificial Sequence
  Description of Artificial Sequence Synthetic probe
atgcgtatcgcaattcc

SEQ ID NO: 1651 - DNA, 19 bp, Artificial Sequence
  Description of Artificial Sequence Synthetic probe
acgcattgcttgaagtagt

SEQ ID NO: 1652 - DNA, 18 bp, Artificial Sequence
  Description of Artificial Sequence Synthetic probe
cagcattcactcaattca

SEQ ID NO: 1653 - DNA, 16 bp, Artificial Sequence
  Description of Artificial Sequence Synthetic probe
caagcgcgatcatttc

SEQ ID NO: 1654 - DNA, 20 bp, Artificial Sequence
  Description of Artificial Sequence Synthetic probe
tcgactggattcatgtttca

SEQ ID NO: 1655 - DNA, 20 bp, Artificial Sequence
  Description of Artificial Sequence Synthetic probe
ttggaatgcaactgtaattg

SEQ ID NO: 1656 - DNA, 17 bp, Artificial Sequence
  Description of Artificial Sequence Synthetic probe
cagcatctggagcaacc

SEQ ID NO: 1657 - DNA, 20 bp, Artificial Sequence
  Description of Artificial Sequence Synthetic probe
ctcgactggatacatgttac

SEQ ID NO: 1658 - DNA, 19 bp, Artificial Sequence
  Description of Artificial Sequence Synthetic probe
ctgcaacgagtcctataac

SEQ ID NO: 1659 - DNA, 21 bp, Artificial Sequence
  Description of Artificial Sequence Synthetic probe
ttgaaatgcaactgtaattgt

SEQ ID NO: 1660 - DNA, 16 bp, Artificial Sequence
  Description of Artificial Sequence Synthetic probe
caccgtccattaattg

SEQ ID NO: 1661 - DNA, 17 bp, Artificial Sequence
  Description of Artificial Sequence Synthetic probe
cagctccgatgttcatc

SEQ ID NO: 1662 - DNA, 19 bp, Artificial Sequence
  Description of Artificial Sequence Synthetic probe
caactagaagcaggaatac

SEQ ID NO: 1663 - DNA, 16 bp, Artificial Sequence
  Description of Artificial Sequence Synthetic probe
actgcatgccgaattt

SEQ ID NO: 1664 - DNA, 16 bp, Artificial Sequence
  Description of Artificial Sequence Synthetic probe
catgggccaatttatt

SEQ ID NO: 1665 - DNA, 18 bp, Artificial Sequence
  Description of Artificial Sequence Synthetic probe
tcaggtagagcggacatc

SEQ ID NO: 1666 - DNA, 14 bp, Artificial Sequence
  Description of Artificial Sequence Synthetic probe
ccgcggaaaaatat

SEQ ID NO: 1667 - DNA, 25 bp, Artificial Sequence
  Description of Artificial Sequence Synthetic probe
caaagattatttttacaccactccc

SEQ ID NO: 1668 - DNA, 22 bp, Artificial Sequence
  Description of Artificial Sequence Synthetic probe
tatcgatattagagcaaaagac

SEQ ID NO: 1669 - DNA, 19 bp, Artificial Sequence
  Description of Artificial Sequence Synthetic probe
cagtctggacaatctcttt

SEQ ID NO: 1670 - DNA, 25 bp, Artificial Sequence
  Description of Artificial Sequence Synthetic probe
ccaaccactaatcaacatttataaa

SEQ ID NO: 1671 - DNA, 17 bp, Artificial Sequence
  Description of Artificial Sequence Synthetic probe
ctgacacgagcaataaa

SEQ ID NO: 1672 - DNA, 18 bp, Artificial Sequence
  Description of Artificial Sequence Synthetic probe
tcgttgctgttttgctcg

SEQ ID NO: 1673 - DNA, 15 bp, Artificial Sequence
  Description of Artificial Sequence Synthetic probe
ccgcggacatctttt

SEQ ID NO: 1674 - DNA, 22 bp, Artificial Sequence
  Description of Artificial Sequence Synthetic probe
ccgttatagctcatcgtatcat

SEQ ID NO: 1675 - DNA, 16 bp, Artificial Sequence
  Description of Artificial Sequence Synthetic probe
acgcggacattaacca

SEQ ID NO: 1676 - DNA, 20 bp, Artificial Sequence
  Description of Artificial Sequence Synthetic probe
cctgaaagtgatgcatttaa

SEQ ID NO: 1677 - DNA, 20 bp, Artificial Sequence
  Description of Artificial Sequence Synthetic probe
aacaaaacagagcaataaaa

SEQ ID NO: 1678 - DNA, 18 bp, Artificial Sequence
  Description of Artificial Sequence Synthetic probe
accactagctcaaacaca

SEQ ID NO: 1679 - DNA, 22 bp, Artificial Sequence
  Description of Artificial Sequence Synthetic probe
atgtcattgtaccaacaactag

SEQ ID NO: 1680 - DNA, 21 bp, Artificial Sequence
  Description of Artificial Sequence Synthetic probe
atgggccaatttttttaaatg

SEQ ID NO: 1681 - DNA, 16 bp, Artificial Sequence
  Description of Artificial Sequence Synthetic probe
ccgctttggaagcttg

SEQ ID NO: 1682 - DNA, 21 bp, Artificial Sequence
  Description of Artificial Sequence Synthetic probe
accgtattcctgcttctaatt

SEQ ID NO: 1683 - DNA, 22 bp, Artificial Sequence
  Description of Artificial Sequence Synthetic probe
ttttcattgtggagcttgatca

SEQ ID NO: 1684 - DNA, 23 bp, Artificial Sequence
  Description of Artificial Sequence Synthetic probe
atgtcaatgctttacctaatcta

SEQ ID NO: 1685 - DNA, 24 bp, Artificial Sequence
  Description of Artificial Sequence Synthetic probe
ctataggatactctttcaaagttg

SEQ ID NO: 1686 - DNA, 21 bp, Artificial Sequence
  Description of Artificial Sequence Synthetic probe
ccgtgtaactataggatactc

SEQ ID NO: 1687 - DNA, 17 bp, Artificial Sequence
  Description of Artificial Sequence Synthetic probe
acgttgcggtatcctag

SEQ ID NO: 1688 - DNA, 16 bp, Artificial Sequence
  Description of Artificial Sequence Synthetic probe
accaccgtttgctttc

SEQ ID NO: 1689 - DNA, 15 bp, Artificial Sequence
  Description of Artificial Sequence Synthetic probe
ccgccgtttgctttc

SEQ ID NO: 1690 - DNA, 19 bp, Artificial Sequence
  Description of Artificial Sequence Synthetic probe
caacgttcaatcgttttct

SEQ ID NO: 1691 - DNA, 17 bp, Artificial Sequence
  Description of Artificial Sequence Synthetic probe
acgttgcggtatcctag

SEQ ID NO: 1692 - DNA, 24 bp, Artificial Sequence
  Description of Artificial Sequence Synthetic probe
atcctatagttatacggatatctt

SEQ ID NO: 1693 - DNA, 20 bp, Artificial Sequence
  Description of Artificial Sequence Synthetic probe
aagcattgacattgaaagca

SEQ ID NO: 1694 - DNA, 24 bp, Artificial Sequence
  Description of Artificial Sequence Synthetic probe
ctataggatactctttcaaagttg

SEQ ID NO: 1695 - DNA, 22 bp, Artificial Sequence
  Description of Artificial Sequence Synthetic probe
ttttcattgtggagcttgatca

SEQ ID NO: 1696 - DNA, 21 bp, Artificial Sequence
  Description of Artificial Sequence Synthetic probe
aacggatggatatgtttcagc

SEQ ID NO: 1697 - DNA, 19 bp, Artificial Sequence
  Description of Artificial Sequence Synthetic probe
caacgttcaatcgttttct

SEQ ID NO: 1698 - DNA, 23 bp, Artificial Sequence
  Description of Artificial Sequence Synthetic probe
atgtcaatgctttacctaatcta

SEQ ID NO: 1699 - DNA, 21 bp, Artificial Sequence
  Description of Artificial Sequence Synthetic probe
aacggatggatatgtttcagc

SEQ ID NO: 1700 - DNA, 23 bp, Artificial Sequence
  Description of Artificial Sequence Synthetic probe
atgctttacctaatctagaatct

SEQ ID NO: 1701 - DNA, 16 bp, Artificial Sequence
  Description of Artificial Sequence Synthetic probe
ctgcaacggtaacatc

SEQ ID NO: 1702 - DNA, 24 bp, Artificial Sequence
  Description of Artificial Sequence Synthetic probe
cactttatagatgactttaatttt

SEQ ID NO: 1703 - DNA, 24 bp, Artificial Sequence
  Description of Artificial Sequence Synthetic probe
atgacaacagattgattatttaac

SEQ ID NO: 1704 - DNA, 18 bp, Artificial Sequence
  Description of Artificial Sequence Synthetic probe
tcgtttgggtcgtatttt

SEQ ID NO: 1705 - DNA, 19 bp, Artificial Sequence
  Description of Artificial Sequence Synthetic probe
aatgtagctttgcaaaatc

SEQ ID NO: 1706 - DNA, 19 bp, Artificial Sequence
  Description of Artificial Sequence Synthetic probe
ccattttgttcggaatctt

SEQ ID NO: 1707 - DNA, 17 bp, Artificial Sequence
  Description of Artificial Sequence Synthetic probe
cccgccactcccgtaat

SEQ ID NO: 1708 - DNA, 18 bp, Artificial Sequence
  Description of Artificial Sequence Synthetic probe
acgtagtcggtaaaagtc

SEQ ID NO: 1709 - DNA, 17 bp, Artificial Sequence
  Description of Artificial Sequence Synthetic probe
ttggctcagggcaaaat

SEQ ID NO: 1710 - DNA, 16 bp, Artificial Sequence
  Description of Artificial Sequence Synthetic probe
cactgtcgcgtttctc

SEQ ID NO: 1711 - DNA, 23 bp, Artificial Sequence
  Description of Artificial Sequence Synthetic probe
actaatgtatcattacaaaatca

SEQ ID NO: 1712 - DNA, 25 bp, Artificial Sequence
  Description of Artificial Sequence Synthetic probe
ttggttgtacacttgtagatattac

SEQ ID NO: 1713 - DNA, 19 bp, Artificial Sequence
  Description of Artificial Sequence Synthetic probe
tcactggatttttaagccc

SEQ ID NO: 1714 - DNA, 23 bp, Artificial Sequence
  Description of Artificial Sequence Synthetic probe
aagccactttatcaggtgacaat

SEQ ID NO: 1715 - DNA, 19 bp, Artificial Sequence
  Description of Artificial Sequence Synthetic probe
caggtggaatggtgatttt

SEQ ID NO: 1716 - DNA, 18 bp, Artificial Sequence
  Description of Artificial Sequence Synthetic probe
atcttgttcggaatcttt

SEQ ID NO: 1717 - DNA, 20 bp, Artificial Sequence
  Description of Artificial Sequence Synthetic probe
tttgataagagcgttttttc

SEQ ID NO: 1718 - DNA, 20 bp, Artificial Sequence
  Description of Artificial Sequence Synthetic probe
acgcctttattagcaaagtt

SEQ ID NO: 1719 - DNA, 16 bp, Artificial Sequence
  Description of Artificial Sequence Synthetic probe
atgcgaatcgaaattt

SEQ ID NO: 1720 - DNA, 20 bp, Artificial Sequence
  Description of Artificial Sequence Synthetic probe
acattggaccgttactttaa

SEQ ID NO: 1721 - DNA, 22 bp, Artificial Sequence
  Description of Artificial Sequence Synthetic probe
ccggaacagctgaaattaatta

SEQ ID NO: 1722 - DNA, 21 bp, Artificial Sequence
  Description of Artificial Sequence Synthetic probe
aaagaagaattcgacaatctt

SEQ ID NO: 1723 - DNA, 22 bp, Artificial Sequence
  Description of Artificial Sequence Synthetic probe
atggtctaaaatcatcaattcc

SEQ ID NO: 1724 - DNA, 20 bp, Artificial Sequence
  Description of Artificial Sequence Synthetic probe
ccgattaaaaaccaaacttc

SEQ ID NO: 1725 - DNA, 16 bp, Artificial Sequence
  Description of Artificial Sequence Synthetic probe
ccacaccaggaaaata

SEQ ID NO: 1726 - DNA, 17 bp, Artificial Sequence
  Description of Artificial Sequence Synthetic probe
accgtgacattaaacgc

SEQ ID NO: 1727 - DNA, 20 bp, Artificial Sequence
  Description of Artificial Sequence Synthetic probe
catttctgaagaacaatttt

SEQ ID NO: 1728 - DNA, 21 bp, Artificial Sequence
  Description of Artificial Sequence Synthetic probe
aagacattatgcgtcacttca

SEQ ID NO: 1729 - DNA, 17 bp, Artificial Sequence
  Description of Artificial Sequence Synthetic probe
aagcagcatcaaacctt

SEQ ID NO: 1730 - DNA, 24 bp, Artificial Sequence
  Description of Artificial Sequence Synthetic probe
tttttcatgaacaactacttttac

SEQ ID NO: 1731 - DNA, 17 bp, Artificial Sequence
  Description of Artificial Sequence Synthetic probe
tccgcccactgttaagt

SEQ ID NO: 1732 - DNA, 19 bp, Artificial Sequence
  Description of Artificial Sequence Synthetic probe
atggaattgatgattttcg

SEQ ID NO: 1733 - DNA, 21 bp, Artificial Sequence
  Description of Artificial Sequence Synthetic probe
aagcatgtaaatcaacaaacg

SEQ ID NO: 1734 - DNA, 25 bp, Artificial Sequence
  Description of Artificial Sequence Synthetic probe
caaagtcatttaaaggtctaaaatc

SEQ ID NO: 1735 - DNA, 16 bp, Artificial Sequence
  Description of Artificial Sequence Synthetic probe
cctcaaagcgaaaatt

SEQ ID NO: 1736 - DNA, 19 bp, Artificial Sequence
  Description of Artificial Sequence Synthetic probe
acgctttttgtgcaatttg

SEQ ID NO: 1737 - DNA, 14 bp, Artificial Sequence
  Description of Artificial Sequence Synthetic probe
accgcggccaaaaa

SEQ ID NO: 1738 - DNA, 18 bp, Artificial Sequence
  Description of Artificial Sequence Synthetic probe
ttgggagtgccacatcat

SEQ ID NO: 1739 - DNA, 18 bp, Artificial Sequence
  Description of Artificial Sequence Synthetic probe
cacaggagttgcttttag

SEQ ID NO: 1740 - DNA, 24 bp, Artificial Sequence
  Description of Artificial Sequence Synthetic probe
ccacatcattaaatcctaaataag

SEQ ID NO: 1741 - DNA, 15 bp, Artificial Sequence
  Description of Artificial Sequence Synthetic probe
accccaggcgaatat

SEQ ID NO: 1742 - DNA, 18 bp, Artificial Sequence
  Description of Artificial Sequence Synthetic probe
ccgtcgctagtagtcact

SEQ ID NO: 1743 - DNA, 20 bp, Artificial Sequence
  Description of Artificial Sequence Synthetic probe
aaacgcagaaaatgatttac

SEQ ID NO: 1744 - DNA, 16 bp, Artificial Sequence
  Description of Artificial Sequence Synthetic probe
ccggatctggaatatt

SEQ ID NO: 1745 - DNA, 16 bp, Artificial Sequence
  Description of Artificial Sequence Synthetic probe
cagctcccataatgcg

SEQ ID NO: 1746 - DNA, 20 bp, Artificial Sequence
  Description of Artificial Sequence Synthetic probe
ttcctggtttagaaagatct

SEQ ID NO: 1747 - DNA, 20 bp, Artificial Sequence
  Description of Artificial Sequence Synthetic probe
caactgttacaagtgatttc

SEQ ID NO: 1748 - DNA, 16 bp, Artificial Sequence
  Description of Artificial Sequence Synthetic probe
atcctacagaaatccc

SEQ ID NO: 1749 - DNA, 18 bp, Artificial Sequence
  Description of Artificial Sequence Synthetic probe
tcgggtctggtattggtg

SEQ ID NO: 1750 - DNA, 20 bp, Artificial Sequence
  Description of Artificial Sequence Synthetic probe
aagcagtgtattcaatttag

SEQ ID NO: 1751 - DNA, 18 bp, Artificial Sequence
  Description of Artificial Sequence Synthetic probe
cacctgttcaagtaaacg

SEQ ID NO: 1752 - DNA, 20 bp, Artificial Sequence
  Description of Artificial Sequence Synthetic probe
atgcagaaaagcaaacatta

SEQ ID NO: 1753 - DNA, 17 bp, Artificial Sequence
  Description of Artificial Sequence Synthetic probe
accagtgacagaagcag

SEQ ID NO: 1754 - DNA, 24 bp, Artificial Sequence
  Description of Artificial Sequence Synthetic probe
accattataactaaggtcaatact

SEQ ID NO: 1755 - DNA, 20 bp, Artificial Sequence
  Description of Artificial Sequence Synthetic probe
catgcactccatcgtattga

SEQ ID NO: 1756 - DNA, 16 bp, Artificial Sequence
  Description of Artificial Sequence Synthetic probe
aacccacttaaattcg

SEQ ID NO: 1757 - DNA, 21 bp, Artificial Sequence
  Description of Artificial Sequence Synthetic probe
ttacaatctgagaataattcc

SEQ ID NO: 1758 - DNA, 21 bp, Artificial Sequence
  Description of Artificial Sequence Synthetic probe
ctaaggtcgatgctagtaact

SEQ ID NO: 1759 - DNA, 22 bp, Artificial Sequence
  Description of Artificial Sequence Synthetic probe
tccatctttaactgtaaatgag

SEQ ID NO: 1760 - DNA, 19 bp, Artificial Sequence
  Description of Artificial Sequence Synthetic probe
aagctaaagcagaaacaag

SEQ ID NO: 1761 - DNA, 25 bp, Artificial Sequence
  Description of Artificial Sequence Synthetic probe
ttgagttctcaaacttataatattc

SEQ ID NO: 1762 - DNA, 21 bp, Artificial Sequence
  Description of Artificial Sequence Synthetic probe
atggtggctcttacactatat

SEQ ID NO: 1763 - DNA, 16 bp, Artificial Sequence
  Description of Artificial Sequence Synthetic probe
cattggtgcagctttt

SEQ ID NO: 1764 - DNA, 16 bp, Artificial Sequence
  Description of Artificial Sequence Synthetic probe
agagcagttgcaagcg

SEQ ID NO: 1765 - DNA, 24 bp, Artificial Sequence
  Description of Artificial Sequence Synthetic primer
ggtagaagagttgaaagcactgtc

SEQ ID NO: 1766 - DNA, 22 bp, Artificial Sequence
  Description of Artificial Sequence Synthetic primer
atggtacctacttgggcaatcg

SEQ ID NO: 1767 - DNA, 18 bp, Artificial Sequence
  Description of Artificial Sequence Synthetic probe
ccttgtgccgatactaaa

SEQ ID NO: 1768 - DNA, 18 bp, Artificial Sequence
  Description of Artificial Sequence Synthetic probe
ccttgtgctgatactaaa

SEQ ID NO: 1769 - DNA, 18 bp, Artificial Sequence
  Description of Artificial Sequence Synthetic probe
acctgtgcagatactaaa